(19) United States
(12) Patent Application Publication
(10) Pub. No.: US 2011/0190156 A1
(43) Pub. Date: Aug. 4, 2011

(54) MOLECULAR SIGNATURES FOR DIAGNOSING SCLERODERMA

(75) Inventors: Michael L. Whitfield, Etna, NH (US); Jennifer L. Sargent, Hanover, NH (US); Sarah A. Pendergrass, Norwich, VT (US); Ausra Milano, Ithaca, NY (US)

(73) Assignee: Trustees of Dartmouth College, Hanover, NH (US)

(21) Appl. No.: 13/054,244

(22) PCT Filed: Jul. 15, 2009
(86) PCT No.: PCT/US09/04089

§ 371 Date: Mar. 30, 2011

Related U.S. Application Data
(60) Provisional application No. 61/080,709, filed on Jul. 15, 2008.
(60) Provisional application No. 61/146,452, filed on Jan. 22, 2009.

(51) Int. Cl. C40B 30/04; C12Q 1/68

(52) U.S. Cl. 506/9; 435/6.1; 435/6.18; 435/6.17; 435/6.16

(57) ABSTRACT

The present invention features methods for classifying, determining severity, and predicting clinical endpoints of scleroderma based upon the expression of selected biomarker genes.

### BACKGROUND OF THE INVENTION

Scleroderma is a systemic autoimmune disease with a heterogeneous and complex phenotype that encompasses several distinct subtypes. The disease has an estimated prevalence of 276 cases per million adults in the United States (Mayes M D (1998) Semin. Cutan. Med. Surg. 17:22-26; Mayes, et al. (2003) Arthritis Rheum. 48:2246-2255). Median age of onset is 45 years of age with the ratio of females to males being approximately 4:1.

Scleroderma is divided into distinct clinical subsets. One subset is the localized form, which affects skin only including morphea, linear scleroderma and eosinophilic fasciitis. The other major type is systemic sclerosis (SSc) and its subsets. The most widely recognized classification system for SSc divides patients into two subtypes, diffuse and limited, a distinction made primarily by the degree of skin involvement (Leroy, et al. (1988) J. Rheumatol. 15:202-205). Patients with SSc with diffuse scleroderma (dSSc) have severe skin involvement (Medsger (2001) In: Koopman, editor. Arthritis and Allied Conditions. 14th ed. Philadelphia: Lippincott Williams & Wilkins. pp. 1590) often characterized by more rapid onset and progressive course with fibrotic skin involvement extending from the hands and arms, trunk, face and lower extremities. Patients with SSc with limited scleroderma (lSSc) have fibrotic skin involvement that is typically limited to the fingers (sclerodactyly), hands and face. Some patients in the limited subset develop significant pulmonary arterial hypertension, pulmonary fibrosis or digital ischemia/ulcerations. Although there are certain disease characteristics that differentiate these two groups, some of the severe vascular and organ manifestations occur across groups and are the cause of significant morbidity and mortality (Masi (1988) J. Rheumatol. 15:894-898).

Skin thickening is one of the earliest manifestations of the disease; it remains the most sensitive and specific finding (Committee. SfSCotARADaTC (1980) Preliminary criteria for the classification of systemic sclerosis (scleroderma). 23:581-590) and is one of the most widely used outcome measures in clinical trials (Seibold & McCloskey (1997) Curr. Opin. Rheumatol. 9:571-575; Clements, et al. (2000) Arthritis Rheum. 43:2445-2454; Clements, et al. (1990) Arthritis Rheum. 33:1256-1263). Several studies have demonstrated that the extent of skin involvement directly correlates with internal organ involvement and prognosis in SSc patients (Barnett, et al. (1988) J. Rheumatol. 15:276-283; Scussel-Lonzetti, et al. (2002) Medicine 81: 154-167; Shand, et al. (2007) Arthritis Rheum. 56:2422-2431). Furthermore, improvement in Modified Rodnan Skin Score (MRSS) is associated with improved survival (Steen & Medsger (2001) Arthritis Rheum. 44:2828-2835). Fibrosis is defined by excessive deposition and contraction of extracellular matrix (ECM) components coupled with down regulation of enzymes essential for ECM remodeling and degradation. These processes are often preceded by chronic inflammation and are mediated by activated fibroblasts (Wynn (2008) J. Pathol. 214(2):199-210). Fibroblasts can be activated by a variety of cytokines, most notably transforming growth factor-beta (TGFβ). Activated fibroblasts secrete numerous collagens including I, III and V in addition to other matrix proteins such as glycoasminoglycans (Wynn (2008) supra). TGFβ has been implicated in SSc pathogenesis (Verrecchia, et al. (2006) Autoimmun. Rev. 5(8):563-9; Leask (2006) Res. Ther. 8(4):213; Varga (2004) Curr. Rheumatol. Rep. 6(2):164-70; Smith & LeRoy (1990) J. Invest. Dermatol. 95(6 Suppl):1255-127S; Leask & Abraham (2004) FASEB J. 18(7):816-27; Cotton, et al. (1998) J. Pathol. 184(1):4-6; Leroy, et al. (1989) Arthritis Rheum. 32(7):817-25). Elevated levels of TGFβ have been observed in SSc skin biopsies (Sfikakis, et al. (1993) Clin. Immunol. Immunopathol. 69(2):199-204; Gabrielli, et al. (1993) Clin. Immunol. Immunopathol. 68(3):340-9). Additionally, high levels of collagen I and collagen III mRNA have been detected in SSc skin (Scharffetter, et al. (1988) Eur. J. Clin. Invest. 18(1):9-17) suggesting that the TGFβ found in SSc skin is biologically active. One clinical trial has been reported utilizing anti-TGFβ therapy in dSSc patients; however, the results of this study were inconclusive (Denton, et al. (2007) Arthritis Rheum. 56(1):323-33).

Conventionally, explanted fibroblasts isolated from SSc patient skin have provided much insight into the phenotypic differences and cellular processes such as fibrosis that have gone awry in skin through the course of the disease. An accumulating body of evidence has been put forward to suggest that SSc fibroblasts show constitutive activation of the canonical TGFβ signaling pathway as evidenced by increased production of ECM components such as collagens, fibrillin, CTGF and COMP (Zhou, et al. (2001) J. Immunol. 167(12):7126-33; Leask (2004) Keio J. Med. 53(2):74-7; Gay, et al. (1980) Arthritis Rheum. 23(2):190-6; Farina, et al. (2006) Matrix Biol. 25(4):213-22).

DNA microarrays have been used to characterize the changes in gene expression that occur in dSSc skin when compared to normal controls (Whitfield, et al. (2003) Proc. Natl. Acad. Sci. USA 100:12319-12324; Gardner, et al. (2006) Arthritis Rheum. 54:1961-1973). However, extensive diversity in the gene expression patterns of SSc were not identified.

### SUMMARY OF THE INVENTION

The present invention provides objective methods useful for the prediction, diagnosis, assessment, classification, study, prognosis, and treatment of scleroderma and complications associated with scleroderma, in subjects having or suspected of having scleroderma. The invention is based, at least in part, on the identification and classification of a relatively small number of genes that are associated with scleroderma and complications associated with scleroderma.

An aspect of the invention is a method for determining scleroderma disease severity in a subject having or suspected of having scleroderma. The method includes the steps of measuring expression of one or more of the genes in Table 6 in a test genetic sample obtained from a subject having or suspected of having scleroderma; and comparing the expression of the one or more genes in the test genetic sample to expression of the one or more genes in a control sample, wherein altered expression of the one or more genes in the test genetic sample compared to the expression in the control sample is indicative of scleroderma disease severity in the subject.

An aspect of the invention is a method for classifying scleroderma in a subject having or suspected of having scleroderma into one of four distinct subtypes described herein, namely, Diffuse-Proliferation, Inflammatory, Limited, or Normal-Like. The method includes the steps of measuring expression of one or more of the intrinsic genes in Table 5 in a test genetic sample obtained from a subject having or suspected of having scleroderma; and comparing the expression of the one or more intrinsic genes in the test genetic sample to expression of the one or more intrinsic genes in a control sample, wherein altered expression of the one or more intrinsic genes in the test genetic sample compared to the expression in the control sample classifies the scleroderma as Diffuse-Proliferation, Inflammatory, Limited, or Normal-Like subtype.

In one embodiment, increased expression of one or more genes selected from ANP32A, APOH, ATAD2, B3GALT6, B3GAT3, C12orf14, C14orf131, CACNG6, CBLL1, CBX8, CDC7, CDT1, CENPE, CGI-90, CLDN6, CREB3L3, CROC4, DDX3Y, DERP6, DJ971N18.2, EHD2, ESPL1, FGF5, FLJ10902, FLJ12438, FLJ12443, FLJ12484, FLJ12572, FLJ20245, FLJ32009, FLJ35757, FXYD2, GABRA2, GATA2, GK, GSG2, HPS3, IKBKG, IL23A, INSIG1, KIAA1509, KIAA1609, KIAA1666, LDLR, LGALS8, LILRB5, LOC123876, LOC128977, LOC153561, LOC283464, LRRIQ2, LY6K, MAC30, ME2, MGC13186, MGC16044, MGC16075, MGC29784, MGC33839, MGC35212, MGC4293, MICB, MLL5, MTRF1L, MUC20, NICN1, NPTX1, OAS3, OGDHL, OPRK1, PCNT2, PDZK1, PITPNC1, PPFIA4, PREB, PRKY, PSMD11, PSPH, PSPHL, PTP4A3, PXMP2, RAB15, RAD51AP1, RIP, RNF121, RPL41, RPS18, RPS4Y1, RPS4Y2, S100P, SORD, SP1, SYMPK, SYT6, TM9SF4, TMOD3, TNFRSF12A, TPRA40, TRIP, TRPM7, TTR, TUBB4, VARS2L, ZNF572, and ZSCAN2 in the test genetic sample compared to the expression in the control sample classifies the scleroderma as the Diffuse-Proliferation subtype.

In one embodiment, decreased expression of one or more genes selected from AADAC, ADAM17, ADH1A, ADH1C, AHNAK, ALG1, ALG5, AMOT, AOX1, AP2A2, ARK5, ARL6IP5, ARMCX1, BECN1, BECN1, BMP8A, BNIP3L, C10orf119, C1orf24, C1orf37, C20orf10, C20orf22, C5orf14, C6orf64, C9orf61, CAPS, CASP4, CASP5, CAST, CAV2, CCDC6, CCNG2, CDC26, CDK2AP1, CDR1, CFHL1, CNTN3, CPNE5, CRTAP, CTNNA1, CTSC, CUTL1, CXCL5, CYBRD1, CYP2R1, DBN1, DCAMKL1, DCL-1, DIAPH2, DKK2, ECHDC3, ECM2, EIF3S7, EMB, EMCN, EMILIN2, ENPP2, EPB41L2, FBLN1, FBLN2, FEM1A, FGL2, FHL5, FKBP7, FLI1, FLJ10986, FLJ20032, FLJ20701, FLJ23861, FLJ34969, FLJ36748, FLJ36888, FLJ43339, FZR1, GABPB2, GARNL4, GHITM, GHR, GIT2, GLYAT, GPM6B, GTPBP5, HELB, HOXB4, IFNA6, IGFBP5, IL13RA1, IL15, KAZALD1, KCNK4, KCNS3, KCTD10, KIAA0232, KIAA0494, KIAA0562, KIAA0870, KIAA1190, KIF25, KLHL18, KLK2, LAMP2, LEPROTL1, LHFP, LMO2, LOC114990, LOC255458, LOC387680, LOC400027, LOC493869, LOC87769, LRBA, MAFB, MAGEH1, MAN2B2, MCCC2, MEGF10, MFAP5, MGC11308, MGC15523, MGC3200, MGC35048, MGC45780, MOGAT3, MPPE1, MPZ, MYO1B, MYOC, NFYC, NIPSNAP3B, OPTN, OSR2, PAM, PBXIP1, PCOLCE2, PDGFC, PDGFRA, PDGFRL, PEX19, PHAX, PIP, PKM2, PKP2, PMP22, POU2F1, PPAP2B, PRAC, PSMA5, PSORS1C1, PTGIS, RECK, RGS11, RGS5, RIMS3, RIPK2, RNASE4, RNF125, RNF13, RNF146, RNF19, ROBO1, ROBO3, RPL7A, SARA1, SAV1, SCGB1D1, SDK1, SECP43, SECTM1, SERPINB2, SGCA, SH3BGRL, SH3GLB1, SH3RF2, SLC10A3, SLC12A2, SLC14A1, SLC39A14, SLC7A7, SLC9A9, SLPI, SMAD1, SMAP1, SMARCE1, SMP1, SNTG2, SNX7, SOCS5, SSPN, STX7, SUMF1, TAS2R10, TDE2, TFAP2B, TGFBR2, THSD2, TM4SF3, TMEM25, TMEM34, TNA, TNKS2, TRAD, TRAF3IP1, TREM4, TRIM35, TRIM9, TTYH2, TUBB1, UBL3, ULK2, URB, USP54, UST, UTRN, UTX, WIF1, WWOX, XG, YPEL5, and ZFHX1B in the test genetic sample compared to the expression in the control sample classifies the scleroderma as the Diffuse-Proliferation subtype.

In one embodiment, increased expression of one or more genes selected from ANP32A, APOH, ATAD2, B3GALT6, B3GAT3, C12orf14, C14orf131, CACNG6, CBLL1, CBX8, CDC7, CDT1, CENPE, CGI-90, CLDN6, CREB3L3, CROC4, DDX3Y, DERP6, DJ971N18.2, EHD2, ESPL1, FGF5, FLJ10902, FLJ12438, FLJ12443, FLJ12484, FLJ12572, FLJ20245, FLJ32009, FLJ35757, FXYD2, GABRA2, GATA2, GK, GSG2, HPS3, IKBKG, IL23A, INSIG1, KIAA1509, KIAA1609, KIAA1666, LDLR, LGALS8, LILRB5, LOC123876, LOC128977, LOC153561, LOC283464, LRRIQ2, LY6K, MAC30, ME2, MGC13186, MGC16044, MGC16075, MGC29784, MGC33839, MGC35212, MGC4293, MICB, MLL5, MTRF1L, MUC20, NICN1, NPTX1, OAS3, OGDHL, OPRK1, PCNT2, PDZK1, PITPNC1, PPFIA4, PREB, PRKY, PSMD11, PSPH, PSPHL, PTP4A3, PXMP2, RAB15, RAD51AP1, RIP, RNF121, RPL41, RPS18, RPS4Y1, RPS4Y2, S100P, SORD, SP1, SYMPK, SYT6, TM9SF4, TMOD3, TNFRSF12A, TPRA40, TRIP, TRPM7, TTR, TUBB4, VARS2L, ZNF572, and ZSCAN2 in the test genetic sample compared to the expression in the control sample, together with decreased expression of one or more genes selected from AADAC, ADAM17, ADH1A, ADH1C, AHNAK, ALG1, ALG5, AMOT, AOX1, AP2A2, ARK5, ARL6IP5, ARMCX1, BECN1, BECN1, BMP8A, BNIP3L, C10orf119, C1orf24, C1orf37, C20orf10, C20orf22, C5orf14, C6orf64, C9orf61, CAPS, CASP4, CASP5, CAST, CAV2, CCDC6, CCNG2, CDC26, CDK2AP1, CDR1, CFHL1, CNTN3, CPNE5, CRTAP, CTNNA1, CTSC, CUTL1, CXCL5, CYBRD1, CYP2R1, DBN1, DCAMKL1, DCL-1, DIAPH2, DKK2, ECHDC3, ECM2, EIF3S7, EMB, EMCN, EMILIN2, ENPP2, EPB41L2, FBLN1, FBLN2, FEM1A, FGL2, FHL5, FKBP7, FLI1, FLJ10986, FLJ20032, FLJ20701, FLJ23861, FLJ34969, FLJ36748, FLJ36888, FLJ43339, FZR1, GABPB2, GARNL4, GHITM, GHR, GIT2, GLYAT, GPM6B, GTPBP5, HELB, HOXB4, IFNA6, IGFBP5, IL13RA1, IL15, KAZALD1, KCNK4, KCNS3, KCTD10, KIAA0232, KIAA0494, KIAA0562, KIAA0870, KIAA1190, KIF25, KLHL18, KLK2, LAMP2, LEPROTL1, LHFP, LMO2, LOC114990, LOC255458, LOC387680, LOC400027, LOC493869, LOC87769, LRBA, MAFB, MAGEH1, MAN2B2, MCCC2, MEGF10, MFAP5, MGC11308, MGC15523, MGC3200, MGC35048, MGC45780, MOGAT3, MPPE1, MPZ, MYO1B, MYOC, NFYC, NIPSNAP3B, OPTN, OSR2, PAM, PBXIP1, PCOLCE2, PDGFC, PDGFRA, PDGFRL, PEX19, PHAX, PIP, PKM2, PKP2, PMP22, POU2F1, PPAP2B, PRAC, PSMA5, PSORS1C1, PTGIS, RECK, RGS11, RGS5, RIMS3, RIPK2, RNASE4, RNF125, RNF13, RNF146, RNF19, ROBOT, ROBO3, RPL7A, SARA1, SAV1, SCGB1D1, SDK1, SECP43, SECTM1, SERPINB2, SGCA, SH3BGRL, SH3GLB1, SH3RF2, SLC10A3, SLC12A2, SLC14A1, SLC39A14, SLC7A7, SLC9A9, SLPI, SMAD1, SMAP1, SMARCE1, SMP1, SNTG2, SNX7, SOCS5, SSPN, STX7, SUMF1, TAS2R10, TDE2, TFAP2B, TGFBR2, THSD2, TM4SF3, TMEM25, TMEM34, TNA, TNKS2, TRAD, TRAF3IP1, TREM4, TRIM35, TRIM9, TTYH2, TUBB1, UBL3, ULK2, URB, USP54, UST, UTRN, UTX, WIF1, WWOX, XG, YPEL5, and ZFHX1B in the test genetic sample compared to the expression in the control sample, classifies the scleroderma as the Diffuse-Proliferation subtype.

In one embodiment, increased expression of one or more genes selected from A2M, AIF1, ALOX5AP, APOL2, APOL3, BATF, BCL3, BIRC1, BTN3A2, C10orf10, C1orf38, C6orf80, CCL2, CCL4, CCR5, CD8A, CDW52, COL6A3, COTL1, CPA3, CPVL, CTAG1B, DDX58, EBI2, EVI2B, F13A1, FAM20A, FAP, FCGR3A, FLJ11259, FLJ22573, FLJ23221, FLJ25200, FYB, GBP1, GBP3, GEM, GIMAP6, GMFG, GZMH, GZMK, HAVCR2, HCLS1, HLA-DMA, HLA-DOA, HLA-DPA1, HLA-DPB1, HLA-DQA1, HLA-DQA2, HLA-DQB1, HLA-DRB1, HLA-DRB5, ICAM2, IFI16, IFIT1, IFIT2, IFITM1, IFITM2, IFITM3, IL10RA, INDO, ITGB2, KIAA0063, LAMB1, LCP1, LGALS2, LGALS9, LILRB2, LOC387763, LOC400759, LUM, LYZ, MARCKS, MFNG, MGC24133, MPEG1, MRC1, MRCL3, MS4A6A, MX1, NNMT, NUP62, PAG, PLAU, PPIC, PTPRC, RAC2, RGS10, RGS16, RSAFD1, SAT, SCGB2A1, SLC20A1, SLCO2B1, SPARC, SULF1, TAP1, TCTEL1, TIMP1, TNFSF4, UBD, VSIG4, and ZFYVE26 in the test genetic sample compared to the expression in the control sample classifies the scleroderma as the Inflammatory subtype.

In one embodiment, increased expression of one or more genes selected from ATP6V1B2, C1orf42, C7 orf19, CKLFSF1, CTAGE4, DICER1, DIRC1, DPCD, DPP3, EMR2, EXOSC6, FLJ90661, FN3KRP, GFAP, GPT, IL27, KCTD15, KIAA0664, LMOD1, LOC147645, LOC400581, LOC441245, MAB21L2, MARCH-II, MGC42157, MRPL43, MT, MT1A, NCKAP1, PGM1, POLD4, RAI16, SAMD10, and UHSKerB in the test genetic sample compared to the expression in the control sample classifies the scleroderma as the Limited subtype.

An aspect of the invention is a method for classifying scleroderma in a subject having or suspected of having scleroderma into the Inflammatory subtype of scleroderma. The method includes the steps of measuring expression of one or more of the genes in Table 12 or Table 13 in a test genetic sample obtained from a subject having or suspected of having scleroderma; and comparing the expression of the one or more genes in the test genetic sample to expression of the one or more genes in a control sample, wherein altered expression of the one or more genes in the test genetic sample compared to the expression in the control sample classifies the scleroderma as Inflammatory subtype. Genes listed in Tables 12 and 13 relate to so-called IL-13 and IL-4 gene signatures, respectively.

An aspect of the invention is a method for assessing risk of a subject developing interstitial lung disease (ILD) or a severe fibrotic skin phenotype, wherein the subject is a subject having or suspected of having scleroderma. The method includes the steps of measuring expression of one or more of the genes in Table 8 in a test genetic sample obtained from a subject having or suspected of having scleroderma; and comparing the expression of the one or more genes in the test genetic sample to expression of the one or more genes in a control sample, wherein altered expression of the one or more genes in the test genetic sample compared to the expression in the control sample is indicative of risk of the subject developing interstitial lung disease or a severe fibrotic skin phenotype.

An aspect of the invention is a method for assessing risk of a subject having or developing interstitial lung disease involvement in scleroderma, wherein the subject is a subject having or suspected of having scleroderma. The method includes the steps of measuring expression of REST Corepressor 3 gene (RCO3) and Alstrom Syndrome 1 gene (ALMS1) in a test genetic sample obtained from a subject having or suspected of having scleroderma; and comparing the expression of RCO3 and ALMS1 in the test genetic sample to expression of RCO3 and ALMS1 in a control sample, wherein altered expression of RCO3 and ALMS1 in the test genetic sample compared to the expression in the control sample is indicative of risk of the subject having or developing interstitial lung disease involvement in scleroderma.

An aspect of the invention is a method for predicting digital ulcer involvement in a subject having or suspected of having scleroderma. The method includes the steps of measuring expression of SERPINB7, FBXO25 and MGC3207 in a test genetic sample obtained from a subject having or suspected of having scleroderma; and comparing the expression of SERPINB7, FBXO25 and MGC3207 genes in the test genetic sample to expression of SERPINB7, FBXO25 and MGC3207 genes in a control sample, wherein altered expression of SERPINB7, FBXO25 and MGC3207 genes in the test genetic sample compared to the expression of SERPINB7, FBXO25 and MGC3207 genes in the control sample is predictive of digital ulcer involvement in the subject having or suspected of having scleroderma.

In accordance with each and every one of the aspects and embodiments of the invention, in one embodiment the measuring includes hybridizing the test genetic sample to a nucleic acid microarray that is capable of hybridizing at least one of the genes, and detecting hybridization of at least one of the genes when present in the test genetic sample to the nucleic acid microarray with a scanner suitable for reading the microarray. In one embodiment the measuring is hybridizing the test genetic sample to a nucleic acid microarray that is capable of hybridizing at least one of the genes, and detecting hybridization of at least one of the genes when present in the test genetic sample to the nucleic acid microarray with a scanner suitable for reading the microarray.

In accordance with each and every one of the aspects and embodiments of the invention, in one embodiment the control sample includes a composite of data derived from a plurality of nucleic acid microarray hybridizations representative of at least one subtype of scleroderma selected from the group consisting of Diffuse-Proliferation, Inflammatory, Limited, and Normal-Like. In one embodiment the control sample is a composite of data derived from a plurality of nucleic acid microarray hybridizations representative of at least one subtype of scleroderma selected from the group consisting of Diffuse-Proliferation, Inflammatory, Limited, and Normal-Like.

In accordance with each and every one of the aspects and embodiments of the invention, in one embodiment the control sample includes a composite of data derived from a plurality of nucleic acid microarray hybridizations representative of each subtype of scleroderma selected from the group consisting of Diffuse-Proliferation, Inflammatory, Limited, and Normal-Like. In one embodiment the control sample is a composite of data derived from a plurality of nucleic acid microarray hybridizations representative of each subtype of scleroderma selected from the group consisting of Diffuse-Proliferation, Inflammatory, Limited, and Normal-Like.

In accordance with each and every one of the aspects and embodiments of the invention, in one embodiment the subject having or suspected of having scleroderma is a subject having scleroderma.

In accordance with each and every one of the aspects and embodiments of the invention, in one embodiment the subject having or suspected of having scleroderma is a subject suspected of having scleroderma.

In accordance with each and every one of the aspects and embodiments of the invention, in one embodiment the subject suspected of having scleroderma is a subject having Raynaud's phenomenon.

### BRIEF DESCRIPTION OF THE DRAWINGS

FIG. 1 is an unsupervised hierarchical clustering dendrogram showing the relationship among the samples using 4,149 probes. Sample names are based upon their clinical diagnosis: dSSc, diffuse scleroderma; lSSc, limited scleroderma; morphea; EF, eosinophilic fasciitis; and Nor, healthy controls. Forearm (FA) and Back (B) are indicated for each sample. Solid arrows indicate the 14 of 22 forearm-back pairs that cluster next to one another; dashed arrows indicate the additional three forearm-back pairs that cluster with only a single sample between them. Technical replicates are indicated by the labels (a), (b) or (c). Nine out of 14 technical replicates cluster immediately beside one another.

FIG. 2 is an experimental sample hierarchical clustering dendrogram. The dendrogram was generated by cluster analysis using the scleroderma intrinsic gene set. The ca. 1000 most “intrinsic” genes were selected from 75 microarray hybridizations analyzing 34 individuals. Two major branches of the dendrogram tree are evident which divide a subset of the dSSc samples from all other samples. Within these major groups are smaller branches with identifiable biological themes, which have been grouped according to the following: diffuse 1, #; diffuse 2, †; inflammatory, ≈; limited, ̂ and normal-like, ′. Statistically significant clusters (p<0.001) identified by SigClust are indicated by an asterisk (*) at the lowest significant branch. Bars indicate forearm-back pairs which cluster together based on this analysis.

FIG. 3 shows quantitative real time polymerase chain reaction (qRT-PCR) analysis of representative biopsies. The mRNA levels of three genes, TNFRSF12A (FIG. 3A), CD8A (FIG. 3B) and WIF1 (FIG. 3C) were analyzed by TAQMAN quantitative real time PCR. Each was analyzed in two representative forearm skin biopsies from each of the major subsets of proliferation, inflammatory, limited and normal controls. In the case of TNFRSF12A, patient dSSc11 was replaced by patient dSSc10, which cluster next to one another in the intrinsic subsets and showed similar clinical characteristics (Table 1). Each qRT-PCR assay was performed in triplicate for each sample. The level of each gene was then normalized against triplicate measurements of glyceraldehyde 3-phosphate dehydrogenase (GAPDH) to control for total mRNA levels (see materials and methods). The relative expression values are displayed as the fold change for each gene relative to the median value of the eight samples analyzed.

FIG. 4 shows that the TGFβ responsive signature is activated in a subset of dSSc patients. The array dendogram shows clustering of 53 dSSc (filled bars) and healthy control (open bars) samples using the 894 probe TGFβ-responsive signature. Two major clusters are present, TGFβ-activated (#) and TGFβ not-activated. Technical replicates are designated by a number following patient and biopsy site identification. Statistically significant clusters as determined by SigClust are marked with * (p<0.001).

FIG. 5 shows linear discriminant analysis (LDA) of “intrinsic” SSc skin subsets found in skin. A single-gene analysis is shown in panels A and B. A multigene analysis is shown in panels C and D. Shown are the plots of LDA score calculated from the gene expression data for 61 patients using the single best genes (Panels A and B) to distinguish the Proliferation group of diffuse SSc from all other groups (CRTAP; Panel A), and the single best gene that differentiates Inflammatory group from all other subgroups (MS4A6A; Panel B). Note the overlapping distributions of the LDA scores in Panels A and B. A multigene analysis shows better separation of the two groups (Panels C and D). The LDA model that incorporates the expression of multiple genes demonstrates that patients in the intrinsic Diffuse-Proliferation group can be separated from all other patients (Panel C) and the Inflammatory group can also be separated (Panel D).

FIG. 6 shows three different models that predict clinical endpoints in using gene expression in SSc skin. A multistep stochastic search process was used to identify combinations of genes that predict clinical endpoints in SSc. Shown are the directed acyclic graphical models of two different solutions generated by SDA. Each node is either a function or a gene. Interstitial lung involvement can be represented by the multiplication of two different genes, while the presence of digital ulcers can be predicted by the multiplicative combination of three different genes.

FIG. 7 is a series of box plot graphs depicting the use of LDA for distinguishing the Diffuse-Proliferation group from all other groups. Panels A-D represent single-gene comparisons for (A) Rabaptin, RAB GTPase binding effector protein 1 (RABEP1), NM_{—}004703; (B) Promethin, NM_{—}020422; (C) Novel gene transcript, ENST00000312412; and (D) Amyotrophic lateral sclerosis 2 (juvenile) chromosome region, candidate 13 (ALS2CR13), NM_{—}173511. Panel E represents LDA Score comparison using the equation LDA Score=−1.902(NM_{—}004703)−1.908(NM_{—}020422)+1.475(ENST00000312412)+1.83(NM_{—}173511).

FIG. 8 is a series of box plot graphs depicting the use of LDA for distinguishing the Inflammatory group from all other groups. Panels A-E represent single-gene comparisons for (A) Major histocompatibility complex, class II, DO alpha (HLA-DOA), NM_{—}002119; (B) GLI pathogenesis-related 1 (glioma) (GLIPR1), NM_{—}006851; (C) 5-oxoprolinase (ATP-hydrolysing) (OPLAH), NM_{—}017570; (D) Mitochondrial ribosomal protein L46 (MRPL46), NM_{—}022163; and (E) Cysteine-rich hydrophobic domain 2 (CHIC2), NM_{—}012110. Panel F represents LDA Score comparison using the equation LDA score=4.365(NM_{—}002119)+2.926(NM_{—}006851)−2.620(NM_{—}017570)+6.601(NM_{—}022163)+2.033(NM_{—}012110).

### DETAILED DESCRIPTION OF THE INVENTION

Using DNA microarrays, a clear relationship between scleroderma disease and gene expression has been identified. The results herein show that the diversity in the gene expression patterns of SSc is much greater than demonstrated in two prior studies of dSSc skin (Whitfield, et al. (2003) supra; Gardner, et al. (2006) supra). The advantage of these biomarkers over prior signatures is the small number of genes and a mathematical model, which emphasizes the differences among patients. This makes these sets of biomarkers more tractable for use in a clinical setting.

In particular, the present invention features a 177-gene signature for scleroderma that is associated the more severe modified Rodnan skin score (MRSS) in systemic sclerosis. MRSS is one of the primary outcome measures in clinical trials evaluating drug efficacy in scleroderma, but is not an objective outcome measure since it can vary from physician-to-physician. Accordingly, all or a portion of the instant 177-gene signature finds application as a diagnostic test for determining scleroderma disease severity. Similar diagnostic tests, e.g., the MammaPrint array in breast cancer, have been validated as reliable diagnostic tools to predict outcome of disease (Glas, et al. (2006) BMC Genomics 7:278).

In addition, the present invention features the classification of scleroderma into multiple distinct subtypes, which can be identified by different gene expression profiles of a set of intrinsic genes. As used herein, an “intrinsic gene” is a gene that shows little variance within repeated samplings of tissue from an individual subject having scleroderma, but which shows high variance across the same tissue in multiple subjects, wherein the multiple subjects include both subjects having scleroderma and subjects not having scleroderma. For example, an intrinsic gene can be a gene that shows little variance within repeated samplings of forearm-back skin pairs in a subject having scleroderma, but which shows high variance across forearm-back skin pairs of other subjects, wherein the other subjects include both subjects having scleroderma and subjects not having scleroderma.

Disclosed herein are genes that can be used as intrinsic genes with the methods disclosed herein. The intrinsic genes disclosed herein can be genes that have less than or equal to 0.00001, 0.0001, 0.001, 0.01, 0.1, 0.2. 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 1,000, 10,000, or 100,000% variation between two samples from the same tissue. It is also understood that these levels of variation can also be applied across 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more tissues, and the level of variation compared. It is also understood that variation can be determined as discussed in the examples using the methods and algorithms as disclosed herein.

An intrinsic gene set is defined herein as a group of genes including one or more intrinsic genes. A minimal intrinsic gene set is defined herein as being derived from an intrinsic gene set, and is comprised of the smallest number of intrinsic genes that can be used to classify a sample.

For the purposes of the present invention, intrinsic gene sets are used to classify scleroderma into a Diffuse-Proliferation group or subtype thereof, Inflammatory group, Limited group or Normal-Like group. The Diffuse-Proliferation group is composed solely of patients with a diagnosis of dSSc. The Inflammatory group includes patients with dSSc, lSSc and morphea. The Limited group is composed solely of patients with lSSc. The Normal-Like group includes healthy controls along with dSSc and lSSc patients. These intrinsic groups or subsets create a more refined division of the disease than current clinical diagnoses and allows for the assessment of patients in different subsets and their likelihood of responding to therapy. For example, it has been shown that patients in the Diffuse-Proliferation group are likely to respond to the drug imatinib mesylate, marketed under the trade name of GLEEVEC® (Novartis Pharmaceuticals, East Hanover, N.J.). Furthermore, selected genes from this gene expression signature provide a basis for identifying patients having, or at risk of having, ILD or digital ulcer involvement.

Based on analysis of the ca. 1000 identified intrinsic genes as disclosed herein, it is possible to categorize non-overlapping sets of genes from within these ca. 1000 intrinsic genes that differentiate the Diffuse-Proliferation group, the Inflammatory group, the Limited group, and the Normal-Like group.

Genes that differentiate the Diffuse-Proliferation group. There are two major sets of genes that differentiate the Diffuse-Proliferation group. One set (Group I) shows higher expression in the Diffuse-Proliferation group and the other set (Group II) shows lower expression in the Diffuse-Proliferation group. The Diffuse-Proliferation group is also defined in part by the general absence of an Inflammatory signature, although there can be some overlap between the Inflammatory and Diffuse-Proliferation signatures.

Group I genes include 138 genes, the increased expression of which is indicative of the Diffuse-Proliferation group. Expression of these genes is decreased in the Inflammatory, Limited, and Normal-Like groups. Referring to Table 5 below, included in the genes of Group I are the following genes, each identified by name: ANP32A, APOH, ATAD2, B3GALT6, B3GAT3, C12orf14, C14orf131, CACNG6, CBLL1, CBX8, CDC7, CDT1, CENPE, CGI-90, CLDN6, CREB3L3, CROC4, DDX3Y, DERP6, DJ971N18.2, EHD2, ESPL1, FGF5, FLJ10902, FLJ12438, FLJ12443, FLJ12484, FLJ12572, FLJ20245, FLJ32009, FLJ35757, FXYD2, GABRA2, GATA2, GK, GSG2, HPS3, IKBKG, IL23A, INSIG1, KIAA1509, KIAA1609, KIAA1666, LDLR, LGALS8, LILRB5, LOC123876, LOC128977, LOC153561, LOC283464, LRRIQ2, LY6K, MAC30, ME2, MGC13186, MGC16044, MGC16075, MGC29784, MGC33839, MGC35212, MGC4293, MICB, MLL5, MTRF1L, MUC20, NICN1, NPTX1, OAS3, OGDHL, OPRK1, PCNT2, PDZK1, PITPNC1, PPFIA4, PREB, PRKY, PSMD11, PSPH, PSPHL, PTP4A3, PXMP2, RAB15, RAD51AP1, RIP, RNF121, RPL41, RPS18, RPS4Y1, RPS4Y2, S100P, SORD, SP1, SYMPK, SYT6, TM9SF4, TMOD3, TNFRSF12A, TPRA40, TRIP, TRPM7, TTR, TUBB4, VARS2L, ZNF572, and ZSCAN2. Also included in the genes of Group I are the following genes, each identified by GenBank accession number only: A_{—}24_BS934268, AB065507, AC007051, AI791206, AK022745, AK022893, AK022997, AK094044, AL391244, AL731541, AL928970, BC010544, BC020847, BM925639, BM928667, ENST00000328708, ENST00000333517, I_{—}1891291, I_{—}3580313, NM_{—}001009569, NM_{—}001024808, NM_{—}172020, NM_{—}173705, NM_{—}178467, NR_{—}001544, THC1434038, THC1484458, THC1504780, U62539, XM_{—}210579, XM_{—}303638, and XM_{—}371684.

Group II genes include 298 genes, the decreased expression of which is also indicative of the Diffuse-Proliferation group. Expression of these genes is increased in the Inflammatory, Limited, and Normal-Like groups. Referring to Table 5 below, included in the genes of Group II are the following genes, each identified by name: AADAC, ADAM17, ADH1A, ADH1C, AHNAK, ALG1, ALG5, AMOT, AOX1, AP2A2, ARK5, ARL6IP5, ARMCX1, BECN1, BECN1, BMP8A, BNIP3L, C10orf119, C1orf24, C1orf37, C20orf10, C20orf22, C5orf14, C6orf64, C9orf61, CAPS, CASP4, CASP5, CAST, CAV2, CCDC6, CCNG2, CDC26, CDK2AP1, CDR1, CFHL1, CNTN3, CPNE5, CRTAP, CTNNA1, CTSC, CUTL1, CXCL5, CYBRD1, CYP2R1, DBN1, DCAMKL1, DCL-1, DIAPH2, DKK2, ECHDC3, ECM2, EIF3S7, EMB, EMCN, EMILIN2, ENPP2, EPB41L2, FBLN1, FBLN2, FEM1A, FGL2, FHL5, FKBP7, FLI1, FLJ10986, FLJ20032, FLJ20701, FLJ23861, FLJ34969, FLJ36748, FLJ36888, FLJ43339, FZR1, GABPB2, GARNL4, GHITM, GHR, GIT2, GLYAT, GPM6B, GTPBP5, HELB, HOXB4, IFNA6, IGFBP5, IL13RA1, IL15, KAZALD1, KCNK4, KCNS3, KCTD10, KIAA0232, KIAA0494, KIAA0562, KIAA0870, KIAA1190, KIF25, KLHL18, KLK2, LAMP2, LEPROTL1, LHFP, LMO2, LOC114990, LOC255458, LOC387680, LOC400027, LOC493869, LOC87769, LRBA, MAFB, MAGEH1, MAN2B2, MCCC2, MEGF10, MFAP5, MGC11308, MGC15523, MGC3200, MGC35048, MGC45780, MOGAT3, MPPE1, MPZ, MYO1B, MYOC, NFYC, NIPSNAP3B, OPTN, OSR2, PAM, PBXIP1, PCOLCE2, PDGFC, PDGFRA, PDGFRL, PEX19, PHAX, PIP, PKM2, PKP2, PMP22, POU2F1, PPAP2B, PRAC, PSMA5, PSORS1C1, PTGIS, RECK, RGS11, RGS5, RIMS3, RIPK2, RNASE4, RNF125, RNF13, RNF146, RNF19, ROBO1, ROBO3, RPL7A, SARA1, SAV1, SCGB1D1, SDK1, SECP43, SECTM1, SERPINB2, SGCA, SH3BGRL, SH3GLB1, SH3RF2, SLC10A3, SLC12A2, SLC14A1, SLC39A14, SLC7A7, SLC9A9, SLPI, SMAD1, SMAP1, SMARCE1, SMP1, SNTG2, SNX7, SOCS5, SSPN, STX7, SUMF1, TAS2R10, TDE2, TFAP2B, TGFBR2, THSD2, TM4SF3, TMEM25, TMEM34, TNA, TNKS2, TRAD, TRAF3IP1, TREM4, TRIM35, TRIM9, TTYH2, TUBB1, UBL3, ULK2, URB, USP54, UST, UTRN, UTX, WIF1, WWOX, XG, YPEL5, and ZFHX1B. Also included in the genes of Group II are the following genes, each identified by GenBank accession number only: A_{—}32_BS169243, A_{—}32_BS200773, A_{—}32_BS53976, AC025463, AF124368, AF161364, AF318337, AF372624, AK001565, AK022793, AK055621, AK056856, AL050042, AL137761, BC035102, BC038761, BC039664, BG252130, BI014689, D80006, ENST00000298643, ENST00000300068, ENST00000305402, ENST00000307901, ENST00000321656, ENST00000322803, ENST00000329246, ENST00000331640, ENST00000332271, ENST00000333784, H16080, I_{—}1861543, I_{—}1882608, I_{—}1985061, I_{—}3335767, I_{—}3551568, I_{—}3588329, I_{—}932413, I_{—}962800, I_{—}966091, NM_{—}001008528, NM_{—}001009555, NM_{—}001013632, NM_{—}001014975, NM_{—}001018006, NM_{—}001018076, NM_{—}001025077, NM_{—}003671, NM_{—}014758, NM_{—}015262, NM_{—}138411, NM_{—}153030, NM_{—}173709, NM_{—}213595, NR_{—}002184, S62210, THC1419743, THC1429821, THC1457118, THC1459712, THC1461073, THC1506312, THC1511927, THC1515028, THC1525318, THC1531579, THC1544941, THC1551463, THC1559236, THC1560798, THC1563147, THC1572906, THC1574967, THC1591470, XM_{—}165930, and XM_{—}209429.

Genes that differentiate the Inflammatory group. The Inflammatory group is identified by increased expression of a group of 119 genes in Group III. These genes show low expression in the Diffuse-Proliferation, Limited, and Normal-Like groups. Referring to Table 5 below, included in the genes of Group III are the following genes, each identified by name: A2M, AIF1, ALOX5AP, APOL2, APOL3, BATF, BCL3, BIRC1, BTN3A2, C10orf10, C1orf38, C6orf80, CCL2, CCL4, CCR5, CD8A, CDW52, COL6A3, COTL1, CPA3, CPVL, CTAG1B, DDX58, EBI2, EVI2B, F13A1, FAM20A, FAP, FCGR3A, FLJ11259, FLJ22573, FLJ23221, FLJ25200, FYB, GBP1, GBP3, GEM, GIMAP6, GMFG, GZMH, GZMK, HAVCR2, HCLS1, HLA-DMA, HLA-DOA, HLA-DPA1, HLA-DPB1, HLA-DQA1, HLA-DQA2, HLA-DQB1, HLA-DRB1, HLA-DRB5, ICAM2, IFI16, IFI16, IFIT1, IFIT2, IFITM1, IFITM2, IFITM3, IL10RA, INDO, ITGB2, KIAA0063, LAMB1, LCP1, LGALS2, LGALS9, LILRB2, LOC387763, LOC400759, LUM, LYZ, MARCKS, MFNG, MGC24133, MPEG1, MRC1, MRCL3, MS4A6A, MX1, NNMT, NUP62, PAG, PLAU, PPIC, PPIC, PTPRC, RAC2, RGS10, RGS16, RSAFD1, SAT, SCGB2A1, SLC20A1, SLCO2B1, SPARC, SULF1, TAP1, TCTEL1, TIMP1, TNFSF4, UBD, VSIG4, and ZFYVE26. Also included in the genes of Group III are the following genes, each identified by GenBank accession number only: AF533936, BQ049338, ENST00000310210, ENST00000313904, ENST00000329660, I_{—}1000437, I_{—}966691, M15073, NM_{—}001010919, NM_{—}001025201, NM_{—}001033569, THC1543691, and XM_{—}291496.

Genes that differentiate the Limited group. The Limited group is distinguished by the increased expression of a set of 47 genes in Group IV. A second defining feature of this subset is reduced expression of the Diffuse-Proliferation-increased genes (Group I), reduced expression of the Inflammatory-increased genes (Group III), and increased expression of the Diffuse-Proliferation-decreased genes (Group II). Referring to Table 5 below, included in the genes of Group IV are the following genes, each identified by name: ATP6V1B2, C1orf42, C7orf19, CKLFSF1, CTAGE4, DICER1, DIRC1, DPCD, DPP3, EMR2, EXOSC6, FLJ90661, FN3KRP, GFAP, GPT, IL27, KCTD15, KIAA0664, LMOD1, LOC147645, LOC400581, LOC441245, MAB21L2, MARCH-II, MGC42157, MRPL43, MT, MT1A, NCKAP1, PGM1, POLD4, RAI16, SAMD10, and UHSKerB. Also included in the genes of Group IV are the following genes, each identified by GenBank accession number only: AC008453, AF086167, AF089746, AJ276555, AL009178, BC031278, BM561346, ENST00000325773, ENST00000331096, THC1562602, X68990, XM_{—}170211, and XM_{—}295760.

Genes that differentiate the Normal-Like group. The Normal-Like group is defined largely by the absence of the other group-specific gene expression signatures. These are the absence of the Diffuse-Proliferation-increased signature (Group I), the absence of the Inflammatory-increased signature (Group III), the absence of the Limited-increased signature (Group IV), and the increased expression of genes in the Diffuse-Proliferation-decreased signature (Group II). Therefore, increased expression of genes in the Diffuse-Proliferation-decreased signature (Group II) could also be considered to be a Normal-Like signature.

The table below summarizes the non-overlapping sets of genes from within the ca. 1000 intrinsic genes that differentiate the Diffuse-Proliferation group, the Inflammatory group, the Limited group, and the Normal-Like group.

TABLE

| | I | II | III | IV |
|---|---|---|---|---|
| Group | (138) | (298) | (119) | (47) |
| Diffuse-Proliferation | ↑ | ↓ | ↓ | |
| Inflammatory | ↓ | ↑ | ↑ | |
| Limited | ↓ | ↑ | ↓ | ↑ |
| Normal-Like | ↓ | ↑ | ↓ | |

In one embodiment the Diffuse-Proliferation group, and likewise a subject that can be categorized as falling within the Diffuse-Proliferation group, can be identified by the increased expression of any one or more genes within Group I.

In one embodiment the Diffuse-Proliferation group, and likewise a subject that can be categorized as falling within the Diffuse-Proliferation group, can be identified by the decreased expression of any one or more genes within Group II.

In one embodiment the Diffuse-Proliferation group, and likewise a subject that can be categorized as falling within the Diffuse-Proliferation group, can be identified by the increased expression of any one or more genes within Group I and the decreased expression of any one or more genes within Group II.

In one embodiment the Diffuse-Proliferation group, and likewise a subject that can be categorized as falling within the Diffuse-Proliferation group, can be identified by the increased expression of any one or more genes within Group I and the decreased expression of any one or more genes within Group III.

In one embodiment the Diffuse-Proliferation group, and likewise a subject that can be categorized as falling within the Diffuse-Proliferation group, can be identified by the increased expression of any one or more genes within Group I, the decreased expression of any one or more genes within Group II, and the decreased expression of any one or more genes in Group III.

In one embodiment the Inflammatory group, and likewise a subject that can be categorized as falling within the Inflammatory group, can be identified by the increased expression of any one or more genes within Group III.

In one embodiment the Inflammatory group, and likewise a subject that can be categorized as falling within the Inflammatory group, can be identified by the increased expression of any one or more genes within Group III and the decreased expression of any one or more genes in Group I.

In one embodiment the Inflammatory group, and likewise a subject that can be categorized as falling within the Inflammatory group, can be identified by the increased expression of any one or more genes within Group III and the increased expression of any one or more genes within Group II.

In one embodiment the Inflammatory group, and likewise a subject that can be categorized as falling within the Inflammatory group, can be identified by the increased expression of any one or more genes within Group III, the decreased expression of any one or more genes in Group I, and the increased expression of any one or more genes within Group II.

In one embodiment the Limited group, and likewise a subject that can be categorized as falling within the Limited group, can be identified by the increased expression of any one or more genes within Group IV.

In one embodiment the Limited group, and likewise a subject that can be categorized as falling within the Limited group, can be identified by the increased expression of any one or more genes within Group IV, the decreased expression of any one or more genes within Group I, the decreased expression of any one or more genes within Group III, and the increased expression of any one or more genes within Group II.

In one embodiment the Normal-Like group, and likewise a subject that can be categorized as falling within the Normal-Like group, can be identified by the increased expression of any one or more genes within Group II.

In each of the foregoing embodiments concerning the Diffuse-Proliferation group, the Inflammatory group, and the Limited group, and likewise a subject that can be categorized as falling within the Diffuse-Proliferation group, the Inflammatory group, or the Limited group, in one embodiment the genes of Group I are limited to any one or more of the following genes, each identified by name: ANP32A, APOH, ATAD2, B3GALT6, B3GAT3, C12orf14, C14orf131, CACNG6, CBLL1, CBX8, CDC7, CDT1, CENPE, CGI-90, CLDN6, CREB3L3, CROC4, DDX3Y, DERP6, DJ971N18.2, EHD2, ESPL1, FGF5, FLJ10902, FLJ12438, FLJ12443, FLJ12484, FLJ12572, FLJ20245, FLJ32009, FLJ35757, FXYD2, GABRA2, GATA2, GK, GSG2, HPS3, IKBKG, IL23A, INSIG1, KIAA1509, KIAA1609, KIAA1666, LDLR, LGALS8, LILRB5, LOC123876, LOC128977, LOC153561, LOC283464, LRRIQ2, LY6K, MAC30, ME2, MGC13186, MGC16044, MGC16075, MGC29784, MGC33839, MGC35212, MGC4293, MICB, MLL5, MTRF1L, MUC20, NICN1, NPTX1, OAS3, OGDHL, OPRK1, PCNT2, PDZK1, PITPNC1, PPFIA4, PREB, PRKY, PSMD11, PSPH, PSPHL, PTP4A3, PXMP2, RAB15, RAD51AP1, RIP, RNF121, RPL41, RPS18, RPS4Y1, RPS4Y2, S100P, SORD, SP1, SYMPK, SYT6, TM9SF4, TMOD3, TNFRSF12A, TPRA40, TRIP, TRPM7, TTR, TUBB4, VARS2L, ZNF572, and ZSCAN2. Similarly, in one embodiment the genes of Group I are limited to any one or more of the following genes, each identified by GenBank accession number only: A_{—}24_BS934268, AB065507, AC007051, AI791206, AK022745, AK022893, AK022997, AK094044, AL391244, AL731541, AL928970, BC010544, BC020847, BM925639, BM928667, ENST00000328708, ENST00000333517, I_{—}1891291, I_{—}3580313, NM_{—}001009569, NM_{—}001024808, NM_{—}172020, NM_{—}173705, NM_{—}178467, NR_{—}001544, THC1434038, THC1484458, THC1504780, U62539, XM_{—}210579, XM_{—}303638, and XM_{—}371684.

In addition, in each of the foregoing embodiments concerning the Diffuse-Proliferation group, the Inflammatory group, the Limited group, and the Normal-Like group, and likewise a subject that can be categorized as falling within the Diffuse-Proliferation group, the Inflammatory group, the Limited group, or the Normal-Like group, in one embodiment the genes of Group II are limited to any one or more of the following genes, each identified by name: AADAC, ADAM17, ADH1A, ADH1C, AHNAK, ALG1, ALG5, AMOT, AOX1, AP2A2, ARK5, ARL6IP5, ARMCX1, BECN1, BECN1, BMP8A, BNIP3L, C10orf119, C1orf24, C1orf37, C20orf10, C20orf22, C5orf14, C6orf64, C9orf61, CAPS, CASP4, CASP5, CAST, CAV2, CCDC6, CCNG2, CDC26, CDK2AP1, CDR1, CFHL1, CNTN3, CPNE5, CRTAP, CTNNA1, CTSC, CUTL1, CXCL5, CYBRD1, CYP2R1, DBN1, DCAMKL1, DCL-1, DIAPH2, DKK2, ECHDC3, ECM2, EIF3S7, EMB, EMCN, EMILIN2, ENPP2, EPB41L2, FBLN1, FBLN2, FEM1A, FGL2, FHL5, FKBP7, FLI1, FLJ10986, FLJ20032, FLJ20701, FLJ23861, FLJ34969, FLJ36748, FLJ36888, FLJ43339, FZR1, GABPB2, GARNL4, GHITM, GHR, GIT2, GLYAT, GPM6B, GTPBP5, HELB, HOXB4, IFNA6, IGFBP5, IL13RA1, IL15, KAZALD1, KCNK4, KCNS3, KCTD10, KIAA0232, KIAA0494, KIAA0562, KIAA0870, KIAA1190, KIF25, KLHL18, KLK2, LAMP2, LEPROTL1, LHFP, LMO2, LOC114990, LOC255458, LOC387680, LOC400027, LOC493869, LOC87769, LRBA, MAFB, MAGEH1, MAN2B2, MCCC2, MEGF10, MFAP5, MGC11308, MGC15523, MGC3200, MGC35048, MGC45780, MOGAT3, MPPE1, MPZ, MYO1B, MYOC, NFYC, NIPSNAP3B, OPTN, OSR2, PAM, PBXIP1, PCOLCE2, PDGFC, PDGFRA, PDGFRL, PEX19, PHAX, PIP, PKM2, PKP2, PMP22, POU2F1, PPAP2B, PRAC, PSMA5, PSORS1C1, PTGIS, RECK, RGS11, RGS5, RIMS3, RIPK2, RNASE4, RNF125, RNF13, RNF146, RNF19, ROBO1, ROBO3, RPL7A, SARA1, SAV1, SCGB1D1, SDK1, SECP43, SECTM1, SERPINB2, SGCA, SH3BGRL, SH3GLB1, SH3RF2, SLC10A3, SLC12A2, SLC14A1, SLC39A14, SLC7A7, SLC9A9, SLPI, SMAD1, SMAP1, SMARCE1, SMP1, SNTG2, SNX7, SOCS5, SSPN, STX7, SUMF1, TAS2R10, TDE2, TFAP2B, TGFBR2, THSD2, TM4SF3, TMEM25, TMEM34, TNA, TNKS2, TRAD, TRAF3IP1, TREM4, TRIM35, TRIM9, TTYH2, TUBB1, UBL3, ULK2, URB, USP54, UST, UTRN, UTX, WIF1, WWOX, XG, YPEL5, and ZFHX1B. Similarly, in one embodiment the genes of Group II are limited to any one or more of the following genes, each identified by GenBank accession number only: A_{—}32_BS169243, A_{—}32_BS200773, A_{—}32_BS53976, AC025463, AF124368, AF161364, AF318337, AF372624, AK001565, AK022793, AK055621, AK056856, AL050042, AL137761, BC035102, BC038761, BC039664, BG252130, BI014689, D80006, ENST00000298643, ENST00000300068, ENST00000305402, ENST00000307901, ENST00000321656, ENST00000322803, ENST00000329246, ENST00000331640, ENST00000332271, ENST00000333784, H16080, I_{—}1861543, I_{—}1882608, I_{—}1985061, I_{—}3335767, I_{—}3551568, I_{—}3588329, I_{—}932413, I_{—}962800, I_{—}966091, NM_{—}001008528, NM_{—}001009555, NM_{—}001013632, NM_{—}001014975, NM_{—}001018006, NM_{—}001018076, NM_{—}001025077, NM_{—}003671, NM_{—}014758, NM_{—}015262, NM_{—}138411, NM_{—}153030, NM_{—}173709, NM_{—}213595, NR_{—}002184, S62210, THC1419743, THC1429821, THC1457118, THC1459712, THC1461073, THC1506312, THC1511927, THC1515028, THC1525318, THC1531579, THC1544941, THC1551463, THC1559236, THC1560798, THC1563147, THC1572906, THC1574967, THC1591470, XM_{—}165930, and XM_{—}209429.

In addition, in each of the foregoing embodiments concerning the Diffuse-Proliferation group, the Inflammatory group, and the Limited group, and likewise a subject that can be categorized as falling within the Diffuse-Proliferation group, the Inflammatory group, or the Limited group, in one embodiment the genes of Group III are limited to any one or more of the following genes, each identified by name: A2M, AIF1, ALOX5AP, APOL2, APOL3, BATF, BCL3, BIRC1, BTN3A2, C10orf10, C1orf38, C6orf80, CCL2, CCL4, CCR5, CD8A, CDW52, COL6A3, COTL1, CPA3, CPVL, CTAG1B, DDX58, EBI2, EVI2B, F13A1, FAM20A, FAP, FCGR3A, FLJ11259, FLJ22573, FLJ23221, FLJ25200, FYB, GBP1, GBP3, GEM, GIMAP6, GMFG, GZMH, GZMK, HAVCR2, HCLS1, HLA-DMA, HLA-DOA, HLA-DPA1, HLA-DPB1, HLA-DQA1, HLA-DQA2, HLA-DQB1, HLA-DRB1, HLA-DRB5, ICAM2, IFI16, IFI16, IFIT1, IFIT2, IFITM1, IFITM2, IFITM3, IL10RA, INDO, ITGB2, KIAA0063, LAMB1, LCP1, LGALS2, LGALS9, LILRB2, LOC387763, LOC400759, LUM, LYZ, MARCKS, MFNG, MGC24133, MPEG1, MRC1, MRCL3, MS4A6A, MX1, NNMT, NUP62, PAG, PLAU, PPIC, PPIC, PTPRC, RAC2, RGS10, RGS16, RSAFD1, SAT, SCGB2A1, SLC20A1, SLCO2B1, SPARC, SULF1, TAP1, TCTEL1, TIMP1, TNFSF4, UBD, VSIG4, and ZFYVE26. Similarly, in one embodiment the genes of Group III are limited to any one or more of the following genes, each identified by GenBank accession number only: AF533936, BQ049338, ENST00000310210, ENST00000313904, ENST00000329660, I_{—}1000437, I_{—}966691, M15073, NM_{—}001010919, NM_{—}001025201, NM_{—}001033569, THC1543691, and XM_{—}291496.

In addition, in each of the foregoing embodiments concerning the Limited group, and likewise a subject that can be categorized as falling within the Limited group, in one embodiment the genes of Group IV are limited to any one or more of the following genes, each identified by name: ATP6V1B2, C1orf42, C7orf19, CKLFSF1, CTAGE4, DICER1, DIRC1, DPCD, DPP3, EMR2, EXOSC6, FLJ90661, FN3KRP, GFAP, GPT, IL27, KCTD15, KIAA0664, LMOD1, LOC147645, LOC400581, LOC441245, MAB21L2, MARCH-II, MGC42157, MRPL43, MT, MT1A, NCKAP1, PGM1, POLD4, RAI16, SAMD10, and UHSKerB. Similarly, in one embodiment the genes of Group IV are limited to any one or more of the following genes, each identified by GenBank accession number only: AC008453, AF086167, AF089746, AJ276555, AL009178, BC031278, BM561346, ENST00000325773, ENST00000331096, THC1562602, X68990, XM_{—}170211, and XM_{—}295760.

Expression of an intrinsic gene, including but not limited to any of the genes of Groups I-IV, is deemed to be increased if its expression is greater than its median expression level as measured across all samples in a reference set of samples, such as the 75 samples described in the examples below. In one embodiment, expression of an intrinsic gene, including but not limited to any of the genes of Groups I-IV, is said to be increased if its expression at least twice the median expression level as measured across all samples in a reference set of samples, such as the 75 samples described in the examples below. In one embodiment, expression of an intrinsic gene, including but not limited to any of the genes of Groups I-IV, is said to be increased if its expression at least four times the median expression level as measured across all samples in a reference set of samples, such as the 75 samples described in the examples below. In one embodiment, expression of an intrinsic gene, including but not limited to any of the genes of Groups I-IV, is said to be increased if its expression at least ten times the median expression level as measured across all samples in a reference set of samples, such as the 75 samples described in the examples below.

Expression of an intrinsic gene, including but not limited to any of the genes of Groups I-IV, is deemed to be decreased if its expression is less than its median expression level as measured across all samples in a reference set of samples, such as the 75 samples described in the examples below. In one embodiment, expression of an intrinsic gene, including but not limited to any of the genes of Groups I-IV, is said to be decreased if its expression at least a factor of two less than (i.e., less than or equal to one half) the median expression level as measured across all samples in a reference set of samples, such as the 75 samples described in the examples below. In one embodiment, expression of an intrinsic gene, including but not limited to any of the genes of Groups I-IV, is said to be decreased if its expression at least a factor of four less than (i.e., less than or equal to one fourth) the median expression level as measured across all samples in a reference set of samples, such as the 75 samples described in the examples below. In one embodiment, expression of an intrinsic gene, including but not limited to any of the genes of Groups I-IV, is said to be decreased if its expression at least a factor of ten less than (i.e., less than or equal to one tenth) the median expression level as measured across all samples in a reference set of samples, such as the 75 samples described in the examples below.

In each of the foregoing embodiments concerning the Diffuse-Proliferation group, the Inflammatory group, the Limited group, and the Normal-Like group, and likewise a subject that can be categorized as falling within the Diffuse-Proliferation group, the Inflammatory group, the Limited group, or the Normal-Like group, in various embodiments “one or more” genes refers to 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, but it is not so limited. In one embodiment “one or more” genes refers to 1 to 4 genes. In one embodiment “one or more” genes refers to 1 to 5 genes. In one embodiment “one or more” genes refers to 1 to 6 genes. In one embodiment “one or more” genes refers to 1 to 7 genes. In one embodiment “one or more” genes refers to 1 to 8 genes. In one embodiment “one or more” genes refers to 1 to 9 genes. In one embodiment “one or more” genes refers to 1 to 10 genes. In one embodiment “one or more” genes refers to 1 to 11 genes. In one embodiment “one or more” genes refers to 1 to 12 genes. Additional embodiments encompassing 1 to 50 genes are also embraced by the invention.

Furthermore, a TGFβ-activated gene expression signature was identified as being predictive of more severe skin disease and co-occurrence of interstitial lung disease in dSSc. Primary dermal fibroblasts derived from patients with dSSc and healthy control skin explants were treated with TGFβ for up to 24 hours. The genome-wide patterns of gene expression were measured and analyzed on DNA microarrays. Nearly 900 genes were identified as TGFβ-responsive in four independent cultures of dermal fibroblasts (two healthy control and two dSSc patients). Expression of the TGFβ-activated genes was examined in forearm and back skin biopsies from 17 dSSc patients and six healthy controls (43 total biopsies). The TGFβ-responsive gene signature was found in 10 of 17 dSSc skin biopsies. Patients that expressed the TGFβ-activated signature showed higher modified Rodnan skin score (p<0.01), and co-occurrence of ILD (p<0.02; Relative Risk=8.0).

The TGFβ-responsive signature disclosed herein is an objective measure of disease severity in dSSc patients. The signature is heterogeneously expressed in dSSc skin and indicates that TGFβ signaling is not a uniform pathogenic mediator in dSSc. This gene expression signature provides a basis for a diagnostic tool for identifying patients at higher risk of developing ILD and a more severe fibrotic skin phenotype and indicates the subset of patients that may be responsive to anti-TGFβ therapy, for example fresolimumab (human anti-TGF-beta monoclonal antibody GC1008) or CAT-192, a recombinant human antibody that neutralizes transforming growth factor beta1 (Denton (2007) supra).

In addition, it was observed that fibrosis in different SSc subsets is driven by different molecular mechanisms tied to either TGFβ or interleukin-13 (IL-13) and interleukin-4 (IL-4). These finding indicate that patient subsetting is necessary in order to target different anti-fibrotic treatments based on molecular subclassifications of SSc patients.

As used herein, the expression of a gene, marker gene or biomarker is intended to refer to the transcription of an RNA molecule and/or translation of a protein or peptide. The expression or lack of expression of a marker gene can indicate a particular physiological or diseased state (e.g., a particular class of scleroderma or phenotype) of a patient, organ, tissue, or cell. The level of expression of a gene, taken alone or in combination with the level of expression of at least one additional gene, can indicate a particular physiological or diseased state (e.g., a particular class of scleroderma or phenotype) of a patient, organ, tissue, or cell. Desirably, the expression or lack of expression, i.e., the level of expression, can be determined using standard techniques such as RT-PCR, immunochemistry, gene chip analysis, oligonucleotide hybridization, ultra high throughput sequencing, etc., that measures the relative or absolute levels of one or more genes. In certain embodiments, the level of expression of a marker gene is quantifiable.

In accordance with the methods of the present invention, a test sample containing at least one cell from clinically involved (i.e., diseased) tissue is provided to obtain a genetic sample. Clinically involved tissue typically can include skin, esophagus, heart, lungs, kidneys, or synovium, but it is not so limited. The test sample may be obtained using any technique known in the art including biopsy, blood sample, sample of bodily fluid (e.g., urine, lymph, ascites, sputum, stool, tears, sweat, pus, etc.), surgical excisions needle biopsy, scraping, etc. In particular embodiments, the test sample is clinically involved skin. From the test sample is obtained a genetic sample or protein sample. The genetic sample contains a nucleic acid, desirably RNA and/or DNA. For example, in determining gene expression one can obtain mRNA from the test sample, and the mRNA may be reverse transcribed into cDNA for further analysis. In another embodiment, the mRNA itself is used in determining the expression of genes of interest. In some embodiments, the expression level of a particular gene can be determined by determining the level or presence of the protein encoded by the mRNA.

The test sample is preferably a sample representative of the scleroderma tissue as a whole. Desirably, there is enough of the test sample to obtain a large enough genetic sample to accurately and reliably determine the expression levels of one or more genes of interest. In certain embodiments, multiple samples can be taken from the same tissue in order to obtain a representative sampling of the tissue.

A genetic sample can be obtained from the test sample using any suitable technique known in the art. See, e.g., Ausubel et al. (1999) Current Protocols in Molecular Biology (John Wiley & Sons, Inc., New York); Molecular Cloning: A Laboratory Manual (1989) 2nd Ed., ed. by Sambrook, Fritsch, and Maniatis (Cold Spring Harbor Laboratory Press); Nucleic Acid Hybridization (1984) B. D. Hames & S. J. Higgins eds. The nucleic acid can be purified from whole cells using DNA or RNA purification techniques. The genetic sample can also be amplified using PCR or in vivo techniques requiring subcloning. In a particular embodiment, the genetic sample is obtained by isolating mRNA from the cells of the test sample and creating cRNA as described herein.

Genetic samples in accordance with the invention are typically obtained from a subject having or suspected of having scleroderma. As used herein, a “subject” is a mammal, e.g., a mouse, rat, hamster, rabbit, goat, sheep, cat, dog, pig, horse, cow, non-human primate, or human. In one embodiment, a “subject” is a human.

As used herein, a “subject having scleroderma” is a subject that has at least one recognized clinical manifestation of scleroderma. In one embodiment, a subject having scleroderma is a subject that has been diagnosed as having scleroderma. Clinical diagnosis of scleroderma is well known in the medical arts. In one embodiment a subject having scleroderma is a subject that has been diagnosed as having scleroderma on the basis, at least in part, of histological (optionally immunohistological) examination.

As used herein, a “subject suspected of having scleroderma” is a subject that has at least one clinical sign or symptom that may suggest that the subject has scleroderma. In one embodiment a subject suspected of having scleroderma is a subject that is suspected to have scleroderma but has not been diagnosed as having scleroderma. In one embodiment a subject suspected of having scleroderma is a subject that is suspected to have scleroderma but has not been diagnosed as having scleroderma on the basis, at least in part, of histological (optionally immunohistological) examination.

Raynaud's phenomenon is the presenting symptom in 30 percent of human subjects with scleroderma. This well-described phenomenon is characterized by episodic digital ischemia, clinically manifested by the sequential development of digital blanching, cyanosis, and rubor (redness) of the fingers or toes following cold exposure and subsequent rewarming. In one embodiment, a subject suspected of having scleroderma is a subject having Raynaud's phenomenon.

Once a genetic sample has been obtained, it can be analyzed for the presence, absence, or level of expression of particular marker genes, e.g., intrinsic genes as disclosed herein. The analysis can be performed using any techniques known in the art including, but not limited to, sequencing, PCR, RT-PCR, quantitative PCR, hybridization techniques, northern blot analysis, microarray technology, DNA microarray technology, etc. In determining the expression level of a biomarker gene or genes in a genetic sample, the level of expression can be normalized by comparison to the expression of another gene such as a well-known, well-characterized gene or a housekeeping gene.

In particular embodiments, expression of a marker gene of interest is determined using microarray technology. Generally, an array is a solid support with peptide or nucleic acid probes attached to the support. Arrays typically include a plurality of different nucleic acid or peptide probes that are coupled to a surface of a substrate in different, known locations. These arrays, also described as microarrays or colloquially “chips”, have been generally described in the art, for example U.S. Pat. Nos. 5,143,854, 5,445,934, 5,744,305, 5,677,195, 6,040,193, 5,424,186 and Fodor, et al. (1991) Science 251:767-777. These arrays may generally be produced using mechanical synthesis methods or light-directed synthesis methods which incorporate a combination of photolithographic methods and solid phase synthesis methods. Techniques for the synthesis of these arrays using mechanical synthesis methods are described in, e.g., U.S. Pat. Nos. 5,384,261 and 6,040,193. Although a planar array surface is preferred, the array can be fabricated on a surface of virtually any shape or even a multiplicity of surfaces. Arrays can be peptides or nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992. Arrays can be packaged in such a manner as to allow for diagnostics or other manipulation of in an all inclusive device, see for example, U.S. Pat. Nos. 5,856,174 and 5,922,591. The use and analysis of arrays is routinely practiced in the art and any conventional scanner and software can be employed.

The expression data from a particular marker gene or group of marker genes can be analyzed using statistical methods described below in the Examples to classify or determine the clinical endpoints of scleroderma patients. In this analysis, the expression of one or more marker genes in the test genetic sample is compared to the expression of the one or more marker genes in a control sample. A control sample can be a sample taken from the same patient, e.g., clinically uninvolved tissue or normal tissue, or can be a sample from a healthy subject. In addition, a control sample can be the average expression of a gene of interest from a cohort of healthy individuals.

In one embodiment, a control sample includes a composite of data derived from a plurality of nucleic acid microarray hybridizations representative of at least one subtype of scleroderma selected from the group consisting of Diffuse-Proliferation, Inflammatory, Limited, and Normal-Like.

In one embodiment, a control sample includes a composite of data derived from a plurality of nucleic acid microarray hybridizations representative of each subtype of scleroderma selected from the group consisting of Diffuse-Proliferation, Inflammatory, Limited, and Normal-Like, for example the 75 microarray hybridizations analyzing 34 individuals described in the Examples below.

Based on data and principles set forth in the Examples below, a subject having or suspected of having scleroderma can be identified as belonging to one category and/or one subcategory of disease (e.g., Diffuse-Proliferative group, Inflammatory group, Limited group, or Normal-Like group) according to the invention. In one embodiment, sample classification is performed by Pearson correlations to the average centroid of the genes shown to be up- or down-regulated in each group. Both up- and down-regulated genes can be important. This profile can be measured in skin biopsies of patients with scleroderma using either a gene expression microarray or, especially for small subsets of genes, by a method such as quantitative PCR.

A centroid is a vector representing the average gene expression of all samples in a group. For example, the average centroid for the Diffuse-Proliferation group is the average of all columns corresponding to the patients classified as the Diffuse-Proliferation group, for all ca. 1000 intrinsic genes. The average centroids for the Inflammatory group, the Limited group, and the Normal-Like group are calculated similarly.

To assign individual patients to groups in the intrinsic subset model, in one embodiment a “nearest centroid predictor” that has been used successfully in breast cancer can be used. This employs training datasets as described herein. The gene expression signatures from the reference datasets are used to create an average centroid for each intrinsic subset (Diffuse-Proliferation, Inflammatory, Limited, and Normal-Like). Centroids from new (patient) samples are individually compared to each average centroid and assigned to the nearest average centroid using a Spearman correlation.

Those skilled in the art will recognize that the expression of one or more genes of interest from the control sample can be input to a database. A relational database is preferred and can be used, but one of skill in the art will recognize that other databases could be used. A relational database is a set of tables containing data fitted into predefined categories. Each table, or relation, contains one or more data categories in columns. Each row contains a unique instance of data for the categories defined by the columns. For example, a typical database for the invention would include a table that describes a sample with columns for age, gender, reproductive status, marker expression level and so forth. Another table would describe the disease: symptoms, level, sample identification, marker expression level and so forth. See, e.g., U.S. Ser. No. 09/354,935.

For the purposes of the present methods, altered expression of a marker gene as compared to the expression of the marker gene in the control sample is indicative of scleroderma disease severity, scleroderma classification, risk of developing interstitial lung disease or a severe fibrotic skin phenotype, interstitial lung disease involvement or digital ulcer involvement, depending on the marker(s) being analyzed. In addition to these identified uses, the analyzed data can also be used to select/profile patients for a particular treatment protocol. For example, the analysis herein provides a signature of genes (e.g., Table 8) expressed in dSSc skin for identifying patients at higher risk of developing ILD and a more severe fibrotic skin phenotype and who may be responsive to anti-TGFβ therapy. In addition, subjects with altered IL-13/IL-4 gene expression patterns include a distinct subset of scleroderma patients that may be responsive to anti-IL-13 therapy. The expression level of one or more of the genes listed in Tables 5, 6, 8, 12 or 13 would desirably be one of several factors used in deciding the prognosis or treatment plan of a patient. In addition, a trained and fully licensed physician would be consulted in determining the patient's prognosis and treatment plan.

The present invention provides selected marker genes that correlate with severity and clinical endpoints of scleroderma. One, two, three, four, five, ten, twenty, thirty, forty, fifty, or more of the marker genes listed in the Examples herein can be employed in the methods of the invention. Particular sets of marker genes can be defined using statistical methods as described in the Examples in order to decrease or increase the specificity or sensitivity of the set.

In addition, different subsets of marker genes can be developed that show optimal function with different races, ethnic groups, sexes, geographic groups, stages of disease, and clinical endpoints such as interstitial lung disease, gastrointestinal involvement, Raynaud's phenomenon and severity of skin disease, etc. Subsets of marker genes can also be developed to be sensitive to the effect of a particular therapeutic regimen on disease progression.

The invention also encompasses kits for use in accordance with the present methods. The kits may include labeled compounds or agents capable of detecting one or more of the markers disclosed herein (e.g., nucleic acid probes to detect nucleic acid markers and/or antibodies to detect protein markers) in a biological sample, a means for determining the amount of markers in the sample, and a means for comparing the amount of markers in the sample with a control. The compounds or agents can be packaged in a suitable container. The kit can further include instructions for using the kit in accordance with a method of the invention.

The gene expression profiles in scleroderma provide a list of markers of disease activity that can be used as surrogate markers in clinical trials. Therefore, the analysis of skin biopsies before and after treatment can also be useful in testing the efficacy of novel therapeutics. For example, amongst the 177-gene signature was TNFRSF12A (Tweak Receptor (TweakR); Fn14), which is a TNF receptor family member expressed on both fibroblasts and in endothelial cells. It is induced by FGF1 and other mitogens, including the proinflammatory cytokine TGFβ. In fibroblasts, increased expression results in decreased adhesion to ECM proteins fibronectin and vitronectin. TNFRSF12A has also been shown to play role in angiogenesis. In vitro cross-linking of the TNFRSF12A in endothelial cells stimulates endothelial cell proliferation, while inhibition prevented endothelial cell migration in vitro and angiogenesis in vivo. Activation of TNFRSF12A in human dermal fibroblasts results in increased production of MMP1, the proinflammatory prostaglandin E2, IL-6, IL-8, RANTES and IL-10. The cytoplasmic domain of TNFRSF12A binds to TRAF1, 2 and 3. A factor downstream of the TRAFs, TRIP (TRAF Interacting Protein), is highly correlated with MRSS. With further refinement, these genes could serve as surrogate markers for disease severity in scleroderma.

The invention is described in greater detail by the following non-limiting examples.

### EXAMPLES

### Example 1

### Molecular Subsets in the Gene Expression Signatures of Scleroderma Skin

All subjects signed consent forms, met the American College of Rheumatology classification criteria for SSc (Committee. SfSCotARADaTC (1980) supra), and were further characterized as the diffuse (dSSc) (Leroy, et al. (1988) supra), or the limited (lSSc) subsets (Mayes M D (1998) supra). LSSc patients had three of the five features of CREST (calcinosis, Raynaud's syndrome, esophageal dysmotility, sclerodactyly and telangiectasias) syndrome, or had Raynaud's phenomenon with abnormal nail fold capillaries and scleroderma-specific autoantibodies. The diffuse systemic sclerosis (dSSc) had wide-spread scleroderma and MRSS ranging from 15 to 35. The lSSc patients had MRSS ranging from 8 to 12. Patients with undifferentiated connective tissue disease (UCTD) were excluded from the study.

Skin biopsies were taken from a total of 34 individuals: 17 patients with dSSc, seven patients with lSSc, three patients with morphea (MORPH), six healthy volunteers (NORM) and one patient with eosinophilic fasciitis (EF) (Table 1). dSSc patients (median age 49±9.4 years) were divided into two groups by their disease duration as defined by first onset of non-Raynaud's symptoms. Eight of the dSSc patients had disease duration<3 years since onset of non-Raynaud's symptoms (median disease duration 2.25±0.8 years) and nine dSSc patients had disease duration>3 years since onset of non-Raynaud's symptoms (median disease duration 9±5.3 years). The seven patients with lSSc had a median disease duration 5±9.7 years. The three patients with morphea had median disease duration 7±6.2 years.

TABLE 1

| | | | Skin | | Digital | | | | ANA/ |
|---|---|---|---|---|---|---|---|---|---|
| | Age/ | Duration | Score | RS | Ulcers | | | | Scl-70/ |
| Subject | Sex | (yrs) | (0-51) | (0-10) | (0-3) | GI | ILD | Renal | ACA |
| dSSc1 | 41/F | 2 | 28 | — | 0 | + | + | − | +/+/− |
| dSSc2 | 49/M | 2.5 | 26 | 3 | 0 | + | − | − | ND |
| dSSc3 | 33/F | 2.5 | 35 | 7 | 0 | − | − | − | +/+/− |
| dSSc4 | 47/F | 3 | 35 | 7 | 0 | + | − | − | +/−/− |
| dSSc5 | 52/F | 1 | 10 | 4 | 1 | + | − | − | +/+/− |
| dSSc6 | 63/F | 0.5 | 26 | 10 | 0 | − | − | − | +/−/− |
| dSSc7 | 42/F | 2.5 | 23 | 10 | 3 | + | − | − | ND |
| dSSc8 | 58/M | 2 | 43 | 7 | 0 | − | − | − | +/−/− |
| dSSc9 | 56/F | 8 | 21 | 5 | 0 | + | + | − | +/−/− |
| dSSc10 | 35/F | 7 | 35 | 8 | 2 | + | + | − | −/−/− |
| dSSc11 | 47/F | 8.5 | 30 | 8 | 1 | + | + | − | +/+/− |
| dSSc12 | 38/M | 9 | 15 | 5 | 0 | + | − | − | −/−/− |
| dSSc13 | 47/F | 6 | 15 | 3 | 0 | + | − | − | +/−/− |
| dSSc14 | 49/F | 10 | 15 | 8 | 0 | − | + | − | +/−/− |
| dSSc15 | 58/F | 20 | 18 | 2 | 1 | + | + | − | ND |
| dSSc16 | 65/F | 10 | 20 | 4 | 0 | + | + | + | ND |
| dSSc17 | 40/F | 20 | 15 | 2 | 1 | + | + | + | ND |
| lSSc1 | 67/F | 3 | 8 | 5 | 0 | + | − | − | +/−/+ |
| lSSc2 | 57/F | 2 | 8 | 2 | 0 | + | − | − | +/−/+ |
| lSSc3 | 35/F | 3 | 6 | 6 | 3 | + | − | − | +/−/− |
| lSSc4 | 63/F | 13 | 8 | 6 | 0 | − | + | − | +/−/− |
| lSSc5 | 60/F | 28 | 9 | 6 | 0 | + | + | + | +/−/− |
| lSSc6 | 55/F | 17 | 9 | 6 | 1 | + | + | − | +/−/− |
| lSSc7 | 67/F | 5 | 8 | 5 | 0 | + | + | − | +/+/− |

Clinical characteristics of the 25 Systemic Sclerosis subjects from which skin biopsies were taken are shown. Indicated for each subject are the age, sex, disease duration since first onset of non-Raynaud's symptoms (RS), modified Rodnan skin score on a 51-point scale, a self-reported Raynaud's severity score on a 10-point scale, and the presence or absence of digital ulcers on a 3-point scale. Also indicated are the presence (+) or absence (−) of gastrointestinal involvement (GI), interstitial lung disease (ILD) as determined by high-resolution computerized tomography (HRCT), and renal disease. The age and sex of subjects with Morphea were: Morph1 (49 year old female, disease duration 16 years), Morph2 (54 year old female, disease duration 7 years), and Morph3 (49 year old female, disease duration 4 years). The age and sex of healthy control subjects were as follows: Nor1, 53 year old female; Nor2, 47 year old female; Nor3, 41 year old female; Nor4, 26 year old female; Nor5, 45 year old male; Nor6, 29 year old female. ND = Not determined.

In most cases, two 5-mm punch biopsies were taken from the lateral forearm, 8 cm proximal to the ulna styloid on the exterior surface non-dominant forearm for clinically involved skin. Two 5-mm punch biopsies were also taken from the lower back (flank or buttock) for clinically uninvolved skin. Thirteen dSSc patients provided forearm and back biopsies; four dSSc patients provided only single forearm biopsies. The seven lSSc patients and all six healthy controls also underwent two 5-mm punch biopsies at the identical forearm and back sites. Three subjects with morphea underwent two 5-mm punch biopsies at the clinically affected areas of the leg (MORPH1), abdomen (MORPH2), and back (MORPH3).

For each patient, one biopsy was immediately stored in 1.5 mL RNALATER (AMBION, Austin, Tex.) and frozen at −80° C., a second biopsy was bisected; half went into 10% formalin for routine histology and half was fresh frozen. In total, 61 biopsies were collected for microarray hybridization: 30 from dSSc, 14 from lSSc, four from morphea, one eosinophilic fasciitis, and 12 from healthy controls (Table 2).

TABLE 2

| Diagnosis | Patients | Biopsies | Microarrays |
|---|---|---|---|
| Diffuse SSc | 17 | 34 | 38 |
| Limited SSc | 7 | 14 | 16 |
| Morphea | 3 | 4 | 5 |
| Normal | 6 | 12 | 15 |
| Eosinophilic fasciitis | 1 | 1 | 1 |
| Total | 34 | 61 | 75 |

RNA was prepared from each biopsy by mechanical disruption with a PowerGen125 tissue homogenizer (Fisher Scientific, Pittsburgh, Pa.) followed by isolation of total RNA using an RNEASY Kit for Fibrous Tissue (QIAGEN, Valencia, Calif.). Approximately 2-5 μg of total RNA was obtained from each biopsy.

cRNA Synthesis, Microarray Hybridization and Data Processing. Two hundred ng of total RNA from each biopsy was converted to Cy3-CTP (PERKIN ELMER, Waltham, Mass.) labeled cRNA, and Universal Human Reference (UHR) RNA (STRATAGENE, La Jolla, Calif.) was converted to Cy5-CTP (PERKIN ELMER) labeled cRNA using a low input linear amplification kit (Agilent Technologies, Santa Clara, Calif.). Labeled cRNA targets were then purified using RNEASY columns (QIAGEN). Cy3-labeled cRNA from each skin biopsy was competitively hybridized against Cy5-CTP labeled cRNA from Universal Human Reference (UHR) RNA pool, to 44,000 element DNA oligonucleotide microarrays (Agilent Technologies) representing more than 33,000 known and novel human genes in a common reference design (Novoradovskaya, et al. (2004) BMC Genomics 5:20). Hybridizations were performed for 17 hours at 65° C. with rotation.

After hybridization, arrays were washed following Agilent 60-mer oligo microarray processing protocols (6×SSC, 0.005% TRITON X-102 for 10 minutes at room temperature; 0.1×SSC, 0, 005% TRITON X-102 for 5 minutes at 4° C., rinse in 0.1×SSC). Microarray hybridizations were performed for each RNA sample resulting in 61 hybridizations. Fourteen replicate hybridizations were added, resulting in a total of 75 microarray hybridizations.

Microarrays were scanned using a dual laser GENEPIX 4000B scanner (Axon Instruments, Union City, Calif.). The pixel intensities of the acquired images were then quantified using GENEPIX Pro 5.0 software. Arrays were visually inspected for defects or technical artifacts, and poor quality spots were manually flagged and excluded from further analysis. Only spots with fluorescent signal at least two-fold greater than local background in both Cy3- and Cy5-channels were included in the analysis. Probes missing more than 20% of their data points were excluded, resulting in 28,495 probes that passed the filtering criteria. The data were displayed as log 2 of the LOWESS-normalized Cy5/Cy3 ratio. Since a common reference experimental design was used, each probe was centered on its median value across all arrays.

Selection of Intrinsic Genes. An intrinsic gene identifier algorithm was used to select a set of intrinsic scleroderma genes. Detailed methods on the selection of intrinsic genes are described in art (Perou, et al. (2000) Nature(London) 406:747-752). A gene was considered ‘intrinsic’ if it showed the most consistent expression between forearm-back pairs and technical replicates for the same patient, but had the highest variance in expression across all samples analyzed. The intrinsic gene identifier computes a weight for each gene, which is inversely related to how intrinsic the gene's expression is across the samples analyzed. A lower weight equals a higher ‘intrinsic’ character. A total of 34 experimental groups were defined, each representing the 34 different subjects in the study. Replicate hybridizations for a given patient were assigned to the same experimental group.

To estimate False Discovery Rate (FDR) at a given intrinsic weight, the analysis was repeated on data randomized in rows (i.e., across each gene). The FDR at a given weight was estimated by determining the number of genes that received the same weight or lower in the randomized data. 995 genes were selected that had an intrinsic weight<0.3; in randomized data 39±7 genes (calculated from 10 independent randomizations) had a weight of 0.3 or less, resulting in an FDR of approximately 4%. It was found that a cutoff of 0.3 balanced the number of genes selected with an acceptable FDR, while retaining reproducible hierarchical clustering of technical replicate samples. Although it was possible to select a more or less restrictive list of genes with FDRs of 5% (weight<0.35; 2071 genes), 3.4% (weight<0.25; 425 genes) or 2.4% (weight<0.20; 171 genes), these smaller lists of genes resulted in less reproducible hierarchical clustering indicating overfitting.

Hierarchical Clustering. Average linkage hierarchical clustering was performed in both the gene and experiment dimensions using either Cluster 3.0 software or X-Cluster using Pearson correlation (uncentered) as a distance metric (Eisen et al. (1998) Proc. Natl. Acad. Sci. USA 95:14863-14868). Clustered trees and gene expression heat maps were viewed using Java TreeView Software (Saldanha (2004) Bioinformatics 20:3246-3248).

Robustness and Statistical Significance of Clustering. The statistical significance of clustering was assessed using Statistical Significance of Clustering (SigClust) (Liu, et al. (2007) J. Am. Stat. Assoc.) and Consensus Cluster (Monti, et al. (2003) Machine Learning 52:91-118). SigClust tests the null hypothesis that the samples form a single cluster. A statistically significant p-value indicates the data came from a non-Gaussian distribution and that there is more than one cluster. Two different p-values were used to identify significant clusters, p<0.01 and p<0.001. The statistical significance of the clusters was first assessed at the root node of the tree derived from hierarchical clustering with the ca. 1000 intrinsic genes. If the cluster was statistically significant, the next node further down the tree was tested. The process ended when a cluster had a p-value greater than the established cutoff.

In addition, the ca. 1000 intrinsic genes were analyzed using Consensus Cluster (Monti, et al. (2003) supra). Consensus Cluster is available through GENEPATTERN (v.1.3.1.114; Reich, et al. (2006) Nat. Genet. 38:500-501). Assessment of sample clustering was performed by consensus clustering with K clusters (K=2, 3, 4 . . . 10) using 1000 iterations with random restart. Samples that clustered together most often in each of the K clusters received a correlation value. The resulting consensus matrix was visualized as a color-coded heat map with varying shades of red, the brighter of which corresponded to higher correlation among samples. Statistics including the empirical consensus distribution function (CDF) vs. the consensus index value were determined. The proportion change (ΔK) under the CDF for each K=2, 3, . . . 10 was also determined. Consensus Cluster assignments for each sample are summarized in Table 3.

TABLE 3

| | | | Consensus Cluster | | |
|---|---|---|---|---|---|
| Patient | Cluster 3.0 | Sig Cluster | Assignment | | |
| Identifier | Assignment | (p < 0.001) | K = 4 | K = 5 | K = 6 |
| dSSc2* | Diffuse 1 | 1 | [1 or 3] | [1 or 5] | [1 or 5] |
| dSSc12 | Diffuse 1 | 1 | 1 | 1 | 1 |
| dSSc1 | Diffuse 2 | 1 | 1 | 1 | 1 |
| dSSc10 | Diffuse 2 | 1 | 1 | 1 | 1 |
| dSSc11 | Diffuse 2 | 1 | 1 | 1 | 1 |
| dSSc15 | Diffuse 2 | 1 | 1 | 1 | 1 |
| dSSc16 | Diffuse 2 | 1 | 1 | 1 | 1 |
| dSSc17 | Diffuse 2 | 1 | 1 | 1 | 1 |
| dSSc3 | Diffuse 2 | 1 | 1 | 1 | 1 |
| dSSc4 | Diffuse 2 | 1 | 1 | 1 | 1 |
| dSSc9 | Diffuse 2 | 1 | 1 | 1 | 1 |
| dSSc8* | Inflammatory | [5] | 2 | 2 | 2 |
| dSSc5 | Inflammatory | 2 | 2 | 2 | 2 |
| dSSc6 | Inflammatory | 2 | 2 | 2 | 2 |
| lSSc6 | Inflammatory | 2 | 2 | 2 | 2 |
| lSSc7 | Inflammatory | 2 | 2 | 2 | 2 |
| Morph1 | Inflammatory | 2 | 2 | 2 | 2 |
| Morph2 | Inflammatory | 2 | 2 | 2 | 2 |
| Morph3 | Inflammatory | 2 | 2 | 2 | 2 |
| lSSc1 | Limited | 4 | 4 | 4 | 4 |
| lSSc4 | Limited | 4 | 4 | 4 | 4 |
| lSSc5 | Limited | 4 | 4 | 4 | 4 |
| Nor1 | Limited | 4 | 4 | 4 | 4 |
| lSSc2 | Normal-Like | 3 | 4 | 4 | 4 |
| Nor2 | Normal-Like | 3 | 4 | 4 | 4 |
| Nor3 | Normal-Like | 3 | 4 | 4 | 4 |
| dSSc14 | Normal-Like | 3 | 3 | 3 | 3 |
| dSSc7 | Normal-Like | 3 | 3 | 3 | 3 |
| lSSc3 | Normal-Like | 3 | 3 | 3 | 3 |
| Nor4 | Normal-Like | 3 | 3 | 3 | 3 |
| Nor5 | Normal-Like | 3 | 3 | 3 | 3 |
| Nor6 | Normal-Like | 3 | 3 | 3 | 3 |
| dSSc13* | Unclassified | 1 | [4] | [4] | [4] |
| EF* | Unclassified | 1 | 1 | 1 | [6] |

*Inconsistently classified.

Principal Component Analysis. Principal Component Analysis was performed using Multiexperiment Viewer (MeV) software version 4.0.01 (Margolin, et al. (2005) Bioinformatics 21:3308-3311). Data was loaded into MeV as a tab delimited text file of log 2-transformed Cy3/Cy5 ratios. For PCA analysis (Raychaudhuri, et al. (2000) Pac. Symp. Biocomput. 455-466), missing data were first estimated using K-nearest neighbors (KNN) imputation with N=4.

Module Maps. Module maps were created using the Genomica software package (Segal, et al. (2004) Nat. Genet. 36:1090-1098; Stuart, et al. (2003) Science 392:249-255). Gene sets containing all human Gene Ontology (GO) Terms were obtained from the Genomica database (Human_go_process.gxa, created Nov. 20, 2006). Additional custom gene sets representing the human cell division cycle (Whitfield, et al. (2002) Mol. Biol. Cell 13:1977-2000) and lymphocyte subsets (Palmer, et al. (2006) BMC Genomics 7:115) were created specifically for this study. The human cell division cycle gene set was created from the genes found to periodically expressed in human HeLa cells (Whitfield, et al. (2002) supra). Genes found to show peak expression at the five different cell cycle phases G1/S, S, G2, G2/M and M/G1 were each put into their own independent gene list. Gene sets representing different lymphocyte populations, T cells (total population, CD4+, CD8+), B cells, and granulocytes, were derived for this study from the genes expressed in isolated lymphocyte subsets by Palmer et al. ((2006) supra).

All 75 microarray experiments and 28,495 DNA probes were included in the module map analysis. The 28,495 probes were collapsed to 14,448 unique LocusLink Ids (LLIDs) (Pruitt & Maglott (2001) Nucl. Acids Res. 29:137-140). Only gene sets with at least three genes but fewer than 1000 genes were analyzed. A gene set was considered enriched on a given array if at least three genes from that set were considered to be significantly up-regulated or down-regulated (minimum two-fold change, p<0.05, hypergeometric distribution) on at least four microarrays. Each gene set was corrected for multiple hypothesis testing using an FDR correction of 0.1%.

Correlation to Clinical Parameters. Pearson correlations were calculated between each clinical parameter and the gene expression data in MICROSOFT EXCEL. Pearson correlations between the diagnosis of dSSc, lSSc and healthy controls and the gene expression data were calculated by creating a ‘diagnosis vector’. The diagnosis vector was created by assigning a value 1.0 to all dSSc samples and 0.0 to all remaining samples for the dSSc vector; lSSc and healthy controls were treated similarly creating a vector for each. Pearson correlations were calculated between the gene expression vector and the diagnosis vector for dSSc, lSSc and healthy controls. Correlations between the gene expression and clinical data were plotted as a moving average of a 10-gene window.

Immunohistochemistry (IHC). IHC was performed on paraffin-embedded sections. All immunostaining was completed via a semi-automated protocol utilizing an automated immunostainer (DAKO Corp, Carpenteria, Calif.). Slides were heated, deparaffinized and then hydrated. Protease digestion was completed followed by antigen retrieval via pressure cooker as per standard protocols. After an endogenous peroxidase block with 3% H₂O₂, slides were loaded on to the automated immunostainer. A primary antibody cycle of 30 minutes was followed by a secondary antibody cycle using the ENVISION+ system. Color development was completed using DAB followed by counterstaining with Gills #2 Hematoxylin. Specific conditions for the antibodies utilized were as follows: anti-CD20 (DAKO Corp.) was used at 1:600 for 30 minutes in citrate buffer (pH 6.0); anti-CD3 (DAKO Corp.) at 1:400 for 30 minutes in Tris buffer (pH 9.0), and anti-Ki67 (MiB1; DAKO Corp.) was used at 1:1000 for 30 minutes in Tris buffer (pH 9.0). Marker positive cells were enumerated by tissue compartment in equal sized images of n skin biopsies, with the observer blinded to disease state and array results of the specimens (Table 4).

TABLE 4

| | | KI67 | | | CD3 | | |
|---|---|---|---|---|---|---|---|
| Patient | Assign.^{a} | Append | Epiderm | Derm | Append | Epiderm | Derm |
| Nor2 | Normal-Like | 10 | 11 | 0 | 14 | 0 | 3 |
| Nor3 | Normal-Like | 0 | 11 | 0 | 22 | 0 | 0 |
| | Normal-Like^{b} | 5 | 11 | 0 | 18 | 0 | 7.5 |
| Morph3 | Inflammatory | 1 | 13 | 0 | 205 | 18 | 107 |
| Morph1 | Inflammatory | 0 | 21 | 0 | 36 | 5 | 14 |
| dSSc5 | Inflammatory | 4 | 11 | 0 | 68 | 1 | 5 |
| dSSc6 | Inflammatory | 7 | 0 | 0 | 83 | 2 | 15 |
| | Inflammatory | 3 | 11.3 | 0 | 98 | 6.5 | 35.3 |
| dSSc1 | Prolif(2) | 4 | 20 | 0 | 56 | 0 | 0 |
| dSSc11 | Prolif(2) | 8 | 14 | 0 | 12 | 0 | 7 |
| dSSc2 | Prolif(1) | 0 | 22 | 1 | 31 | 0 | 2 |
| dSSc12 | Prolif(1) | 2 | 85 | 0 | 55 | 10 | 16 |
| | Prolif | 3.5 | 35.3 | 0.3 | 38.5 | 2.5 | 6.3 |

Shown is the summary of total counts per skin biopsy as determined by IHC staining for KI67, which stains cycling cells, and CD3, which stains T cells. Each biopsy was also analyzed for CD20 and only a small number of cells were found around dermal appendages for Morph3 (3), dSSc6 (2) and dSSc12 (2). All other samples were negative for CD20 cells. (Append = dermal appendages (hair follicles, vascular structures, eccrine glands); Epiderm = epidermis; Derm = dermis).
^{a}Intrinsic group to which each sample was assigned.
^{b}Average of total counts per category.

Quantitative Real-Time PCR (qRT-PCR). Each quantitative real-time PCR assay (Heid, et al. (1996) Genome Res. 6:986-994) was performed with 100-200 ng of total RNA. Each sample was reverse-transcribed into single-stranded cDNA using SUPERSCRIPT II reverse transcriptase (INVITROGEN, San Diego, Calif.). Ninety-six-well optical plates were loaded with 25 μl of reaction mixture which contained: 1.25 μl of TAQMAN pre-designed Primers and Probes, 12.5 μl of TAQMAN PCR Master Mix, and 1.25 ng of cDNA. Each measurement was carried out in triplicate with a 7300 Real-Time PCR System (Applied Biosystems, Foster City, Calif.). Each sample was analyzed under the following conditions: 50° C. for 2 minutes and 95° C. for 10 minutes, and then cycled at 95° C. for 15 seconds and 60° C. for 1 minute for 40 cycles. Output data was generated by the instrument onboard software 7300 System version 1.2.2 (Applied Biosystems). The number of cycles required to generate a detectable fluorescence above background (CT) was measured for each sample. Fold difference between the initial mRNA levels of target genes (TNFRSF12A, CD8A and WIF1) in the experimental samples were calculated with the comparative CT method using formula 2-ΔΔCT (Livak & Schmittgen (2001) Methods 25:402-408) and median centered across all samples analyzed.

Overview of the Gene Expression Profiles. Previous studies have demonstrated that the skin of patients with dSSc can be easily distinguished from normal controls at the level of gene expression (Whitfield, et al. (2003) supra; Gardner, et al. (2006) supra). These findings have been extended herein to identify distinct subsets of scleroderma within the existing clinical classifications by gene expression profiling of skin biopsies using DNA microarrays.

Skin biopsies from 34 subjects were analyzed: twenty-four patients with SSc (17 dSSc and 7 lSSc), three patients with morphea and six healthy controls (Tables 1-2). A single biopsy was analyzed from a patient with eosinophilic fasciitis (EF). Skin biopsies were taken from two different anatomical sites for 27 subjects: a forearm site, and a lower back site. In dSSc, the forearm site was clinically affected and the back site was clinically unaffected. In lSSc, both forearm and back sites were clinically unaffected. Seven subjects provided single biopsies resulting in a total of 61 biopsies. Total RNA was prepared from each skin biopsy and analyzed on whole-genome DNA microarrays. In addition, fourteen technical replicates were analyzed for a total of 75 microarray hybridizations.

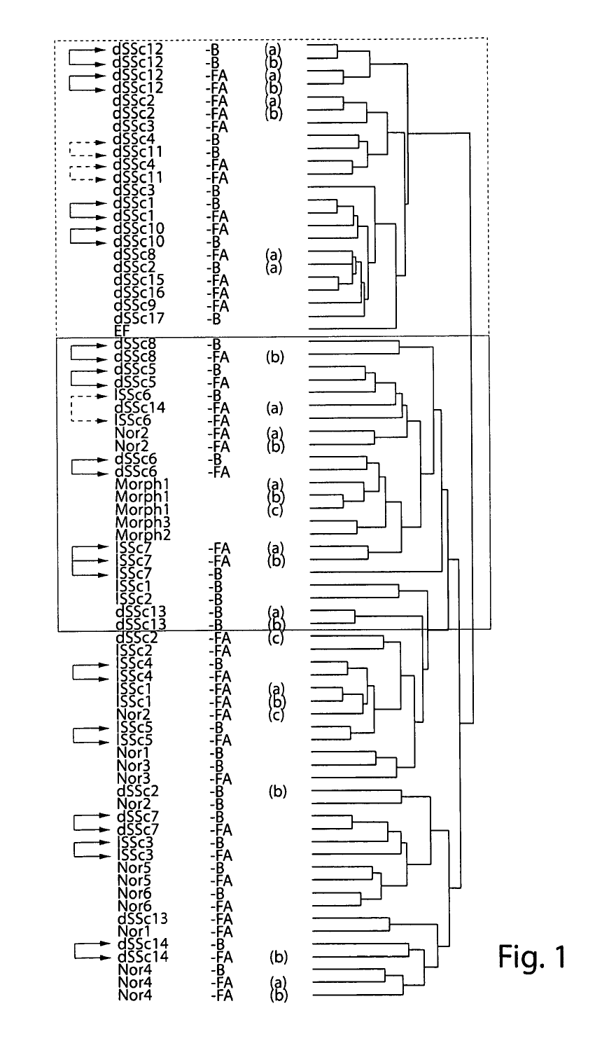

This analysis identified 4,149 probes whose expression varied from their median values in these samples by more than two-fold in at least two of the 75 arrays. These probes were analyzed by two-dimensional hierarchical clustering (Eisen, et al. (1998) Proc. Natl. Acad. Sci. USA 95:14863-14868) and the resulting sample dendrogram (FIG. 1) showed that the samples separated into two main branches that, in part, stratified patients by their clinical diagnosis. The branch lengths in the tree were inversely proportional to the correlation between samples or groups of samples. The diversity in gene expression among the patients with scleroderma was greater than previously shown (Whitfield, et al. (2003) supra; Gardner, et al. (2006) supra) as distinct subsets of scleroderma were evident in the gene expression patterns. Some of these delineated existing classifications, such as the distinction between limited and diffuse, while others reflected new groups. One subset of dSSc patients clustered on the left branch (indicated by box with dashed line; FIG. 1) and had gene expression profiles that were distinct from both healthy controls and patients with lSSc, while a second subset of dSSc skin clustered in the middle of the dendrogram tree (indicated by box with solid line; FIG. 1), and a third set clustered with healthy controls. It was observed that lSSc samples formed a group in the middle portion of the dendrogram and could be associated with a distinct, but heterogeneous gene expression signature that also showed high expression in a subset of dSSc patients (i.e., UTS2R, GALR3, PARD6G, PSEN1, PHOX2A, CENTG3, HCN4, KLF16, and GPR150). LSSc samples were partially intermixed with normal controls on the right boundary and with dSSc on the left boundary of the tree, illustrating that their gene expression phenotype was highly variable (FIG. 1). Samples taken from individuals with morphea also grouped together with a gene expression signature that overlapped with those of dSSc and lSSc (FIG. 1). Although nodes could be flipped, the nodes of the dendrogram were left as originally organized by the clustering software, which placed nodes with the most similar samples next to one another. The assignment of samples into particular clusters (Table 3) would not change, however, even if nodes were flipped.

Multiple distinct gene expression programs were evident in each subgroup. Some of these recapitulated the major themes in microarray analysis of dSSc skin (Whitfield, et al. (2003) supra), while others reflected gene expression programs not previously observed. For example, immunoglobulins typically associated with B lymphocytes and plasma cells were expressed in a subset of the dSSc skin biopsies (i.e., IGLC2, CCL4, CCR2, IGH, IGJ, IGLL1, IGKC, F7, IGHG4, and MT1X). Dense clusters of infiltrating B cells in dSSc have been identified by immunohistochemistry (IHC), indicating that these genes may be from infiltrating CD20+ B cells rather than from a small number of infiltrating plasma cells (Whitfield, et al. (2003) supra).

Infiltrating T cells have been identified in the skin of dSSc patients (Sakkas, et al. (2002) J. Immunol. 168:3649-3659; Kraling, et al. (1996) Pathobiology 64:99-114; Kraling, et al. (1995) Pathobiology 63:48-56; Yurovsky, et al. (1994) J. Immunol. 153:881-891; Fleischmajer, et al. (1977) Arthritis Rheum. 20:975-984), although an association between T cell gene expression and dSSc has not been demonstrated in the art (Whitfield, et al. (2003) supra). The instant results indicate that genes typically associated with T cells are more highly expressed in a subset of the patients. These genes included the PTPRC (CD45; Leukocyte Common Antigen Precursor), which is required for T-cell activation through the antigen receptor (Trowbridge & Thomas (1994) Annu. Rev. Immunol. 12:85-116; Trowbridge, et al. (1991) Biochim. Biophys. Acta 1095:46-56; Koretzky, et al. (1990) Nature(London) 346:66-68), as well as CD2 (Sewell, et al. (1989) Transplant. Proc. 21:41-43; Sewell, et al. (1986) Proc. Natl. Acad. Sci. USA 83:8718-8722) and CDW52 (Hale, et al. (1990) Tissue Antigens 35:118-127) that are expressed on the surface of T lymphocytes. Also found were CD8A, Granzyme K, Granzyme H, and Granzyme B that are typically expressed in cytotoxic T lymphocytes (Ledbetter, et al. (1981) J. Exp. Med. 153:310-323; Sayers, et al. (1996) J. Leukoc. Biol. 59:763-768; Przetak, et al. (1995) FEBS Lett. 364:268-271; Smyth, et al. (1995) Immunogenetics 42:101-111; Baker, et al. (1994) Immunogenetics 40:235-237), and CCR7, which is expressed in B and T lymphocytes (Yoshida, et al. (1997) J. Biol. Chem. 272:13803-13809). Genes induced by interferon (IFIT2, GBP1), genes involved in antigen presentation (HLA-DRB1, HLA-DPA1 and HLA-DMB) and CD74, the receptor for Macrophage Inhibitory factor (MIF), are also present (Jensen, et al. (1999) Immunol. Res. 20:195-205; Jensen, et al. (1999) Immunol. Rev. 172:229-238; Cresswell (1994) Annu. Rev. Immunol. 12:259-293; Gore, et al. (2007) J. Biol. Chem. 283:2784-2792; Lantner, et al. (2007) Blood 110:4303-4311). Genes typically associated with the monocyte/macrophage lineage, B cells and dendritic cells (DCs) were also found in this cluster including Leukocyte immunoglobulinlike receptor B2 and B3 (LILRB2 and LILRB3; Wagtmann, et al. (1997) Curr. Biol. 7:615-618; Arm, et al. (1997) J. Immunol. 159:2342-2349). Furthermore, chemokine receptor 5 (CCR5), interleukin 10 receptor alpha (IL10RA), integrin beta 2 (ITGB2), V-rel reticuloendotheliosis viral oncogene B (RELB), Janus kinase 3 (JAK3), tumor necrosis factor ligand superfamily 13b (TNFSF13B), and leukocyte specific transcript 1 (LST1) are expressed in this group of genes, as are genes specific to the monocyte/macrophage lineage, e.g., CD163 (Sulahian, et al. (2000) Cytokine 12:1312-1321).

Genes typically associated with the process of fibrosis were co-expressed with markers of T lymphocytes and macrophages. These genes showed increased expression in the central group of samples that included patients with dSSc, lSSc and morphea. Included in this set of genes were the collagens (COL5A2, COL8A1, COL10A1, COL12A1), and collagen triple helix repeat containing 1 (CTHRC1), which is typically expressed in vascular calcifications of diseased arteries and has been shown to inhibit TGFβ signaling (LeClair, et al. (2007) Circ. Res. 100:826-833; Pyagay, et al. (2005) Circ. Res. 96:261-268). Also found in this cluster was lumican (LUM), peptidylprolyl isomerase C (PPIC), integrin beta-like 1 (ITGBL1), raft-linking protein (RAFTLIN), anthrax toxin receptor 1 (ANTXR1), secreted frizzled-related protein 2 (SFRP2) and fibrillin-1 (FBN1). The phenotype of the TSK1 mouse, a model of scleroderma, results from a partial in-frame duplication of the FBN1 gene and defects in FBN1 are the cause of Marfan's syndrome (OMIM: 154700).

A surprising result in this study was the differential expression of a ‘proliferation signature’. The proliferation signature was defined as genes that were expressed only when cells were dividing (Whitfield, et al. (2006) Nat. Rev. Cancer 6:99-106). It has been shown that proliferation signatures, originally identified in breast cancer (Perou, et al. (2000) supra; Perou, et al. (1999) Proc. Natl. Acad. Sci. USA 96:9212-9217), are composed almost completely of cell cycle-regulated genes (Whitfield, et al. (2002) supra). Genes showing increased expression in the cluster identified herein included the cell cycle-regulated genes CKS1B, CDKS2, CDC2, MCM8, E2F7, FGL1, RAD51AP1, ASPM, FBXO5, KNTC2, ECT2, DONSON, FGG, ANLN, Spc25, DLG7, ASK, DCC1, FANCA, IMP-1, RIS1, CDCA2, RAD54L, OIP5, ZWINT, DNMT3B, TMSNB, HLXB9, CDCA8, TOPK, EGLN1, HIST1H2BM, SMARCA3, and SAA4. The existence of a proliferation signature was consistent with reports demonstrating that a subset of cells in dSSc skin biopsies show high levels of tritiated thymidine uptake indicative of cells undergoing DNA replication (Fleischmajer & Perlish (1977) J. Invest. Dermatol. 69:379-382; Kazandjian, et al. (1982) Acta Derm. Venereol. 62:425-429); and studies showing increased expression of the cell cycle-regulated gene PCNA in a perivascular distribution (Rajkumar, et al. (2005) Arthritis Res. Ther. 7:R1113-1123). IHC of dSSc skin biopsies with the proliferation marker KI67 also showed proliferating cells primarily in the epidermis.

Another cluster of genes was expressed at low levels in the dSSc skin biopsies but at higher levels in all other biopsies, however it was not clearly associated with a single biological function or process. Included in this cluster were the genes IL17D, MFAP4, RECK, PCOLCE2, WISP2, TNXB, FBLN1, PDGFRL, GALNTL2, FBLN2, SGCA, CTSG, DCN, and KAZALD1. Also, included in this cluster were WIF1, Tetranectin, IGFBP6, and IGFBP5 identified by Whitfield, et al. (2003) supra with similar patterns of expression.

Since the skin of lSSc patients does not show any clinical or histologic manifestations at the biopsy site, it was possible that the skin of those patients would not show significant differences in gene expression when compared to normal controls. In fact, lSSc skin showed a distinct, disease-specific gene expression profile. This novel finding demonstrates that microarrays are sensitive enough to identify the limited subset of SSc even when discernable skin fibrosis was not present. There was a signature of genes that was expressed at high levels in a subset of lSSc patients, and variably expressed in dSSc and normal controls. Included in this signature was GALR3, PARD6G, PSEN1, PHOX2A, CENTG3, HCN4, KLF16, GPR150 and the urotensin 2 receptor (UTS2R). The ligand for this receptor, urotensin 2, was considered to be one of the most potent vasoconstrictors yet identified (Douglas, et al. (2000) Br. J. Pharmacol. 131:1262-1274; Ames, et al. (1999) Nature 401:282-286; Grieco, et al. (2005) J. Med. Chem. 48:7290-7297). This finding indicates that this vasoactive peptide may be involved in the vascular pathogenesis of lSSc.

It has been demonstrated that skin biopsies from patients with early dSSc show nearly identical patterns of gene expression at a clinically affected forearm site and a clinically unaffected back site, and the gene expression profiles are distinct from those found in healthy controls (Whitfield, et al. (2003) supra). This finding was confirmed in instant larger cohort of patients analyzed on a different microarray platform. Fourteen of 22 forearm-back pairs clustered immediately next to one another indicating that these samples were more similar to each other than to any other sample (FIG. 1). An additional three forearm-back pairs grouped together with only a single sample between them (FIG. 1). In total, 17 of 22 (77%) forearm-back pairs showed nearly identical patterns of gene expression. This result held true for patients with lSSc even though neither the forearm nor back biopsy sites in lSSc patients are defined as clinically affected (Whitfield, et al. (2003) supra). Nine out of 14 technical replicates were observed to cluster next to one another. The five technical replicates that did not cluster together were likely misclassified as a result of noise in the genes selected by fold change.

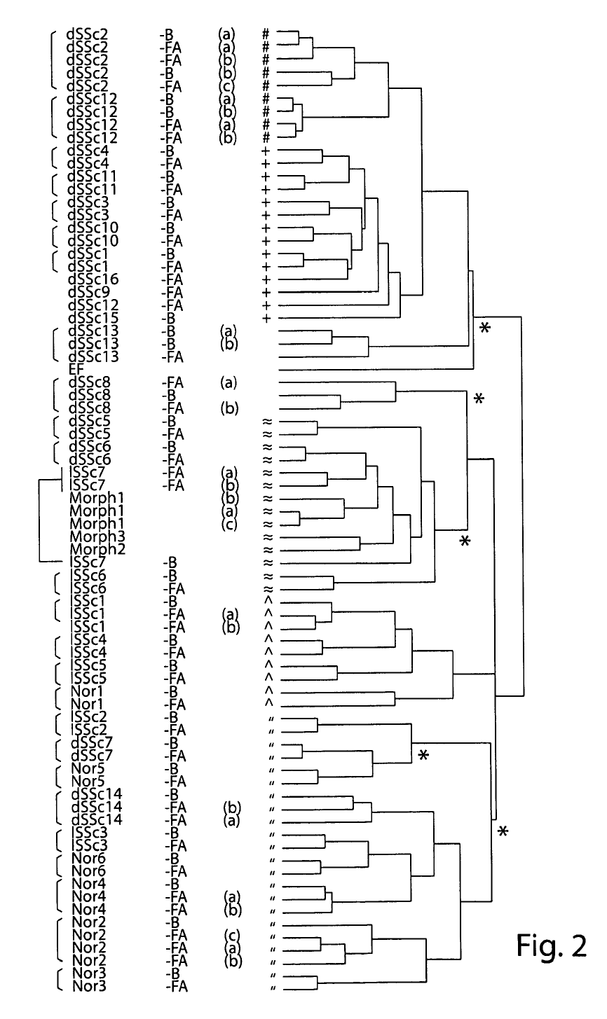

Classification of Scleroderma Via Intrinsic Genes. A list of genes selected by their fold change alone is typically not ideal for classifying samples because they emphasize differences between samples rather than the intrinsic differences between patients (Perou, et al. (2000) supra; Sorlie, et al. (2001) Proc. Natl. Acad. Sci. USA 98:10869-10874). To select genes that captured the intrinsic differences between patients, the observation that the forearm-back pairs from each SSc patient showed nearly identical patterns of gene expression was exploited to select the ‘intrinsic’ genes in SSc. Nearly 1000 genes with the most consistent expression between each forearm-back pair and technical replicates, but with the highest variance across all samples analyzed were selected (Perou, et al. (2000) supra; Sorlie, et al. (2001) supra) (Table 5). Each of the ca. 1000 intrinsic genes selected was centered on its median value across all experiments, and the data clustered hierarchically in both the gene and experiment dimension using average linkage hierarchical clustering. The dendrogram presented in FIG. 2 summarizes the relationship among the samples and shows their clear separation into distinct groups. As a direct result of this gene selection, all forearm-back pairs clustered together and all technical replicate hybridizations clustered together when using the intrinsic genes. Sample identifiers have been indicated according to the patient diagnosis: dSSc with †, lSSc with ̂, morphea and EF have no symbols, and normal controls are marked with ″. The dendrogram has been demarcated to reflect the signatures of gene expression that were an inherent feature of the biopsies.

The gene expression signatures further subdivided samples within existing clinical groups. A consistent set of genes was found that was highly expressed in a subset of the dSSc samples, which occupy the left branch of the dendrogram tree. These groups were designated diffuse 1 (FIG. 2; # branches) and diffuse 2 (FIG. 2; † branches) as they consistently clustered as two separate groups (FIGS. 1 and 2) and had distinct signatures of gene expression. The most consistent biological program expressed across the diffuse 1 and diffuse 2 scleroderma samples was that of proliferation (i.e., LILRB5, CLDN6, OAS3, TPRA40, TMOD3, GATA2, NICN1, CROC4, SP1, TRPM7, MTRF1L, ANP32A, OPRK1, PTP4A3, ESPL1, SYT6, MICB, PSMD11, CDT1, FGF5, CDC7, APOH, FXYD2, OGDHL, PPFIA4, PCNT2, ME2 M, HPS3, TNFRSF12A, SYMPK, CACNG6, TRIP, CENPE, RAD51AP1, and IL23A). This group is broadly referred to herein as the Diffuse-Proliferation group, or, equivalently, the Diffuse-Proliferative subtype.

A second group contained dSSc, lSSc and morphea samples on a single branch of the dendrogram tree (FIG. 2, ∞ branches). The genes most highly expressed in this group were those typically associated with the presence of inflammatory lymphocyte infiltrates (i.e., HLA-DQB1, HLA-DQA1, HLA-DQA2, HLA-DPB1, HLA-DRB1, LGALS2, EVI2B, CPVL, AIF1, IFI16, FAP, EBI2, IFIT2, GBP1, CCL2, A2M, ITGB2, LGALS9, GZMK, GZMH, CCR5, IL10RA, ALOX5AP, MRC1, HLA-DOA, HLA-DMA, HLA-DPA1, MPEG1, LILRB2, CPA3, CDW52, CD8A, PTPRC, CCL4, COL6A3, ICAM2, IFIT1, and MX1) as described above. This group is referred to herein as the Inflammatory group, or, equivalently, the Inflammatory subtype.

A third group contained primarily lSSc samples (FIG. 2, ̂), which had low expression of the proliferation and T cell signatures but had high expression of a distinct signature found heterogeneously across the samples (i.e., NCKAP1, MAB21L2, SAMD10, GPT, GFAP, MT, IL27, RAI16, DIRC1, MT1A, DICER1, PGM1, EXOSC6, DPP3, CKLFSF1, EMR2, and LMOD1). This group is referred to herein as the Limited group, or, equivalently, the Limited subtype.

A branch of samples which primarily included healthy controls (FIG. 2, ″) also contained samples from one patient with a diagnosis of dSSc and a patient with lSSc. This group was labeled the Normal-Like group, or, equivalently, the Normal-Like subtype, since the gene expression signatures in these samples more closely resembled and clustered with normal skin.

Significance and Reproducibility of Intrinsic Clustering. To examine the robustness of these groups, two separate analyses were performed: Statistical Significance of Clustering (SigClust) (Liu, et al. (2007) supra) and consensus clustering (Monti, et al. (2003) supra). SigClust analysis was performed with the ca. 1000 intrinsic genes. At a p-value<0.001, five statistically significant clusters were found. The four major groups of Diffuse-Proliferation, Inflammatory, Limited and Normal-Like groups were each found to be statistically significant (FIG. 2); samples of patient dSSc8 formed a statistically significant group of their own in the SigClust analysis (Table 3). Thus, the major groups identified in the hierarchical clustering using the ca. 1000 intrinsic genes were statistically significant and could not be reasonably divided into smaller clusters with the current set of data. The two branches within the Diffuse-Proliferation group did not reach statistical significance in this analysis even though there were identifiable differences in their gene expression profile.

To perform a second validation of the intrinsic groups, consensus clustering was used (Monti, et al. (2003) supra), which performs a K-means clustering analysis on randomly selected subsets of the data by resampling without replacement over 1,000 iterations using different values of K. To determine the number of clusters present in the data, the area under the Consensus Distribution Function (CDF) was examined. The point at which the area under the CDF ceased to show significant changes indicates the probable number of clusters. The largest change occurred between three and four clusters with a slight change between four and five clusters.

Based on this analysis and the SigClust analysis, it appeared that there were approximately four to five statistically significant clusters in the data. The statistically significant cluster assignments from both SigClust and consensus clustering are summarized in Table 3. These are (1) Diffuse-Proliferation, composed completely of patients with dcSSc, (2) Inflammatory, which includes a subset of dSSc, lSSc and morphea, (3) Limited, characterized by the inclusion of lSSc patients and (4) Normal-Like, which includes five of six healthy controls along with two dSSc patients and one lSSc patient. Notably, three samples were not consistently classified into the primary clusters. These were: dSSc2 which was assigned to the either the Diffuse-Proliferation, Normal-Like or into a single cluster by itself; dSSc13 which was assigned to either Diffuse-Proliferation or the Limited groups; and patient EF which clustered either on the peripheral edge of the Diffuse-Proliferation cluster or was assigned to a cluster by itself.

To determine how sensitive the clustering was to the selection of the intrinsic genes, the clustering results were analyzed using a larger list of 2071 intrinsic genes. These clustering results were compared to that obtained with the ca. 1000 intrinsic genes. Although slight differences in the ordering of the samples were observed, the major subsets of Diffuse-Proliferation, Inflammatory, and Limited were again identified. The Normal-Like group was split onto two different branches using this larger set of genes. Samples that showed inconsistent clustering were from patient dSSc2, dSSc8, dSSc13, and the single array for patient EF. The samples for each of these patients were also inconsistently classified in the SigClust and consensus clustering analysis using the ca. 1000 intrinsic gene set.

Principal Component Analysis (PCA) was used to confirm the sample grouping found by hierarchical clustering. PCA is an analytic technique used to reduce high dimensional data into more easily interpretable principal components by determining the direction of maximum variation in the data (Raychaudhuri, et al. 2000) supra). The ca. 1000 intrinsic genes were analyzed by PCA using the MultiExperiment Viewer (MeV) software (Margolin, et al. (2005) supra). The first and second principal components that captured the most variability in the data, and the first and third principle components were plotted in 2-dimensional space. The 2D projection showed that the samples grouped in a manner similar to that found by hierarchical clustering analysis: normal controls and limited samples grouped together and the two different groups of diffuse scleroderma grouped together. Notably, the first and second principal components separated the Diffuse-Proliferation, the Inflammatory and the Normal-Like/Limited groups. When the first and third principal components were analyzed, a distinction between dSSc group 1 and dSSc group 2 was clearly delineated, as was the distinction between Normal-Like and Limited. The PCA analysis provided further evidence, in addition to the hierarchical clustering analysis, that the gene expression groups were stable features of the data.

TABLE 5

| Gene Symbol | Gene Name | Accession |
|---|---|---|
| A2M | Alpha-2-macroglobulin | M36501 |
| AADAC | Arylacetamide deacetylase (esterase) | NM_001086 |
| ACTB | Actin, beta | NM_001101 |
| ADAM17 | A disintegrin and metalloproteinase domain 17 | NM_003183 |
| | (tumor necrosis factor, alpha, converting enzyme) | |
| ADH1A | Alcohol dehydrogenase 1A (class I), alpha | NM_000667 |
| | polypeptide | |
| ADH1C | Alcohol dehydrogenase 1C (class I), gamma | NM_000669 |
| | polypeptide | |
| AHNAK | AHNAK nucleoprotein (desmoyokin) | NM_024060 |
| AIF1 | Allograft inflammatory factor 1 | NM_004847 |
| AKAP13 | A kinase (PRKA) anchor protein 13 | AF406992 |
| ALG1 | Asparagine-linked glycosylation 1 homolog | NM_019109 |
| | (yeast, beta-1,4-mannosyltransferase) | |
| ALG2 | Asparagine-linked glycosylation 2 homolog | NM_033087 |
| | (yeast, alpha-1,3-mannosyltransferase) | |
| ALG5 | Asparagine-linked glycosylation 5 homolog | NM_013338 |
| | (yeast, dolichyl-phosphate beta- | |
| | glucosyltransferase) | |
| ALOX5AP | Arachidonate 5-lipoxygenase-activating protein | NM_001629 |
| ALS2CR13 | Amyotrophic lateral sclerosis 2 (juvenile) | NM_004703 |
| | chromosome region, candidate 13 | |
| ALX3 | Aristaless-like homeobox 3 | NM_006492 |
| AMFR | Autocrine motility factor receptor | NM_138958 |
| AMOT | Angiomotin | NM_133265 |
| ANP32A | Acidic (leucine-rich) nuclear phosphoprotein 32 | AK021784 |
| | family, member A | |
| AOX1 | Aldehyde oxidase 1 | NM_001159 |
| AP2A2 | aptor-related protein complex 2, alpha 2 | NM_012305 |
| | subunit | |
| APOH | Apolipoprotein H (beta-2-glycoprotein I) | NM_000042 |
| APOL2 | Apolipoprotein L, 2 | NM_030882 |
| APOL3 | Apolipoprotein L, 3 | NM_145640 |
| ARHGEF10 | Rho guanine nucleotide exchange factor (GEF) | NM_014629 |
| | 10 | |
| ARK5 | AMP-activated protein kinase family member 5 | NM_014840 |
| ARL6IP5 | ADP-ribosylation-like factor 6 interacting protein 5 | NM_006407 |
| ARMCX1 | Armadillo repeat containing, X-linked 1 | NM_016608 |
| ARX | Aristaless related homeobox | NM_139058 |
| ASCL3 | Achaete-scute complex (Drosophila) homolog- | NM_020646 |
| | like 3 | |
| ATAD2 | ATPase family, AAA domain containing 2 | NM_014109 |
| ATP1A4 | ATPase, Na+/K+ transporting, alpha 4 | NM_144699 |
| | polypeptide | |
| ATP6V1B2 | ATPase, H+ transporting, lysosomal 56/58 kDa, | NM_001693 |
| | V1 subunit B, isoform 2 | |
| AVPI1 | Arginine vasopressin-induced 1 | NM_021732 |
| AXL | AXL receptor tyrosine kinase | NM_001699 |
| B3GALT6 | UDP-Gal:betaGal beta 1,3-galactosyltransferase | NM_080605 |
| | polypeptide 6 | |
| B3GAT3 | Beta-1,3-glucuronyltransferase 3 | NM_012200 |
| | (glucuronosyltransferase I) | |
| B3GTL | Beta 3-glycosyltransferase-like | BC032021 |
| BAALC | Brain and acute leukemia, cytoplasmic | NM_024812 |
| BATF | Basic leucine zipper transcription factor, ATF- | NM_006399 |
| | like | |
| BCAR1 | Breast cancer anti-estrogen resistance 1 | NM_014567 |
| BCKDHB | Branched chain keto acid dehydrogenase E1, beta | NM_183050 |
| | polypeptide (maple syrup urine disease) | |
| BCL3 | B-cell CLL/lymphoma 3 | NM_005178 |
| BECN1 | Beclin 1 (coiled-coil, myosin-like BCL2 | NM_003766 |
| | interacting protein) | |
| BECN1 | Beclin 1 (coiled-coil, myosin-like BCL2 | NM_003766 |
| | interacting protein) | |
| BEXL1 | Brain expressed X-linked-like 1 | XM_043653 |
| BIRC1 | Baculoviral IAP repeat-containing 1 | NM_004536 |
| Bles03 | Basophilic leukemia expressed protein BLES03 | NM_031450 |
| BMP8A | Bone morphogenetic protein 8a | AK093659 |
| BNIP3L | BCL2/adenovirus E1B 19 kDa interacting protein | AF067396 |
| | 3-like | |
| BNIP3L | BCL2/adenovirus E1B 19 kDa interacting protein | NM_004331 |
| | 3-like | |
| BTN3A2 | Butyrophilin, subfamily 3, member A2 | NM_007047 |
| C10orf10 | Chromosome 10 open reading frame 10 | NM_007021 |
| C10orf119 | Chromosome 10 open reading frame 119 | NM_024834 |
| C10orf9 | Chromosome 10 open reading frame 9 | NM_145012 |
| C12orf14 | Chromosome 12 open reading frame 14 | NM_021238 |
| C14orf131 | Chromosome 14 open reading frame 131 | NM_018335 |
| C1orf24 | Chromosome 1 open reading frame 24 | NM_052966 |
| C1orf37 | Chromosome 1 open reading frame 37 | CR591805 |
| C1orf38 | Chromosome 1 open reading frame 38 | NM_004848 |
| C1orf42 | Chromosome 1 open reading frame 42 | NM_019060 |
| C20orf10 | Chromosome 20 open reading frame 10 | NM_014477 |
| C20orf22 | Chromosome 20 open reading frame 22 | NM_015600 |
| C4.4A | GPI-anchored metastasis-associated protein | NM_014400 |
| | homolog | |
| C5orf14 | Chromosome 5 open reading frame 14 | NM_024715 |
| C6orf27 | Chromosome 6 open reading frame 27 | NM_025258 |
| C6orf64 | Chromosome 6 open reading frame 64 | NM_018322 |
| C6orf80 | Chromosome 6 open reading frame 80 | NM_015439 |
| C7orf19 | Chromosome 7 open reading frame 19 | NM_032831 |
| C9orf61 | Chromosome 9 open reading frame 61 | NM_004816 |
| CABP7 | Calcium binding protein 7 | NM_182527 |
| CACNA2D1 | Calcium channel, voltage-dependent, alpha | NM_000722 |
| | 2/delta subunit 1 | |
| CACNG6 | Calcium channel, voltage-dependent, gamma | NM_145814 |
| | subunit 6 | |
| CAPN10 | Calpain 10 | NM_021251 |
| CAPS | Calcyphosine | NM_004058 |
| CASP4 | Caspase 4, apoptosis-related cysteine protease | NM_033307 |
| CASP5 | Caspase 5, apoptosis-related cysteine protease | NM_004347 |
| CAST | Calpastatin | NM_173060 |
| CAV2 | Caveolin 2 | NM_001233 |
| CBLL1 | Cas-Br-M (murine) ecotropic retroviral | NM_024814 |
| | transforming sequence-like 1 | |
| CBX8 | Chromobox homolog 8 (Pc class homolog, | NM_020649 |
| | Drosophila) | |
| CCDC6 | Coiled-coil domain containing 6 | S72869 |
| CCL2 | Chemokine (C-C motif) ligand 2 | NM_002982 |
| CCL4 | Chemokine (C-C motif) ligand 4 | NM_002984 |
| CCNG2 | Cyclin G2 | NM_004354 |
| CCNG2 | Cyclin G2 | NM_004354 |
| CCNT2 | Cyclin T2 | NM_058241 |
| CCR5 | Chemokine (C-C motif) receptor 5 | NM_000579 |
| CCT5 | Chaperonin containing TCP1, subunit 5 (epsilon) | NM_012073 |
| CD33 | CD33 antigen (gp67) | NM_001772 |
| CD86 | CD86 antigen (CD28 antigen ligand 2, B7-2 | NM_006889 |
| | antigen) | |
| CD8A | CD8 antigen, alpha polypeptide (p32) | NM_001768 |
| CDC26 | Cell division cycle 26 | NM_139286 |
| CDC37 | CDC37 cell division cycle 37 homolog (S. cerevisiae) | NM_007065 |
| CDC7 | CDC7 cell division cycle 7 (S. cerevisiae) | NM_003503 |
| CDK2AP1 | CDK2-associated protein 1 | NM_004642 |
| CDR1 | Cerebellar degeneration-related protein 1, 34 kDa | NM_004065 |
| CDT1 | DNA replication factor | NM_030928 |
| CDW52 | CDW52 antigen (CAMPATH-1 antigen) | NM_001803 |
| CEBPD | CCAAT/enhancer binding protein (C/EBP), delta | NM_005195 |
| CENPE | Centromere protein E, 312 kDa | NM_001813 |
| CFHL1 | Complement factor H-related 1 | NM_002113 |
| CGI-111 | CGI-111 protein | NM_016048 |
| CGI-90 | CGI-90 protein | NM_016033 |
| CISH | Cytokine inducible SH2-containing protein | NM_145071 |
| CKLFSF1 | Chemokine-like factor super family 1 | NM_181294 |
| CLDN6 | Claudin 6 | NM_021195 |
| CLIPR-59 | CLIP-170-related protein | BC013116 |
| CLYBL | Citrate lyase beta like | NM_138280 |
| CNFN | Cornifelin | NM_032488 |
| CNTN3 | Contactin 3 (plasmacytoma associated) | AB040929 |
| COL1A2 | Collagen, type I, alpha 2 | NM_000089 |
| COL6A2 | Collagen, type VI, alpha 2 | NM_001849 |
| COL6A3 | Collagen, type VI, alpha 3 | NM_057165 |
| COMMD2 | COMM domain containing 2 | NM_016094 |
| COTL1 | Coactosin-like 1 (Dictyostelium) | NM_021149 |
| COX5A | Cytochrome c oxidase subunit Va | AA129107 |
| CPA3 | Carboxypeptidase A3 (mast cell) | NM_001870 |
| CPNE5 | Copine V | NM_020939 |
| CPVL | Carboxypeptidase, vitellogenic-like | NM_019029 |
| CRBN | Cereblon | AF130117 |
| CREB3L3 | CAMP responsive element binding protein 3-like 3 | NM_032607 |
| CRLF1 | Cytokine receptor-like factor 1 | NM_004750 |
| CROC4 | Transcriptional activator of the c-fos promoter | NM_006365 |
| CRTAP | Cartilage associated protein | NM_006371 |
| CTAG1B | Cancer/testis antigen 1B | NM_139250 |
| CTAGE4 | CTAGE family, member 4 | XM_496933 |
| CTNNA1 | Catenin (cadherin-associated protein), alpha 1, | NM_001903 |
| | 102 kDa | |
| CTSC | Cathepsin C | NM_001814 |
| CTSH | Cathepsin H | NM_148979 |
| CUTL1 | Cut-like 1, CCAAT displacement protein | NM_181500 |
| | (Drosophila) | |
| CXCL5 | Chemokine (C—X—C motif) ligand 5 | NM_002994 |
| CYBRD1 | Cytochrome b reductase 1 | NM_024843 |
| CYP2R1 | Cytochrome P450, family 2, subfamily R, | NM_024514 |
| | polypeptide 1 | |
| CYP4V2 | Cytochrome P450, family 4, subfamily V, | NM_207352 |
| | polypeptide 2 | |
| DBN1 | Drebrin 1 | NM_004395 |
| DCAMKL1 | Doublecortin and CaM kinase-like 1 | NM_004734 |
| DCL-1 | Type I transmembrane C-type lectin receptor | NM_014880 |
| | DCL-1 | |
| DDX3Y | DEAD (Asp-Glu-Ala-Asp) box polypeptide 3, Y- | NM_004660 |
| | linked | |
| DDX58 | DEAD (Asp-Glu-Ala-Asp) box polypeptide 58 | NM_014314 |
| DDX6 | DEAD (Asp-Glu-Ala-Asp) box polypeptide 6 | AK021715 |
| DERP6 | S-phase 2 protein | NM_015362 |
| DIAPH2 | Diaphanous homolog 2 (Drosophila) | NM_006729 |
| DICER1 | Dicer1, Dcr-1 homolog (Drosophila) | NM_177438 |
| DIRC1 | Disrupted in renal carcinoma 1 | NM_052952 |
| DJ971N18.2 | Hypothetical protein DJ971N18.2 | NM_021156 |
| DJ971N18.2 | Hypothetical protein DJ971N18.2 | NM_021156 |
| DKFZp761C169 | Vasculin | CR621588 |
| DKK2 | Dickkopf homolog 2 (Xenopus laevis) | NM_014421 |
| DNCL1 | Dynein, cytoplasmic, light polypeptide 1 | NM_003746 |
| DPCD | Deleted in a mouse model of primary ciliary | AF264625 |
| | dyskinesia | |
| DPP3 | Dipeptidylpeptidase 3 | NM_005700 |
| DREV1 | DORA reverse strand protein 1 | NM_016025 |
| EBI2 | Epstein-Barr virus induced gene 2 (lymphocyte- | NM_004951 |
| | specific G protein-coupled receptor) | |
| ECHDC3 | Enoyl Coenzyme A hydratase domain containing 3 | NM_024693 |
| ECM2 | Extracellular matrix protein 2, female organ and | NM_001393 |
| | adipocyte specific | |
| EDG4 | Endothelial differentiation, lysophosphatidic acid | NM_004720 |
| | G-protein-coupled receptor, 4 | |
| EGFL3 | EGF-like-domain, multiple 3 | NM_001409 |
| EHD2 | EH-domain containing 2 | BC062554 |
| EIF3S3 | Eukaryotic translation initiation factor 3, subunit | NM_003756 |
| | 3 gamma, 40 kDa | |
| EIF3S7 | Eukaryotic translation initiation factor 3, subunit | NM_003753 |
| | 7 zeta, 66/67 kDa | |
| EIF3S8 | Eukaryotic translation initiation factor 3, subunit | NM_003752 |
| | 8, 110 kDa | |
| EIF4B | Eukaryotic translation initiation factor 4B | NM_001417 |
| ELA1 | Elastase 1, pancreatic | NM_001971 |
| EMB | Embigin homolog (mouse) | U52054 |
| EMCN | Endomucin | AL133118 |
| EMILIN2 | Elastin microfibril interfacer 2 | NM_032048 |
| EMR2 | Egf-like module containing, mucin-like, hormone | NM_152918 |
| | receptor-like 2 | |
| ENPP2 | Ectonucleotide | NM_006209 |
| | pyrophosphatase/phosphodiesterase 2 (autotaxin) | |
| EPB41L2 | Erythrocyte membrane protein band 4.1-like 2 | NM_001431 |
| ESM1 | Endothelial cell-specific molecule 1 | NM_007036 |
| ESPL1 | Extra spindle poles like 1 (S. cerevisiae) | NM_012291 |
| ESRRB | Estrogen-related receptor beta | NM_004452 |
| ET | Hypothetical protein ET | NM_024311 |
| EVI2B | Ecotropic viral integration site 2B | NM_006495 |
| EXOSC6 | Exosome component 6 | NM_058219 |
| F13A1 | Coagulation factor XIII, A1 polypeptide | NM_000129 |
| F7 | Coagulation factor VII (serum prothrombin | AF272774 |
| | conversion accelerator) | |
| FABP7 | Fatty acid binding protein 7, brain | NM_001446 |
| FAM12A | Family with sequence similarity 12, member A | NM_006683 |
| FAM20A | Family with sequence similarity 20, member A | NM_017565 |
| FAP | Fibroblast activation protein, alpha | NM_004460 |
| FBLN1 | Fibulin 1 | NM_006486 |
| FBLN2 | Fibulin 2 | NM_001998 |
| FCGR3A | Fc fragment of IgG, low affinity IIIb, receptor for | NM_000569 |
| | (CD16) | |
| FEM1A | Fem-1 homolog a (C. elegans) | NM_018708 |
| FER1L3 | Fer-1-like 3, myoferlin (C. elegans) | NM_133337 |
| FGF19 | Fibroblast growth factor 19 | NM_005117 |
| FGF5 | Fibroblast growth factor 5 | NM_004464 |
| FGL2 | Fibrinogen-like 2 | NM_006682 |
| FHL5 | Four and a half LIM domains 5 | NM_020482 |
| FKBP5 | FK506 binding protein 5 | NM_004117 |
| FKBP7 | FK506 binding protein 7 | NM_181342 |
| FKSG2 | Apoptosis inhibitor | NM_021631 |
| FLI1 | Friend leukemia virus integration 1 | NM_002017 |
| FLJ10647 | Hypothetical protein FLJ10647 | NM_018166 |
| FLJ10781 | Hypothetical protein FLJ10781 | NM_018215 |
| FLJ10902 | Hypothetical protein FLJ10902 | BC021277 |
| FLJ10986 | Hypothetical protein FLJ10986 | NM_018291 |
| FLJ11259 | Hypothetical protein FLJ11259 | NM_018370 |
| FLJ12363 | Hypothetical protein FLJ12363 | NM_032167 |
| FLJ12438 | Hypothetical protein FLJ12438 | NM_021933 |
| FLJ12443 | Hypothetical protein FLJ12443 | NM_024830 |
| FLJ12484 | Hypothetical protein FLJ12484 | NM_022767 |
| FLJ12572 | Hypothetical protein FLJ12572 | AF411456 |
| FLJ12748 | Hypothetical protein FLJ12748 | NM_024871 |
| FLJ20032 | Hypothetical protein FLJ20032 | AK000039 |
| FLJ20245 | Hypothetical protein FLJ20245 | NM_017723 |
| FLJ20701 | Hypothetical protein FLJ20701 | NM_017933 |
| FLJ21616 | Hypothetical protein FLJ21616 | NM_024567 |
| FLJ22573 | Hypothetical protein FLJ22573 | NM_024660 |
| FLJ23221 | Hypothetical protein FLJ23221 | NM_024579 |
| FLJ23861 | Hypothetical protein FLJ23861 | NM_152519 |
| FLJ25200 | Hypothetical protein FLJ25200 | NM_144715 |
| FLJ25222 | CXYorf1-related protein | NM_199163 |
| FLJ31882 | Hypothetical protein FLJ31882 | NM_152460 |
| FLJ32009 | Hypothetical protein FLJ32009 | NM_152718 |
| FLJ34969 | Hypothetical protein FLJ34969 | NM_152678 |
| FLJ35390 | Hypothetical protein FLJ35390 | XM_379820 |
| FLJ35757 | Hypothetical protein FLJ35757 | NM_152598 |
| FLJ35775 | Hypothetical protein FLJ35775 | NM_152418 |
| FLJ36748 | Hypothetical protein FLJ36748 | NM_152406 |
| FLJ36888 | Hypothetical protein FLJ36888 | NM_178830 |
| FLJ38379 | Hypothetical protein FLJ38379 | NM_178530 |
| FLJ39441 | Hypothetical protein FLJ39441 | NM_194285 |
| FLJ43339 | FLJ43339 protein | CR749408 |
| FLJ44896 | FLJ44896 protein | BQ189189 |
| FLJ90661 | Hypothetical protein FLJ90661 | NM_173502 |
| FN3KRP | Fructosamine-3-kinase-related protein | NM_024619 |
| FXN | Frataxin | NM_000144 |
| FXYD2 | FXYD domain containing ion transport regulator 2 | NM_021603 |
| FYB | FYN binding protein (FYB-120/130) | NM_001465 |
| FZR1 | Fizzy/cell division cycle 20 related 1 | NM_016263 |
| | (Drosophila) | |
| G1P2 | Interferon, alpha-inducible protein (clone IFI- | NM_005101 |
| | 15K) | |
| G1P3 | Interferon, alpha-inducible protein (clone IFI-6- | NM_022873 |
| | 16) | |
| GABPB2 | GA binding protein transcription factor, beta | BC009935 |
| | subunit 2, 47 kDa | |
| GABRA2 | Gamma-aminobutyric acid (GABA) A receptor, | NM_000807 |
| | alpha 2 | |
| GARNL4 | GTPase activating Rap/RanGAP domain-like 4 | NM_015085 |
| GATA2 | GATA binding protein 2 | NM_032638 |
| GBP1 | Guanylate binding protein 1, interferon-inducible, | NM_002053 |
| | 67 kDa | |
| GBP3 | Guanylate binding protein 3 | NM_018284 |
| GEM | GTP binding protein overexpressed in skeletal | NM_005261 |
| | muscle | |
| GFAP | Glial fibrillary acidic protein | NM_002055 |
| GH1 | Growth hormone 1 | NM_000515 |
| GHITM | Growth hormone inducible transmembrane | NM_014394 |
| | protein | |
| GHR | Growth hormone receptor | NM_000163 |
| GIMAP6 | GTPase, IMAP family member 6 | NM_024711 |
| GIT2 | G protein-coupled receptor kinase interactor 2 | NM_057170 |
| GK | Glycerol kinase | NM_203391 |
| GLIPR1 | GLI pathogenesis-related 1 (glioma) | NM_006851 |
| GLYAT | Glycine-N-acyltransferase | NM_005838 |
| GMFG | Glia maturation factor, gamma | NM_004877 |
| GPM6B | Glycoprotein M6B | NM_005278 |
| GPSM1 | G-protein signalling modulator 1 (AGS3-like, C. elegans) | AL117478 |
| GPT | Glutamic-pyruvate transaminase (alanine | NM_005309 |
| | aminotransferase) | |
| GPX7 | Glutathione peroxidase 7 | NM_015696 |
| GRINL1A | Glutamate receptor, ionotropic, N-methyl D- | AK074767 |
| | aspartate-like 1A | |
| GRIPAP1 | GRIP1 associated protein 1 | AB032993 |
| GSG2 | Haspin | AK056691 |
| GSPT2 | G1 to S phase transition 2 | NM_018094 |
| GSTM1 | Glutathione S-transferase M1 | NM_000561 |
| GSTM3 | Glutathione S-transferase M3 (brain) | NM_000849 |
| GSTT1 | Glutathione S-transferase theta 1 | NM_000853 |
| GSTT1 | Glutathione S-transferase theta 1 | NM_000853 |
| GSTT2 | Glutathione S-transferase theta 2 | NM_000854 |
| GSTT2 | Glutathione S-transferase theta 2 | NM_000854 |
| GTF3C5 | General transcription factor IIIC, polypeptide 5, | NM_012087 |
| | 63 kDa | |
| GTPBP5 | GTP binding protein 5 (putative) | NM_015666 |
| GTPBP6 | GTP binding protein 6 (putative) | NM_012227 |
| GZMH | Granzyme H (cathepsin G-like 2, protein h- | NM_033423 |
| | CCPX) | |
| GZMK | Granzyme K (serine protease, granzyme 3; | NM_002104 |
| | tryptase II) | |
| H1F0 | H1 histone family, member 0 | NM_005318 |
| HARS | Histidyl-tRNA synthetase | NM_002109 |
| HAVCR2 | Hepatitis A virus cellular receptor 2 | NM_032782 |
| HCLS1 | Hematopoietic cell-specific Lyn substrate 1 | NM_005335 |
| HELB | Helicase (DNA) B | NM_033647 |
| HEPH | Hephaestin | NM_138737 |
| HERPUD1 | Homocysteine-inducible, endoplasmic reticulum | NM_014685 |
| | stress-inducible, ubiquitin-like domain member 1 | |
| HLA-A | Major histocompatibility complex, class I, A | BC020891 |
| HLA-B | Major histocompatibility complex, class I, B | NM_005514 |
| HLA-DMA | Major histocompatibility complex, class II, DM | NM_006120 |
| | alpha | |
| HLA-DOA | Major histocompatibility complex, class II, DO | M38054 |
| | alpha | |
| HLA-DOA | Major histocompatibility complex, class II, DO | NM_002119 |
| | alpha | |
| HLA-DPA1 | Major histocompatibility complex, class II, DP | NM_033554 |
| | alpha 1 | |
| HLA-DPB1 | Major histocompatibility complex, class II, DP | NM_002121 |
| | beta 1 | |
| HLA-DQA1 | Major histocompatibility complex, class II, DQ | NM_002122 |
| | alpha 1 | |
| HLA-DQA2 | Major histocompatibility complex, class II, DQ | NM_020056 |
| | alpha 2 | |
| HLA-DQB1 | Major histocompatibility complex, class II, DQ | M20432 |
| | beta 1 | |
| HLA-DRB1 | Major histocompatibility complex, class II, DR | NM_002124 |
| | beta 4 | |
| HLA-DRB5 | Major histocompatibility complex, class II, DR | NM_002125 |
| | beta 4 | |
| HLA-E | Major histocompatibility complex, class I, E | NM_005516 |
| HLA-E | Major histocompatibility complex, class I, E | NM_005516 |
| HLA-E | Major histocompatibility complex, class I, E | NM_005516 |
| HLA-F | Major histocompatibility complex, class I, F | NM_018950 |
| HLA-G | HLA-G histocompatibility antigen, class I, G | NM_002127 |
| HOXB4 | Homeo box B4 | NM_024015 |
| HPS3 | Hermansky-Pudlak syndrome 3 | NM_032383 |
| HRAS | V-Ha-ras Harvey rat sarcoma viral oncogene | NM_176795 |
| | homolog | |
| HSPBP1 | Hsp70-interacting protein | NM_012267 |
| ICAM2 | Intercellular adhesion molecule 2 | NM_000873 |
| IFI16 | Interferon, gamma-inducible protein 16 | BC017059 |
| IFI16 | Interferon, gamma-inducible protein 16 | NM_005531 |
| IFIT1 | Interferon-induced protein with tetratricopeptide | NM_001001887 |
| | repeats 1 | |
| IFIT2 | Interferon-induced protein with tetratricopeptide | NM_001547 |
| | repeats 2 | |
| IFITM1 | Interferon induced transmembrane protein 1 (9- | NM_003641 |
| | 27) | |
| IFITM2 | Interferon induced transmembrane protein 2 (1- | NM_006435 |
| | 8D) | |
| IFITM3 | Interferon induced transmembrane protein 3 (1- | NM_021034 |
| | 8U) | |
| IFNA6 | Interferon, alpha 6 | NM_021002 |
| IGFBP5 | Insulin-like growth factor binding protein 5 | NM_000599 |
| IGH@ | Immunoglobulin heavy locus | BC040042 |
| IGHG4 | Immunoglobulin heavy constant gamma 4 (G4m | BC025985 |
| | marker) | |
| IGKC | Immunoglobulin kappa constant | AJ399872 |
| IGKC | Immunoglobulin kappa constant | BC030813 |
| IGLL1 | Immunoglobulin lambda-like polypeptide 1 | NM_152855 |
| IGLL1 | Immunoglobulin lambda-like polypeptide 1 | NM_152855 |
| IKBKG | Inhibitor of kappa light polypeptide gene | NM_003639 |
| | enhancer in B-cells, kinase gamma | |
| IL10RA | Interleukin 10 receptor, alpha | NM_001558 |
| IL13RA1 | Interleukin 13 receptor, alpha 1 | NM_001560 |
| IL15 | Interleukin 15 | NM_172175 |
| IL23A | Interleukin 23, alpha subunit p19 | NM_016584 |
| IL27 | Interleukin 27 | NM_145659 |
| INDO | Indoleamine-pyrrole 2,3 dioxygenase | NM_002164 |
| INSIG1 | Insulin induced gene 1 | NM_005542 |
| IQCF2 | IQ motif containing F2 | NM_203424 |
| IRF5 | Interferon regulatory factor 5 | NM_032643 |
| IRF7 | Interferon regulatory factor 7 | NM_004030 |
| IRX3 | Iroquois homeobox protein 3 | NM_024336 |
| IRX5 | Iroquois homeobox protein 5 | NM_005853 |
| ITGAL | Integrin, alpha L (antigen CD11A (p180), | NM_002209 |
| | lymphocyte function-associated antigen 1; alpha | |
| | polypeptide) | |
| ITGB1 | Integrin, beta 1 (fibronectin receptor, beta | NM_033666 |
| | polypeptide, antigen CD29 includes MDF2, | |
| | MSK12) | |
| ITGB1BP1 | Integrin beta 1 binding protein 1 | NM_004763 |
| ITGB2 | Integrin, beta 2 (antigen CD18 (p95), lymphocyte | NM_000211 |
| | function-associated antigen 1; macrophage | |
| | antigen 1 (mac-1) beta subunit) | |
| ITLN1 | Intelectin 1 (galactofuranose binding) | NM_017625 |
| KAZALD1 | Kazal-type serine protease inhibitor domain 1 | NM_030929 |
| KCNK4 | Potassium channel, subfamily K, member 4 | NM_016611 |
| KCNS3 | Potassium voltage-gated channel, delayed- | NM_002252 |
| | rectifier, subfamily S, member 3 | |
| KCTD10 | Potassium channel tetramerisation domain | NM_031954 |
| | containing 10 | |
| KCTD15 | Potassium channel tetramerisation domain | NM_024076 |
| | containing 15 | |
| KEL | Kell blood group | NM_000420 |
| KIAA0063 | KIAA0063 gene product | NM_014876 |
| KIAA0232 | KIAA0232 gene product | NM_014743 |
| KIAA0467 | KIAA0467 protein | NM_015284 |
| KIAA0494 | KIAA0494 gene product | NM_014774 |
| KIAA0562 | Glycine-, glutamate-, | NM_014704 |
| | thienylcyclohexylpiperidine-binding protein | |
| KIAA0664 | KIAA0664 protein | NM_015229 |
| KIAA0676 | KIAA0676 protein | NM_015043 |
| KIAA0870 | KIAA0870 protein | NM_014957 |
| KIAA1190 | Hypothetical protein KIAA1190 | NM_145166 |
| KIAA1463 | KIAA1463 protein | NM_173602 |
| KIAA1509 | KIAA1509 | XM_029353 |
| KIAA1609 | KIAA1609 protein | NM_020947 |
| KIAA1666 | KIAA1666 protein | XM_371429 |
| KIAA1683 | KIAA1683 | NM_025249 |
| KIF25 | Kinesin family member 25 | NM_005355 |
| KLF9 | Kruppel-like factor 9 | NM_001206 |
| KLHL18 | Kelch-like 18 (Drosophila) | AB018338 |
| KLK2 | Kallikrein 2, prostatic | NM_005551 |
| KRT20 | Keratin 20 | NM_019010 |
| LAMB1 | Laminin, beta 1 | NM_002291 |
| LAMP2 | Lysosomal-associated membrane protein 2 | NM_013995 |
| LAMR1P15 | Laminin receptor 1 pseudogene 15 | AF284768 |
| LCP1 | Lymphocyte cytosolic protein 1 (L-plastin) | NM_002298 |
| LDLR | Low density lipoprotein receptor (familial | M28219 |
| | hypercholesterolemia) | |
| LEPR | Leptin receptor | NM_017526 |
| LEPROTL1 | Leptin receptor overlapping transcript-like 1 | AF359269 |
| LGALS2 | Lectin, galactoside-binding, soluble, 2 (galectin | NM_006498 |
| | 2) | |
| LGALS8 | Lectin, galactoside-binding, soluble, 8 (galectin | NM_201543 |
| | 8) | |
| LGALS9 | Lectin, galactoside-binding, soluble, 9 (galectin | NM_002308 |
| | 9) | |
| LHFP | Lipoma HMGIC fusion partner | NM_005780 |
| LILRB2 | Leukocyte immunoglobulin-like receptor, | NM_005874 |
| | subfamily B (with TM and ITIM domains), | |
| | member 2 | |
| LILRB5 | Leukocyte immunoglobulin-like receptor, | NM_006840 |
| | subfamily B (with TM and ITIM domains), | |
| | member 5 | |
| LMO2 | LIM domain only 2 (rhombotin-like 1) | NM_005574 |
| LMOD1 | Leiomodin 1 (smooth muscle) | AW939148 |
| LMOD1 | Leiomodin 1 (smooth muscle) | NM_012134 |
| LOC114990 | Vasorin | NM_138440 |
| LOC123876 | Xenobiotic/medium-chain fatty acid:CoA ligase | NM_182617 |
| LOC128977 | Hypothetical protein LOC128977 | NM_173793 |
| LOC142678 | Skeletrophin | NM_080875 |
| LOC147645 | Hypothetical protein LOC147645 | XM_085831 |
| LOC153561 | Hypothetical LOC389295 | NM_207331 |
| LOC255458 | Hypothetical protein LOC255458 | BC009038 |
| LOC283464 | Hypothetical protein LOC283464 | XM_290597 |
| LOC284323 | Hypothetical protein LOC284323 | AK091274 |
| LOC339834 | Hypothetical protein LOC339834 | NM_178173 |
| LOC387680 | Similar to KIAA0592 protein | NM_001005751 |
| LOC387763 | Hypothetical LOC387763 | XM_373497 |
| LOC400027 | Hypothetical gene supported by BC047417 | XM_378350 |
| LOC400581 | GRB2-related adaptor protein-like | BC026233 |
| LOC400759 | Similar to Interferon-induced guanylate-binding | XM_375747 |
| | protein 1 (GTP-binding protein 1) (Guanine | |
| | nucleotide-binding protein 1) (HuGBP-1) | |
| LOC401565 | Similar to 4931415M17 protein | NM_001001710 |
| LOC441245 | Hypothetical LOC441245 | XM_496889 |
| LOC493869 | Similar to RIKEN cDNA 2310016C16 | AK022110 |
| LOC51035 | ORF | NM_015853 |
| LOC87769 | Hypothetical protein BC004360 | XM_373431 |
| LOC91689 | Hypothetical gene supported by AL449243 | NM_033318 |
| LPXN | Leupaxin | NM_004811 |
| LRAP | Leukocyte-derived arginine aminopeptidase | NM_022350 |
| LRBA | LPS-responsive vesicle trafficking, beach and | NM_006726 |
| | anchor containing | |
| LRRC14 | Leucine rich repeat containing 14 | NM_014665 |
| LRRC2 | Leucine rich repeat containing 2 | NM_024512 |
| LRRIQ2 | Leucine-rich repeats and IQ motif containing 2 | NM_024548 |
| LTBP4 | Latent transforming growth factor beta binding | AF051344 |
| | protein 4 | |
| LTBP4 | Latent transforming growth factor beta binding | NM_003573 |
| | protein 4 | |
| LUM | Lumican | NM_002345 |
| LY6K | Lymphocyte antigen 6 complex, locus K | NM_017527 |
| LY6K | Lymphocyte antigen 6 complex, locus K | NM_017527 |
| LYZ | Lysozyme (renal amyloidosis) | NM_000239 |
| MAB21L2 | Mab-21-like 2 (C. elegans) | NM_006439 |
| MAC30 | Hypothetical protein MAC30 | NM_014573 |
| MAFB | V-maf musculoaponeurotic fibrosarcoma | NM_005461 |
| | oncogene homolog B (avian) | |
| MAGEH1 | Melanoma antigen, family H, 1 | NM_014061 |
| MAN2B2 | Mannosidase, alpha, class 2B, member 2 | NM_015274 |
| MARCH-II | Membrane-associated RING-CH protein II | NM_016496 |
| MARCKS | Myristoylated alanine-rich protein kinase C | NM_002356 |
| | substrate | |
| MCCC1 | Methylcrotonoyl-Coenzyme A carboxylase 1 | NM_020166 |
| | (alpha) | |
| MCCC2 | Methylcrotonoyl-Coenzyme A carboxylase 2 | AK001948 |
| | (beta) | |
| ME2 | Malic enzyme 2, NAD(+)-dependent, | BC000147 |
| | mitochondrial | |
| MED19 | Mediator of RNA polymerase II transcription, | NM_153450 |
| | subunit 19 homolog (yeast) | |
| MEGF10 | MEGF10 protein | BC020198 |
| MERTK | C-mer proto-oncogene tyrosine kinase | U08023 |
| MFAP5 | Microfibrillar associated protein 5 | NM_003480 |
| MFNG | Manic fringe homolog (Drosophila) | NM_002405 |
| MGC10772 | Hypothetical protein MGC10772 | NM_030567 |
| MGC11308 | Hypothetical protein MGC11308 | NM_032889 |
| MGC13186 | Hypothetical protein MGC13186 | NM_032324 |
| MGC15523 | Hypothetical protein MGC15523 | BC020925 |
| MGC15875 | Hypothetical protein MGC15875 | AK090397 |
| MGC16044 | Hypothetical protein MGC16044 | NM_138371 |
| MGC16075 | Hypothetical protein MGC16075 | XM_498434 |
| MGC21654 | Unknown MGC21654 product | NM_145647 |
| MGC23918 | Hypothetical protein MGC23918 | NM_144716 |
| MGC24133 | Hypothetical protein MGC24133 | NM_174896 |
| MGC27165 | Hypothetical protein MGC27165 | AK027379 |
| MGC29784 | Hypothetical protein MGC29784 | NM_173659 |
| MGC29937 | Hypothetical protein MGC29937 | NM_144597 |
| MGC3169 | Hypothetical protein MGC3169 | NM_024074 |
| MGC3200 | Hypothetical protein LOC284615 | NM_032305 |
| MGC33839 | Hypothetical protein MGC33839 | NM_152353 |
| MGC35045 | Chromosome 19 open reading frame 16 | AL834316 |
| MGC35048 | Hypothetical protein MGC35048 | NM_153208 |
| MGC35212 | Hypothetical protein MGC35212 | NM_152764 |
| MGC39584 | Hypothetical gene supported by BC029568 | BC029568 |
| MGC42157 | Hypothetical locus MGC42157 | XM_499573 |
| MGC4293 | Hypothetical protein MGC4293 | NM_031304 |
| MGC45428 | Hypothetical protein MGC45428 | NM_152619 |
| MGC45780 | Hypothetical protein MGC45780 | NM_173833 |
| MGC8721 | Hypothetical protein MGC8721 | NM_016127 |
| MGC9515 | Hypothetical protein MGC9515 | BC036263 |
| MICB | MHC class I polypeptide-related sequence B | NM_005931 |
| MIS12 | MIS12 homolog (yeast) | NM_024039 |
| MKRN1 | Makorin, ring finger protein, 1 | NM_013446 |
| MLL5 | Myeloid/lymphoid or mixed-lineage leukemia 5 | NM_182931 |
| | (trithorax homolog, Drosophila) | |
| MNS1 | Meiosis-specific nuclear structural protein 1 | NM_018365 |
| MOBKL2A | MOB1, Mps One Binder kinase activator-like 2A | AK024373 |
| | (yeast) | |
| MOGAT3 | Monoacylglycerol O-acyltransferase 3 | NM_178176 |
| MPEG1 | Macrophage expressed gene 1 | AK074166 |
| MPP1 | Membrane protein, palmitoylated 1, 55 kDa | NM_002436 |
| MPP2 | Membrane protein, palmitoylated 2 (MAGUK | NM_005374 |
| | p55 subfamily member 2) | |
| MPPE1 | Metallophosphoesterase 1 | NM_138608 |
| MPZ | Myelin protein zero (Charcot-Marie-Tooth | NM_000530 |
| | neuropathy 1B) | |
| MRC1 | Mannose receptor, C type 1 | NM_002438 |
| MRCL3 | Myosin regulatory light chain MRCL3 | NM_006471 |
| MRPL43 | Mitochondrial ribosomal protein L43 | NM_176794 |
| MRPL46 | Mitochondrial ribosomal protein L46 | NM_022163 |
| MS4A6A | Membrane-spanning 4-domains, subfamily A, | NM_022349 |
| | member 6A | |
| MSN | Moesin | NM_002444 |
| MT | Malonyl-CoA:acyl carrier protein transacylase, | NM_014507 |
| | mitochondrial | |
| MT1A | Metallothionein 1A (functional) | NM_005946 |
| MT1E | Metallothionein 1E (functional) | NM_175617 |
| MT1H | Metallothionein 1H | NM_005951 |
| MT1J | Metallothionein 1J | NM_175622 |
| MT1K | Metallothionein 1K | NM_176870 |
| MT1L | Metallothionein 1L | X97261 |
| MT1X | Metallothionein 1X | BC032338 |
| MT1X | Metallothionein 1X | NM_005952 |
| MT1X | Metallothionein 1X | NM_005952 |
| MT2A | Metallothionein 2A | BC007034 |
| MT2A | Metallothionein 2A | NM_005953 |
| MT2A | Metallothionein 2A | NM_005953 |
| MTCBP-1 | Membrane-type 1 matrix metalloproteinase | NM_018269 |
| | cytoplasmic tail binding protein-1 | |
| MTCH2 | Mitochondrial carrier homolog 2 (C. elegans) | NM_014342 |
| MTRF1L | Mitochondrial translational release factor 1-like | NM_019041 |
| MUC20 | Mucin 20 | NM_152673 |
| MUC3A | Mucin 3A, intestinal | M55405 |
| MX1 | Myxovirus (influenza virus) resistance 1, | NM_002462 |
| | interferon-inducible protein p78 (mouse) | |
| MYO1B | Myosin IB | NM_012223 |
| MYOC | Myocilin, trabecular meshwork inducible | NM_000261 |
| | glucocorticoid response | |
| NAP1L4 | Nucleosome assembly protein 1-like 4 | NM_005969 |
| NCKAP1 | NCK-associated protein 1 | NM_205842 |
| NFE2L3 | Nuclear factor (erythroid-derived 2)-like 3 | NM_004289 |
| NFYC | Nuclear transcription factor Y, gamma | NM_014223 |
| NICN1 | Nicolin 1 | NM_032316 |
| NINJ1 | Ninjurin 1 | NM_004148 |
| NIPSNAP3B | Nipsnap homolog 3B (C. elegans) | NM_018376 |
| NISCH | Nischarin | NM_007184 |
| NNMT | Nicotinamide N-methyltransferase | NM_006169 |
| NOL6 | Nucleolar protein family 6 (RNA-associated) | NM_130793 |
| NOSIP | Nitric oxide synthase interacting protein | NM_015953 |
| NPTX1 | Neuronal pentraxin I | NM_002522 |
| NUDT2 | Nudix (nucleoside diphosphate linked moiety X)- | NM_001161 |
| | type motif 2 | |
| NUP62 | Nucleoporin 62 kDa | NM_172374 |
| NXPH4 | Neurexophilin 4 | NM_007224 |
| NYREN18 | NEDD8 ultimate buster-1 | BC034716 |
| OAS3 | 2′-5′-oligoadenylate synthetase 3, 100 kDa | NM_006187 |
| OAS3 | 2′-5′-oligoadenylate synthetase 3, 100 kDa | NM_006187 |
| OCA2 | Oculocutaneous albinism II (pink-eye dilution | NM_000275 |
| | homolog, mouse) | |
| OGDHL | Oxoglutarate dehydrogenase-like | NM_018245 |
| OPLAH | 5-oxoprolinase(ATP-hydrolysing) | NM_017570 |
| OPRK1 | Opioid receptor, kappa 1 | NM_000912 |
| OPTN | Optineurin | NM_021980 |
| OSR2 | Odd-skipped related 2 (Drosophila) | NM_053001 |
| OSTbeta | Organic solute transporter beta | NM_178859 |
| P8 | P8 protein (candidate of metastasis 1) | NM_012385 |
| PAG | Phosphoprotein associated with | NM_018440 |
| | glycosphingolipid-enriched microdomains | |
| PAM | Peptidylglycine alpha-amidating monooxygenase | NM_000919 |
| PAX8 | Paired box gene 8 | AK056052 |
| PBXIP1 | Pre-B-cell leukemia transcription factor | NM_020524 |
| | interacting protein 1 | |
| PCNT2 | Pericentrin 2 (kendrin) | NM_006031 |
| PCOLCE2 | Procollagen C-endopeptidase enhancer 2 | NM_013363 |
| PDGFC | Platelet derived growth factor C | NM_016205 |
| PDGFRA | Platelet-derived growth factor receptor, alpha | NM_006206 |
| | polypeptide | |
| PDGFRL | Platelet-derived growth factor receptor-like | NM_006207 |
| PDK4 | Pyruvate dehydrogenase kinase, isoenzyme 4 | NM_002612 |
| PDZK1 | PDZ domain containing 1 | NM_002614 |
| PERLD1 | Per1-like domain containing 1 | NM_033419 |
| PEX19 | Peroxisomal biogenesis factor 19 | NM_002857 |
| PGM1 | Phosphoglucomutase 1 | NM_002633 |
| PGRMC1 | Progesterone receptor membrane component 1 | NM_006667 |
| PHAX | RNA U, small nuclear RNA export adaptor | AF086448 |
| | (phosphorylation regulated) | |
| PHCA | Phytoceramidase, alkaline | NM_018367 |
| PIP | Prolactin-induced protein | NM_002652 |
| PITPNC1 | Phosphatidylinositol transfer protein, cytoplasmic 1 | NM_012417 |
| PKM2 | Pyruvate kinase, muscle | NM_182471 |
| PKP2 | Plakophilin 2 | X97675 |
| PLAU | Plasminogen activator, urokinase | NM_002658 |
| PMP22 | Peripheral myelin protein 22 | NM_000304 |
| PNPLA4 | Patatin-like phospholipase domain containing 4 | NM_004650 |
| POLD4 | Polymerase (DNA-directed), delta 4 | NM_021173 |
| POLR2L | Polymerase (RNA) II (DNA directed) | NM_021128 |
| | polypeptide L, 7.6 kDa | |
| POU2F1 | POU domain, class 2, transcription factor 1 | S66901 |
| PP3856 | Similar to CG3714 gene product | NM_145201 |
| PPAP2B | Phosphatidic acid phosphatase type 2B | NM_003713 |
| PPFIA4 | Protein tyrosine phosphatase, receptor type, f | NM_015053 |
| | polypeptide (PTPRF), interacting protein (liprin), | |
| | alpha 4 | |
| PPIC | Peptidylprolyl isomerase C (cyclophilin C) | NM_000943 |
| PPIC | Peptidylprolyl isomerase C (cyclophilin C) | NM_000943 |
| PPIL3 | Peptidylprolyl isomerase (cyclophilin)-like 3 | NM_131916 |
| PPM1F | Protein phosphatase 1F (PP2C domain | NM_014634 |
| | containing) | |
| PRAC | Small nuclear protein PRAC | NM_032391 |
| PREB | Prolactin regulatory element binding | BE395450 |
| PRIC285 | Peroxisomal proliferator-activated receptor A | NM_033405 |
| | interacting complex 285 | |
| PRKD2 | Protein kinase D2 | NM_016457 |
| PRKY | Protein kinase, Y-linked | NM_002760 |
| PRSS15 | Protease, serine, 15 | NM_004793 |
| PSMA5 | Protpeeasome (prosome, macropain) subunit, alpha | NM_002790 |
| | type, 5 | |
| PSMB9 | Proteasome (prosome, macropain) subunit, beta | NM_148954 |
| | type, 9 (large multifunctional protease 2) | |
| PSMD11 | Proteasome (prosome, macropain) 26S subunit, | NM_002815 |
| | non-ATPase, 11 | |
| PSORS1C1 | Psoriasis susceptibility 1 candidate 1 | NM_014068 |
| PSPH | Phosphoserine phosphatase | NM_004577 |
| PSPHL | Phosphoserine phosphatase-like | AJ001612 |
| PTAFR | Platelet-activating factor receptor | S52624 |
| PTGIS | Prostaglandin I2 (prostacyclin) synthase | NM_000961 |
| PTOV1 | Prostate tumor overexpressed gene 1 | NM_017432 |
| PTP4A3 | Protein tyrosine phosphatase type IVA, member 3 | NM_007079 |
| PTPRC | Protein tyrosine phosphatase, receptor type, C | NM_080922 |
| PVRL2 | Poliovirus receptor-related 2 (herpesvirus entry | NM_002856 |
| | mediator B) | |
| PXMP2 | Peroxisomal membrane protein 2, 22 kDa | NM_018663 |
| R30953_1 | Interferon inducible GTPase 5 | NM_019612 |
| RAB15 | RAB15, member RAS onocogene family | NM_198686 |
| RABEP1 | Rabaptin, RAB GTPase binding effector protein 1 | NM_004703 |
| RAC2 | Ras-related C3 botulinum toxin substrate 2 (rho | NM_002872 |
| | family, small GTP binding protein Rac2) | |
| RAC2 | Ras-related C3 botulinum toxin substrate 2 (rho | NM_002872 |
| | family, small GTP binding protein Rac2) | |
| RAD51AP1 | RAD51 associated protein 1 | NM_006479 |
| RAI16 | Retinoic acid induced 16 | NM_022749 |
| RAPH1 | Ras association (RalGDS/AF-6) and pleckstrin | NM_213589 |
| | homology domains 1 | |
| RECK | Reversion-inducing-cysteine-rich protein with | NM_021111 |
| | kazal motifs | |
| RGL2 | Ral guanine nucleotide dissociation stimulator- | NM_004761 |
| | like 2 | |
| RGS10 | Regulator of G-protein signalling 10 | NM_001005339 |
| RGS11 | Regulator of G-protein signalling 11 | BC040504 |
| RGS16 | Regulator of G-protein signalling 16 | NM_002928 |
| RGS5 | Regulator of G-protein signalling 5 | NM_003617 |
| RHBDF1 | Rhomboid family 1 (Drosophila) | NM_022450 |
| RHOT2 | Ras homolog gene family, member T2 | NM_138769 |
| RIMS3 | Regulating synaptic membrane exocytosis 3 | NM_014747 |
| RIP | RPA interacting protein | NM_032308 |
| RIPK2 | Receptor-interacting serine-threonine kinase 2 | NM_003821 |
| RLN3 | Relaxin 3 | NM_080864 |
| RNASE4 | Angiogenin, ribonuclease, RNase A family, 5 | NM_001145 |
| RNASE4 | Angiogenin, ribonuclease, RNase A family, 5 | NM_194431 |
| RNF121 | Ring finger protein 121 | AK023139 |
| RNF125 | Ring finger protein 125 | NM_017831 |
| RNF13 | Ring finger protein 13 | NM_007282 |
| RNF138P1 | Ring finger protein 138 pseudogene 1 | AW975013 |
| RNF146 | Ring finger protein 146 | NM_030963 |
| RNF19 | Ring finger protein 19 | NM_183419 |
| ROBO1 | Roundabout, axon guidance receptor, homolog 1 | NM_002941 |
| | (Drosophila) | |
| ROBO3 | Roundabout, axon guidance receptor, homolog 3 | NM_022370 |
| | (Drosophila) | |
| RPL10A | Ribosomal protein L10a | NM_007104 |
| RPL41 | Ribosomal protein L41 | NM_021104 |
| RPL7A | Ribosomal protein L7a | NM_000972 |
| RPS10 | Ribosomal protein S10 | NM_001014 |
| RPS16 | Ribosomal protein S16 | NM_001020 |
| RPS18 | Ribosomal protein S18 | NM_022551 |
| RPS4X | Ribosomal protein S4, X-linked | NM_001007 |
| RPS4Y1 | Ribosomal protein S4, Y-linked 1 | NM_001008 |
| RPS4Y2 | Ribosomal protein S4, Y-linked 2 | NM_138963 |
| RRAGD | Ras-related GTP binding D | NM_021244 |
| RSAFD1 | Radical S-adenosyl methionine and flavodoxin | NM_018264 |
| | domains 1 | |
| RTN4 | Reticulon 4 | NM_153828 |
| RUTBC3 | RUN and TBC1 domain containing 3 | NM_015705 |
| S100P | S100 calcium binding protein P | NM_005980 |
| SAMD10 | Sterile alpha motif domain containing 10 | NM_080621 |
| SARA1 | SAR1a gene homolog 1 (S. cerevisiae) | NM_020150 |
| SARA1 | SAR1a gene homolog 1 (S. cerevisiae) | NM_020150 |
| SAT | Spermidine/spermine N1-acetyltransferase | NM_002970 |
| SAV1 | Salvador homolog 1 (Drosophila) | NM_021818 |
| SCAP | SREBP CLEAVAGE-ACTIVATING PROTEIN | NM_012235 |
| SCGB1D1 | Secretoglobin, family 1D, member 1 | NM_006552 |
| SCGB2A1 | Secretoglobin, family 2A, member 1 | NM_002407 |
| SCUBE3 | Signal peptide, CUB domain, EGF-like 3 | NM_152753 |
| SDK1 | Sidekick homolog 1 (chicken) | AF052150 |
| SECP43 | TRNA selenocysteine associated protein | NM_017846 |
| SECTM1 | Secreted and transmembrane 1 | NM_003004 |
| SEMA3B | Sema domain, immunoglobulin domain (Ig), | NM_004636 |
| | short basic domain, secreted, (semaphorin) 3B | |
| SERPINB2 | Serine (or cysteine) proteinase inhibitor, clade B | BC012609 |
| | (ovalbumin), member 2 | |
| SESN1 | Sestrin 1 | NM_014454 |
| SESN2 | Sestrin 2 | NM_031459 |
| SF4 | Splicing factor 4 | NM_172231 |
| SGCA | Sarcoglycan, alpha (50 kDa dystrophin-associated | NM_000023 |
| | glycoprotein) | |
| SH3BGRL | SH3 domain binding glutamic acid-rich protein | NM_003022 |
| | like | |
| SH3GLB1 | SH3-domain GRB2-like endophilin B1 | NM_016009 |
| SH3GLB2 | SH3-domain GRB2-like endophilin B2 | NM_020145 |
| SH3RF2 | SH3 domain containing ring finger 2 | NM_152550 |
| ShrmL | Shroom-related protein | NM_020859 |
| SIRPB2 | Signal-regulatory protein beta 2 | NM_018556 |
| SLAMF9 | SLAM family member 9 | NM_033438 |
| SLC10A3 | Solute carrier family 10 (sodium/bile acid | NM_019848 |
| | cotransporter family), member 3 | |
| SLC12A2 | Solute carrier family 12 | NM_001046 |
| | (sodium/potassium/chloride transporters), | |
| | member 2 | |
| SLC12A9 | Solute carrier family 12 (potassium/chloride | NM_020246 |
| | transporters), member 9 | |
| SLC14A1 | Solute carrier family 14 (urea transporter), | L36121 |
| | member 1 (Kidd blood group) | |
| SLC20A1 | Solute carrier family 20 (phosphate transporter), | NM_005415 |
| | member 1 | |
| SLC39A14 | Solute carrier family 39 (zinc transporter), | BC000068 |
| | member 14 | |
| SLC6A15 | Solute carrier family 6 (neurotransmitter | NM_018057 |
| | transporter), member 15 | |
| SLC7A1 | Solute carrier family 7 (cationic amino acid | NM_003045 |
| | transporter, y+ system), member 1 | |
| SLC7A7 | Solute carrier family 7 (cationic amino acid | NM_003982 |
| | transporter, y+ system), member 7 | |
| SLC9A3R2 | Solute carrier family 9 (sodium/hydrogen | NM_004785 |
| | exchanger), isoform 3 regulator 2 | |
| SLC9A9 | Solute carrier family 9 (sodium/hydrogen | NM_173653 |
| | exchanger), isoform 9 | |
| SLCO2B1 | Solute carrier organic anion transporter family, | NM_007256 |
| | member 2B1 | |
| SLPI | Secretory leukocyte protease inhibitor | NM_003064 |
| | (antileukoproteinase) | |
| SLPI | Secretory leukocyte protease inhibitor | NM_003064 |
| | (antileukoproteinase) | |
| SMAD1 | SMAD, mothers against DPP homolog 1 | NM_005900 |
| | (Drosophila) | |
| SMAP1 | Stromal membrane-associated protein 1 | NM_021940 |
| SMARCA4 | SWI/SNF related, matrix associated, actin | NM_003072 |
| | dependent regulator of chromatin, subfamily a, | |
| | member 4 | |
| SMARCE1 | SWI/SNF related, matrix associated, actin | NM_003079 |
| | dependent regulator of chromatin, subfamily e, | |
| | member 1 | |
| SMC5L1 | SMC5 structural maintenance of chromosomes 5- | NM_015110 |
| | like 1 (yeast) | |
| SMN2 | Survival of motor neuron 1, telomeric | NM_022877 |
| SMP1 | NPD014 protein | NM_014313 |
| SMTN | Smoothelin | NM_134269 |
| SNTG2 | Syntrophin, gamma 2 | NM_018968 |
| SNX7 | Sorting nexin 7 | NM_015976 |
| SOCS5 | Suppressor of cytokine signaling 5 | NM_014011 |
| SORD | Sorbitol dehydrogenase | NM_003104 |
| SP1 | Sp1 transcription factor | NM_138473 |
| SPARC | Secreted protein, acidic, cysteine-rich | NM_003118 |
| | (osteonectin) | |
| SRD5A2L | Steroid 5 alpha-reductase 2-like | NM_024592 |
| SRGAP3 | SLIT-ROBO Rho GTPase activating protein 3 | AF086321 |
| SRPK2 | SFRS protein kinase 2 | NM_182691 |
| SSB3 | SPRY domain-containing SOCS box protein | NM_080861 |
| | SSB-3 | |
| SSPN | Sarcospan (Kras oncogene-associated gene) | NM_005086 |
| STAT6 | Signal transducer and activator of transcription 6, | NM_003153 |
| | interleukin-4 induced | |
| STX7 | Syntaxin 7 | NM_003569 |
| SULF1 | Sulfatase 1 | NM_015170 |
| SUMF1 | Sulfatase modifying factor 1 | NM_182760 |
| SYAP1 | Synapse associated protein 1, SAP47 homolog | NM_032796 |
| | (Drosophila) | |
| SYMPK | Symplekin | NM_004819 |
| SYNGR2 | Synaptogyrin 2 | NM_004710 |
| SYT6 | Synaptotagmin VI | NM_205848 |
| TAP1 | Transporter 1, ATP-binding cassette, sub-family | NM_000593 |
| | B (MDR/TAP) | |
| TAS2R10 | Taste receptor, type 2, member 10 | NM_023921 |
| TCTEL1 | T-complex-associated-testis-expressed 1-like 1 | NM_006519 |
| TDE2 | Tumor differentially expressed 2 | NM_020755 |
| TETRAN | Tetracycline transporter-like protein | NM_001120 |
| TFAP2B | Transcription factor AP-2 beta (activating | NM_003221 |
| | enhancer binding protein 2 beta) | |
| TFCP2L3 | Transcription factor CP2-like 3 | NM_024915 |
| TGFB1I1 | Transforming growth factor beta 1 induced | NM_015927 |
| | transcript 1 | |
| TGFBR2 | Transforming growth factor, beta receptor II | NM_003242 |
| | (70/80 kDa) | |
| TGM4 | Transglutaminase 4 (prostate) | U79008 |
| THSD2 | Thrombospondin, type I, domain containing 2 | NM_032784 |
| TIFA | TRAF-interacting protein with a forkhead- | NM_052864 |
| | associated domain | |
| TIMP1 | Tissue inhibitor of metalloproteinase 1 (erythroid | NM_003254 |
| | potentiating activity, collagenase inhibitor) | |
| TLR1 | Toll-like receptor 1 | NM_003263 |
| TM4SF3 | Transmembrane 4 superfamily member 3 | NM_004616 |
| TM9SF4 | Transmembrane 9 superfamily protein member 4 | NM_014742 |
| TMEM25 | Transmembrane protein 25 | NM_032780 |
| TMEM34 | Transmembrane protein 34 | NM_018241 |
| TMOD3 | Tropomodulin 3 (ubiquitous) | NM_014547 |
| Tmp21-II | Tmp21-II, transcribed pseudogene | AJ004914 |
| TNA | Tetranectin (plasminogen binding protein) | NM_003278 |
| TNFRSF12A | Tumor necrosis factor receptor superfamily, | NM_016639 |
| | member 12A | |
| TNFRSF18 | Tumor necrosis factor receptor superfamily, | NM_148902 |
| | member 18 | |
| TNFSF4 | Tumor necrosis factor (ligand) superfamily, | NM_003326 |
| | member 4 (tax-transcriptionally activated | |
| | glycoprotein 1, 34 kDa) | |
| TNKS2 | Tankyrase, TRF1-interacting ankyrin-related | NM_025235 |
| | ADP-ribose polymerase 2 | |
| TPRA40 | Seven transmembrane domain orphan receptor | NM_016372 |
| TRAD | Serine/threonine kinase with Dbl- and pleckstrin | AL137629 |
| | homology domains | |
| TRAF3IP1 | TNF receptor-associated factor 3 interacting | BC059174 |
| | protein 1 | |
| TREM4 | Triggering receptor expressed on myeloid cells 4 | NM_145273 |
| TRIM35 | Tripartite motif-containing 35 | NM_015066 |
| TRIM9 | Tripartite motif-containing 9 | NM_015163 |
| TRIP | TRAF interacting protein | NM_005879 |
| TRPM5 | Transient receptor potential cation channel, | NM_014555 |
| | subfamily M, member 5 | |
| TRPM7 | Transient receptor potential cation channel, | NM_017672 |
| | subfamily M, member 7 | |
| TTC19 | Tetratricopeptide repeat domain 19 | BC066344 |
| TTR | Transthyretin (prealbumin, amyloidosis type I) | NM_000371 |
| TTYH2 | Tweety homolog 2 (Drosophila) | NM_032646 |
| TUBA1 | Tubulin, alpha 1 (testis specific) | NM_006000 |
| TUBB1 | Tubulin, beta 1 | NM_030773 |
| TUBB4 | Tubulin, beta 4 | NM_006087 |
| TXNIP | Thioredoxin interacting protein | NM_006472 |
| UBD | Ubiquitin D | NM_006398 |
| UBE2V1 | Ubiquitin-conjugating enzyme E2 variant 1 | NM_199144 |
| UBE3A | Ubiquitin protein ligase E3A (human papilloma | AF037219 |
| | virus E6-associated protein, Angelman | |
| | syndrome) | |
| UBL3 | Ubiquitin-like 3 | NM_007106 |
| UHSKerB | Keratin, ultrahigh sulfur, B | NM_021046 |
| ULK2 | Unc-51-like kinase 2 (C. elegans) | NM_014683 |
| URB | Steroid sensitive gene 1 | NM_199511 |
| USP54 | Ubiquitin specific protease 54 | NM_152586 |
| UST | Uronyl-2-sulfotransferase | NM_005715 |
| UTRN | Utrophin (homologous to dystrophin) | AK023675 |
| UTX | Ubiquitously transcribed tetratricopeptide repeat, | NM_021140 |
| | X chromosome | |
| VARS2L | Valyl-tRNA synthetase 2-like | NM_020442 |
| VAV1 | Vav 1 oncogene | NM_005428 |
| VGLL4 | Vestigial like 4 (Drosophila) | BQ013066 |
| VN1R1 | Vomeronasal 1 receptor 1 | NM_020633 |
| VSIG4 | V-set and immunoglobulin domain containing 4 | NM_007268 |
| WDR22 | WD repeat domain 22 | NM_003861 |
| WIF1 | WNT inhibitory factor 1 | NM_007191 |
| WWOX | WW domain containing oxidoreductase | AK094336 |
| XG | Xg blood group (pseudoautosomal boundary- | NM_175569 |
| | divided on the X chromosome) | |
| XIST | X (inactive)-specific transcript | AK025198 |
| XYLT2 | Xylosyltransferase II | NM_022167 |
| YPEL5 | Yippee-like 5 (Drosophila) | NM_016061 |
| ZBTB7 | Zinc finger and BTB domain containing 7 | NM_015898 |
| ZFHX1B | Zinc finger homeobox 1b | NM_014795 |
| ZFYVE26 | Zinc finger, FYVE domain containing 26 | NM_015346 |
| ZNF516 | Zinc finger protein 516 | D86975 |
| ZNF552 | Zinc finger protein 552 | AK023769 |
| ZNF572 | Zinc finger protein 572 | NM_152412 |
| ZP3 | Zona pellucida glycoprotein 3 (sperm receptor) | NM_007155 |
| ZSCAN2 | Zinc finger and SCAN domain containing 2 | NM_017894 |
| | No Annotation | A_23_BS113762 |
| | No Annotation | A_24_BS784213 |
| | No Annotation | A_24_BS926155 |
| | No Annotation | A_24_BS927614 |
| | No Annotation | A_24_BS934268 |
| | No Annotation | A_32_BS169243 |
| | No Annotation | A_32_BS200773 |
| | No Annotation | A_32_BS53976 |
| | No Annotation | A_32_BS73184 |
| | No Annotation | A_32_BS74588 |
| | No Annotation | AB065507 |
| | No Annotation | AC007051 |
| | No Annotation | AC007066 |
| | No Annotation | AC008453 |
| | No Annotation | AC025463 |
| | No Annotation | AC060234 |
| | No Annotation | AC087071 |
| | No Annotation | AC096677 |
| | Full length insert cDNA clone ZB81F12 | AF086167 |
| | No Annotation | AF089746 |
| | Amyloid lambda 6 light chain variable region | AF121762 |
| | SAR | |
| | IMAGE Consortium ID 839832, mRNA | AF124368 |
| | sequence | |
| | Clone FLB4246 PRO1102 mRNA, complete cds | AF130105 |
| | HSPC101 | AF161364 |
| | LOC440135 | AF318337 |
| | No Annotation | AF372624 |
| | No Annotation | AF533936 |
| | MRNA (fetal brain cDNA g6_1g) | AI791206 |
| | Hypothetical protein (ORF1), clone 00275 | AJ276555 |
| | No Annotation | AK001565 |
| | Hypothetical LOC388796 | AK022745 |
| | Homo sapiens, clone IMAGE: 4401608, mRNA | AK022793 |
| | Homo sapiens, clone IMAGE: 4214313, mRNA | AK022893 |
| | Homo sapiens, clone IMAGE: 5277945, mRNA | AK022997 |
| | CDNA: FLJ22769 fis, clone KAIA1316 | AK026422 |
| | CDNA FLJ31059 fis, clone HSYRA2000832 | AK055621 |
| | CDNA FLJ32177 fis, clone PLACE6001294 | AK056856 |
| | Homo sapiens, clone IMAGE: 5575764, mRNA | AK090500 |
| | Homo sapiens, clone IMAGE: 5575764, mRNA | AK092921 |
| | CDNA FLJ36725 fis, clone UTERU2012230 | AK094044 |
| | CDNA FLJ38235 fis, clone FCBBF2005428 | AK095554 |
| | CDNA FLJ25794 fis, clone TST07014 | AK098660 |
| | No Annotation | AL009178 |
| | MRNA; cDNA DKFZp566L0824 (from clone | AL050042 |
| | DKFZp566L0824) | |
| | No Annotation | AL109935 |
| | No Annotation | AL132874 |
| | Full-length cDNA clone CS0DJ001YJ05 of T | AL137761 |
| | cells (Jurkat cell line) Cot 10-normalized of | |
| | Homo sapiens (human) | |
| | No Annotation | AL391244 |
| | No Annotation | AL445486 |
| | No Annotation | AL591806 |
| | No Annotation | AL731541 |
| | No Annotation | AL928970 |
| | No Annotation | AY062331 |
| | No Annotation | AY372690 |
| | No Annotation | BC009051 |
| | LOC441164 | BC009220 |
| | CDNA clone IMAGE: 3462401, partial cds | BC010544 |
| | No Annotation | BC011367 |
| | No Annotation | BC015531 |
| | LOC440441 | BC020847 |
| | Homo sapiens, clone IMAGE: 5295565, mRNA, | BC031278 |
| | partial cds | |
| | Similar to jumonji domain containing 1A; testis- | BC035102 |
| | specific protein A; zinc finger protein | |
| | Homo sapiens, clone IMAGE: 5575764, mRNA | BC035647 |
| | Hypothetical LOC197387 | BC038761 |
| | Hypothetical gene supported by BC039664 | BC039664 |
| | No Annotation | BC107852 |
| | No Annotation | BG252130 |
| | Full-length cDNA clone CS0DI009YA14 of | BG327427 |
| | Placenta Cot 25-normalized of Homo sapiens | |
| | (human) | |
| | Hypothetical LOC339352 | BG620990 |
| | Similar to PI-3-kinase-related kinase SMG-1 | BI014689 |
| | isoform 2; lambda/iota protein kinase C- | |
| | interacting protein; phosphatidylinositol 3-kinase- | |
| | related protein kinase | |
| | Similar to D(1B) dopamine receptor (D(5) | BM561346 |
| | dopamine receptor) (D1beta dopamine receptor) | |
| | No Annotation | BM839360 |
| | Transcribed locus | BM925639 |
| | No Annotation | BM928667 |
| | Transcribed locus | BQ049338 |
| | No Annotation | BQ346290 |
| | Homo sapiens, clone IMAGE: 4838137, mRNA | BU587941 |
| | LOC441139 | BX118328 |
| | No Annotation | D80006 |
| | No Annotation | DQ101103 |
| | No Annotation | DQ188807 |
| | No Annotation | ENST00000242479 |
| | No Annotation | ENST00000246627 |
| | No Annotation | ENST00000259219 |
| | No Annotation | ENST00000259550 |
| | No Annotation | ENST00000293569 |
| | No Annotation | ENST00000296448 |
| | No Annotation | ENST00000298643 |
| | No Annotation | ENST00000299756 |
| | No Annotation | ENST00000300068 |
| | No Annotation | ENST00000305402 |
| | No Annotation | ENST00000305824 |
| | No Annotation | ENST00000307901 |
| | No Annotation | ENST00000308307 |
| | No Annotation | ENST00000310210 |
| | No Annotation | ENST00000312401 |
| | No Annotation | ENST00000312412 |
| | No Annotation | ENST00000312966 |
| | No Annotation | ENST00000313904 |
| | No Annotation | ENST00000318669 |
| | No Annotation | ENST00000321112 |
| | No Annotation | ENST00000321656 |
| | No Annotation | ENST00000322114 |
| | No Annotation | ENST00000322404 |
| | No Annotation | ENST00000322803 |
| | No Annotation | ENST00000324770 |
| | No Annotation | ENST00000325204 |
| | No Annotation | ENST00000325773 |
| | No Annotation | ENST00000327591 |
| | No Annotation | ENST00000327870 |
| | No Annotation | ENST00000328059 |
| | No Annotation | ENST00000328708 |
| | No Annotation | ENST00000329246 |
| | No Annotation | ENST00000329358 |
| | No Annotation | ENST00000329491 |
| | No Annotation | ENST00000329660 |
| | No Annotation | ENST00000330875 |
| | No Annotation | ENST00000331096 |
| | No Annotation | ENST00000331577 |
| | No Annotation | ENST00000331640 |
| | No Annotation | ENST00000332271 |
| | No Annotation | ENST00000332944 |
| | No Annotation | ENST00000332989 |
| | No Annotation | ENST00000333517 |
| | No Annotation | ENST00000333784 |
| | Transcribed locus, weakly similar to | H16080 |
| | NP_808455.1 hypothetical protein 9830102E05 | |
| | [Mus musculus] | |
| | No Annotation | I_1000437 |
| | No Annotation | I_1100650 |
| | No Annotation | I_1221777 |
| | No Annotation | I_1861543 |
| | No Annotation | I_1879042 |
| | No Annotation | I_1882608 |
| | No Annotation | I_1891291 |
| | No Annotation | I_1893151 |
| | No Annotation | I_1980505 |
| | No Annotation | I_1985061 |
| | No Annotation | I_3335767 |
| | No Annotation | I_3344109 |
| | No Annotation | I_3551568 |
| | No Annotation | I_3575384 |
| | No Annotation | I_3576071 |
| | No Annotation | I_3580313 |
| | No Annotation | I_3588329 |
| | No Annotation | I_930906 |
| | No Annotation | I_932413 |
| | No Annotation | I_943866 |
| | No Annotation | I_944092 |
| | No Annotation | I_962800 |
| | No Annotation | I_964340 |
| | No Annotation | I_966091 |
| | No Annotation | I_966691 |
| | No Annotation | M15073 |
| | No Annotation | M64260 |
| | No Annotation | NG_001019 |
| | No Annotation | NM_001005360 |
| | No Annotation | NM_001008528 |
| | No Annotation | NM_001009555 |
| | No Annotation | NM_001009569 |
| | No Annotation | NM_001010919 |
| | No Annotation | NM_001011708 |
| | No Annotation | NM_001013632 |
| | No Annotation | NM_001013680 |
| | No Annotation | NM_001014975 |
| | No Annotation | NM_001018006 |
| | No Annotation | NM_001018011 |
| | No Annotation | NM_001018076 |
| | No Annotation | NM_001024227 |
| | No Annotation | NM_001024465 |
| | No Annotation | NM_001024808 |
| | No Annotation | NM_001025077 |
| | No Annotation | NM_001025201 |
| | No Annotation | NM_001031677 |
| | No Annotation | NM_001033044 |
| | No Annotation | NM_001033569 |
| | No Annotation | NM_003671 |
| | No Annotation | NM_014758 |
| | No Annotation | NM_015262 |
| | No Annotation | NM_018350 |
| | No Annotation | NM_018506 |
| | No Annotation | NM_080432 |
| | No Annotation | NM_138411 |
| | No Annotation | NM_153030 |
| | No Annotation | NM_153237 |
| | No Annotation | NM_172020 |
| | No Annotation | NM_173705 |
| | No Annotation | NM_173709 |
| | No Annotation | NM_178429 |
| | No Annotation | NM_178467 |
| | No Annotation | NM_213595 |
| | No Annotation | NR_001544 |
| | No Annotation | NR_002184 |
| | No Annotation | NR_002225 |
| | Anti-HIV-1 gp120 V3 loop antibody DO142-10 | S62210 |
| | light chain variable region | |
| | No Annotation | S80864 |
| | No Annotation | THC1409898 |
| | No Annotation | THC1419743 |
| | No Annotation | THC1429821 |
| | No Annotation | THC1434038 |
| | No Annotation | THC1438453 |
| | No Annotation | THC1441583 |
| | No Annotation | THC1448600 |
| | No Annotation | THC1457058 |
| | No Annotation | THC1457118 |
| | No Annotation | THC1459712 |
| | No Annotation | THC1461073 |
| | No Annotation | THC1469536 |
| | No Annotation | THC1475763 |
| | No Annotation | THC1477639 |
| | No Annotation | THC1484458 |
| | No Annotation | THC1490378 |
| | No Annotation | THC1493219 |
| | No Annotation | THC1504780 |
| | No Annotation | THC1505917 |
| | No Annotation | THC1506312 |
| | No Annotation | THC1511927 |
| | No Annotation | THC1515028 |
| | No Annotation | THC1525318 |
| | No Annotation | THC1531579 |
| | No Annotation | THC1537124 |
| | No Annotation | THC1543691 |
| | No Annotation | THC1544941 |
| | No Annotation | THC1551463 |
| | No Annotation | THC1555359 |
| | No Annotation | THC1559236 |
| | No Annotation | THC1560798 |
| | No Annotation | THC1562602 |
| | No Annotation | THC1563147 |
| | No Annotation | THC1564329 |
| | No Annotation | THC1572906 |
| | No Annotation | THC1572972 |
| | No Annotation | THC1574967 |
| | No Annotation | THC1578318 |
| | No Annotation | THC1581022 |
| | No Annotation | THC1584122 |
| | No Annotation | THC1589164 |
| | No Annotation | THC1591470 |
| | Hypothetical gene LOC133874 | U31733 |
| | No Annotation | U62539 |
| | No Annotation | X68990 |
| | No Annotation | XM_065006 |
| | No Annotation | XM_165930 |
| | No Annotation | XM_170211 |
| | Similar to ARHQ protein | XM_209429 |
| | No Annotation | XM_210579 |
| | No Annotation | XM_291496 |
| | No Annotation | XM_291718 |
| | No Annotation | XM_295760 |
| | No Annotation | XM_301448 |
| | No Annotation | XM_303638 |
| | No Annotation | XM_305652 |
| | Similar to Tubulin beta-4q chain | XM_371684 |
| | Similar to CXYorf1-related protein | XM_377073 |
| | Similar to immunoglobulin M chain | Y11328 |

Biological Processes Differentially Expressed in the Intrinsic Groups. To systematically investigate the biological processes found in the gene expression profiles of SSc, a module map was created using Genomica software (Segal, et al. (2004) supra; Stuart, et al. (2003) supra). A module map shows arrays that have co-expressed genes that map to specific gene sets. In this case, each gene set represents a specific biological process derived from Gene Ontology (GO) Biological process annotations (Ashburner, et al. (2000) The Gene Ontology Consortium 25:25-29), or from previously published microarray datasets (Whitfield, et al. (2002) supra; Palmer, et al. (2006) supra).

Modules with significantly enriched genes (p<0.05, hypergeometric distribution) and corrected for multiple hypothesis testing with an FDR of 0.1% were identified. Expressed among the group Diffuse-Proliferation were the biological processes of cytokinesis, cell cycle checkpoint, regulation of mitosis, cell cycle, DNA repair, S phase, and DNA replication, consistent with the presence of dividing cells. Decreased in this group were genes associated with fatty acid biosynthesis, lipid biosynthesis, oxidoreductase activity and decreased electron transport activity. The decrease in genes associated with fatty acid and lipid biosynthesis was notable given the loss of subcutaneous fat observed in dSSc patients (Medsger (2001) supra).

Expressed in the Inflammatory group were biological processes indicative of an increased immune response, including the GO biological processes of immune response, response to pathogen, humoral defense, lymphocyte proliferation, chemokine binding, chemokine receptor activity, and response to virus. Biological processes of icosanoid and prostanoid metabolism, which represent synthesis of prostaglandin lipid second messengers, have been associated with immune responses (Funk (2001) Science 294:1871-1875), found to be highly expressed in rheumatoid arthritis (Crofford, et al. (1994) J. Clin. Invest. 93:1095-1101; Kojima, et al. (2003) Arthritis Rheum. 48:2819-2828; Westman, et al. (2004) Arthritis Rheum. 50:1774-1780) and associated with severity in collagen-induced arthritis in mice (Trebino, et al. (2003) Proc. Natl. Acad. Sci. USA 100:9044-9049; Sheibanie, et al. (2007) Arthritis Rheum. 56:2608-26). Also expressed in the Inflammatory group were processes associated with fibrosis including trypsin activity, collagen and extracellular matrix.

To better define the proliferation signature observed, gene sets were created representing the genes periodically expressed in the human cell division cycle as defined by Whitfield, et al. (2002) supra). Gene sets were created that included the genes with peak expression at each of the five different cell cycle phases, G1/S, S, G2, G2/M and M/G1 (Whitfield, et al. (2002) supra). The enrichment of each of these five gene sets was statistically significant (p<0.05 using the hypergeometric distribution) and more highly expressed in the Diffuse-Proliferation group.

To better characterize the lymphocyte infiltrates, gene sets were generated representing lymphocyte subsets from Palmer, et al. (2006) supra. Using isolated populations of lymphocytes and DNA microarray hybridization, the genes specifically expressed in different lymphocyte subsets were identified. Subsets included T cells (total lymphocyte and CD8+), B cells, and granulocytes. Four of these gene sets, B cells, T cells, CD8+ T cells and granulocytes, were found to have a statistically significant over-representation in the Inflammatory group. This indicated that the gene expression signature expressed in this group was determined by the presence of infiltrating lymphocytes and specifically implied the infiltrating cells included T cells, B cells and granulocytes. Although a gene expression signature representative of macrophages or dendritic cells was not included in this analysis, the macrophage marker CD163 was highly expressed in this group, indicating innate immune responses may play an important role in disease pathogenesis.

Immunohistochemistry (IHC). To verify that the gene expression reflected increased numbers of infiltrating lymphocytes or proliferating cells, IHC was performed for T cells (anti-CD3), B cells (anti-CD20) and cycling cells (anti-KI67). Summarized in Table 4 is a full enumeration of marker positive cells counted from representative fields of all biopsies analyzed by IHC, with the observer blinded to disease state. Analysis of biopsies from each of the major intrinsic groups confirmed the results found in the gene expression signatures. The presence of infiltrating T cells was confirmed in the Inflammatory group (Table 4). The largest numbers of T cells were found in perivascular and perifollicular distributions, as well as in the dermis, of two dSSc patients (dSSc5, dSSc6) assigned to the Inflammatory group (Table 4). IHC was also performed on skin biopsies from two patients with morphea (Morph1, Morph3) and each showed large numbers of infiltrating T cells. Only a small number of T cells were observed in two healthy controls analyzed (Nor2 and Nor3). A slight increase in T cells was observed in a perivascular distribution in the four patients assigned to Diffuse-Proliferation (dSSc1, dSSc2, dSSc11, dSSc12; Table 4), which had a lower expression of the T cell signature.

Few CD20+ B cells were observed in the SSc skin biopsies. The immunoglobulin gene expression signature was observed in eight diffuse patients (dSSc1, dSSc3, dSSc6, dSSc7, dSSc8, dSSc10, dSSc11, dSSc12) and one limited patient (lSSc7). Of the six patients analyzed by IHC (dSSc1, dSSc2, dSSc5, dSSc6, dSSc11, dSSc12), two samples (dSSc1 and dSSc12) showed small numbers of CD20+ B cells.

The presence of the proliferation signature has been correlated with an increase in the mitotic index or number of dividing cells in microarray studies of cancer (Whitfield, et al. (2006) supra; Perou, et al. (2000) supra; Perou, et al. (1999) supra; Whitfield, et al. (2002) supra; Ross, et al. (2000) Nat. Genet. 24:227-235). To confirm the presence of proliferating cells in the dSSc skin biopsies, IHC staining was performed for KI67, a standard marker of cycling cells. Analysis of skin from healthy controls (Nor2, Nor3), morphea (Morph1, Morph3), and diffuse patients in the Inflammatory group (dSSc5, dSSc6), showed no proliferating cells in the dermis, and a small number of proliferating cells surrounding dermal appendages and in the epidermal layer (Table 4). In contrast, analysis of the skin from four patients in the Diffuse-Proliferation subgroup (dSSc1, dSSc2, dSSc11 and dSSc12) showed higher numbers of proliferating cells primarily in the epidermis (Table 4). Therefore, it was concluded that the proliferation signature was likely the result of an increased number of proliferating cells in the epidermal compartment of the SSc skin biopsies. The identity of these cells was very likely to be keratinocytes.

Intrinsic Gene Expression Maps to Identifiable Clinical Covariates. To map the intrinsic groups to specific clinical covariates, Pearson correlations were calculated between the gene expression of each of the ca. 1000 intrinsic genes and different clinical covariates. Shown are the results for three different covariates: the modified Rodnan skin score (MRSS; 0-51 scale), a self-reported Raynaud's severity score (0-10 scale), and the extent of skin involvement (dSSc, lSSc and unaffected). Each group was analyzed for correlation to each of the clinical parameters listed in Table 1. Pearson correlation coefficients were calculated between each of the clinical parameters and the expression of each gene. The moving average (10-gene window) of the resultant correlation coefficients was plotted for MRSS, Raynaud's severity and degree of skin involvement. Areas of high positive correlation between a clinical parameter and the expression of a group of genes indicated that increased expression of those genes was associated with an increase in that clinical covariate; a negative correlation indicated a relationship between a decrease in expression of the genes and an increase in a clinical covariate.

Areas of high positive or high negative correlation were identified. Each of the three clinical covariates showed high positive correlations to a subset of gene expression signatures. Most notably, the MRSS skin score showed a high positive correlation to the ‘proliferation signature’ with correlations ranging from 0.5 and 0.6. This signature was highly expressed in Diffuse-Proliferation samples but had low expression in the Inflammatory group. The Raynaud's severity score had a high positive correlation to genes expressed at higher levels in the Limited group and heterogeneously expressed in patients with dSSc. The genes highly correlated with MRSS also showed a high positive correlation with diffuse skin involvement. While this signature associated with diffuse skin involvement, it was important to note that a subset of dSSc skin biopsies did not express this signature and had low skin scores. Similarly, the genes that had a high positive correlation with Raynaud's severity and a high positive correlation with the Limited group, which typically has more severe vascular involvement, were uncorrelated with the diagnosis of dSSc and were expressed at low levels in healthy control samples. Moving averages of the Pearson correlation between the intrinsic genes and other clinical covariates (digital ulcers, ILD, or GI involvement) were also calculated but did not reveal significant regions of positive or negative correlation to the gene expression profiles.

One initial hypothesis was that there would be an obvious trend in the gene expression data reflecting the progressive nature of SSc in some patients. To examine this more carefully, disease duration in years since first onset of non-Raynaud's symptoms was plotted along the X-axis of the heat map. The mean disease duration for the Diffuse-Proliferation group was 8.4±6.4 yrs, whereas mean disease duration for the Inflammatory group, which includes dSSc and lSSc, was 6.5±6.1 yrs. Using a Student's t-test with a two-tailed distribution, this difference was not found to be statistically significant. To test the hypothesis that a subset of the patients was grouping by disease duration, the disease duration was analyzed between the dSSc patients in the Diffuse-Proliferation group and the dSSc patients that were classified as either Inflammatory or Normal-Like (Table 3). The Diffuse-Proliferation group had a mean disease duration of 8.4±6.4 years, and the dSSc patients in the Inflammatory and Normal-Like groups had a mean disease duration of 3.2±3.9 years (p=0.12, t-test). The difference in the means between these two groups was clear, but outliers in each reduced the significance of the result. Dropping the two outliers resulted in p=0.0042 (unequal variance two sample t-test, two-sided)). Therefore, it was concluded that there was a significant association between disease duration and the intrinsic groups for dSSc samples.

Since no obvious clinical covariate was identified that differentiated the dSSc group 1 from dSSc group 2, the genes that most differentiated the two groups were selected using a non-parametric t-test implemented in Significance Analysis of Microarrays (SAM) (Tusher, et al. (2001) Proc. Natl. Acad. Sci. USA 98:5116-5121). 329 genes were selected that were differentially expressed between these two groups with an FDR of 0.19%. These 329 genes were analyzed for correlation to clinical covariates. Three clinical covariates were found associated with these two groups. The genes highly expressed in the dSSc group 2 (nine patients) were highly correlated with the presence of digital ulcers (DU) and the presence of interstitial lung disease (ILD) at the time the skin biopsies were taken. In contrast, dSSc group 1 (two patients, both male) did not have DU or ILD at the time of biopsy. Although this grouping could result simply from stratification by sex, it also may reflect a true difference in disease presentation. Only 18 of the 329 genes mapped to either the X or Y chromosomes and thus were expected to be differentially expressed, indicating the remainder may represent biology underlying these groups.

A Subset of Genes is Associated With Increased Modified Rodnan Skin Score. To identify genes associated with MRSS, the subset of genes most highly correlated with each covariate from the intrinsic list were selected using Pearson correlations. 177 genes were selected from the ca. 1000 intrinsic genes that had Pearson correlations with MRSS>0.5 or <−0.5 (Table 6). This list of 177 genes was then used to organize the skin biopsies by average linkage hierarchical clustering. It was found that both forearm and back skin biopsies from 14 patients with dSSc (mean MRSS of 26.34±9.42) clustered onto a single branch of the dendrogram. All other samples, including the forearm-back pairs of four patients with dSSc (mean MRSS 18.11±6.45) clustered onto a separate branch of the dendrogram. Using a two-tailed Student's t-test, it was found that the difference in skin score between the two groups of dSSc was statistically significant (p=0.0197).

From this analysis, 62 genes were expressed at high levels and 115 genes were expressed at low levels in the patients with the highest skin score (Table 6). Genes highly expressed included the cell cycle genes CENPE, CDC7 and CDT1, the mitogen Fibroblast Growth Factor 5 (FGF5), the immediate early gene Tumor Necrosis Factor Receptor Superfamily member 12A (TNFRSF12A) and TRAF interacting protein (TRIP). Since skin score is considered to be an effective measure for disease outcome, this 177-gene signature is contemplated to contain genes of use as surrogate markers for skin score.

TABLE 6

| | | | | |
|---|---|---|---|---|
| Gene | | | High Skin | Low Skin |
| Symbol | Gene Name | Accession | Score | Score |

GENES WITH HIGH EXPRESSION CORRELATED WITH MRSS

| | | | | |
|---|---|---|---|---|
| ALG2 | Asparagine-linked glycosylation 2 | NM_033087 | 0.13 | −0.14 |
| | homolog (yeast, alpha-1,3- | | | |
| | mannosyltransferase) | | | |
| APOH | Apolipoprotein H (beta-2-glycoprotein | NM_000042 | 1.12 | −0.46 |
| | I) | | | |
| ATAD2 | ATPase family, AAA domain | NM_014109 | 0.52 | −0.28 |
| | containing 2 | | | |
| B3GALT6 | UDP-Gal:betaGal beta 1,3- | NM_080605 | 0.17 | −0.10 |
| | galactosyltransferase polypeptide 6 | | | |
| C12orf14 | Chromosome 12 open reading frame 14 | NM_021238 | 0.58 | −0.17 |
| CBLL1 | Cas-Br-M (murine) ecotropic retroviral | NM_024814 | 0.29 | −0.10 |
| | transforming sequence-like 1 | | | |
| CDC7 | CDC7 cell division cycle 7 (S. cerevisiae) | NM_003503 | 0.46 | −0.30 |
| CDT1 | DNA replication factor | NM_030928 | 0.45 | −0.23 |
| CENPE | Centromere protein E, 312 kDa | NM_001813 | 0.16 | −0.13 |
| CGI-90 | CGI-90 protein | NM_016033 | 0.37 | −0.27 |
| CROC4 | Transcriptional activator of the c-fos | NM_006365 | 0.32 | −0.10 |
| | promoter | | | |
| FGF5 | Fibroblast growth factor 5 | NM_004464 | 0.28 | −0.14 |
| FLJ10902 | Hypothetical protein FLJ10902 | BC021277 | 0.35 | −0.11 |
| FLJ12438 | Hypothetical protein FLJ12438 | NM_021933 | 0.60 | −0.21 |
| FLJ12443 | Hypothetical protein FLJ12443 | NM_024830 | 0.66 | −0.34 |
| FLJ12484 | Hypothetical protein FLJ12484 | NM_022767 | 0.67 | −0.20 |
| FLJ20245 | Hypothetical protein FLJ20245 | NM_017723 | 0.32 | −0.14 |
| FLJ32009 | Hypothetical protein FLJ32009 | NM_152718 | 0.50 | −0.24 |
| FLJ35757 | Hypothetical protein FLJ35757 | NM_152598 | 0.25 | −0.07 |
| FXYD2 | FXYD domain containing ion transport | NM_021603 | 0.50 | −0.15 |
| | regulator 2 | | | |
| GSG2 | Haspin | AK056691 | 0.18 | −0.14 |
| HPS3 | Hermansky-Pudlak syndrome 3 | NM_032383 | 0.38 | −0.16 |
| KIAA1666 | KIAA1666 protein | XM_371429 | 0.26 | −0.15 |
| LGALS8 | Lectin, galactoside-binding, soluble, 8 | NM_201543 | 0.17 | −0.13 |
| | (galectin 8) | | | |
| LILRB5 | Leukocyte immunoglobulin-like | NM_006840 | 0.18 | −0.13 |
| | receptor, subfamily B (with TM and | | | |
| | ITIM domains), member 5 | | | |
| LOC128977 | Hypothetical protein LOC128977 | NM_173793 | 0.40 | −0.14 |
| LRRIQ2 | Leucine-rich repeats and IQ motif | NM_024548 | 0.29 | −0.09 |
| | containing 2 | | | |
| MGC13186 | Hypothetical protein MGC13186 | NM_032324 | 0.20 | −0.15 |
| MGC16044 | Hypothetical protein MGC16044 | NM_138371 | 0.29 | −0.09 |
| MGC29784 | Hypothetical protein MGC29784 | NM_173659 | 0.36 | −0.16 |
| MICB | MHC class I polypeptide-related | NM_005931 | 0.35 | −0.17 |
| | sequence B | | | |
| MTRF1L | Mitochondrial translational release | NM_019041 | 0.21 | −0.08 |
| | factor 1-like | | | |
| NICN1 | Nicolin 1 | NM_032316 | 0.22 | −0.10 |
| OAS3 | 2′-5′-oligoadenylate synthetase 3, | NM_006187 | 0.41 | −0.07 |
| | 100 kDa | | | |
| OGDHL | Oxoglutarate dehydrogenase-like | NM_018245 | 0.92 | −0.27 |
| OPRK1 | Opioid receptor, kappa 1 | NM_000912 | 0.16 | −0.04 |
| PCNT2 | Pericentrin 2 (kendrin) | NM_006031 | 0.36 | −0.07 |
| PPFIA4 | Protein tyrosine phosphatase, receptor | NM_015053 | 0.40 | −0.18 |
| | type, f polypeptide (PTPRF), | | | |
| | interacting protein (liprin), alpha 4 | | | |
| PSMD11 | Proteasome (prosome, macropain) 26S | NM_002815 | 0.29 | −0.10 |
| | subunit, non-ATPase, 11 | | | |
| PSPHL | Phosphoserine phosphatase-like | AJ001612 | 1.08 | −0.08 |
| RPS18 | Ribosomal protein S18 | NM_022551 | 0.21 | −0.11 |
| SYT6 | Synaptotagmin VI | NM_205848 | 0.26 | −0.20 |
| TMOD3 | Tropomodulin 3 (ubiquitous) | NM_014547 | 0.31 | −0.08 |
| TNFRSF12A | Tumor necrosis factor receptor | NM_016639 | 0.62 | −0.25 |
| | superfamily, member 12A | | | |
| TRIP | TRAF interacting protein | NM_005879 | 0.34 | −0.18 |
| TTR | Transthyretin (prealbumin, amyloidosis | NM_000371 | 0.52 | −0.44 |
| | type I) | | | |
| TUBB4 | Tubulin, beta 4 | NM_006087 | 0.26 | −0.18 |
| ZSCAN2 | Zinc finger and SCAN domain | NM_017894 | 0.31 | −0.09 |
| | containing 2 | | | |
| | | AB065507 | 0.44 | −0.10 |
| | Homo sapiens, clone IMAGE: 5277945, | AK022997 | 0.32 | −0.11 |
| | mRNA | | | |
| | CDNA FLJ36725 fis, clone | AK094044 | 0.54 | −0.20 |
| | UTERU2012230 | | | |
| | | AL391244 | 0.22 | −0.18 |
| | | AL928970 | 0.36 | −0.12 |
| | CDNA clone IMAGE: 3462401, partial | BC010544 | 0.40 | −0.24 |
| | cds | | | |
| | | BM928667 | 0.69 | −0.38 |
| | | ENST00000328708 | 0.19 | −0.15 |
| | | NM_001009569 | 0.31 | −0.08 |
| | | NM_172020 | 0.24 | −0.14 |
| | | NM_178467 | 0.44 | −0.29 |
| | | THC1504780 | 0.45 | −0.10 |
| | | XM_210579 | 0.22 | −0.14 |
| | Similar to Tubulin beta-4q chain | XM_371684 | 0.18 | −0.14 |

GENES WITH LOW EXPRESSION CORRELATED WITH MRSS

| | | | | |
|---|---|---|---|---|
| ADH1A | Alcohol dehydrogenase 1A (class I), | NM_000667 | −0.64 | 0.60 |
| | alpha polypeptide | | | |
| ADH1C | Alcohol dehydrogenase 1C (class I), | NM_000669 | −0.56 | 0.22 |
| | gamma polypeptide | | | |
| AMOT | Angiomotin | NM_133265 | −0.45 | 0.17 |
| AP2A2 | Adaptor-related protein complex 2, | NM_012305 | −0.23 | 0.12 |
| | alpha 2 subunit | | | |
| ARK5 | AMP-activated protein kinase family | NM_014840 | −0.23 | 0.17 |
| | member 5 | | | |
| ARMCX1 | Armadillo repeat containing, X-linked 1 | NM_016608 | −0.56 | 0.31 |
| BMP8A | Bone morphogenetic protein 8a | AK093659 | −0.40 | 0.17 |
| C1orf24 | Chromosome 1 open reading frame 24 | NM_052966 | −0.53 | 0.23 |
| C9orf61 | Chromosome 9 open reading frame 61 | NM_004816 | −0.71 | 0.56 |
| CAPS | Calcyphosine | NM_004058 | −0.24 | 0.15 |
| CAST | Calpastatin | NM_173060 | −0.35 | 0.16 |
| CDR1 | Cerebellar degeneration-related protein | NM_004065 | −0.42 | 0.25 |
| | 1, 34 kDa | | | |
| CFHL1 | Complement factor H-related 1 | NM_002113 | −0.57 | 0.29 |
| CRTAP | Cartilage associated protein | NM_006371 | −0.33 | 0.26 |
| CXCL5 | Chemokine (C—X—C motif) ligand 5 | NM_002994 | −0.24 | 0.09 |
| CYBRD1 | Cytochrome b reductase 1 | NM_024843 | −0.57 | 0.39 |
| DBN1 | Drebrin 1 | NM_004395 | −0.33 | 0.36 |
| DCAMKL1 | Doublecortin and CaM kinase-like 1 | NM_004734 | −0.55 | 0.28 |
| DKK2 | Dickkopf homolog 2 (Xenopus laevis) | NM_014421 | −0.59 | 0.36 |
| ECM2 | Extracellular matrix protein 2, female | NM_001393 | −0.26 | 0.30 |
| | organ and adipocyte specific | | | |
| EMCN | Endomucin | AL133118 | −0.33 | 0.14 |
| EPB41L2 | Erythrocyte membrane protein band | NM_001431 | −0.38 | 0.06 |
| | 4.1-like 2 | | | |
| FBLN1 | Fibulin 1 | NM_006486 | −0.69 | 0.43 |
| FBLN2 | Fibulin 2 | NM_001998 | −0.51 | 0.20 |
| FEM1A | Fem-1 homolog a (C. elegans) | NM_018708 | −1.15 | 0.18 |
| FER1L3 | Fer-1-like 3, myoferlin (C. elegans) | NM_133337 | −0.44 | 0.05 |
| FGL2 | Fibrinogen-like 2 | NM_006682 | −0.38 | 0.46 |
| FHL5 | Four and a half LIM domains 5 | NM_020482 | −0.39 | 0.09 |
| FLJ20701 | Hypothetical protein FLJ20701 | NM_017933 | −0.54 | 0.29 |
| FLJ23861 | Hypothetical protein FLJ23861 | NM_152519 | −0.29 | 0.14 |
| FLJ36748 | Hypothetical protein FLJ36748 | NM_152406 | −0.39 | 0.21 |
| GHR | Growth hormone receptor | NM_000163 | −0.62 | 0.20 |
| GTPBP5 | GTP binding protein 5 (putative) | NM_015666 | −0.43 | 0.14 |
| IGFBP5 | Insulin-like growth factor binding | NM_000599 | −0.38 | 0.25 |
| | protein 5 | | | |
| IL15 | Interleukin 15 | NM_172175 | −0.39 | 0.25 |
| KAZALD1 | Kazal-type serine protease inhibitor | NM_030929 | −0.44 | 0.47 |
| | domain 1 | | | |
| KCNK4 | Potassium channel, subfamily K, | NM_016611 | −0.16 | 0.11 |
| | member 4 | | | |
| KCNS3 | Potassium voltage-gated channel, | NM_002252 | −0.22 | 0.13 |
| | delayed-rectifier, subfamily S, member 3 | | | |
| KIAA0494 | KIAA0494 gene product | NM_014774 | −0.37 | 0.16 |
| KIAA0870 | KIAA0870 protein | NM_014957 | −0.53 | 0.13 |
| KIAA1190 | Hypothetical protein KIAA1190 | NM_145166 | −0.37 | 0.41 |
| KLHL18 | Kelch-like 18 (Drosophila) | AB018338 | −0.33 | 0.11 |
| LAMP2 | Lysosomal-associated membrane | NM_013995 | −0.44 | 0.18 |
| | protein 2 | | | |
| LHFP | Lipoma HMGIC fusion partner | NM_005780 | −0.30 | 0.25 |
| LTBP4 | Latent transforming growth factor beta | NM_003573 | −0.38 | 0.18 |
| | binding protein 4 | | | |
| MAN2B2 | Mannosidase, alpha, class 2B, member 2 | NM_015274 | −0.32 | 0.11 |
| MCCC2 | Methylcrotonoyl-Coenzyme A | AK001948 | −0.26 | 0.09 |
| | carboxylase 2 (beta) | | | |
| MGC15523 | Hypothetical protein MGC15523 | BC020925 | −0.24 | 0.13 |
| MGC45780 | Hypothetical protein MGC45780 | NM_173833 | −0.68 | 0.30 |
| MYOC | Myocilin, trabecular meshwork | NM_000261 | −0.67 | 0.48 |
| | inducible glucocorticoid response | | | |
| NFYC | Nuclear transcription factor Y, gamma | NM_014223 | −0.36 | 0.14 |
| OPTN | Optineurin | NM_021980 | −0.41 | 0.30 |
| OSR2 | Odd-skipped related 2 (Drosophila) | NM_053001 | −1.06 | 0.74 |
| PAM | Peptidylglycine alpha-amidating | NM_000919 | −0.24 | 0.22 |
| | monooxygenase | | | |
| PBXIP1 | Pre-B-cell leukemia transcription factor | NM_020524 | | |
| | interacting protein 1 | | | |
| PCOLCE2 | Procollagen C-endopeptidase enhancer 2 | NM_013363 | −0.32 | 0.59 |
| PDGFRA | Platelet-derived growth factor receptor, | NM_006206 | −0.73 | 0.36 |
| | alpha polypeptide | | | |
| PDGFRL | Platelet-derived growth factor receptor- | NM_006207 | −0.48 | 0.24 |
| | like | | | |
| PERLD1 | Per1-like domain containing 1 | NM_033419 | −0.26 | 0.18 |
| PKP2 | Plakophilin 2 | X97675 | −0.27 | 0.14 |
| PPAP2B | Phosphatidic acid phosphatase type 2B | NM_003713 | −0.38 | 0.35 |
| PTGIS | Prostaglandin I2 (prostacyclin) synthase | NM_000961 | −0.80 | 0.17 |
| RECK | Reversion-inducing-cysteine-rich | NM_021111 | −0.47 | 0.36 |
| | protein with kazal motifs | | | |
| RIMS3 | Regulating synaptic membrane | NM_014747 | −0.22 | 0.17 |
| | exocytosis 3 | | | |
| RNASE4 | Angiogenin, ribonuclease, RNase A | NM_001145 | −0.47 | 0.32 |
| | family, 5 | | | |
| ROBO3 | Roundabout, axon guidance receptor, | NM_022370 | −0.47 | 0.33 |
| | homolog 3 (Drosophila) | | | |
| SAV1 | Salvador homolog 1 (Drosophila) | NM_021818 | −0.51 | 0.13 |
| SCGB1D1 | Secretoglobin, family 1D, member 1 | NM_006552 | −0.49 | 0.16 |
| SGCA | Sarcoglycan, alpha (50 kDa dystrophin- | NM_000023 | −0.20 | 0.22 |
| | associated glycoprotein) | | | |
| SH3RF2 | SH3 domain containing ring finger 2 | NM_152550 | −0.35 | 0.19 |
| SLC12A2 | Solute carrier family 12 | NM_001046 | −0.23 | 0.19 |
| | (sodium/potassium/chloride | | | |
| | transporters), member 2 | | | |
| SLC14A1 | Solute carrier family 14 (urea | L36121 | −0.32 | 0.18 |
| | transporter), member 1 (Kidd blood | | | |
| | group) | | | |
| SLC9A9 | Solute carrier family 9 | NM_173653 | −0.94 | 0.53 |
| | (sodium/hydrogen exchanger), isoform 9 | | | |
| SMAD1 | SMAD, mothers against DPP homolog | NM_005900 | −0.34 | 0.23 |
| | 1 (Drosophila) | | | |
| SOCS5 | Suppressor of cytokine signaling 5 | NM_014011 | −0.49 | 0.15 |
| SSPN | Sarcospan (Kras oncogene-associated | NM_005086 | −0.74 | 0.61 |
| | gene) | | | |
| STX7 | Syntaxin 7 | NM_003569 | −0.67 | 0.26 |
| TDE2 | Tumor differentially expressed 2 | NM_020755 | −0.40 | 0.37 |
| TM4SF3 | Transmembrane 4 superfamily member 3 | NM_004616 | −0.51 | 1.12 |
| TMEM25 | Transmembrane protein 25 | NM_032780 | −0.18 | 0.14 |
| TMEM34 | Transmembrane protein 34 | NM_018241 | −0.44 | 0.23 |
| TNA | Tetranectin (plasminogen binding | NM_003278 | −0.25 | 0.22 |
| | protein) | | | |
| TRAD | Serine/threonine kinase with Dbl- and | AL137629 | −0.34 | 0.13 |
| | pleckstrin homology domains | | | |
| UBL3 | Ubiquitin-like 3 | NM_007106 | −0.48 | 0.27 |
| ULK2 | Unc-51-like kinase 2 (C. elegans) | NM_014683 | −0.41 | 0.21 |
| UST | Uronyl-2-sulfotransferase | NM_005715 | −0.33 | 0.13 |
| WIF1 | WNT inhibitory factor 1 | NM_007191 | −1.01 | 0.38 |
| XG | Xg blood group (pseudoautosomal | NM_175569 | −0.90 | 0.48 |
| | boundary-divided on the X | | | |
| | chromosome) | | | |
| ZFHX1B | Zinc finger homeobox 1b | NM_014795 | −0.30 | 0.16 |
| | | A_32_BS53976 | −0.31 | 0.18 |
| | | AC025463 | −0.33 | 0.32 |
| | LOC440135 | AF318337 | −0.33 | 0.13 |
| | Homo sapiens, clone IMAGE: 4401608, | AK022793 | −0.50 | 0.10 |
| | mRNA | | | |
| | CDNA FLJ32177 fis, clone | AK056856 | −0.24 | 0.10 |
| | PLACE6001294 | | | |
| | MRNA; cDNA DKFZp566L0824 | AL050042 | −0.35 | 0.08 |
| | (from clone DKFZp566L0824) | | | |
| | Similar to jumonji domain containing | BC035102 | −0.33 | 0.09 |
| | 1A; testis-specific protein A; zinc | | | |
| | finger protein | | | |
| | | BG252130 | −0.37 | 0.14 |
| | | D80006 | −0.50 | 0.27 |
| | | ENST00000333784 | −0.20 | 0.17 |
| | Transcribed locus, weakly similar to | H16080 | −0.33 | 0.15 |
| | NP_808455.1 hypothetical protein | | | |
| | 9830102E05 [Mus musculus] | | | |
| | | I_1861543 | −0.42 | 0.30 |
| | | I_1882608 | −0.76 | 0.27 |
| | | I_1985061 | −0.43 | 0.17 |
| | | I_3335767 | −0.18 | 0.19 |
| | | I_3551568 | −0.57 | 0.37 |
| | | I_966091 | −0.23 | 0.08 |
| | | NM_001009555 | −0.53 | 0.24 |
| | | NM_001014975 | −0.89 | 0.42 |
| | | NM_001018076 | −0.79 | 0.20 |
| | | NM_138411 | −0.29 | 0.16 |
| | | NM_173709 | −0.48 | 0.22 |
| | | THC1429821 | −0.58 | 0.38 |
| | | THC1511927 | −0.38 | 0.08 |
| | | THC1544941 | −0.34 | 0.07 |
| | | THC1574967 | −0.65 | 0.60 |

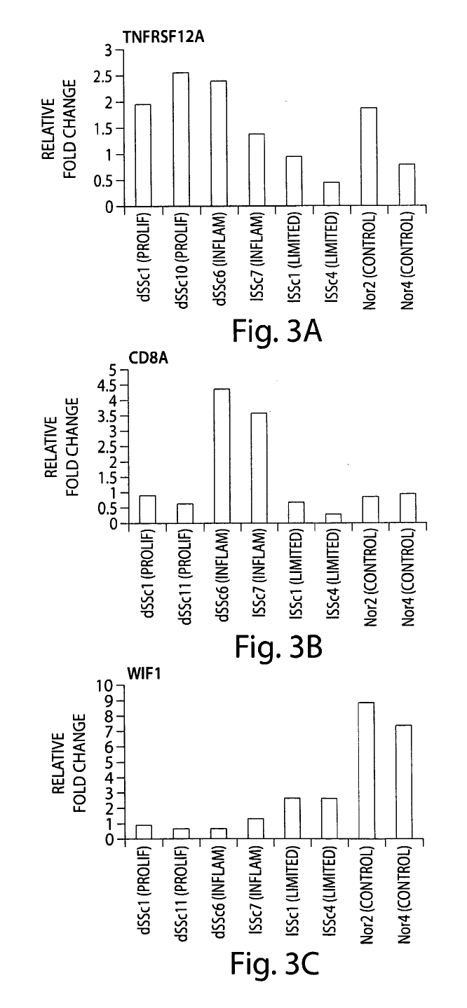

Quantitative Real-Time PCR. To validate the gene expression in the major groups found in this study, quantitative real time PCR (qRT-PCR) was performed on three genes selected from the intrinsic subsets (FIG. 3). These included TNFRSF12A, which was highly expressed in the dSSc patients and showed high expression in patients with increased MRSS; WIF1, which showed low expression in SSc and an association with increased MRSS; and CD8A, which was highly expressed in CD8+ T cells and was highly expressed in the inflammatory subset of patients. A representative sampling of patients from the intrinsic subsets was analyzed for expression of these three genes. Each was analyzed in triplicate and standardized to the expression of GAPDH. Each gene was shown with the fold change relative to the median value for the eight samples analyzed. TNFRSF12A showed highest expression in the patients with dSSc and the lowest in patients with limited SSc and normal controls. The three patients with highest expression were dSSc and included the proliferation group (FIG. 3A). CD8A showed highest expression in the inflammatory subgroup as predicted by the gene expression subsets (FIG. 3B). WIF1 showed highest expression in the healthy controls with approximately 4- to -8 fold relative decrease in patients with SSc (FIG. 3C). The most dramatic decrease was in patients with dSSc with smaller fold changes in patients with lSSc.

The gene expression groups disclosed herein were not likely to result from technical artifacts or heterogeneity at the site of biopsy because a standardized sample-processing pipeline was created, which was extensively tested on skin collected from surgical discards prior to beginning this study and included strict protocols that were used throughout with the goal of eliminating variability in sample handling and preparation. All gene expression groups were analyzed for correlation to date of hybridization, date of sample collection and other technical variables that might have affected the groupings. Also, heterogeneity at the site of biopsy was unlikely to account for the findings presented herein as the signatures used to classify the samples were selected by virtue of their being expressed in both the forearm and back samples of each patient. The inflammatory group was unlikely to be a result of active infection in patients as individuals with active infections were excluded from the study. Moreover, the gene expression signatures were verified by both immunohistochemical analysis and quantitative real-time PCR.

In addition, the gene expression signatures were found to be associated with changes in specific cell markers. We have confirmed infiltration of T cells in the dermis of the ‘inflammatory’ subgroup, and have confirmed an increase in the number of proliferating cells in the epidermis in the ‘proliferation’ group. The increase in the number of proliferating cells in the epidermis could result from paracrine influences on the resident keratinocytes, possibly activated by the profibrotic cytokine TGFβ. We were not able to find significant numbers of CD20 positive B cells.

### Example 2

### TGFβ-Activated Gene Expression Signature in Diffuse Scleroderma

Cells and Cell Culture. Clonetics primary adult human dermal fibroblasts were purchased from Cambrex Bio Science Walkersville, Inc. (Walkersville, Md.). Primary adult dermal fibroblasts were isolated from explant cultures of healthy and SSc forearm skin biopsies were cultured for at least three passages in Dulbecco's modified Eagle's medium (DMEM), 10% (v/v) fetal bovine serum (FBS), penicillin-streptomycin (100 IU/ml). Cells were passaged approximately every seven days for 7-10 passages prior to use in time course experiments. All incubations were conducted at 37° C. in a humidified atmosphere with 5% CO₂.

BrdU Staining. Cells were grown on coverslips as and cell proliferation assessed using a 5-Bromo-2′-deoxy-uridine Labeling and Detection Kit I (Roche Applied Sciences, Indianapolis, Ind.). Briefly, at appropriate time points, cells were labeled by incubating coverslips in DMEM supplemented with 0.1% FBS and 1× Streptomycin/Penicillin, at 37° C. in 5% CO₂ with 1×BrdU for 30 minutes. Cells were then fixed onto coverslips with an ethanol fixative solution and stored at −20° C. for up to 48 hours. BrdU incorporation was detected as per the manufacturer's instructions and counterstained with DAPI. Fluorescently labeled cells were then visualized.

Preparation of Samples for Microarray Hybridization. For time course experiments, 4×10⁵ cells were plated and cultured in DMEM-10% FBS for 48 hours. Cells were brought to quiescence by culturing in low serum media (DMEM-0.1% FBS) for 24 hours. Fifty pM of human TGFβ (R&D Systems, Minneapolis, Minn.)) in fresh low serum media or fresh low serum media alone was added to cells for 0, 2, 4, 8, 12 and 24 hours. Following each incubation with TGFβ, cells were fixed in RLT supplemented with β-mercaptoethanol and flash frozen to preserve RNA integrity. The cells were mechanically lysed and total RNA isolated using RNEASY minikits (QIAGEN, Valencia, Calif.).

Microarray Procedures. Each experimental sample RNA was hybridized against Universal Human Reference RNA (STRAGENE) onto Agilent Whole Human Genome Oligonucleotide microarrays of approximately 44,000 elements representing 41,000 human genes. For both experimental and reference RNAs, 300-500 ng of total RNA was amplified and labeled according to Agilent Low RNA Input Fluorescent Linear Amplification protocols.

Microarray Data Processing. Microarrays were scanned using a dual laser GENEPIX 4000B scanner (Axon Instruments, Foster City, Calif.). The pixel intensities of the acquired images were then quantified using GENEPIX Pro 5.1 software (Axon Instruments). Arrays were first visually inspected for defects or technical artifacts, poor quality spots were manually flagged and excluded from further analysis. The data was uploaded to the UNC Microarray Database. Spots with fluorescent signal at least 1.5 greater than local background in both channels and present in at least 80% of arrays were selected for further analysis.

Data Analysis. The data were downloaded from the UNC Microarray Database as log 2 of the lowess-normalized Cy5/Cy3 ratio. Each time course was TO transformed using the average of triplicate 0 hour samples. For Genomica analysis, where multiple probes were present for a single gene as annotated by Locus Link ID (LLID), the expression values were averaged. Genes without a LLID annotation were excluded from this analysis. Gene lists were downloaded and additional cell cycle-related gene lists were created using the data from Whitfield et al. (2003) supra. GOTerm Finder (Boyle, et al. (2004) Bioinformatics 20(18):3710-5) analysis was performed using implementation developed at the Lewis-Sigler Institute (Princeton, N.J.).

Quantitative Real Time PCR. For real-time polymerase chain reaction (PCR) assay 100-200 ng of total RNA samples were reverse-transcribed into single-stranded cDNA using SUPERSCRIPT II reverse transcriptase (INVITROGEN, San Diego, Calif.). cDNA samples were then diluted to the concentration of 250 pg/μL and 96-well optical plates were loaded with 20 μl of reaction mixture which contained: 1.25 μl of TAQMAN Primers and Probes mix, 12.5 μl of TAQMAN PCR Master Mix and 6.25 μl of nuclease-free water. Five ng of cDNA (5 μl of 1 ng/μl cDNA) was added to each well in duplicate. Reactions were performed using Applied Biosystems 7300 Real-Time PCR System (Applied Biosystems) by an initial incubation at 50° C. for 2 minutes and 95° C. for 10 minutes, and then cycled at 95° C. for 15 seconds and 60° C. for 1 minute for 40 cycles. Output data were generated by the instrument onboard software 7300 System version 1.2.2 (Applied Biosystems). The number of cycles required to generate a detectable fluorescence above background (CT) was measured for each sample. Fold difference between the initial mRNA levels of target genes (PAI-1, Coll1a1) in the experimental samples and Universal Human Reference RNA (UHR) (Stratagene) were calculated with the comparative CT method using formula 2-ΔΔCT. Here, ΔCT stands for the difference between the target gene and the housekeeping control, 18S rRNA, and ΔΔCT equals to the difference between the ΔCT value of the target gene in the experimental sample and in UHR.

The TGFβ-Responsive Signature in Adult Dermal Fibroblasts. Genes responsive to TGFβ exposure on a genome-wide scale were identified with DNA microarrays in adult dermal fibroblasts isolated from healthy individuals and patients with systemic sclerosis with dSSc. Four independent primary fibroblast cultures were isolated from forearm skin biopsies of either healthy controls or dSSc patients. Each time course was performed using cells cultured for 7-9 passages in 0.1% serum for 24 hours. It was reasoned that quiescent cells more closely approximated the state of fibroblasts in skin biopsies in vivo than asynchronously growing cells. Quiescent cells were exposed to 50 pM TGFβ and total RNA collected at six points over a period of 24 hours. The induction of a response to TGFβ was confirmed by measuring changes in PAI1 expression using TAQMAN quantitative real-time PCR (qRT-PCR). Total RNA from each sample was then amplified, labeled and hybridized against a common reference RNA (UHR) on whole genome DNA microarrays.

It was first sought to determine whether the genome-wide response to TGFβ in disease fibroblasts differed from that in fibroblasts from healthy controls. Significance Analysis of Microarrays (SAM) (Tusher, et al. (2001) Proc. Natl. Acad. Sci. USA 98(9):5116-21) was implemented using both slope and area functions in a 2-class unpaired time course analysis and found only a single gene that showed significant differences at an FDR of 0.05 or less between the two groups. This gene was the Early Growth Response 1 gene (EGR1). Upon detailed examination of the microarray data and qRT-PCR confirmation, this gene was found to be induced in three of four fibroblasts lines (two controls and one dSSc) upon TGFβ exposure. In a single SSc fibroblast line it was observed that the EGR1 gene was not induced.

As large numbers of genes that showed statistically significant differences in the responses of healthy and SSc fibroblasts to TGFβ exposure were not detected, it was reasoned that data from all experimental lines could be grouped together to characterize the genome-wide response to this potent cytokine. Furthermore, a study examining the response of pulmonary fibroblasts to TGFβ also found no discernable differences between SSc and healthy fibroblasts (Chambers, et al. (2003) Am. J. Pathol. 162(2):533-46). To identify the general TGF response across the time courses, probes were selected that changed at least a 1.74-fold in at least eight of the 32 arrays. The fold change threshold cutoff was determined by comparing genes induced or repressed in the presence of TGFβ over a range of cutoff values to a list of 26 known TGFβ targets compiled from published studies (Table 7).

TABLE 7

| Gene Symbol | Unigene Number | Tissue |
|---|---|---|
| COL1A1 | Z74615 | Gingiva; Foreskin |
| FN1 | NM_212482 | Gingiva; Foreskin |
| AGT1R | NM_031850 | Fetal Lung |
| SPHK1 | AK095578 | Fetal Lung; adult dermal; |
| | | foreskin |
| | | Fetal Lung |
| ACTSA | BX647362 | Gingiva |
| TIMP1 | BM913048 | A549 Cells (lung) |
| c-JUN | NM_002228 | A549 Cells (lung) |
| JUNB | CR601699 | A549 Cells (lung) |
| c-FOS | BX647104 | |
| COMP | BC033676 | |
| TGFB1 | X02812 | |
| CTFG | NM_0091001 | |
| PAI1 | M14083 | HEK293 Cell Line |
| P15^{Ink4B}/CDKN2B | NM_78487 | MC3T3-E1 cells^{a} |
| ITGB5 | AK091595 | HepG2 |
| APOC3 | BI521580 | Renal MECs |
| PDGFA | NM_002067 | Renal MECs |
| PDGFB | M12783 | Gingiva |
| SPARC | CR609946 | Gingiva |
| MMP2 | NM_004530 | |
| P21/Waf1 | NM_004780 | |
| COL7A1 | L02870 | |
| Id1 | BQ943400 | |
| Id2 | NM_010496 | |
| Id3 | BY703322 | |
| Id4 | NM_031166 | |
| THBS1 | NM_003246 | |

Genes previously reported as being TGFβ responsive in fibroblasts. Criteria for inclusion where defined as northern blot or qRT-PCR evidence for up or down regulation in response to TGFβ exposure. All targets were characterized in H. sapiens fibroblast cells unless otherwise indicated.
^{a}M. musculus osteoblast cell line.

In total, 894 TGFβ-responsive probes were selected that represented 674 unique annotated genes (Table 8). To ensure the capture of the most comprehensive biological response to TGFβ, all 894 probes were included in analyses where possible. Assessment of expression of these probes in the no treatment control showed that the observed changes in gene expression were specifically due to TGFβ induction or repression.

TABLE 8

| Gene | | |
|---|---|---|
| Symbol | Gene Name | Accession |
| ABTB2 | Ankyrin repeat and BTB (POZ) domain containing 2 | NM_145804 |
| ACAS2L | Acetyl-Coenzyme A synthetase 2 (AMP forming)-like | NM_032501 |
| ACOX1 | Acyl-Coenzyme A oxidase 1, palmitoyl | NM_004035 |
| ACOX2 | Acyl-Coenzyme A oxidase 2, branched chain | NM_003500 |
| ACTA2 | Actin, alpha 2, smooth muscle, aorta | NM_001613 |
| ACTC | Actin, alpha, cardiac muscle | NM_005159 |
| ACTN1 | Actinin, alpha 1 | NM_001102 |
| ACTN3 | Actinin, alpha 3 | NM_001104 |
| ACYP1 | Acylphosphatase 1, erythrocyte (common) type | NM_203488 |
| ADAM12 | A disintegrin and metalloproteinase domain 12 (meltrin | NM_003474 |
| | alpha) | |
| ADAM19 | A disintegrin and metalloproteinase domain 19 (meltrin beta) | NM_033274 |
| ADAMTS4 | A disintegrin-like and metalloprotease (reprolysin type) with | NM_005099 |
| | thrombospondin type 1 motif, 4 | |
| ADAMTS5 | A disintegrin-like and metalloprotease (reprolysin type) with | NM_007038 |
| | thrombospondin type 1 motif, 5 (aggrecanase-2) | |
| ADCY7 | Adenylate cyclase 7 | NM_001114 |
| ADH5 | Alcohol dehydrogenase 5 (class III), chi polypeptide | NM_000671 |
| ADM | Adrenomedullin | NM_001124 |
| AHR | Aryl hydrocarbon receptor | NM_001621 |
| AK3 | Adenylate kinase 3 | NM_001005353 |
| AK3 | Adenylate kinase 3 | AW467174 |
| AK5 | Adenylate kinase 5 | NM_174858 |
| AKAP12 | A kinase (PRKA) anchor protein (gravin) 12 | NM_144497 |
| AKR1C1 | Aldo-keto reductase family 1, member C2 (dihydrodiol | NM_001353 |
| | dehydrogenase 2; bile acid binding protein; 3-alpha | |
| | hydroxysteroid dehydrogenase, type III) | |
| AKR1C1 | Aldo-keto reductase family 1, member C2 (dihydrodiol | NM_001353 |
| | dehydrogenase 2; bile acid binding protein; 3-alpha | |
| | hydroxysteroid dehydrogenase, type III) | |
| AKR1C3 | Aldo-keto reductase family 1, member C3 (3-alpha | NM_003739 |
| | hydroxysteroid dehydrogenase, type II) | |
| ALS2CR4 | Amyotrophic lateral sclerosis 2 (juvenile) chromosome | NM_152388 |
| | region, candidate 4 | |
| ALS2CR4 | Amyotrophic lateral sclerosis 2 (juvenile) chromosome | BX538000 |
| | region, candidate 4 | |
| AMID | Apoptosis-inducing factor (AIF)-like mitochondrion- | NM_032797 |
| | associated inducer of death | |
| AMIGO2 | Amphoterin induced gene 2 | NM_181847 |
| AMOTL2 | Angiomotin like 2 | NM_016201 |
| AMSH-LP | Associated molecule with the SH3 domain of STAM | NM_020799 |
| | (AMSH) like protein | |
| ANGPTL2 | Angiopoietin-like 2 | NM_012098 |
| ANGPTL4 | Angiopoietin-like 4 | NM_139314 |
| ANTXR2 | Anthrax toxin receptor 2 | NM_058172 |
| ANXA11 | Annexin A11 | NM_145869 |
| APBB1IP | Amyloid beta (A4) precursor protein-binding, family B, | NM_019043 |
| | member 1 interacting protein | |
| APCDD1 | Adenomatosis polyposis coli down-regulated 1 | NM_153000 |
| APOL3 | Apolipoprotein L, 3 | NM_145641 |
| AQP1 | Aquaporin 1 (channel-forming integral protein, 28 kDa) | NM_198098 |
| AQP1 | Aquaporin 1 (channel-forming integral protein, 28 kDa) | NM_198098 |
| AR | Androgen receptor (dihydrotestosterone receptor; testicular | NM_000044 |
| | feminization; spinal and bulbar muscular atrophy; Kennedy | |
| | disease) | |
| ARG99 | ARG99 protein | NM_175861 |
| ARG99 | ARG99 protein | NM_175861 |
| ARHE | Ras homolog gene family, member E | NM_005168 |
| ARHGAP18 | Rho GTPase activating protein 18 | NM_033515 |
| ARKS | AMP-activated protein kinase family member 5 | NM_014840 |
| ARL4A | ADP-ribosylation factor-like 4A | NM_005738 |
| ARL4A | ADP-ribosylation factor-like 4A | NM_005738 |
| ARL6IP5 | ADP-ribosylation-like factor 6 interacting protein 5 | NM_006407 |
| ARL6IP5 | ADP-ribosylation-like factor 6 interacting protein 5 | NM_006407 |
| ARL7 | ADP-ribosylation factor-like 7 | NM_005737 |
| ARMCX1 | Armadillo repeat containing, X-linked 1 | NM_016608 |
| ARNT2 | Aryl-hydrocarbon receptor nuclear translocator 2 | NM_014862 |
| ARNTL | Aryl hydrocarbon receptor nuclear translocator-like | NM_001178 |
| ASB13 | Ankyrin repeat and SOCS box-containing 13 | NM_024701 |
| ASE-1 | CD3-epsilon-associated protein; antisense to ERCC-1 | NM_012099 |
| ASE-1 | CD3-epsilon-associated protein; antisense to ERCC-1 | NM_012099 |
| ASNS | Asparagine synthetase | NM_001673 |
| ASPM | Asp (abnormal spindle)-like, microcephaly associated | NM_018136 |
| | (Drosophila) | |
| ATOH8 | Atonal homolog 8 (Drosophila) | NM_032827 |
| ATP10A | ATPase, Class V, type 10A | NM_024490 |
| ATP1B1 | ATPase, Na+/K+ transporting, beta 1 polypeptide | NM_001677 |
| ATP1B1 | ATPase, Na+/K+ transporting, beta 1 polypeptide | NM_001677 |
| ATP2B4 | ATPase, Ca++ transporting, plasma membrane 4 | NM_001001396 |
| AVP | Arginine vasopressin (neurophysin II, antidiuretic hormone, | NM_000490 |
| | diabetes insipidus, neurohypophyseal) | |
| AVPI1 | Arginine vasopressin-induced 1 | NM_021732 |
| AXIN2 | Axin 2 (conductin, axil) | NM_004655 |
| AXUD1 | AXIN1 up-regulated 1 | NM_033027 |
| B3GALT4 | UDP-Gal: betaGlcNAc beta 1,3-galactosyltransferase, | NM_003782 |
| | polypeptide 4 | |
| B3GALT4 | UDP-Gal: betaGlcNAc beta 1,3-galactosyltransferase, | NM_003782 |
| | polypeptide 4 | |
| B4GALT1 | UDP-Gal: betaGlcNAc beta 1,4-galactosyltransferase, | NM_001497 |
| | polypeptide 1 | |
| BAG3 | BCL2-associated athanogene 3 | NM_004281 |
| BBC3 | BCL2 binding component 3 | NM_014417 |
| BCL2 | B-cell CLL/lymphoma 2 | NM_000633 |
| BCL3 | B-cell CLL/lymphoma 3 | NM_005178 |
| BDKRB1 | Bradykinin receptor B1 | NM_000710 |
| BDKRB1 | Bradykinin receptor B1 | NM_000710 |
| BDKRB2 | Bradykinin receptor B2 | NM_000623 |
| BFAR | Bifunctional apoptosis regulator | NM_016561 |
| BHLHB2 | Basic helix-loop-helix domain containing, class B, 2 | NM_003670 |
| BIN1 | Bridging integrator 1 | NM_139346 |
| BLOC1S2 | Biogenesis of lysosome-related organelles complex-1, subunit 2 | NM_001001342 |
| BLOC1S2 | Biogenesis of lysosome-related organelles complex-1, subunit 2 | NM_001001342 |
| BM039 | Uncharacterized bone marrow protein BM039 | AK023669 |
| BMP6 | Bone morphogenetic protein 6 | NM_001718 |
| BMPR2 | Bone morphogenetic protein receptor, type II | NM_001204 |
| | (serine/threonine kinase) | |
| BMPR2 | Bone morphogenetic protein receptor, type II | NM_001204 |
| | (serine/threonine kinase) | |
| BNC2 | Basonuclin 2 | NM_017637 |
| C10orf10 | Chromosome 10 open reading frame 10 | NM_007021 |
| C10orf22 | Chromosome 10 open reading frame 22 | NM_032804 |
| C10orf30 | Chromosome 10 open reading frame 30 | BC031618 |
| C14orf138 | Chromosome 14 open reading frame 138 | NM_024558 |
| C14orf139 | Chromosome 14 open reading frame 139 | BC008299 |
| C14orf31 | Chromosome 14 open reading frame 31 | NM_152330 |
| C16orf30 | Chromosome 16 open reading frame 30 | NM_024600 |
| C18orf1 | Chromosome 18 open reading frame 1 | NM_181482 |
| C1orf21 | Chromosome 1 open reading frame 21 | NM_030806 |
| C1orf21 | Chromosome 1 open reading frame 21 | NM_030806 |
| C20orf139 | Chromosome 20 open reading frame 139 | NM_080725 |
| C20orf161 | Chromosome 20 open reading frame 161 | NM_033421 |
| C20orf161 | Chromosome 20 open reading frame 161 | NM_033421 |
| C20orf19 | Chromosome 20 open reading frame 19 | NM_018474 |
| C20orf39 | Chromosome 20 open reading frame 39 | NM_024893 |
| C21orf93 | Chromosome 21 open reading frame 93 | NM_145179 |
| C2orf31 | Chromosome 2 open reading frame 31 | NM_003468 |
| C5orf13 | Chromosome 5 open reading frame 13 | NM_004772 |
| C5orf4 | Chromosome 5 open reading frame 4 | NM_032385 |
| C6orf145 | Chromosome 6 open reading frame 145 | NM_183373 |
| C6orf145 | Chromosome 6 open reading frame 145 | AI669333 |
| C6orf85 | Chromosome 6 open reading frame 85 | BC022217 |
| C9orf125 | Chromosome 9 open reading frame 125 | NM_032342 |
| C9orf150 | Chromosome 9 open reading frame 150 | NM_203403 |
| C9orf19 | Chromosome 9 open reading frame 19 | NM_022343 |
| C9orf3 | Chromosome 9 open reading frame 3 | NM_032823 |
| C9orf40 | Chromosome 9 open reading frame 40 | NM_017998 |
| C9orf62 | Chromosome 9 open reading frame 62 | BC034752 |
| CA12 | Carbonic anhydrase XII | NM_001218 |
| CABLES1 | Cdk5 and Abl enzyme substrate 1 | NM_138375 |
| CALM2 | Calmodulin 2 (phosphorylase kinase, delta) | NM_001743 |
| CAMK2G | Calcium/calmodulin-dependent protein kinase (CaM kinase) | NM_172171 |
| | II gamma | |
| CaMKIINalpha | Calcium/calmodulin-dependent protein kinase II | NM_018584 |
| CaMKIINalpha | Calcium/calmodulin-dependent protein kinase II | BC020630 |
| CAPS | Calcyphosine | NM_004058 |
| CARD10 | Caspase recruitment domain family, member 10 | NM_014550 |
| CARD4 | Caspase recruitment domain family, member 4 | NM_006092 |
| CASP1 | Caspase 1, apoptosis-related cysteine protease (interleukin 1, | NM_033292 |
| | beta, convertase) | |
| CAT | Catalase | NM_001752 |
| CAV1 | Caveolin 1, caveolae protein, 22 kDa | NM_001753 |
| CBFB | Core-binding factor, beta subunit | NM_022845 |
| CBFB | Core-binding factor, beta subunit | NM_001755 |
| CBX7 | Chromobox homolog 7 | NM_175709 |
| CBX7 | Chromobox homolog 7 | NM_175709 |
| CCDC8 | Coiled-coil domain containing 8 | NM_032040 |
| CCL2 | Chemokine (C-C motif) ligand 2 | NM_002982 |
| CCNB1 | Cyclin B1 | NM_031966 |
| CCNB2 | Cyclin B2 | NM_004701 |
| CD44 | CD44 antigen (homing function and Indian blood group | NM_000610 |
| | system) | |
| CDC42EP2 | CDC42 effector protein (Rho GTPase binding) 2 | NM_006779 |
| CDCA2 | Cell division cycle associated 2 | NM_152562 |
| CDCA8 | Cell division cycle associated 8 | NM_018101 |
| CDH18 | Cadherin 18, type 2 | NM_004934 |
| CDH2 | Cadherin 2, type 1, N-cadherin (neuronal) | NM_001792 |
| CDKN2B | Cyclin-dependent kinase inhibitor 2B (p15, inhibits CDK4) | NM_078487 |
| CDKN2D | Cyclin-dependent kinase inhibitor 2D (p19, inhibits CDK4) | NM_001800 |
| CDON | Cell adhesion molecule-related/down-regulated by oncogenes | NM_016952 |
| CDT1 | DNA replication factor | NM_030928 |
| CEBPA | CCAAT/enhancer binding protein (C/EBP), alpha | NM_004364 |
| CEBPD | CCAAT/enhancer binding protein (C/EBP), delta | NM_005195 |
| CENPF | Centromere protein F, 350/400ka (mitosin) | NM_016343 |
| CFL2 | Cofilin 2 (muscle) | NM_021914 |
| CGI-14 | CGI-14 protein | AL833099 |
| CH25H | Cholesterol 25-hydroxylase | NM_003956 |
| CHIC2 | Cysteine-rich hydrophobic domain 2 | NM_012110 |
| CHST11 | Carbohydrate (chondroitin 4) sulfotransferase 11 | AF131762 |
| CHST2 | Carbohydrate (N-acetylglucosamine-6-O) sulfotransferase 2 | NM_004267 |
| CHST5 | Carbohydrate (N-acetylglucosamine 6-O) sulfotransferase 5 | BC010609 |
| CHSY1 | Carbohydrate (chondroitin) synthase 1 | NM_014918 |
| CIT | Citron (rho-interacting, serine/threonine kinase 21) | NM_007174 |
| CITED2 | Cbp/p300-interacting transactivator, with Glu/Asp-rich | NM_006079 |
| | carboxy-terminal domain, 2 | |
| CLC | Cardiotrophin-like cytokine | NM_013246 |
| CLECSF2 | C-type (calcium dependent, carbohydrate-recognition | BF213738 |
| | domain) lectin, superfamily member 2 (activation-induced) | |
| CMKOR1 | Chemokine orphan receptor 1 | NM_020311 |
| CNAP1 | Chromosome condensation-related SMC-associated protein 1 | NM_014865 |
| CNN3 | Calponin 3, acidic | NM_001839 |
| CNN3 | Calponin 3, acidic | BM668321 |
| COL4A1 | Collagen, type IV, alpha 1 | NM_001845 |
| COL4A2 | Collagen, type IV, alpha 2 | NM_001846 |
| COL5A1 | Collagen, type V, alpha 1 | NM_000093 |
| COL5A2 | Collagen, type V, alpha 2 | NM_000393 |
| COLEC12 | Collectin sub-family member 12 | NM_030781 |
| COMP | Cartilage oligomeric matrix protein | NM_000095 |
| COMP | Cartilage oligomeric matrix protein | NM_000095 |
| CREB3L2 | CAMP responsive element binding protein 3-like 2 | BC063666 |
| CRLF1 | Cytokine receptor-like factor 1 | NM_004750 |
| CRY1 | Cryptochrome 1 (photolyase-like) | NM_004075 |
| CRYZ | Crystallin, zeta (quinone reductase) | NM_001889 |
| CSRP1 | Cysteine and glycine-rich protein 1 | NM_004078 |
| CSRP2 | Cysteine and glycine-rich protein 2 | NM_001321 |
| CTGF | Connective tissue growth factor | NM_001901 |
| CTPS | CTP synthase | NM_001905 |
| CTSC | Cathepsin C | NM_148170 |
| CXCL12 | Chemokine (C—X—C motif) ligand 12 (stromal cell-derived | AK090482 |
| | factor 1) | |
| CXXC5 | CXXC finger 5 | NM_016463 |
| CXXC5 | CXXC finger 5 | NM_016463 |
| CYB5 | Cytochrome b-5 | NM_001914 |
| CYP1B1 | Cytochrome P450, family 1, subfamily B, polypeptide 1 | NM_000104 |
| CYR61 | Cysteine-rich, angiogenic inducer, 61 | NM_001554 |
| DACT1 | Dapper homolog 1, antagonist of beta-catenin (xenopus) | NM_016651 |
| DCAMKL1 | Doublecortin and CaM kinase-like 1 | NM_004734 |
| DDIT4 | DNA-damage-inducible transcript 4 | NM_019058 |
| DIPA | Hepatitis delta antigen-interacting protein A | NM_006848 |
| DKFZP434I216 | DKFZP434I216 protein | NM_015432 |
| DKFZp434L142 | Hypothetical protein DKFZp434L142 | NM_016613 |
| DKFZP586A0522 | DKFZP586A0522 protein | NM_014033 |
| DKFZp762O076 | Hypothetical protein DKEZp762O076 | NM_018710 |
| DKK1 | Dickkopf homolog 1 (Xenopus laevis) | NM_012242 |
| DLC1 | Deleted in liver cancer 1 | NM_182643 |
| DLX2 | Distal-less homeo box 2 | NM_004405 |
| DNAJB4 | DnaJ (Hsp40) homolog, subfamily B, member 4 | NM_007034 |
| DNAJB5 | DnaJ (Hsp40) homolog, subfamily B, member 5 | NM_012266 |
| DNAJB9 | DnaJ (Hsp40) homolog, subfamily B, member 9 | NM_012328 |
| DOK5L | Docking protein 5-like | NM_152721 |
| DSCR1L1 | Down syndrome critical region gene 1-like 1 | NM_005822 |
| DSP | Desmoplakin | NM_004415 |
| DTR | Diphtheria toxin receptor (heparin-binding epidermal growth | NM_001945 |
| | factor-like growth factor) | |
| DUSP1 | Dual specificity phosphatase 1 | NM_004417 |
| DUSP6 | Dual specificity phosphatase 6 | NM_001946 |
| DYRK2 | Dual-specificity tyrosine-(Y)-phosphorylation regulated | NM_006482 |
| | kinase 2 | |
| DYRK2 | Dual-specificity tyrosine-(Y)-phosphorylation regulated | NM_006482 |
| | kinase 2 | |
| DYRK2 | Dual-specificity tyrosine-(Y)-phosphorylation regulated | CR612226 |
| | kinase 2 | |
| E2F7 | E2F transcription factor 7 | NM_203394 |
| EBF | Early B-cell factor | AK123757 |
| EFNA1 | Ephrin-A1 | NM_004428 |
| EFNB2 | Ephrin-B2 | NM_004093 |
| EGR1 | Early growth response 1 | NM_001964 |
| EHBP1 | EH domain binding protein 1 | NM_015252 |
| EIF4EBP1 | Eukaryotic translation initiation factor 4E binding protein 1 | NM_004095 |
| ELN | Elastin (supravalvular aortic stenosis, Williams-Beuren | NM_000501 |
| | syndrome) | |
| ELN | Elastin (supravalvular aortic stenosis, Williams-Beuren | AK075554 |
| | syndrome) | |
| ENC1 | Ectodermal-neural cortex (with BTB-like domain) | NM_003633 |
| ENC1 | Ectodermal-neural cortex (with BTB-like domain) | NM_003633 |
| ENPP1 | Ectonucleotide pyrophosphatase/phosphodiesterase 1 | NM_006208 |
| EPHB3 | EPH receptor B3 | NM_004443 |
| EPHX2 | Epoxide hydrolase 2, cytoplasmic | NM_001979 |
| ERN1 | Endoplasmic reticulum to nucleus signalling 1 | NM_152461 |
| EYA2 | Eyes absent homolog 2 (Drosophila) | NM_172113 |
| FAM46A | Family with sequence similarity 46, member A | NM_017633 |
| FANCE | Fanconi anemia, complementation group E | NM_021922 |
| FBXO32 | F-box protein 32 | NM_058229 |
| FDXR | Ferredoxin reductase | NM_024417 |
| FGF18 | Fibroblast growth factor 18 | NM_003862 |
| FGF2 | Fibroblast growth factor 2 (basic) | NM_002006 |
| FGF9 | Fibroblast growth factor 9 (glia-activating factor) | NM_002010 |
| FGFR3 | Fibroblast growth factor receptor 3 (achondroplasia, | NM_000142 |
| | thanatophoric dwarfism) | |
| FGFRL1 | Fibroblast growth factor receptor-like 1 | NM_001004356 |
| FHL2 | Four and a half LIM domains 2 | NM_201555 |
| FLJ10350 | Hypothetical protein FLJ10350 | NM_018067 |
| FLJ10357 | Hypothetical protein FLJ10357 | NM_018071 |
| FLJ10378 | FLJ10378 protein | NM_032239 |
| FLJ12118 | Hypothetical protein FLJ12118 | NM_024537 |
| FLJ12436 | Hypothetical protein FLJ12436 | NM_024661 |
| FLJ12584 | Hypothetical protein FLJ12584 | NM_025139 |
| FLJ14054 | Hypothetical protein FLJ14054 | NM_024563 |
| FLJ20245 | Hypothetical protein FLJ20245 | NM_017723 |
| FLJ20364 | Hypothetical protein FLJ20364 | NM_017785 |
| FLJ20366 | Hypothetical protein FLJ20366 | NM_017786 |
| FLJ20701 | Hypothetical protein FLJ20701 | NM_017933 |
| FLJ22938 | Hypothetical protein FLJ22938 | NM_024676 |
| FLJ23091 | Putative NFkB activating protein 373 | NM_024911 |
| FLJ23221 | Hypothetical protein FLJ23221 | NM_024579 |
| FLJ25124 | Hypothetical protein FLJ25124 | NM_144698 |
| FLJ32009 | Hypothetical protein FLJ32009 | NM_152718 |
| FLJ33674 | Hypothetical protein FLJ33674 | NM_207351 |
| FLJ34389 | Hypothetical protein FLJ34389 | NM_152649 |
| FLJ37970 | Hypothetical protein FLJ37970 | NM_032251 |
| FLJ39370 | Hypothetical protein FLJ39370 | NM_152400 |
| FLJ39370 | Hypothetical protein FLJ39370 | NM_152400 |
| FLJ43339 | FLJ43339 protein | NM_207380 |
| FLJ45248 | FLJ45248 protein | NM_207505 |
| FN5 | FN5 protein | NM_020179 |
| FNBP1 | Formin binding protein 1 | NM_015033 |
| FOS | V-fos FBJ murine osteosarcoma viral oncogene homolog | NM_005252 |
| FOXP1 | Forkhead box P1 | NM_032682 |
| FOXP1 | Forkhead box P1 | NM_032682 |
| FSTL3 | Follistatin-like 3 (secreted glycoprotein) | NM_005860 |
| FUS | Fusion (involved in t(12;16) in malignant liposarcoma) | NM_004960 |
| FZD8 | Frizzled homolog 8 (Drosophila) | NM_031866 |
| GABRE | Gamma-aminobutyric acid (GABA) A receptor, epsilon | NM_021990 |
| GADD45B | Growth arrest and DNA-damage-inducible, beta | NM_015675 |
| GADD45B | Growth arrest and DNA-damage-inducible, beta | NM_015675 |
| GALM | Galactose mutarotase (aldose 1-epimerase) | NM_138801 |
| GALT | Galactose-1-phosphate uridylyltransferase | NM_000155 |
| GARS | Glycyl-tRNA synthetase | NM_002047 |
| GAS1 | Growth arrest-specific 1 | NM_002048 |
| GAS7 | Growth arrest-specific 7 | NM_201433 |
| GAS7 | Growth arrest-specific 7 | NM_201433 |
| GATA6 | GATA binding protein 6 | NM_005257 |
| GCNT1 | Glucosaminyl (N-acetyl) transferase 1, core 2 (beta-1,6-N- | NM_001490 |
| | acetylglucosaminyltransferase) | |
| GDF15 | Growth differentiation factor 15 | NM_004864 |
| GDF6 | Growth differentiation factor 6 | NM_001001557 |
| GEM | GTP binding protein overexpressed in skeletal muscle | NM_005261 |
| GGA2 | Golgi associated, gamma adaptin ear containing, ARF | NM_015044 |
| | binding protein 2 | |
| GGH | Gamma-glutamyl hydrolase (conjugase, | NM_003878 |
| | folylpolygammaglutamyl hydrolase) | |
| GLI3 | GLI-Kruppel family member GLI3 (Greig | NM_000168 |
| | cephalopolysyndactyly syndrome) | |
| GLS | Glutaminase | NM_014905 |
| GLS | Glutaminase | NM_014905 |
| GLS | Glutaminase | AF158555 |
| GNAI1 | Guanine nucleotide binding protein (G protein), alpha | NM_002069 |
| | inhibiting activity polypeptide 1 | |
| GNPNAT1 | Glucosamine-phosphate N-acetyltransferase 1 | NM_198066 |
| GNPNAT1 | Glucosamine-phosphate N-acetyltransferase 1 | NM_198066 |
| GOPC | Golgi associated PDZ and coiled-coil motif containing | NM_020399 |
| GOPC | Golgi associated PDZ and coiled-coil motif containing | NM_020399 |
| GPAM | Glycerol-3-phosphate acyltransferase, mitochondrial | NM_020918 |
| GPR30 | G protein-coupled receptor 30 | NM_001505 |
| GPR68 | G protein-coupled receptor 68 | NM_003485 |
| GPSM2 | G-protein signalling modulator 2 (AGS3-like, C. elegans) | NM_013296 |
| GPT2 | Glutamic pyruvate transaminase (alanine aminotransferase) 2 | NM_133443 |
| GRASP | GRP1 (general receptor for phosphoinositides 1)-associated | NM_181711 |
| | scaffold protein | |
| GRK5 | G protein-coupled receptor kinase 5 | NM_005308 |
| GSC | Goosecoid | NM_173849 |
| GSTT2 | Glutathione S-transferase theta 2 | NM_000854 |
| GULP1 | GULP, engulfment adaptor PTB domain containing 1 | NM_016315 |
| HBLD1 | HESB like domain containing 1 | NM_194279 |
| HCAP-G | Chromosome condensation protein G | NM_022346 |
| HCMOGT-1 | Sperm antigen HCMOGT-1 | NM_152904 |
| HEBP1 | Heme binding protein 1 | NM_015987 |
| HES1 | Hairy and enhancer of split 1, (Drosophila) | NM_005524 |
| HES1 | Hairy and enhancer of split 1, (Drosophila) | NM_005524 |
| HIBCH | 3-hydroxyisobutyryl-Coenzyme A hydrolase | NM_014362 |
| HIF1A | Hypoxia-inducible factor 1, alpha subunit (basic helix-loop- | NM_181054 |
| | helix transcription factor) | |
| HIF1A | Hypoxia-inducible factor 1, alpha subunit (basic helix-loop- | BG108194 |
| | helix transcription factor) | |
| HILS1 | Spermatid-specific linker histone H1-like protein | NM_194072 |
| HIP1R | Huntingtin interacting protein-1-related | NM_003959 |
| HMGB2 | High-mobility group box 2 | NM_002129 |
| HNRPAB | Heterogeneous nuclear ribonucleoprotein A/B | NM_004499 |
| HNRPK | Heterogeneous nuclear ribonucleoprotein K | BG058000 |
| HOMER1 | Homer homolog 1 (Drosophila) | NM_004272 |
| HOM-TES- | HOM-TES-103 tumor antigen-like | NM_080731 |
| 103 | | |
| HOXA7 | Homeo box A7 | NM_006896 |
| HOXB2 | Homeo box B2 | NM_002145 |
| HOXC8 | Homeo box C8 | NM_022658 |
| HSPA5 | Heat shock 70 kDa protein 5 (glucose-regulated protein, | NM_005347 |
| | 78 kDa) | |
| HSPB7 | Heat shock 27 kDa protein family, member 7 (cardiovascular) | NM_014424 |
| HSXIAPAF1 | XIAP associated factor-1 | NM_017523 |
| ID1 | Inhibitor of DNA binding 1, dominant negative helix-loop- | NM_002165 |
| | helix protein | |
| ID1 | Inhibitor of DNA binding 1, dominant negative helix-loop- | CN479126 |
| | helix protein | |
| ID2 | Inhibitor of DNA binding 2, dominant negative helix-loop- | NM_002166 |
| | helix protein | |
| ID2 | Inhibitor of DNA binding 2, dominant negative helix-loop- | NM_002166 |
| | helix protein | |
| ID3 | Inhibitor of DNA binding 3, dominant negative helix-loop- | NM_002167 |
| | helix protein | |
| ID3 | Inhibitor of DNA binding 3, dominant negative helix-loop- | AW327568 |
| | helix protein | |
| ID4 | Inhibitor of DNA binding 4, dominant negative helix-loop- | NM_001546 |
| | helix protein | |
| IDH1 | Isocitrate dehydrogenase 1 (NADP+), soluble | NM_005896 |
| IER2 | Immediate early response 2 | NM_004907 |
| IER2 | Immediate early response 2 | NM_004907 |
| IER3 | Immediate early response 3 | NM_003897 |
| IER5L | Immediate early response 5-like | NM_203434 |
| IFIT1 | Interferon-induced protein with tetratricopeptide repeats 1 | NM_001548 |
| IFIT2 | Interferon-induced protein with tetratricopeptide repeats 2 | NM_001547 |
| IGF1 | Insulin-like growth factor 1 (somatomedin C) | NM_000618 |
| IL11 | Interleukin 11 | NM_000641 |
| IL21R | Interleukin 21 receptor | NM_181078 |
| IL4R | Interleukin 4 receptor | NM_000418 |
| IL6 | Interleukin 6 (interferon, beta 2) | NM_000600 |
| IL6R | Interleukin 6 receptor | NM_000565 |
| INHBB | Inhibin, beta B (activin AB beta polypeptide) | NM_002193 |
| IRF2 | Interferon regulatory factor 2 | NM_002199 |
| ITR | Intimal thickness-related receptor | NM_180989 |
| IVNS1ABP | Influenza virus NS1A binding protein | NM_016389 |
| IVNS1ABP | Influenza virus NS1A binding protein | NM_016389 |
| JUN | V-jun sarcoma virus 17 oncogene homolog (avian) | NM_002228 |
| JUNB | Jun B proto-oncogene | NM_002229 |
| JUNB | Jun B proto-oncogene | NM_002229 |
| K-ALPHA-1 | Tubulin, alpha, ubiquitous | AI608782 |
| KCNE4 | Potassium voltage-gated channel, Isk-related family, member 4 | NM_080671 |
| KCNG1 | Potassium voltage-gated channel, subfamily G, member 1 | NM_002237 |
| KCNK1 | Potassium channel, subfamily K, member 1 | NM_002245 |
| KCNN4 | Potassium intermediate/small conductance calcium-activated | NM_002250 |
| | channel, subfamily N, member 4 | |
| KCNS3 | Potassium voltage-gated channel, delayed-rectifier, subfamily | NM_002252 |
| | S, member 3 | |
| KCTD11 | Potassium channel tetramerisation domain containing 11 | NM_001002914 |
| KIAA0033 | KIAA0033 protein | BC035034 |
| KIAA0101 | KIAA0101 | NM_014736 |
| KIAA0280 | KIAA0280 protein | D87470 |
| KIAA0802 | KIAA0802 | BC040542 |
| KIAA1102 | KIAA1102 protein | NM_014988 |
| KIAA1199 | KIAA1199 | NM_018689 |
| KIAA1199 | KIAA1199 | NM_018689 |
| KIAA1644 | KIAA1644 protein | AB051431 |
| KIAA1666 | KIAA1666 protein | BC035246 |
| KIAA1683 | KIAA1683 | NM_025249 |
| KIAA1754 | KIAA1754 | NM_033397 |
| KIF20A | Kinesin family member 20A | NM_005733 |
| KIT | V-kit Hardy-Zuckerman 4 feline sarcoma viral oncogene | NM_000222 |
| | homolog | |
| KITLG | KIT ligand | NM_000899 |
| KLF10 | Kruppel-like factor 10 | NM_005655 |
| KLF13 | Kruppel-like factor 13 | NM_015995 |
| KLF2 | Kruppel-like factor 2 (lung) | NM_016270 |
| KNTC2 | Kinetochore associated 2 | NM_006101 |
| KRTAP1-5 | Keratin associated protein 1-5 | NM_031957 |
| KUB3 | Ku70-binding protein 3 | NM_033276 |
| LDHA | Lactate dehydrogenase A | NM_005566 |
| LDHA | Lactate dehydrogenase A | NM_005566 |
| LGALS3 | Lectin, galactoside-binding, soluble, 3 (galectin 3) | NM_002306 |
| LHFPL2 | Lipoma HMGIC fusion partner-like 2 | NM_005779 |
| LIF | Leukemia inhibitory factor (cholinergic differentiation factor) | NM_002309 |
| LIM | LIM protein (similar to rat protein kinase C-binding enigma) | NM_006457 |
| LIM | LIM protein (similar to rat protein kinase C-binding enigma) | NM_006457 |
| LIMK1 | LIM domain kinase 1 | NM_002314 |
| LIMK2 | LIM domain kinase 2 | NM_016733 |
| LIMS3 | LIM and senescent cell antigen-like domains 3 | NM_033514 |
| LMCD1 | LIM and cysteine-rich domains 1 | NM_014583 |
| LMNA | Lamin A/C | NM_005572 |
| LMNB1 | Lamin B1 | NM_005573 |
| LMO4 | LIM domain only 4 | NM_006769 |
| LOC112476 | Similar to lymphocyte antigen 6 complex, locus G5B; G5b | NM_145239 |
| | protein; open reading frame 31 | |
| LOC134147 | Hypothetical protein BC001573 | NM_138809 |
| LOC143903 | Layilin | NM_178834 |
| LOC222171 | Hypothetical protein LOC222171 | NM_175887 |
| LOC283824 | Hypothetical protein LOC283824 | BC045778 |
| LOC284454 | Hypothetical protein LOC284454 | AL832183 |
| LOC339047 | Hypothetical protein LOC339047 | BC008178 |
| LOC51149 | Truncated calcium binding protein | NM_016175 |
| LOC51161 | G20 protein | NM_016210 |
| LOC51333 | Mesenchymal stem cell protein DSC43 | NM_016643 |
| LOC57146 | Promethin | NM_020422 |
| LOC81558 | C/EBP-induced protein | NM_030802 |
| LPIN1 | Lipin 1 | NM_145693 |
| LRIG1 | Leucine-rich repeats and immunoglobulin-like domains 1 | NM_015541 |
| LRIG3 | Leucine-rich repeats and immunoglobulin-like domains 3 | NM_153377 |
| LRRC20 | Leucine rich repeat containing 20 | NM_018205 |
| LRRC8 | Leucine rich repeat containing 8 | NM_019594 |
| LSS | Lanosterol synthase (2,3-oxidosqualene-lanosterol cyclase) | NM_002340 |
| LSS | Lanosterol synthase (2,3-oxidosqualene-lanosterol cyclase) | NM_002340 |
| LTBP2 | Latent transforming growth factor beta binding protein 2 | NM_000428 |
| LY6K | Lymphocyte antigen 6 complex, locus K | NM_017527 |
| LY6K | Lymphocyte antigen 6 complex, locus K | NM_017527 |
| MAFB | V-maf musculoaponeurotic fibrosarcoma oncogene homolog | NM_005461 |
| | B (avian) | |
| MAGI1 | Membrane associated guanylate kinase interacting protein- | NM_173515 |
| | like 1 | |
| MAN1C1 | Mannosidase, alpha, class 1C, member 1 | NM_020379 |
| MAP3K2 | Mitogen-activated protein kinase kinase kinase 2 | NM_006609 |
| MAP3K2 | Mitogen-activated protein kinase kinase kinase 2 | NM_006609 |
| MAP3K8 | Mitogen-activated protein kinase kinase kinase 8 | NM_005204 |
| MAPRE2 | Microtubule-associated protein, RP/EB family, member 2 | NM_014268 |
| MBD4 | Methyl-CpG binding domain protein 4 | NM_003925 |
| MCCC1 | Methylcrotonoyl-Coenzyme A carboxylase 1 (alpha) | NM_020166 |
| MEIS2 | Meis1, myeloid ecotropic viral integration site 1 homolog 2 | NM_170677 |
| | (mouse) | |
| MEIS2 | Meis1, myeloid ecotropic viral integration site 1 homolog 2 | NM_170676 |
| | (mouse) | |
| MGC14376 | Hypothetical protein MGC14376 | NM_032895 |
| MGC15476 | Thymus expressed gene 3-like | NM_145056 |
| MGC16121 | Hypothetical protein MGC16121 | BC007360 |
| MGC29875 | Hypothetical protein MGC29875 | NM_014388 |
| MGC33584 | Hypothetical protein MGC33584 | NM_173680 |
| MGC39325 | Hypothetical protein MGC39325 | NM_147189 |
| MGC4504 | Hypothetical protein MGC4504 | NM_024111 |
| MGC4562 | Hypothetical protein MGC4562 | NM_133375 |
| MGC45871 | Hypothetical protein MGC45871 | NM_182705 |
| MGC45871 | Hypothetical protein MGC45871 | BC014203 |
| MGC5576 | Hypothetical protein MGC5576 | NM_024056 |
| MGC7036 | Hypothetical protein MGC7036 | NM_145058 |
| MGC8685 | Tubulin, beta polypeptide paralog | NM_178012 |
| MGLL | Monoglyceride lipase | NM_007283 |
| MICAL2 | Flavoprotein oxidoreductase MICAL2 | NM_014632 |
| MICAL-L1 | MICAL-like 1 | NM_033386 |
| MID1 | Midline 1 (Opitz/BBB syndrome) | NM_033290 |
| MITF | Microphthalmia-associated transcription factor | NM_198159 |
| MITF | Microphthalmia-associated transcription factor | NM_198159 |
| MKL2 | MKL/myocardin-like 2 | NM_014048 |
| MKNK2 | MAP kinase interacting serine/threonine kinase 2 | NM_017572 |
| MLPH | Melanophilin | NM_024101 |
| MMP1 | Matrix metalloproteinase 1 (interstitial collagenase) | NM_002421 |
| MONDOA | Mlx interactor | AB020674 |
| MRC2 | Mannose receptor, C type 2 | BC033590 |
| MRGPRF | MAS-related GPR, member F | NM_145015 |
| MRPS24 | Mitochondrial ribosomal protein S24 | NM_032014 |
| MSX1 | Msh homeo box homolog 1 (Drosophila) | NM_002448 |
| MT1K | Metallothionein 1K | NM_176870 |
| MTCH1 | Mitochondrial carrier homolog 1 (C. elegans) | NM_014341 |
| MTCH1 | Mitochondrial carrier homolog 1 (C. elegans) | NM_014341 |
| MTHFD2 | Methylene tetrahydrofolate dehydrogenase (NAD+ | NM_006636 |
| | dependent), methenyltetrahydrofolate cyclohydrolase | |
| MTHFR | 5,10-methylenetetrahydrofolate reductase (NADPH) | NM_005957 |
| MTHFR | 5,10-methylenetetrahydrofolate reductase (NADPH) | NM_005957 |
| MTHFR | 5,10-methylenetetrahydrofolate reductase (NADPH) | NM_005957 |
| MTMR4 | Myotubularin related protein 4 | NM_004687 |
| MYCBP2 | MYC binding protein 2 | NM_015057 |
| MYLIP | Myosin regulatory light chain interacting protein | NM_013262 |
| NEDD4 | Neural precursor cell expressed, developmentally down- | NM_006154 |
| | regulated 4 | |
| NEDD9 | Neural precursor cell expressed, developmentally down- | NM_006403 |
| | regulated 9 | |
| NET1 | Neuroepithelial cell transforming gene 1 | NM_005863 |
| NFATC1 | Nuclear factor of activated T-cells, cytoplasmic, calcineurin- | NM_172387 |
| | dependent 1 | |
| NFIA | Nuclear factor I/A | NM_005595 |
| NFYC | Nuclear transcription factor Y, gamma | AK094323 |
| NGEF | Neuronal guanine nucleotide exchange factor | NM_019850 |
| NID67 | Putative small membrane protein NID67 | NM_032947 |
| NKD2 | Naked cuticle homolog 2 (Drosophila) | NM_033120 |
| NLF1 | Nuclear localized factor 1 | NM_207322 |
| NNMT | Nicotinamide N-methyltransferase | NM_006169 |
| NOL3 | Nucleolar protein 3 (apoptosis repressor with CARD domain) | NM_003946 |
| NOV | Nephroblastoma overexpressed gene | NM_002514 |
| NP | Nucleoside phosphorylase | NM_000270 |
| NPAS1 | Neuronal PAS domain protein 1 | NM_002517 |
| NPEPPS | Aminopeptidase puromycin sensitive | NM_006310 |
| NPTX1 | Neuronal pentraxin I | NM_002522 |
| NR0B1 | Nuclear receptor subfamily 0, group B, member 1 | NM_000475 |
| NR1D2 | Nuclear receptor subfamily 1, group D, member 2 | BC015929 |
| NR2F2 | Nuclear receptor subfamily 2, group F, member 2 | NM_021005 |
| NR3C1 | Nuclear receptor subfamily 3, group C, member 1 | NM_000176 |
| | (glucocorticoid receptor) | |
| NRBF2 | Nuclear receptor binding factor 2 | NM_030759 |
| NRG1 | Neuregulin 1 | NM_013957 |
| NRP1 | Neuropilin 1 | NM_003873 |
| NTHL1 | Nth endonuclease III-like 1 (E. coli) | NM_002528 |
| NUP98 | Nucleoporin 98 kDa | NM_005387 |
| ODC1 | Ornithine decarboxylase 1 | NM_002539 |
| OSR2 | Odd-skipped related 2 (Drosophila) | NM_053001 |
| OSR2 | Odd-skipped related 2 (Drosophila) | NM_053001 |
| P4HA2 | Procollagen-proline, 2-oxoglutarate 4-dioxygenase (proline 4- | BC013423 |
| | hydroxylase), alpha polypeptide II | |
| P4HA3 | Procollagen-proline, 2-oxoglutarate 4-dioxygenase (proline 4- | NM_182904 |
| | hydroxylase), alpha polypeptide III | |
| PACSIN2 | Protein kinase C and casein kinase substrate in neurons 2 | NM_007229 |
| PARG1 | PTPL1-associated RhoGAP 1 | NM_004815 |
| PARP4 | Poly (ADP-ribose) polymerase family, member 4 | NM_006437 |
| PAWR | PRKC, apoptosis, WT1, regulator | NM_002583 |
| PC | Pyruvate carboxylase | NM_000920 |
| PCYOX1 | Prenylcysteine oxidase 1 | NM_016297 |
| PDCD6 | Programmed cell death 6 | AB033060 |
| PDGFA | Platelet-derived growth factor alpha polypeptide | NM_002607 |
| PDGFRA | Platelet-derived growth factor receptor, alpha polypeptide | NM_006206 |
| PDLIM4 | PDZ and LIM domain 4 | NM_003687 |
| PDZRN3 | PDZ domain containing RING finger 3 | AK130896 |
| PFKP | Phosphofructokinase, platelet | NM_002627 |
| PGK1 | Phosphoglycerate kinase 1 | NM_000291 |
| PGK1 | Phosphoglycerate kinase 1 | NM_000291 |
| PGM2L1 | Phosphoglucomutase 2-like 1 | NM_173582 |
| PGM2L1 | Phosphoglucomutase 2-like 1 | NM_173582 |
| PGM3 | Phosphoglucomutase 3 | NM_015599 |
| PGPEP1 | Pyroglutamyl-peptidase I | NM_017712 |
| PHF17 | PHD finger protein 17 | NM_024900 |
| PHF17 | PHD finger protein 17 | AK127326 |
| PHF17 | PHD finger protein 17 | AK127326 |
| PHLDA1 | Pleckstrin homology-like domain, family A, member 1 | NM_007350 |
| PHLDA1 | Pleckstrin homology-like domain, family A, member 1 | NM_007350 |
| PHLDA2 | Pleckstrin homology-like domain, family A, member 2 | NM_003311 |
| PHLDB1 | Pleckstrin homology-like domain, family B, member 1 | NM_015157 |
| PICALM | Phosphatidylinositol binding clathrin assembly protein | NM_007166 |
| PIK3C2B | Phosphoinositide-3-kinase, class 2, beta polypeptide | NM_002646 |
| PIK3R1 | Phosphoinositide-3-kinase, regulatory subunit 1 (p85 alpha) | NM_181523 |
| PIM1 | Pim-1 oncogene | NM_002648 |
| PIM3 | Serine/threonine-protein kinase pim-3 | NM_001001852 |
| PITX2 | Paired-like homeodomain transcription factor 2 | NM_153426 |
| PKM2 | Pyruvate kinase, muscle | CA420826 |
| PLAU | Plasminogen activator, urokinase | NM_002658 |
| PLAUR | Plasminogen activator, urokinase receptor | NM_001005377 |
| PLCE1 | Phospholipase C, epsilon 1 | NM_016341 |
| PLD1 | Phospholipase D1, phophatidylcholine-specific | NM_002662 |
| PLEKHA1 | Pleckstrin homology domain containing, family A | NM_001001974 |
| | (phosphoinositide binding specific) member 1 | |
| PLEKHA5 | Pleckstrin homology domain containing, family A member 5 | NM_019012 |
| PLEKHF1 | Pleckstrin homology domain containing, family F (with | NM_024310 |
| | FYVE domain) member 1 | |
| PLEKHG3 | Pleckstrin homology domain containing, family G (with | NM_015549 |
| | RhoGef domain) member 3 | |
| PLK2 | Polo-like kinase 2 (Drosophila) | NM_006622 |
| PLK3 | Polo-like kinase 3 (Drosophila) | NM_004073 |
| PLOD2 | Procollagen-lysine, 2-oxoglutarate 5-dioxygenase (lysine | NM_182943 |
| | hydroxylase) 2 | |
| PNMA1 | Paraneoplastic antigen MA1 | NM_006029 |
| PODXL | Podocalyxin-like | NM_005397 |
| POFUT2 | Protein O-fucosyltransferase 2 | NM_015227 |
| PP1665 | Hypothetical protein PP1665 | NM_030792 |
| pp9099 | PH domain-containing protein | NM_025201 |
| PPARG | Peroxisome proliferative activated receptor, gamma | NM_138711 |
| PPP1R13L | Protein phosphatase 1, regulatory (inhibitor) subunit 13 like | NM_006663 |
| PPP1R14C | Protein phosphatase 1, regulatory (inhibitor) subunit 14C | NM_030949 |
| PPP1R3B | Protein phosphatase 1, regulatory (inhibitor) subunit 3B | AK091994 |
| PPT1 | Palmitoyl-protein thioesterase 1 (ceroid-lipofuscinosis, | NM_000310 |
| | neuronal 1, infantile) | |
| PRICKLE2 | Prickle-like 2 (Drosophila) | NM_198859 |
| PRIM2A | Primase, polypeptide 2A, 58 kDa | NM_000947 |
| PRKAB2 | Protein kinase, AMP-activated, beta 2 non-catalytic subunit | NM_005399 |
| PRO1855 | Hypothetical protein PRO1855 | NM_018509 |
| PRPS1 | Phosphoribosyl pyrophosphate synthetase 1 | NM_002764 |
| PRPS1 | Phosphoribosyl pyrophosphate synthetase 1 | NM_002764 |
| PRPS1L1 | Phosphoribosyl pyrophosphate synthetase 1-like 1 | NM_175886 |
| PRRX2 | Paired related homeobox 2 | NM_016307 |
| PSAT1 | Phosphoserine aminotransferase 1 | NM_058179 |
| PSD3 | Pleckstrin and Sec7 domain containing 3 | NM_015310 |
| PSEN2 | Presenilin 2 (Alzheimer disease 4) | NM_000447 |
| PTDSR | Phosphatidylserine receptor | NM_015167 |
| PTPNS1 | Protein tyrosine phosphatase, non-receptor type substrate 1 | NM_080792 |
| PTX3 | Pentaxin-related gene, rapidly induced by IL-1 beta | NM_002852 |
| PYCARD | PYD and CARD domain containing | NM_013258 |
| RAB3D | RAB3D, member RAS oncogene family | BC007960 |
| RAB9A | RAB9A, member RAS oncogene family | NM_004251 |
| RABGAP1 | RAB GTPase activating protein 1 | NM_012197 |
| RACGAP1 | Rac GTPase activating protein 1 | NM_013277 |
| RACGAP1 | Rac GTPase activating protein 1 | NM_013277 |
| RAI14 | Retinoic acid induced 14 | NM_015577 |
| RAI17 | Retinoic acid induced 17 | NM_020338 |
| RASL11B | RAS-like, family 11, member B | NM_023940 |
| RDH5 | Retinol dehydrogenase 5 (11-cis and 9-cis) | NM_002905 |
| REV3L | REV3-like, catalytic subunit of DNA polymerase zeta (yeast) | NM_002912 |
| RGN | Regucalcin (senescence marker protein-30) | NM_004683 |
| RGS3 | Regulator of G-protein signalling 3 | NM_134427 |
| RHOBTB3 | Rho-related BTB domain containing 3 | NM_014899 |
| RIN1 | Ras and Rab interactor 1 | NM_004292 |
| RIS1 | Ras-induced senescence 1 | NM_015444 |
| RKHD3 | Ring finger and KH domain containing 3 | NM_032246 |
| RKHD3 | Ring finger and KH domain containing 3 | NM_032246 |
| RNF126 | Ring finger protein 126 | NM_194460 |
| ROR1 | Receptor tyrosine kinase-like orphan receptor 1 | NM_005012 |
| RPL10A | Ribosomal protein L10a | AK022044 |
| RPL21 | Ribosomal protein L21 | AA114874 |
| RPL5 | Ribosomal protein L5 | BF570356 |
| RTTN | Rotatin | NM_173630 |
| RUNX1 | Runt-related transcription factor 1 (acute myeloid leukemia 1; | NM_001001890 |
| | aml1 oncogene) | |
| RUNX2 | Runt-related transcription factor 2 | NM_004348 |
| RUSC2 | RUN and SH3 domain containing 2 | NM_014806 |
| S100A16 | S100 calcium binding protein A16 | NM_080388 |
| SALL2 | Sal-like 2 (Drosophila) | NM_005407 |
| SAMD11 | Sterile alpha motif domain containing 11 | NM_152486 |
| SAP30 | Sin3-associated polypeptide, 30 kDa | NM_003864 |
| SARS | Seryl-tRNA synthetase | AK022339 |
| SASH1 | SAM and SH3 domain containing 1 | NM_015278 |
| SATB1 | Special AT-rich sequence binding protein 1 (binds to nuclear | NM_002971 |
| | matrix/scaffold-associating DNA's) | |
| SAV1 | Salvador homolog 1 (Drosophila) | NM_021818 |
| SCD | Stearoyl-CoA desaturase (delta-9-desaturase) | NM_005063 |
| SCD | Stearoyl-CoA desaturase (delta-9-desaturase) | NM_005063 |
| SCD | Stearoyl-CoA desaturase (delta-9-desaturase) | AF132203 |
| SCHIP1 | Schwannomin interacting protein 1 | NM_014575 |
| SDFR1 | Stromal cell derived factor receptor 1 | BM982926 |
| SECTM1 | Secreted and transmembrane 1 | NM_003004 |
| SELENBP1 | Selenium binding protein 1 | NM_003944 |
| SEPP1 | Selenoprotein P, plasma, 1 | NM_005410 |
| SERP1 | Stress-associated endoplasmic reticulum protein 1 | NM_014445 |
| SERPINE1 | Serine (or cysteine) proteinase inhibitor, clade E (nexin, | NM_000602 |
| | plasminogen activator inhibitor type 1), member 1 | |
| SERTAD1 | SERTA domain containing 1 | NM_013376 |
| SERTAD4 | SERTA domain containing 4 | NM_019605 |
| SETDB2 | SET domain, bifurcated 2 | NM_031915 |
| SETDB2 | SET domain, bifurcated 2 | NM_031915 |
| SGCG | Sarcoglycan, gamma (35 kDa dystrophin-associated | NM_000231 |
| | glycoprotein) | |
| SGK | Serum/glucocorticoid regulated kinase | NM_005627 |
| SH3MD1 | SH3 multiple domains 1 | NM_014631 |
| SIAT4A | Sialyltransferase 4A (beta-galactoside alpha-2,3- | NM_003033 |
| | sialyltransferase) | |
| SIAT4A | Sialyltransferase 4A (beta-galactoside alpha-2,3- | NM_003033 |
| | sialyltransferase) | |
| SKIL | SKI-like | NM_005414 |
| SLC10A3 | Solute carrier family 10 (sodium/bile acid cotransporter | NM_019848 |
| | family), member 3 | |
| SLC16A3 | Solute carrier family 16 (monocarboxylic acid transporters), | NM_004207 |
| | member 3 | |
| SLC19A2 | Solute carrier family 19 (thiamine transporter), member 2 | NM_006996 |
| SLC1A5 | Solute carrier family 1 (neutral amino acid transporter), | NM_005628 |
| | member 5 | |
| SLC20A1 | Solute carrier family 20 (phosphate transporter), member 1 | NM_005415 |
| SLC20A1 | Solute carrier family 20 (phosphate transporter), member 1 | NM_005415 |
| SLC25A29 | Solute carrier family 25, member 29 | NM_152333 |
| SLC26A1 | Solute carrier family 26 (sulfate transporter), member 1 | NM_022042 |
| SLC2A1 | Solute carrier family 2 (facilitated glucose transporter), | NM_006516 |
| | member 1 | |
| SLC38A5 | Solute carrier family 38, member 5 | NM_033518 |
| SLC39A14 | Solute carrier family 39 (zinc transporter), member 14 | NM_015359 |
| SLC40A1 | Solute carrier family 40 (iron-regulated transporter), member 1 | NM_014585 |
| SLC4A2 | Solute carrier family 4, anion exchanger, member 2 | NM_003040 |
| | (erythrocyte membrane protein band 3-like 1) | |
| SLC6A6 | Solute carrier family 6 (neurotransmitter transporter, taurine), | NM_003043 |
| | member 6 | |
| SLC7A11 | Solute carrier family 7, (cationic amino acid transporter, y+ | NM_014331 |
| | system) member 11 | |
| SLC7A5 | Solute carrier family 7 (cationic amino acid transporter, y+ | NM_003486 |
| | system), member 5 | |
| SLC9A9 | Solute carrier family 9 (sodium/hydrogen exchanger), | NM_173653 |
| | isoform 9 | |
| SMAD3 | SMAD, mothers against DPP homolog 3 (Drosophila) | U68019 |
| SMAD3 | SMAD, mothers against DPP homolog 3 (Drosophila) | NM_005902 |
| SMAD7 | SMAD, mothers against DPP homolog 7 (Drosophila) | NM_005904 |
| SMARCA3 | SWI/SNF related, matrix associated, actin dependent | NM_003071 |
| | regulator of chromatin, subfamily a, member 3 | |
| SMARCB1 | SWI/SNF related, matrix associated, actin dependent | NM_003073 |
| | regulator of chromatin, subfamily b, member 1 | |
| SNAI1 | Snail homolog 1 (Drosophila) | NM_005985 |
| SNF1LK | SNF1-like kinase | NM_173354 |
| SNF1LK | SNF1-like kinase | NM_173354 |
| SNTB2 | Syntrophin, beta 2 (dystrophin-associated protein A1, 59 kDa, | NM_006750 |
| | basic component 2) | |
| SNX24 | Sorting nexing 24 | NM_014035 |
| SOCS2 | Suppressor of cytokine signaling 2 | NM_003877 |
| SOX4 | SRY (sex determining region Y)-box 4 | NM_003107 |
| SOX4 | SRY (sex determining region Y)-box 4 | NM_003107 |
| SOX4 | SRY (sex determining region Y)-box 4 | AW946823 |
| SOX9 | SRY (sex determining region Y)-box 9 (campomelic | NM_000346 |
| | dysplasia, autosomal sex-reversal) | |
| SPARC | Secreted protein, acidic, cysteine-rich (osteonectin) | NM_003118 |
| SPHK1 | Sphingosine kinase 1 | NM_021972 |
| SRF | Serum response factor (c-fos serum response element-binding | NM_003131 |
| | transcription factor) | |
| SRF | Serum response factor (c-fos serum response element-binding | NM_003131 |
| | transcription factor) | |
| SSBP4 | Single stranded DNA binding protein 4 | NM_032627 |
| STAT2 | Signal transducer and activator of transcription 2, 113 kDa | BE825944 |
| STC2 | Stanniocalcin 2 | NM_003714 |
| STCH | Stress 70 protein chaperone, microsome-associated, 60 kDa | NM_006948 |
| STEAP | Six transmembrane epithelial antigen of the prostate | NM_012449 |
| STK38L | Serine/threonine kinase 38 like | NM_015000 |
| STMN1 | Stathmin 1/oncoprotein 18 | NM_203401 |
| STXBP6 | Syntaxin binding protein 6 (amisyn) | NM_014178 |
| SUSD3 | Sushi domain containing 3 | NM_145006 |
| SYNJ2 | Synaptojanin 2 | NM_003898 |
| SYVN1 | Synovial apoptosis inhibitor 1, synoviolin | NM_172230 |
| TBC1D8 | TBC1 domain family, member 8 (with GRAM domain) | NM_007063 |
| TBX3 | T-box 3 (ulnar mammary syndrome) | NM_016569 |
| TCEA3 | Transcription elongation factor A (SII), 3 | NM_003196 |
| TCEA3 | Transcription elongation factor A (SII), 3 | NM_003196 |
| TCEA3 | Transcription elongation factor A (SII), 3 | NM_003196 |
| TD-60 | RCC1-like | NM_018715 |
| TD-60 | RCC1-like | BQ233242 |
| TES | Testis derived transcript (3 LIM domains) | NM_152829 |
| TFPI | Tissue factor pathway inhibitor (lipoprotein-associated | NM_006287 |
| | coagulation inhibitor) | |
| TGFB1 | Transforming growth factor, beta 1 (Camurati-Engelmann | NM_000660 |
| | disease) | |
| TGFBR1 | Transforming growth factor, beta receptor I (activin A | AI537201 |
| | receptor type II-like kinase, 53 kDa) | |
| TGFBR3 | Transforming growth factor, beta receptor III (betaglycan, | NM_003243 |
| | 300 kDa) | |
| TGM2 | Transglutaminase 2 (C polypeptide, protein-glutamine- | NM_004613 |
| | gamma-glutamyltransferase) | |
| THBD | Thrombomodulin | NM_000361 |
| TIGD2 | Tigger transposable element derived 2 | NM_145715 |
| TIMP3 | Tissue inhibitor of metalloproteinase 3 (Sorsby fundus | NM_000362 |
| | dystrophy, pseudoinflammatory) | |
| TIMP3 | Tissue inhibitor of metalloproteinase 3 (Sorsby fundus | AA837799 |
| | dystrophy, pseudoinflammatory) | |
| TIPARP | TCDD-inducible poly(ADP-ribose) polymerase | NM_015508 |
| TK1 | Thymidine kinase 1, soluble | NM_003258 |
| TMEM25 | Transmembrane protein 25 | NM_032780 |
| TMEPAI | Transmembrane, prostate androgen induced RNA | NM_020182 |
| TMEPAI | Transmembrane, prostate androgen induced RNA | NM_020182 |
| TMPO | Thymopoietin | AW291149 |
| TNC | Tenascin C (hexabrachion) | NM_002160 |
| TNFAIP2 | Tumor necrosis factor, alpha-induced protein 2 | NM_006291 |
| TNFAIP8 | Tumor necrosis factor, alpha-induced protein 8 | NM_014350 |
| TNFRSF11B | Tumor necrosis factor receptor superfamily, member 11b | NM_002546 |
| | (osteoprotegerin) | |
| TNFRSF11B | Tumor necrosis factor receptor superfamily, member 11b | NM_002546 |
| | (osteoprotegerin) | |
| TNFRSF12A | Tumor necrosis factor receptor superfamily, member 12A | NM_016639 |
| TNFRSF14 | Tumor necrosis factor receptor superfamily, member 14 | NM_003820 |
| | (herpesvirus entry mediator) | |
| TNFRSF19L | Tumor necrosis factor receptor superfamily, member 19-like | NM_032871 |
| TPM1 | Tropomyosin 1 (alpha) | NM_000366 |
| TRERF1 | Transcriptional regulating factor 1 | NM_033502 |
| TREX1 | Three prime repair exonuclease 1 | NM_016381 |
| TRIB1 | Tribbles homolog 1 (Drosophila) | NM_025195 |
| TRIB2 | Tribbles homolog 2 (Drosophila) | NM_021643 |
| TRIB3 | Tribbles homolog 3 (Drosophila) | NM_021158 |
| TRIM2 | Tripartite motif-containing 2 | NM_015271 |
| TRIM7 | Tripartite motif-containing 7 | NM_033342 |
| TRPV2 | Transient receptor potential cation channel, subfamily V, | NM_016113 |
| | member 2 | |
| TSK | Likely ortholog of chicken tsukushi | NM_015516 |
| TUBA3 | Tubulin, alpha 3 | NM_006009 |
| TUBA6 | Tubulin alpha 6 | NM_032704 |
| TUBB2 | Tubulin, beta 2 | NM_001069 |
| TUBB3 | Tubulin, beta 3 | NM_006086 |
| TUBB3 | Tubulin, beta 3 | NM_006086 |
| TUBB4 | Tubulin, beta 4 | NM_006087 |
| TUBB6 | Tubulin, beta 6 | NM_032525 |
| TUFT1 | Tuftelin 1 | NM_020127 |
| TWIST1 | Twist homolog 1 (acrocephalosyndactyly 3; Saethre-Chotzen | NM_000474 |
| | syndrome) (Drosophila) | |
| TXNIP | Thioredoxin interacting protein | NM_006472 |
| TYMS | Thymidylate synthetase | NM_001071 |
| UAP1 | UDP-N-acteylglucosamine pyrophosphorylase 1 | NM_003115 |
| UBE2C | Ubiquitin-conjugating enzyme E2C | NM_181803 |
| UCK2 | Uridine-cytidine kinase 2 | NM_012474 |
| UGCG | UDP-glucose ceramide glucosyltransferase | NM_003358 |
| UGDH | UDP-glucose dehydrogenase | NM_003359 |
| ULK1 | Unc-51-like kinase 1 (C. elegans) | NM_003565 |
| ULK1 | Unc-51-like kinase 1 (C. elegans) | NM_003565 |
| UNC5B | Unc-5 homolog B (C. elegans) | NM_170744 |
| UPP1 | Uridine phosphorylase 1 | NM_181597 |
| UPP1 | Uridine phosphorylase 1 | BC047030 |
| USP35 | Ubiquitin specific protease 35 | AB037793 |
| USP53 | Ubiquitin specific protease 53 | BC017382 |
| USP53 | Ubiquitin specific protease 53 | AF085848 |
| VEGF | Vascular endothelial growth factor | NM_003376 |
| VLDLR | Very low density lipoprotein receptor | NM_003383 |
| VMP1 | Likely ortholog of rat vacuole membrane protein 1 | BC024020 |
| WASF2 | WAS protein family, member 2 | NM_006990 |
| WNT5B | Wingless-type MMTV integration site family, member 5B | NM_030775 |
| XBP1 | X-box binding protein 1 | NM_005080 |
| XBP1 | X-box binding protein 1 | NM_005080 |
| YPEL2 | Yippee-like 2 (Drosophila) | NM_001005404 |
| YPEL4 | Yippee-like 4 (Drosophila) | NM_145008 |
| ZBED3 | Zinc finger, BED domain containing 3 | NM_032367 |
| ZC3HDC6 | Zinc finger CCCH type domain containing 6 | AK131416 |
| ZFHX1B | Zinc finger homeobox 1b | NM_014795 |
| ZFP36 | Zinc finger protein 36, C3H type, homolog (mouse) | NM_003407 |
| ZFP36L2 | Zinc finger protein 36, C3H type-like 2 | NM_006887 |
| ZNF161 | Zinc finger protein 161 | NM_007146 |
| ZNF281 | Zinc finger protein 281 | NM_012482 |
| ZNF336 | Zinc finger protein 336 | NM_022482 |
| ZNF395 | Zinc finger protein 395 | NM_018660 |
| ZNF395 | Zinc finger protein 395 | NM_018660 |
| ZNF462 | Zinc finger protein 462 | NM_021224 |
| ZNF469 | Zinc finger protein 469 | AB058761 |
| ZNF537 | Zinc finger protein 537 | NM_020856 |
| ZNF589 | Zinc finger protein 589 | NM_016089 |
| | | A_23_P123234 |
| | | A_23_P170719 |
| | | A_23_P347100 |
| | | A_23_P57836 |
| | | A_24_P110591 |
| | | A_24_P144314 |
| | | A_24_P170283 |
| | | A_24_P178167 |
| | | A_24_P221485 |
| | | A_24_P234871 |
| | | A_24_P247169 |
| | | A_24_P256063 |
| | | A_24_P401090 |
| | | A_24_P401663 |
| | | A_24_P471099 |
| | | A_24_P541482 |
| | | A_24_P562242 |
| | | A_24_P745960 |
| | | A_32_P100338 |
| | | A_32_P101844 |
| | | A_32_P105865 |
| | | A_32_P116219 |
| | | A_32_P182135 |
| | | A_32_P49035 |
| | | A_32_P75141 |
| | Clone 24841 mRNA sequence | AF131834 |
| | | AF159295 |
| | | AF187554 |
| | LOC440502 | AF218008 |
| | | AF271776 |
| | Clone pp9372 unknown mRNA | AF289610 |
| | Hypothetical gene supported by BX647608 | AK021804 |
| | CDNA: FLJ22642 fis, clone HSI06970 | AK026295 |
| | | AK055387 |
| | MRNA (clone ICRFp507I1077) | AK092450 |
| | Hypothetical gene supported by BX647608 | AK095791 |
| | CDNA FLJ41489 fis, clone BRTHA2004582 | AK123483 |
| | CDNA clone IMAGE: 4077090, partial cds | AK124426 |
| | CDNA FLJ44441 fis, clone UTERU2020242 | AK126405 |
| | MRNA full length insert cDNA clone EUROIMAGE 966164 | AK129879 |
| | | AX721087 |
| | | BC000206 |
| | | BC009078 |
| | Hypothetical gene supported by AK001829 | BC017654 |
| | Homo sapiens, clone IMAGE: 3869276, mRNA | BC018597 |
| | Homo sapiens, clone IMAGE: 5299642, mRNA | BC041913 |
| | | BC089451 |
| | | BC090889 |
| | | BE004814 |
| | | BF366211 |
| | Transcribed locus, moderately similar to NP_055301.1 | BG182941 |
| | neuronal thread protein AD7c-NTP [Homo sapiens] | |
| | Transcribed locus | BG777521 |
| | Similar to D(1B) dopamine receptor (D(5) dopamine | BM561346 |
| | receptor) (D1beta dopamine receptor) | |
| | Transcribed locus, moderately similar to XP_497060.1 | BM989848 |
| | similar to FKSG60 [Homo sapiens] | |
| | Similar to phosducin-like 3; phosducin-like 2; IAP-associated | BU783246 |
| | factor VIAF1 | |
| | Homo sapiens, clone IMAGE: 3868989, mRNA, partial cds | CR595668 |
| | Similar to centaurin, gamma-like family, member 1; ARF | CR613654 |
| | GTPase-activating protein; Em: AC012044.1 | |
| | | CX788817 |
| | | ENST00000229270 |
| | | ENST00000258884 |
| | | ENST00000261569 |
| | | ENST00000297145 |
| | | ENST00000304963 |
| | | ENST00000308603 |
| | | ENST00000310006 |
| | | ENST00000310692 |
| | | ENST00000330777 |
| | | ENST00000336283 |
| | | ENST00000339446 |
| | | ENST00000343505 |
| | | ENST00000354185 |
| | | ENST00000358293 |
| | | ENST00000367385 |
| | | ENST00000368503 |
| | | ENST00000372583 |
| | | ENST00000374279 |
| | | ENST00000375377 |
| | | ENST00000377003 |
| | | ENST00000378953 |
| | | ENST00000379731 |
| | | ENST00000382327 |
| | | NM_001010911 |
| | | NM_001012271 |
| | | NM_001012426 |
| | | NM_001012507 |
| | | NM_001012507 |
| | | NM_001014373 |
| | | NM_001017535 |
| | | NM_001018004 |
| | | NM_001018004 |
| | | NM_001018004 |
| | | NM_001025100 |
| | | NM_001025295 |
| | | NM_001025366 |
| | | NM_001025366 |
| | | NM_001030059 |
| | | NM_001031716 |
| | | NM_001033053 |
| | | NM_001039212 |
| | | NM_001040167 |
| | | NM_001620 |
| | | NM_001620 |
| | | NM_004052 |
| | | NM_012454 |
| | | NM_014732 |
| | | NM_015009 |
| | | NM_015012 |
| | | NM_015088 |
| | | NM_015137 |
| | | NM_015262 |
| | | NM_015262 |
| | | NM_015326 |
| | | NM_133374 |
| | | NM_153698 |
| | | NR_000039 |
| | | NR_002802 |
| | | NR_002819 |
| | | NR_002819 |
| | | THC2311186 |
| | | THC2340670 |
| | | THC2363646 |
| | | THC2375353 |
| | | THC2376027 |
| | | THC2378689 |
| | | THC2381535 |
| | | THC2392192 |
| | | THC2395355 |
| | | THC2401540 |
| | | THC2408398 |
| | | THC2429183 |
| | | THC2433066 |
| | | THC2433340 |
| | | THC2438327 |
| | | THC2453189 |
| | | W31297 |
| | | W95609 |
| | | X66610 |
| | Hypothetical LOC145853 | XM_096885 |
| | Hypothetical LOC400890 | XM_379036 |
| | | XM_928728 |
| | | XM_937741 |
| | | XM_941152 |
| | | XR_000986 |

The pleiotropic effects of TGFβ on regulation of cellular processes are highly dependent on both the cell type and the biological microenvironment in which the cells are resident. The tool DAVID (Dennis, et al. (2003) Genome Biol. 4(5):P3) was used to identify groups of Gene Ontology (G0) terms enriched in each of the lists of genes classified as either induced or repressed by TGFβ in cultured adult dermal fibroblasts under these experimental conditions. The biological themes coordinately up-regulated by TGFβ are summarized in Table 9. Functional categories with the highest enrichment scores were broad groups that included proteins containing LIM-domains, growth factors, cell-signaling, DNA-binding proteins and membrane proteins, signifying the global effects that the potent cytokine TGFβ has on multiple cellular processes and signaling pathways. Enrichment of G0 terms associated with collagen production and ECM deposition and remodeling, processes known to be heavily regulated and induced by TGFβ, were also found. Surprisingly, the number of genes induced by TGFβ that contribute to these ECM-related-enriched G0 terms were found to be lower than expected. One possible explanation that would account for this discrepancy would be that many of the expected genes including a number of collagens are post-transcriptionally regulated by TGF through mechanisms of both increase collagen synthesis and a complementary decrease in degradation (McAnulty, et al. (1991) Biochim. Biophys. Acta 1091(2):231-5).

TABLE 9

| | | Enrichment | # Genes in |
|---|---|---|---|
| Cluster | Biological Theme | Score | Cluster |
| 1 | Lim domain containing proteins | 5.51 | 13 |
| 2 | Growth factors | 2.91 | 4 |
| 3 | Cell Signaling | 2.42 | 20 |
| 4 | DNA-binding proteins | 2.17 | 53 |
| 5 | Membrane Proteins | 1.78 | 22 |
| 6 | Tubulin-Associated | 1.52 | 6 |
| 7 | Collagens | 1.40 | 4 |
| 8 | Carbohydrate Synthesis | 1.34 | 5 |
| 9 | Solute Transporters | 1.28 | 19 |
| 10 | Metalloproteases | 1.19 | 5 |
| 11 | Extracellular Matrix Proteins | 1.19 | 7 |
| 12 | Heat Shock Proteins | 0.91 | 5 |

Conversely, the functional categories identified by DAVID for down-regulated in response to TGFβ genes are shown in Table 10. Similar to the genes that showed positive regulation by TGFβ, functional categories that showed greatest enrichment in the down-regulated in response to TGFβ were those associated with global biological processes, including transcription factors, membrane proteins and Ras small GTPases.

TABLE 10

| | | Enrichment | # Genes in |
|---|---|---|---|
| Cluster | Biological Theme | Score | Cluster |
| 1 | Cell cycle | 3.58 | 6 |
| 2 | Transcription factors | 3.41 | 65 |
| 3 | DNA repair | 2.06 | 4 |
| 4 | Lysosome associated proteins | 1.34 | 4 |
| 5 | Membrane proteins | 1.06 | 14 |
| 6 | Ras small GTPases | 1.06 | 4 |
| 7 | Tubulin-associated | 0.93 | 4 |
| 8 | Ribosomal proteins | 0.93 | 4 |
| 9 | Glycoprotein metabolism | 0.85 | 4 |
| 10 | Ion transport | 0.60 | 4 |
| 11 | TPR containing proteins | 0.59 | 4 |
| 12 | Surface expressed receptors | 0.54 | 18 |

It was also noted that genes associated with cell cycle processes, CCBN1, CCBN2, KNTC2, CNAP1, HCAP-G, CDCA2, CDCA8, MAPRE-2 were repressed under these conditions (Table 10). The expression of many of these genes was also reduced in the no treatment control, indicating that the experimental conditions and not the response to TGF is the driving force behind the observed decrease in mRNA levels of these genes. It should however be noted that the magnitude of the decrease in the TGFβ treated cells was much greater than that in the no treatment control, thus TGFβ may contribute in some way to the observed down-regulation of these genes. Additionally, TGFβ induced increased expression of p15^{INK4B}, previously characterized as mediating cell cycle arrest in fibroblasts in G1 phase (Hannon & Beach (1994) Nature 371(6494):257-61). The proliferation status of the fibroblasts cultures following TGFβ treatment was also monitored. Proliferation was assessed over 24 hours by BrdU incorporation into S phase cells. No increase in the number of cells was observed with detectable BrdU incorporation, thus fibroblasts grown in low serum media were not driven into cell cycle when exposed to TGFβ.

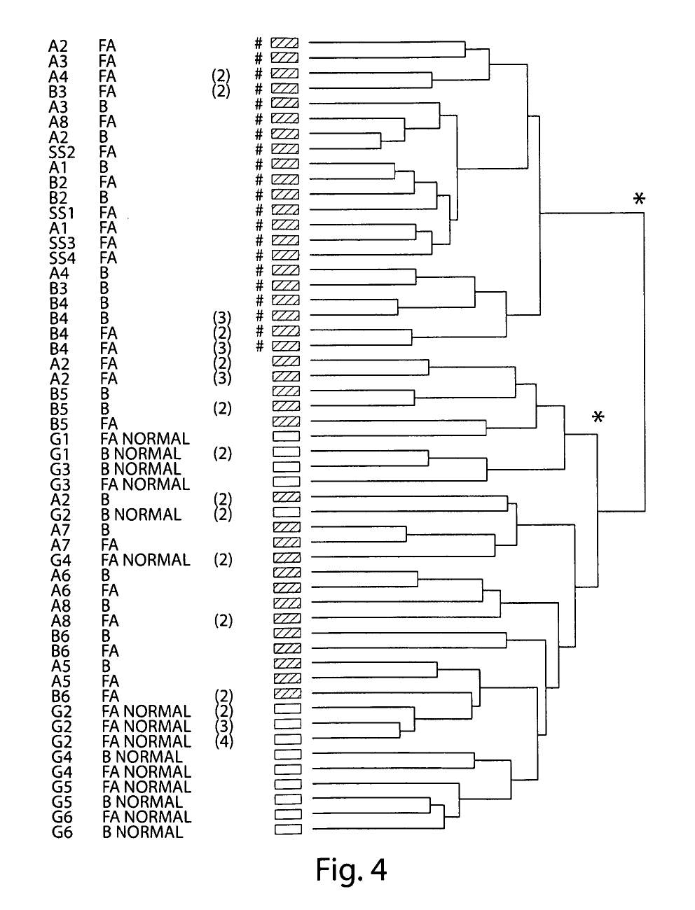

The TGFβ-Responsive Signature is Activated in a Subset of dSSc Patients. The expression of the TGFβ signature was examined in a published microarray dataset including gene expression data from healthy and dSSc skin biopsies as described in Example 1. Expression data for the 894 probes identified as TGFβ-responsive were extracted from the skin biopsy microarray dataset previously described. Organization of the microarrays by hierarchical clustering using only the TGFβ-responsive probes resulted in a clear bifurcation of the samples (FIG. 4). One branch of the array dendogram (#) was composed solely of dSSc patient samples, while the remaining branch contained both dSSc patient samples and those from healthy control skin biopsies. SigClust analysis was used to test the robustness of the sample bifurcation and highly significant (p<0.001) clustering was found. The clustering of one additional subgroup of samples was also found to be significant at this level, however this was not investigated any further given the relatively small size of this cluster (nine arrays) and the inclusion of two samples in this group from patient A8, who was inconclusively classified in this analysis.

Alignment and clustering of the skin biopsy gene expression data with that from the in vitro TGFβ time courses, revealed that expression of the signature was very heterogeneous throughout all samples in both groups (FIG. 2B). It was then determined which of the 894 probes was driving the observed bifurcation of samples into the two groups. A 2-class unpaired SAM analysis identified 484 probes that were significantly differentially expressed between the two groups. The centroid values for the 484 differentially expressed probes were calculated. The extent of activation of the TGFβ-responsive signature in each of the patient samples was determined by calculating the Pearson correlation coefficients between the centroid and the each of the microarray skin biopsy sample gene expression values. The Pearson correlation scores were graphed. Based on the trend of the Pearson correlations for each of the two groups that resulted from clustering the samples, the group indicated with #, which that was composed solely of dSSc samples, was termed “TGFβ-3-activated” as this group demonstrated a positive correlation with the centroid. The remaining group in which there was a mix of dSSc and healthy volunteer samples was termed “TGFβ-not activated,” owing to the predominantly negative correlation coefficients of this group with the TGFβ-responsive signature centroid.

Patients that Showed TGFβ-Activation had Higher Skin Scores and Increased Incidence of ILD. It was reasoned that the presence of the TGFβ-responsive gene signature may define a clinically distinct group of patients and could therefore be used as markers of disease activity. The severity and incidence of a number of clinical parameters was analyzed to determine if the TGFβ-activated group of dSSc patients showed phenotypic differences from those that clustered together with healthy controls. The two patients SSc2 and SSc8 that could not be conclusively assigned to either group were excluded from these statistical analyses, resulting in a total of 10 patients in the TGFβ-activated group and 5 patients in the TGFβ-not activated group. To determine if any differences in the groups existed for clinical parameters with continuous data, including MRSS (score from 0-53), Raynaud's phenomenon (0-10), incidence of digital ulcers, patient age and disease duration (as defined by onset of first non-Raynaud's symptoms), Student's T-tests were conducted. Patients in the TGFβ-activated group showed statistically significant higher skin scores (mean=26.33±8.16) than those in the TGFβ-not activated group (mean=17.80±6.16) (Table 11). Other clinical parameters such as incidence of ILD, impaired renal function, gastrointestinal (GI) involvement and pulmonary arterial hypertension (PAH) were scored as either present or absent and a chi-squared test implemented to assess any differences between the groups (Table 11). It was found that ILD was significantly more prevalent in the group of TGFβ-activated patients (p<0.02) with the calculated odds ratio for ILD in this group being≈8.00. No significant associations of the TGFβ-activated group were observed with any of the other clinical variables assessed (Table 11).

TABLE 11

| | Activated | Not Activated | |
|---|---|---|---|
| Clinical Parameter | (n = 10) | (n = 5) | p-value |
| MRSS | 26.33 ± 8.16 | 17.80 ± 6.16 | <0.01 |
| ILD | 7/10 | 1/5 | <0.02 |
| Disease Duration (years) | 7.93 ± 5.69 | 4.40 ± 4.07 | <0.10 |
| GI Involvement | 9/10 | 3/5 | <0.13 |
| PAH | 0/10 | 1/5 | <0.13 |
| Renal Disease | 2/10 | 0/5 | <0.21 |
| Patient Age (years) | 45.73 ± 11.04 | 50.60 ± 7.38 | <0.23 |
| Raynaud's Phenomenon | 5.85 ± 2.19 | 7.00 ± 3.13 | <0.31 |
| Digital Ulcers | 0.89 ± 1.13 | 0.80 ± 1.22 | <0.89 |

Statistical associations of clinical parameters to the TGFβ-activated and TGFβ-not activated groups of patients. Clinical parameters assessed were modified Rodnan skin score (MRSS) on a 51-point scale, disease duration since first onset of non-Raynaud's symptoms, a self-reported Raynaud's severity score on a 10-point scale, and the presence or absence of digital ulcers on a 3-point scale. Also indicated are the presence (+) or absence (−) of gastrointestinal involvement(GI), interstitial lung disease (ILD) and pulmonary arterial hypertension (PAH) as determined by high resolution computerized tomography (HRCT) and renal disease. Associations with MRSS, disease duration, patient age Raynaud's phenomenon and digital ulcers were calculated using Student's T-tests. A chi-squared test was performed to determine if any associations were significant with ILD, GI involvement, renal disease and PAH.

### Example 3

### Computational Framework for Identifying Individual Biomarkers

Due to inherent complexity of peripheral blood samples, computational tools have been developed to extract the maximum amount of information from the PBC datasets. The goal of these computational approaches is to identify the minimum number of genes that will classify samples into groups based on clinical parameters or predefined groupings, when their gene expression patterns are combined. One way to determine the relationship between the expression of multiple genes and a clinical observation is to use linear discriminant analysis (LDA). LDA is a method to classify patients into groups based on features that describe each patient, such as the gene-expression of specific genes. A combination of variables and constants are found that generate an effective discriminant score that separate two groups. The general equation is in the following form, where Cₖ is a constant and Geneₖ is the expression of level of gene k in a sample:

LDA Score=(C₁)(Gene₁)+(C₂)(Gene₂)+ . . . +(Cₖ)(Geneₖ)

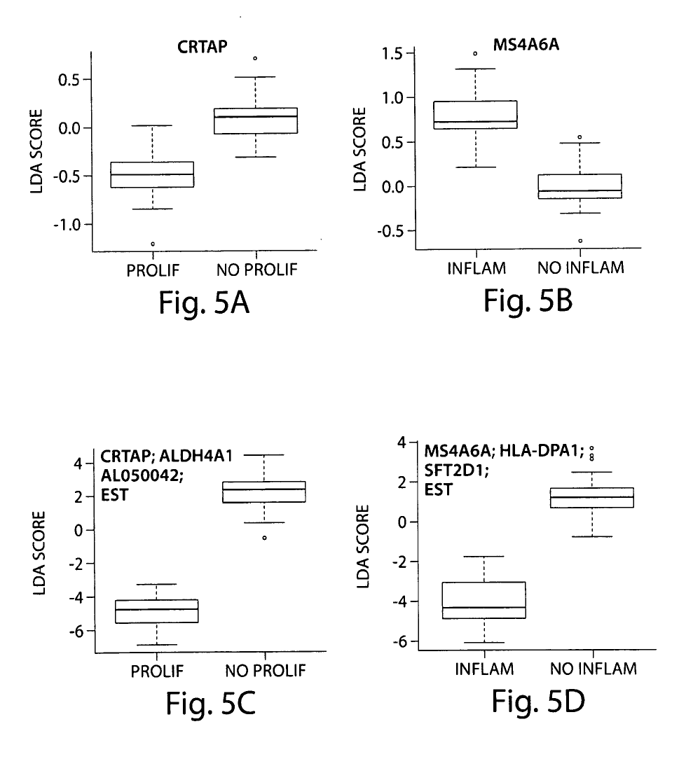

Using the skin biopsy dataset, LDA was used to identify genes that distinguish the ‘intrinsic’ subgroups. Genes for the proliferation and the inflammatory intrinsic groups are shown in FIG. 5. When LDA analysis was performed with single genes, single genes alone were able to distinguish between the classification groups (such as proliferation and no proliferation), however, there was overlap between the distributions (FIG. 5A, FIG. 5B). The multivariable LDA analysis resulted in a greater separation between LDA scores for the two groups than by using the gene expression of single genes alone (FIG. 5C, FIG. 5D). The multivariate analysis resulted in clear separation of the two groups without overlap. This analysis provides one or more of CRTAP, ALDH4A1, AL050042, and EST as potential biomarkers in the skin for identifying the intrinsic Proliferation group and one or more of MS4A6A, HLA-DPA1, SFT2D1, and EST as potential biomarkers in the skin for identifying the intrinsic Inflammatory group in SSc.

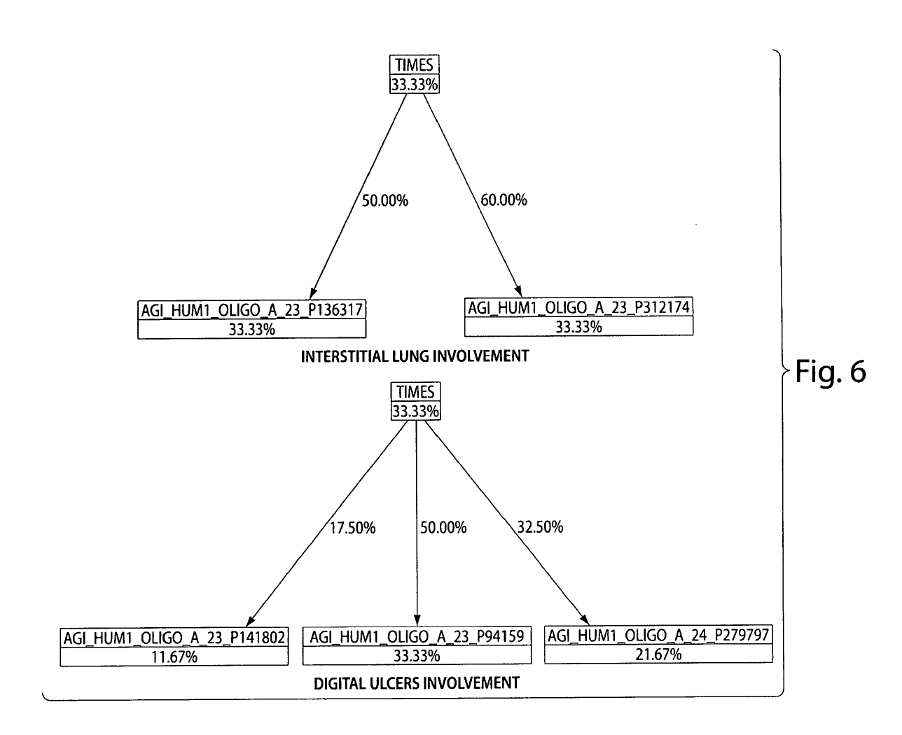

Symbolic Discriminant Analysis (SDA) has been developed to select gene expression variables and discriminant functions that are not limited to a linear form. This is accomplished by providing a list of mathematical functions (e.g., +, −, *, /) and a list of gene expression values to build discriminant functions using a stochastic search algorithm. The symbolic discriminant functions are represented as expression trees, and accuracy of the resulting discriminant functions is determined by how well they separate patients by clinical parameter or gene expression subtype (FIG. 6).

Determination of expression trees for SDA requires a more computationally complex framework than LDA. The first step of the process focuses on choosing the optimal parameters for the stochastic algorithm. The number of possible combinations of mathematical functions and genes is very large, so determining a more limited search space is necessary. Different population sizes, generation lengths, and tree depths were considered. In addition, seven different sets of mathematical functions including arithmetic operators (+, −, *, /), relational operators (=, !=, <, >, <=, >=, max, min), Boolean operators (AND, OR, NOT, NOR, IF, XOR), in all 189 possible combinations were considered. Each combination was analyzed 10 different times using random seeds (a total 1890 runs) and best model along with its accuracy was recorded. All results were considered statistically significant at a p<0.05.

After the determination of the best factors for the stochastic search algorithm, the stochastic search algorithm was run 100,000 times with different random seeds, each time saving the best SDA model. Then these 100,000 best models were ranked according to their accuracy (how often they predicted the correct sample distribution) and from this group the best 100 models were selected for further consideration.

A graphical model of the 100 best SDA models was generated. Across the 100 best trees, the percentage of time each single element or each adjacent pair of genes was present was recorded. This information was used to draw a directed acyclic graph. The directed graph indicates which functions and attributes show up most frequently. The edges (connections) in the graph connect genes with a mathematical function. A threshold of 2% was employed to show only the most frequent connections between nodes.

For two clinical covariates, Interstitial Lung Disease (ILD) and Digital Ulcers (DU), the resultant directed graphs were simple enough that they are final models for classifying patients, and further processing steps are not necessary. ILD can be distinguished by the equal multiplicative combination of two different genes, REST Corepressor 3 (RCO3) and Alstrom Syndrome 1. RCO3 is uncharacterized but shows highest expression in the heart and blood vessels. ALMS1 was identified by positional cloning as a gene in which sequence variations cosegregated with Alstrom syndrome. ALMS1 deletion has been shown to result in defective cilia and abnormal calcium transport in mice. Individuals with Alstrom syndrome develop a wide range of systemic disease including renal failure, pulmonary, hepatic and urologic dysfunction, and systemic fibrosis develops with age in these patients (OMIM:203800). DU can be predicted by multiplicative combination of three genes (SERPINB7, FBXO25 and MGC3207).

### Example 4

### Use of Linear Discriminant Analysis (LDA) to Distinguish the Diffuse-Proliferation and Inflammatory Groups

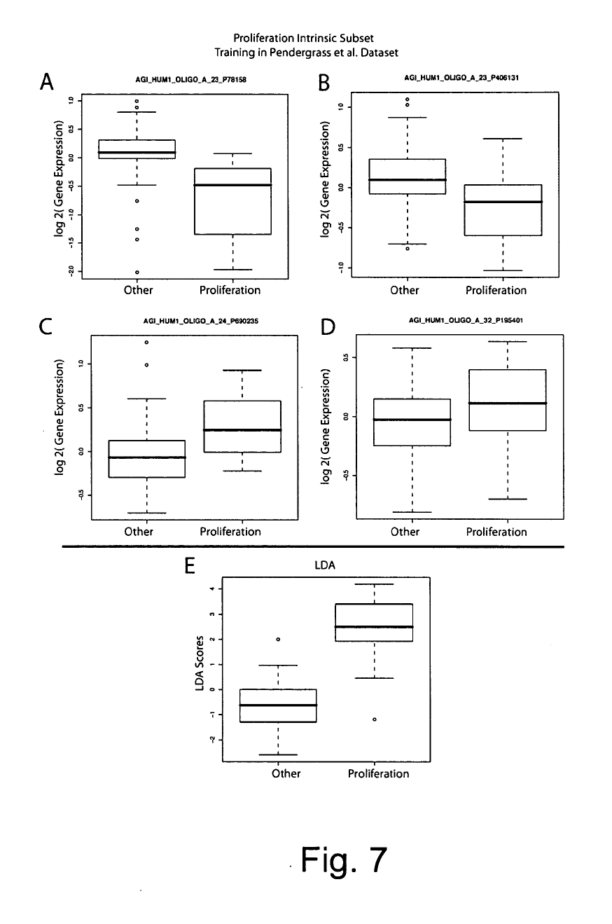

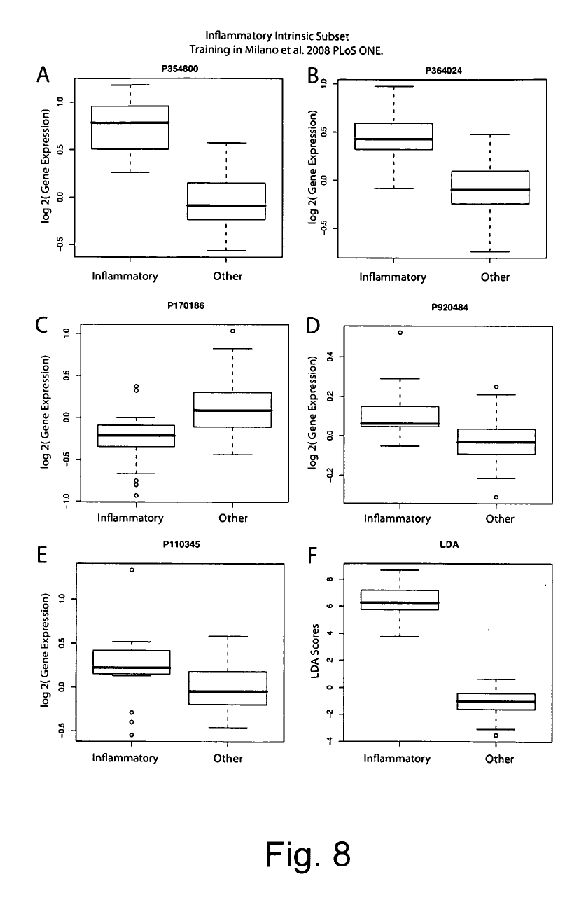

Genes that distinguished samples in the Diffuse-Proliferation and Inflammatory groups were selected using Linear Discriminant Analysis (LDA), described in Example 3, and the initial skin biopsy gene expression datasets. Examples of genes found using the LDA approach are shown in FIG. 7 and FIG. 8. Examination of the expression data for single genes shows that the expression any one single gene may not always clearly distinguish between the groups of proliferation and no proliferation. In contrast, the multivariable LDA analysis results in LDA scores that separated the two groups more than by using the gene expression of single genes alone (FIG. 7E). Particularly in the case of testing the results of the LDA equation for the Inflammatory group in a separate dataset (FIG. 8E), the multivariate analysis resulted in clear separation of the two groups. This analysis therefore provides potential biomarkers in the skin for identifying the intrinsic subsets in SSc in new skin biopsies.

For the Diffuse-Proliferation group, LDA Score=−1.902(NM_{—}004703)−1.908(NM_{—}020422)+1.475(AGI_HUM1_OLIGO_A_{—}24_P690235)+1.83(NM_{—}173511), where NM_{—}004703 corresponds to RABEP1, NM_{—}020422 corresponds to promethin, AGI_HUM1_OLIGO_A_{—}24_P690235 refers to novel gene transcript ENST00000312412, and NM_{—}173511 refers to ALS2CR13.

For the Inflammatory group, LDA score=4.365(NM_{—}002119)+2.926(NM_{—}006851)−2.620(NM_{—}017570)+6.601(NM_{—}022163)+2.033(NM_{—}012110), where NM_{—}002119 refers to HLA-DOA, NM_{—}006851 refers to GLIPR1, NM_{—}017570 refers to OPLAH, NM_{—}022163 refers to MRPL46, and NM_{—}012110 refers to CHIC2.

### Example 5

### IL-13 and IL-4 Gene Signatures Identify the Inflammatory Subset

In addition to TGFβ, gene expression signatures associated with pro-fibrotic cytokines IL-13 (NM_{—}002188) and IL-4 (NM_{—}000589) were determined in cultured adult human dermal fibroblasts. The 490 genes of the IL-13 gene signature are presented in Table 12. The genes of the IL-4 gene signature are presented in Table 13. This analysis indicated that IL-13 and IL-4 share an approximately 60% overlap of inducible genes. In contrast, the TGFβ inducible signature was composed of a distinct set of gene expression targets demonstrating a 5% overlap with the IL-13 and IL-4 signatures.

Gene expression signatures were used to determine the potential drivers of fibrosis in a large well-controlled gene expression dataset of SSc skin biopsies, which were demonstrated herein as molecular subsets in scleroderma skin. The TGFβ signature was largely expressed in a subset of diffuse patients and was more highly expressed in patients with more severe skin disease (p<0.01) and scleroderma lung disease (p<0.01). The IL-13 and IL-4 gene expression signatures showed increased expression in the Inflammatory subset of SSc patients biopsies, and represent the earliest disease stages.

It is contemplated that fibrosis in different SSc subsets is driven by different molecular mechanisms tied to either TGFβ or IL-13 and IL-4. These finding indicate that patient subsetting is necessary in order to target different anti-fibrotic treatments based on molecular subclassifications of SSc patients.

TABLE 12

| Gene Symbol | Gene Name | Accession No. |
|---|---|---|
| ABCA6 | ATP-binding cassette, sub-family A (ABC1), member 6 | NM_080284 |
| ACTA1 | Actin, alpha 1, skeletal muscle | NM_001100 |
| ADAMTS1 | A disintegrin-like and metalloprotease (reprolysin type) | NM_006988 |
| | with thrombospondin type 1 motif, 1 | |
| ADCY4 | Adenylate cyclase 4 | NM_139247 |
| ADH1A | Alcohol dehydrogenase 1A (class I), alpha polypeptide | NM_000667 |
| ADRA2C | Adrenergic, alpha-2C-, receptor | NM_000683 |
| AHR | Aryl hydrocarbon receptor | NM_001621 |
| AKAP12 | A kinase (PRKA) anchor protein (gravin) 12 | NM_144497 |
| AMPH | Amphiphysin (Stiff-Man syndrome with breast cancer | NM_001635 |
| | 128 kDa autoantigen) | |
| ANGPTL4 | Angiopoietin-like 4 | NM_139314 |
| ANK1 | Ankyrin 1, erythrocytic | NM_020478 |
| ANLN | Anillin, actin binding protein (scraps homolog, | NM_018685 |
| | Drosophila) | |
| ANXA3 | Annexin A3 | NM_005139 |
| APCDD1 | Adenomatosis polyposis coli down-regulated 1 | NM_153000 |
| APOD | Apolipoprotein D | NM_001647 |
| APOH | Apolipoprotein H (beta-2-glycoprotein I) | NM_000042 |
| ARHGAP18 | Rho GTPase activating protein 18 | NM_033515 |
| ARHGDIB | Rho GDP dissociation inhibitor (GDI) beta | NM_001175 |
| ARNT2 | Aryl-hydrocarbon receptor nuclear translocator 2 | NM_014862 |
| ARRDC4 | Arrestin domain containing 4 | NM_183376 |
| ASB9 | Ankyrin repeat and SOCS box-containing 9 | NM_024087 |
| ASCL2 | Achaete-scute complex-like 2 (Drosophila) | NM_005170 |
| ASPA | Aspartoacylase (aminoacylase 2, Canavan disease) | NM_000049 |
| ASPM | Asp (abnormal spindle)-like, microcephaly associated | NM_018136 |
| | (Drosophila) | |
| ASPM | Asp (abnormal spindle)-like, microcephaly associated | NM_018136 |
| | (Drosophila) | |
| ATF3 | Activating transcription factor 3 | NM_004024 |
| ATF7IP2 | Activating transcription factor 7 interacting protein 2 | CR626222 |
| BCL11A | B-cell CLL/lymphoma 11A (zinc finger protein) | BU540282 |
| BDKRB1 | Bradykinin receptor B1 | NM_000710 |
| BDKRB1 | Bradykinin receptor B1 | NM_000710 |
| BDKRB2 | Bradykinin receptor B2 | NM_000623 |
| BIRC5 | Baculoviral IAP repeat-containing 5 (survivin) | BC007606 |
| BNC1 | Basonuclin 1 | NM_001717 |
| BNC2 | Basonuclin 2 | BC020879 |
| BNC2 | Basonuclin 2 | NM_017637 |
| BNC2 | Basonuclin 2 | NM_017637 |
| BSPRY | B-box and SPRY domain containing | NM_017688 |
| BUB1 | BUB1 budding uninhibited by benzimidazoles 1 | NM_004336 |
| | homolog (yeast) | |
| C10orf10 | Chromosome 10 open reading frame 10 | NM_007021 |
| C10orf3 | Chromosome 10 open reading frame 3 | NM_018131 |
| C10orf72 | Chromosome 10 open reading frame 72 | AK001062 |
| C13orf3 | Chromosome 13 open reading frame 3 | BC013418 |
| C18orf11 | Chromosome 18 open reading frame 11 | NM_022751 |
| C18orf11 | Chromosome 18 open reading frame 11 | NM_022751 |
| C18orf4 | Chromosome 18 open reading frame 4 | NM_032160 |
| C20orf129 | Chromosome 20 open reading frame 129 | NM_030919 |
| C21orf81 | Chromosome 21 open reading frame 81 | NM_153750 |
| C4BPA | Complement component 4 binding protein, alpha | NM_000715 |
| C5orf13 | Chromosome 5 open reading frame 13 | NM_004772 |
| C5orf4 | Chromosome 5 open reading frame 4 | NM_032385 |
| C8orf22 | Chromosome 8 open reading frame 22 | NM_001007176 |
| C9orf58 | Chromosome 9 open reading frame 58 | NM_001002260 |
| C9orf58 | Chromosome 9 open reading frame 58 | NM_001002260 |
| CA8 | Carbonic anhydrase VIII | NM_004056 |
| CAV1 | Caveolin 1, caveolae protein, 22 kDa | NM_001753 |
| CAV1 | Caveolin 1, caveolae protein, 22 kDa | NM_001753 |
| CCL2 | Chemokine (C-C motif) ligand 2 | NM_002982 |
| CCL26 | Chemokine (C-C motif) ligand 26 | NM_006072 |
| CCNB1 | Cyclin B1 | NM_031966 |
| CCNB2 | Cyclin B2 | NM_004701 |
| CCR1 | Chemokine (C-C motif) receptor 1 | NM_001295 |
| CCRL1 | Chemokine (C-C motif) receptor-like 1 | NM_178445 |
| CD200 | CD200 antigen | NM_001004196 |
| CD33 | CD33 antigen (gp67) | NM_001772 |
| CD38 | CD38 antigen (p45) | NM_001775 |
| CD3G | CD3G antigen, gamma polypeptide (TiT3 complex) | NM_000073 |
| CDC2 | Cell division cycle 2, G1 to S and G2 to M | NM_001786 |
| CDC20 | CDC20 cell division cycle 20 homolog (S. cerevisiae) | NM_001255 |
| CDC25C | Cell division cycle 25C | NM_001790 |
| CDC37L1 | Cell division cycle 37 homolog (S. cerevisiae)-like 1 | NM_017913 |
| CDCA2 | Cell division cycle associated 2 | NM_152562 |
| CDCA5 | Cell division cycle associated 5 | NM_080668 |
| CDCA8 | Cell division cycle associated 8 | NM_018101 |
| CDH1 | Cadherin 1, type 1, E-cadherin (epithelial) | NM_004360 |
| CDH18 | Cadherin 18, type 2 | NM_004934 |
| CDKN3 | Cyclin-dependent kinase inhibitor 3 (CDK2-associated | NM_005192 |
| | dual specificity phosphatase) | |
| CEACAM1 | Carcinoembryonic antigen-related cell adhesion | NM_001712 |
| | molecule 1 (biliary glycoprotein) | |
| CENPF | Centromere protein F, 350/400ka (mitosin) | NM_016343 |
| CGA | Glycoprotein hormones, alpha polypeptide | NM_000735 |
| CH25H | Cholesterol 25-hydroxylase | NM_003956 |
| CHST6 | Carbohydrate (N-acetylglucosamine 6-O) | NM_021615 |
| | sulfotransferase 6 | |
| CISH | Cytokine inducible SH2-containing protein | NM_145071 |
| CITED4 | Cbp/p300-interacting transactivator, with Glu/Asp-rich | NM_133467 |
| | carboxy-terminal domain, 4 | |
| CKLFSF8 | Chemokine-like factor super family 8 | NM_178868 |
| CLDN11 | Claudin 11 (oligodendrocyte transmembrane protein) | AF085871 |
| CMKOR1 | Chemokine orphan receptor 1 | NM_020311 |
| CNIH3 | Cornichon homolog 3 (Drosophila) | NM_152495 |
| COL4A6 | Collagen, type IV, alpha 6 | NM_033641 |
| COL8A2 | Collagen, type VIII, alpha 2 | NM_005202 |
| CP | Ceruloplasmin (ferroxidase) | NM_000096 |
| CPB2 | Carboxypeptidase B2 (plasma, carboxypeptidase U) | NM_001872 |
| CPXM2 | Carboxypeptidase X (M14 family), member 2 | NM_198148 |
| CTGF | Connective tissue growth factor | NM_001901 |
| CTNNAL1 | Catenin (cadherin-associated protein), alpha-like 1 | NM_003798 |
| CX3CL1 | Chemokine (C—X3—C motif) ligand 1 | NM_002996 |
| CX3CR1 | Chemokine (C—X3—C motif) receptor 1 | NM_001337 |
| CXCL1 | Chemokine (C—X—C motif) ligand 1 (melanoma growth | NM_001511 |
| | stimulating activity, alpha) | |
| CXCL14 | Chemokine (C—X—C motif) ligand 14 | NM_004887 |
| CXCR4 | chemokine (C—X—C motif) receptor 4 | NM_001008540 |
| CYP2F1 | Cytochrome P450, family 2, subfamily F, polypeptide 1 | NM_000774 |
| DCAMKL1 | Doublecortin and CaM kinase-like 1 | NM_004734 |
| DCN | Decorin | BQ004014 |
| DKFZP434B061 | DKFZP434B061 protein | AL117481 |
| DKFZP434I216 | DKFZP434I216 protein | NM_015432 |
| DKFZp564I1922 | Adlican | NM_015419 |
| DKFZP586A0522 | DKFZP586A0522 protein | NM_014033 |
| DKFZP586A0522 | DKFZP586A0522 protein | NM_014033 |
| DKFZP586K1520 | DKFZP586K1520 protein | AL050153 |
| DLG7 | Discs, large homolog 7 (Drosophila) | NM_014750 |
| DMD | Dystrophin (muscular dystrophy, Duchenne and Becker | NM_004010 |
| | types) | |
| DOK1 | Docking protein 1, 62 kDa (downstream of tyrosine | NM_001381 |
| | kinase 1) | |
| DRCTNNB1A | Down-regulated by Ctnnb1, a | NM_032581 |
| DUSP6 | Dual specificity phosphatase 6 | NM_001946 |
| ECHDC3 | Enoyl Coenzyme A hydratase domain containing 3 | NM_024693 |
| ECM2 | Extracellular matrix protein 2, female organ and | NM_001393 |
| | adipocyte specific | |
| EDN1 | Endothelin 1 | NM_001955 |
| EFNB2 | Ephrin-B2 | NM_004093 |
| EGLN3 | Eg1 nine homolog 3 (C. elegans) | NM_022073 |
| EGR1 | Early growth response 1 | NM_001964 |
| EN1 | Engrailed homolog 1 | NM_001426 |
| ENC1 | Ectodermal-neural cortex (with BTB-like domain) | NM_003633 |
| ENC1 | Ectodermal-neural cortex (with BTB-like domain) | NM_003633 |
| EPHA4 | EPH receptor A4 | NM_004438 |
| EPHX2 | Epoxide hydrolase 2, cytoplasmic | NM_001979 |
| EXOSC8 | Exosome component 8 | NM_181503 |
| EXOSC8 | Exosome component 8 | NM_181503 |
| FABP1 | Fatty acid binding protein 1, liver | NM_001443 |
| FADS1 | Fatty acid desaturase 1 | NM_013402 |
| FBXO32 | F-box protein 32 | NM_058229 |
| FCGR2A | Fc fragment of IgG, low affinity IIa, receptor for | NM_021642 |
| | (CD32) | |
| FGF7 | Galactokinase 2 | NM_002009 |
| FGF7 | Galactokinase 2 | NM_002009 |
| FGF7 | Galactokinase 2 | NM_002009 |
| FHL2 | Four and a half LIM domains 2 | NM_201555 |
| FKSG14 | Leucine zipper protein FKSG14 | NM_022145 |
| FLJ10156 | Hypothetical protein FLJ10156 | NM_019013 |
| FLJ13391 | Hypothetical protein FLJ13391 | NM_032181 |
| FLJ14712 | Hypothetical protein FLJ14712 | AK027618 |
| FLJ20255 | Hypothetical protein FLJ20255 | AK000262 |
| FLJ31340 | Hypothetical protein FLJ31340 | NM_152748 |
| FLJ35767 | FLJ35767 protein | NM_207459 |
| FLJ36031 | Hypothetical protein FLJ36031 | AK098422 |
| FLJ36031 | Hypothetical protein FLJ36031 | NM_175884 |
| FLJ37478 | Hypothetical protein FLJ37478 | NM_178557 |
| FLJ40629 | Hypothetical protein FLJ40629 | NM_152515 |
| FMN | Formin (limb deformity) | BC029107 |
| FOXQ1 | Forkhead box Q1 | NM_033260 |
| FZD10 | Frizzled homolog 10 (Drosophila) | NM_007197 |
| FZD4 | Frizzled homolog 4 (Drosophila) | NM_012193 |
| G2 | G2 protein | U10991 |
| GAL | Galanin | NM_015973 |
| GAS1 | Growth arrest-specific 1 | NM_002048 |
| GATA6 | GATA binding protein 6 | NM_005257 |
| GDF3 | Growth differentiation factor 3 | NM_020634 |
| GEM | GTP binding protein overexpressed in skeletal muscle | NM_005261 |
| GLCCI1 | Glucocorticoid induced transcript 1 | NM_138426 |
| GNG11 | Guanine nucleotide binding protein (G protein), gamma | NM_004126 |
| | 11 | |
| GPR68 | G protein-coupled receptor 68 | NM_003485 |
| GREM1 | Gremlin 1 homolog, cysteine knot superfamily | NM_013372 |
| | (Xenopus laevis) | |
| GSG1 | Germ cell associated 1 | NM_031289 |
| GTSE1 | G-2 and S-phase expressed 1 | NM_016426 |
| HAS3 | Hyaluronan synthase 3 | NM_005329 |
| HCAP-G | Chromosome condensation protein G | NM_022346 |
| HES1 | Hairy and enhancer of split 1, (Drosophila) | NM_005524 |
| HIST1H4B | Histone 1, H4b | NM_003544 |
| HIST1H4C | Histone 1, H4c | NM_003542 |
| HIST1H4L | Histone 1, H4l | NM_003546 |
| HLF | Hepatic leukemia factor | NM_002126 |
| HMMR | Hyaluronan-mediated motility receptor (RHAMM) | NM_012484 |
| HRH1 | Histamine receptor H1 | NM_000861 |
| HT008 | Uncharacterized hypothalamus protein HT008 | NM_018469 |
| ICA1 | Islet cell autoantigen 1, 69 kDa | NM_004968 |
| ICAM5 | Intercellular adhesion molecule 5, telencephalin | NM_003259 |
| ID1 | Inhibitor of DNA binding 1, dominant negative helix- | NM_002165 |
| | loop-helix protein | |
| IFI44 | Interferon-induced protein 44 | NM_006417 |
| IL6 | Interleukin 6 (interferon, beta 2) | NM_000600 |
| INSIG2 | Insulin induced gene 2 | NM_016133 |
| INSIG2 | Insulin induced gene 2 | NM_016133 |
| IRF5 | Interferon regulatory factor 5 | NM_002200 |
| JAG1 | Jagged 1 (Alagille syndrome) | NM_000214 |
| KCNH2 | Potassium voltage-gated channel, subfamily H (eag- | NM_000238 |
| | related), member 2 | |
| KCNMB4 | Potassium large conductance calcium-activated | NM_014505 |
| | channel, subfamily M, beta member 4 | |
| KCTD12 | Potassium channel tetramerisation domain containing | NM_138444 |
| | 12 | |
| KIAA0101 | KIAA0101 | NM_014736 |
| KIAA1199 | KIAA1199 | NM_018689 |
| KIAA1199 | KIAA1199 | NM_018689 |
| KIAA1217 | KIAA1217 | AK022045 |
| KIAA1217 | KIAA1217 | NM_019590 |
| KIAA1509 | KIAA1509 | AB040942 |
| KIAA1644 | KIAA1644 protein | AB051431 |
| KIAA1666 | KIAA1666 protein | BC035246 |
| KIAA1913 | KIAA1913 | BC044246 |
| KIF18A | Kinesin family member 18A | NM_031217 |
| KIF20A | Kinesin family member 20A | NM_005733 |
| KIF2C | Kinesin family member 2C | NM_006845 |
| KIF4A | Kinesin family member 4A | NM_012310 |
| KLF2 | Kruppel-like factor 2 (lung) | NM_016270 |
| KLK8 | Kallikrein 8 (neuropsin/ovasin) | NM_144505 |
| KLRC1 | Killer cell lectin-like receptor subfamily C, member 1 | NM_002259 |
| KNTC2 | Kinetochore associated 2 | NM_006101 |
| KRT23 | Keratin 23 (histone deacetylase inducible) | NM_015515 |
| KRTAP1-5 | Keratin associated protein 1-5 | NM_031957 |
| LAD1 | Ladinin 1 | NM_005558 |
| LAMA2 | Laminin, alpha 2 (merosin, congenital muscular | NM_000426 |
| | dystrophy) | |
| LEF1 | Lymphoid enhancer-binding factor 1 | NM_016269 |
| LHX2 | LIM homeobox 2 | NM_004789 |
| LIPE | Lipase, hormone-sensitive | NM_005357 |
| LMNB1 | Lamin B1 | NM_005573 |
| LOC126755 | Hypothetical protein LOC126755 | CR622769 |
| LOC150166 | Hypothetical protein LOC150166 | AK056836 |
| LOC150271 | Hypothetical LOC388889 | AK098753 |
| LOC199964 | Hypothetical protein LOC199964 | NM_182532 |
| LOC222171 | Hypothetical protein LOC222171 | NM_175887 |
| LOC255480 | Hypothetical protein LOC255480 | AK091766 |
| LOC284018 | Hypothetical protein LOC284018 | NM_181655 |
| LOC285733 | Hypothetical protein LOC285733 | AK091900 |
| LOC286254 | Hypothetical protein LOC286254 | AK092751 |
| LOC51334 | Mesenchymal stem cell protein DSC54 | NM_016644 |
| LOXL3 | Lysyl oxidase-like 3 | NM_032603 |
| LOXL3 | Lysyl oxidase-like 3 | NM_032603 |
| LPXN | Leupaxin | NM_004811 |
| LRP8 | Low density lipoprotein receptor-related protein 8, | NM_033300 |
| | apolipoprotein e receptor | |
| LYZ | Lysozyme (renal amyloidosis) | NM_000239 |
| LZTS1 | Leucine zipper, putative tumor suppressor 1 | NM_021020 |
| MAD2L1 | MAD2 mitotic arrest deficient-like 1 (yeast) | NM_002358 |
| MAFB | V-maf musculoaponeurotic fibrosarcoma oncogene | NM_005461 |
| | homolog B (avian) | |
| MAGEA1 | Melanoma antigen, family A, 1 (directs expression of | NM_004988 |
| | antigen MZ2-E) | |
| MAL2 | Mal, T-cell differentiation protein 2 | NM_052886 |
| MAOB | Monoamine oxidase B | NM_000898 |
| MAP3K8 | Mitogen-activated protein kinase kinase kinase 8 | NM_005204 |
| MARLIN1 | Multiple coiled-coil GABABR1-binding protein | NM_144720 |
| MEST | Mesoderm specific transcript homolog (mouse) | NM_002402 |
| MGAT3 | Mannosyl (beta-1,4-)-glycoprotein beta-1,4-N- | AK125361 |
| | acetylglucosaminyltransferase | |
| MGC13040 | Hypothetical protein MGC13040 | NM_032930 |
| MGC22265 | Hypothetical protein MGC22265 | BC048193 |
| MGC2574 | Hypothetical protein MGC2574 | NM_024098 |
| MGC2574 | Hypothetical protein MGC2574 | NM_024098 |
| MGC33365 | Hypothetical protein MGC33365 | NM_173552 |
| MLANA | Melan-A | NM_005511 |
| MMP12 | Matrix metalloproteinase 12 (macrophage elastase) | NM_002426 |
| MSX1 | Msh homeo box homolog 1 (Drosophila) | NM_002448 |
| MT1B | Metallothionein 1B (functional) | NM_005947 |
| MT1E | Metallothionein 1E (functional) | NM_175617 |
| MT1G | Metallothionein 1G | NM_005950 |
| MT1K | Metallothionein 1K | NM_176870 |
| MT1L | Metallothionein 1L | X97261 |
| MT1X | Metallothionein 1X | NM_005952 |
| MT2A | Metallothionein 2A | NM_005953 |
| MT2A | Metallothionein 2A | NM_005953 |
| MTL5 | Metallothionein-like 5, testis-specific (tesmin) | NM_004923 |
| MYCN | V-myc myelocytomatosis viral related oncogene, | NM_005378 |
| | neuroblastoma derived (avian) | |
| MYO10 | Myosin X | NM_012334 |
| MYO10 | Myosin X | NM_012334 |
| MYO5B | Myosin VB | AK025336 |
| MYO5C | Myosin VC | NM_018728 |
| MYRIP | Myosin VIIA and Rab interacting protein | NM_015460 |
| NAV2 | Neuron navigator 2 | NM_182964 |
| NET1 | Neuroepithelial cell transforming gene 1 | NM_005863 |
| NETO2 | Neuropilin (NRP) and tolloid (TLL)-like 2 | NM_018092 |
| NFE2 | Nuclear factor (erythroid-derived 2), 45 kDa | NM_006163 |
| NFIL3 | Nuclear factor, interleukin 3 regulated | NM_005384 |
| NGEF | Neuronal guanine nucleotide exchange factor | NM_019850 |
| NID2 | Nidogen 2 (osteonidogen) | NM_007361 |
| NOSTRIN | Nitric oxide synthase trafficker | NM_052946 |
| NOV | Nephroblastoma overexpressed gene | NM_002514 |
| NR0B1 | Nuclear receptor subfamily 0, group B, member 1 | NM_000475 |
| NR0B2 | Nuclear receptor subfamily 0, group B, member 2 | NM_021969 |
| NSE1 | NSE1 | NM_145175 |
| NTN4 | Netrin 4 | NM_021229 |
| NTS | Neurotensin | NM_006183 |
| ODZ3 | Odz, odd Oz/ten-m homolog 3 (Drosophila) | AB040888 |
| ODZ3 | Odz, odd Oz/ten-m homolog 3 (Drosophila) | AB040888 |
| OIP5 | Opa-interacting protein 5 | NM_007280 |
| OLFML2A | Olfactomedin-like 2A | NM_182487 |
| OR7E140P | Olfactory receptor, family 7, subfamily E, member 140 | BC073935 |
| | pseudogene | |
| OVOS2 | Ovostatin 2 | BC039117 |
| PAG | Phosphoprotein associated with glycosphingolipid- | NM_018440 |
| | enriched microdomains | |
| PBEF1 | Pre-B-cell colony enhancing factor 1 | NM_005746 |
| PBEF1 | Pre-B-cell colony enhancing factor 1 | NM_182790 |
| PCANAP6 | Prostate cancer associated protein 6 | NM_033102 |
| PCSK5 | Proprotein convertase subtilisin/kexin type 5 | NM_006200 |
| PDGFA | Platelet-derived growth factor alpha polypeptide | NM_002607 |
| PDGFC | Platelet derived growth factor C | NM_016205 |
| PDGFD | DNA-damage inducible protein 1 | NM_025208 |
| PHACTR1 | Phosphatase and actin regulator 1 | NM_030948 |
| PHLDA1 | Pleckstrin homology-like domain, family A, member 1 | NM_007350 |
| PHLDA1 | Pleckstrin homology-like domain, family A, member 1 | NM_007350 |
| PHLDB2 | Pleckstrin homology-like domain, family B, member 2 | NM_145753 |
| PIK3R1 | Phosphoinositide-3-kinase, regulatory subunit 1 (p85 | NM_181523 |
| | alpha) | |
| PIM1 | Pim-1 oncogene | NM_002648 |
| PKD1L2 | Polycystic kidney disease 1-like 2 | NM_052892 |
| PKD2 | Polycystic kidney disease 2 (autosomal dominant) | NM_000297 |
| PLAC8 | Placenta-specific 8 | NM_016619 |
| PLAC8 | Placenta-specific 8 | NM_016619 |
| PLD1 | Phospholipase D1, phophatidylcholine-specific | NM_002662 |
| PLK2 | Polo-like kinase 2 (Drosophila) | NM_006622 |
| PLP1 | Proteolipid protein 1 (Pelizaeus-Merzbacher disease, | M54927 |
| | spastic paraplegia 2, uncomplicated) | |
| PMAIP1 | Phorbol-12-myristate-13-acetate-induced protein 1 | NM_021127 |
| PPP1R1A | Protein phosphatase 1, regulatory (inhibitor) subunit 1A | NM_006741 |
| PPP1R3B | Protein phosphatase 1, regulatory (inhibitor) subunit 3B | AK091994 |
| PPP2R3A | Protein phosphatase 2 (formerly 2A), regulatory subunit | NM_002718 |
| | B″, alpha | |
| PRC1 | Protein regulator of cytokinesis 1 | NM_003981 |
| PREX1 | Phosphatidylinositol 3,4,5-trisphosphate-dependent | NM_020820 |
| | RAC exchanger 1 | |
| PRKCB1 | Protein kinase C, beta 1 | NM_002738 |
| PRKCB1 | Protein kinase C, beta 1 | NM_002738 |
| PROC | Protein C (inactivator of coagulation factors Va and | NM_000312 |
| | VIIIa) | |
| PSCDBP | Pleckstrin homology, Sec7 and coiled-coil domains, | NM_004288 |
| | binding protein | |
| PSD3 | Pleckstrin and Sec7 domain containing 3 | NM_015310 |
| PSG11 | Pregnancy specific beta-1-glycoprotein 11 | NM_002785 |
| PSG3 | Pregnancy specific beta-1-glycoprotein 3 | NM_021016 |
| PTGER4 | Prostaglandin E receptor 4 (subtype EP4) | NM_000958 |
| PTGFR | Prostaglandin F receptor (FP) | NM_000959 |
| PTTG1 | Pituitary tumor-transforming 1 | NM_004219 |
| PTTG2 | Pituitary tumor-transforming 2 | NM_006607 |
| RAB11FIP2 | RAB11 family interacting protein 2 (class I) | NM_014904 |
| RACGAP1 | Rac GTPase activating protein 1 | NM_013277 |
| RAD52B | RAD52 homolog B (S. cerevisiae) | NM_145654 |
| RAMP1 | Receptor (calcitonin) activity modifying protein 1 | NM_005855 |
| RANBP9 | RAN binding protein 9 | NM_005493 |
| RANBP9 | RAN binding protein 9 | NM_005493 |
| RANBP9 | RAN binding protein 9 | NM_005493 |
| RASD1 | RAS, dexamethasone-induced 1 | NM_016084 |
| REV3L | REV3-like, catalytic subunit of DNA polymerase zeta | NM_002912 |
| | (yeast) | |
| RGS2 | Regulator of G-protein signalling 2, 24 kDa | NM_002923 |
| RIMS3 | Regulating synaptic membrane exocytosis 3 | NM_014747 |
| RIPK3 | Receptor-interacting serine-threonine kinase 3 | NM_006871 |
| RIPK4 | Receptor-interacting serine-threonine kinase 4 | NM_020639 |
| ROBO3 | Roundabout, axon guidance receptor, homolog 3 | NM_022370 |
| | (Drosophila) | |
| RPESP | RPE-spondin | NM_153225 |
| RRM2 | Ribonucleotide reductase M2 polypeptide | NM_001034 |
| RTN4R | Reticulon 4 receptor | NM_023004 |
| SALL2 | Sal-like 2 (Drosophila) | NM_005407 |
| SAMSN1 | SAM domain, SH3 domain and nuclear localisation | NM_022136 |
| | signals, 1 | |
| SATB1 | Special AT-rich sequence binding protein 1 (binds to | NM_002971 |
| | nuclear matrix/scaffold-associating DNA's) | |
| SCIN | Scinderin | NM_033128 |
| SECTM1 | Secreted and transmembrane 1 | NM_003004 |
| SEMA6A | Sema domain, transmembrane domain (TM), and | NM_020796 |
| | cytoplasmic domain, (semaphorin) 6A | |
| SEPP1 | Selenoprotein P, plasma, 1 | NM_005410 |
| SERPINA5 | Serine (or cysteine) proteinase inhibitor, clade A | NM_000624 |
| | (alpha-1 antiproteinase, antitrypsin), member 5 | |
| SERPINA7 | Serine (or cysteine) proteinase inhibitor, clade A | NM_000354 |
| | (alpha-1 antiproteinase, antitrypsin), member 7 | |
| SH2D1A | SH2 domain protein 1A, Duncan's disease | NM_002351 |
| | (lymphoproliferative syndrome) | |
| SLC16A6 | Solute carrier family 16 (monocarboxylic acid | NM_004694 |
| | transporters), member 6 | |
| SLC1A1 | Solute carrier family 1 (neuronal/epithelial high affinity | NM_004170 |
| | glutamate transporter, system Xag), member 1 | |
| SLC20A1 | Solute carrier family 20 (phosphate transporter), | NM_005415 |
| | member 1 | |
| SLC2A1 | Solute carrier family 2 (facilitated glucose transporter), | NM_006516 |
| | member 1 | |
| SLC39A8 | Solute carrier family 39 (zinc transporter), member 8 | NM_022154 |
| SLC40A1 | Solute carrier family 40 (iron-regulated transporter), | NM_014585 |
| | member 1 | |
| SLC7A5 | Solute carrier family 7 (cationic amino acid transporter, | NM_003486 |
| | y+ system), member 5 | |
| SLC9A9 | Solute carrier family 9 (sodium/hydrogen exchanger), | NM_173653 |
| | isoform 9 | |
| SLIT3 | Slit homolog 3 (Drosophila) | BC032027 |
| SLPI | Secretory leukocyte protease inhibitor | NM_003064 |
| | (antileukoproteinase) | |
| SMOC1 | SPARC related modular calcium binding 1 | NM_022137 |
| SMOC2 | SPARC related modular calcium binding 2 | NM_022138 |
| SNAI2 | Snail homolog 2 (Drosophila) | NM_003068 |
| SNFT | Jun dimerization protein p21SNFT | NM_018664 |
| SOCS1 | Suppressor of cytokine signaling 1 | NM_003745 |
| SORL1 | Sortilin-related receptor, L(DLR class) A repeats- | NM_003105 |
| | containing | |
| SOX4 | SRY (sex determining region Y)-box 4 | AW946823 |
| SOX4 | SRY (sex determining region Y)-box 4 | NM_003107 |
| SOX4 | SRY (sex determining region Y)-box 4 | NM_003107 |
| SP5 | Sp5 transcription factor | NM_001003845 |
| Spc25 | Kinetochore protein Spc25 | NM_020675 |
| SPHK1 | Sphingosine kinase 1 | NM_021972 |
| SPINT2 | Serine protease inhibitor, Kunitz type, 2 | NM_021102 |
| SRC | V-src sarcoma (Schmidt-Ruppin A-2) viral oncogene | NM_005417 |
| | homolog (avian) | |
| STAC | SH3 and cysteine rich domain | NM_003149 |
| STC2 | Stanniocalcin 2 | NM_003714 |
| STMN1 | Stathmin 1/oncoprotein 18 | NM_203401 |
| T3JAM | TRAF3-interacting Jun N-terminal kinase (JNK)- | NM_025228 |
| | activating modulator | |
| TCEAL7 | Transcription elongation factor A (SII)-like 7 | NM_152278 |
| TCF4 | Transcription factor 4 | AK021980 |
| TIGD2 | Tigger transposable element derived 2 | NM_145715 |
| TIMP3 | Tissue inhibitor of metalloproteinase 3 (Sorsby fundus | AA837799 |
| | dystrophy, pseudoinflammatory) | |
| TK1 | Thymidine kinase 1, soluble | NM_003258 |
| TM4SF1 | Transmembrane 4 superfamily member 1 | NM_014220 |
| TMPRSS4 | Transmembrane protease, serine 4 | NM_019894 |
| TMSNB | Thymosin, beta, identified in neuroblastoma cells | NM_021992 |
| TNC | Tenascin C (hexabrachion) | NM_002160 |
| TncRNA | Trophoblast-derived noncoding RNA | U60873 |
| TNFAIP6 | Tumor necrosis factor, alpha-induced protein 6 | NM_007115 |
| TNFRSF17 | Tumor necrosis factor receptor superfamily, member 17 | NM_001192 |
| TOP2A | Topoisomerase (DNA) II alpha 170 kDa | NM_001067 |
| TOPK | T-LAK cell-originated protein kinase | NM_018492 |
| TPD52 | Tumor protein D52 | NM_005079 |
| TPM1 | Tropomyosin 1 (alpha) | NM_000366 |
| TPX2 | TPX2, microtubule-associated protein homolog | NM_012112 |
| | (Xenopus laevis) | |
| TRIB1 | Tribbles homolog 1 (Drosophila) | NM_025195 |
| TRIB2 | Tribbles homolog 2 (Drosophila) | NM_021643 |
| TROAP | Trophinin associated protein (tastin) | NM_005480 |
| TRPS1 | Trichorhinophalangeal syndrome I | NM_014112 |
| TTK | TTK protein kinase | NM_003318 |
| TXNIP | Thioredoxin interacting protein | NM_006472 |
| TYRP1 | Tyrosinase-related protein 1 | NM_000550 |
| UAP1 | UDP-N-acteylglucosamine pyrophosphorylase 1 | NM_003115 |
| UBD | Ubiquitin D | NM_006398 |
| UBE2C | Ubiquitin-conjugating enzyme E2C | NM_181803 |
| UGT2B11 | UDP glycosyltransferase 2 family, polypeptide B11 | NM_001073 |
| UST | Uronyl-2-sulfotransferase | NM_005715 |
| UTS2 | Urotensin 2 | NM_021995 |
| UTS2 | Urotensin 2 | NM_021995 |
| VIL1 | Villin 1 | NM_007127 |
| YPEL4 | Yippee-like 4 (Drosophila) | NM_145008 |
| ZAP70 | Zeta-chain (TCR) associated protein kinase 70 kDa | NM_001079 |
| ZNF179 | Zinc finger protein 179 | NM_007148 |
| ZNF503 | Zinc finger protein 503 | NM_032772 |
| | | A_23_P15226 |
| | | A_23_P170719 |
| | | A_23_P43744 |
| | | A_24_P290087 |
| | | A_24_P686014 |
| | | A_24_P927205 |
| | | A_32_P182135 |
| | | A_32_P205792 |
| | | A_32_P225328 |
| | | A_32_P232647 |
| | | A_32_P55438 |
| | | AF256215 |
| | Hypothetical gene supported by AK026189 | AK022865 |
| | CDNA: FLJ22994 fis, clone KAT11918 | AK026647 |
| | CDNA: FLJ23131 fis, clone LNG08502 | AK026784 |
| | CDNA FLJ31059 fis, clone HSYRA2000832 | AK055621 |
| | Hypothetical LOC388397 | AK057167 |
| | Homo sapiens, clone IMAGE: 4214962, mRNA | AK091547 |
| | CDNA FLJ41489 fis, clone BRTHA2004582 | AK123483 |
| | MRNA full length insert cDNA clone EUROIMAGE | AK124841 |
| | 51148 | |
| | CDNA F1143172 fis, clone FCBBF3007242 | AK125162 |
| | CDNA FLJ26031 fis, clone PNC08078 | AK129542 |
| | Homo sapiens, clone IMAGE: 5285282, mRNA | AK129982 |
| | Similar to bA110H4.2 (similar to membrane protein) | AK130705 |
| | Transcribed locus | AW972815 |
| | Hypothetical gene supported by AY007155 | AY007155 |
| | Homo sapiens, clone IMAGE: 3869276, mRNA | BC018597 |
| | CDNA clone MGC: 65154 IMAGE: 5122136, complete | BC056907 |
| | cds | |
| | | BE893137 |
| | Transcribed locus, moderately similar to XP_497060.1 | BM989848 |
| | similar to FKSG60 [Homo sapiens] | |
| | Full-length cDNA clone CS0DJ001YJ05 of T cells | CR601458 |
| | (Jurkat cell line) Cot 10-normalized of Homo sapiens | |
| | (human) | |
| | Full-length cDNA clone CS0DC002YA18 of | CR624517 |
| | Neuroblastoma Cot 25-normalized of Homo sapiens | |
| | (human) | |
| | | CR936791 |
| | | CR936791 |
| | | CX788817 |
| | | ENST00000245185 |
| | | ENST00000261569 |
| | | ENST00000312275 |
| | | ENST00000314238 |
| | | ENST00000343505 |
| | | ENST00000371256 |
| | | ENST00000371655 |
| | | ENST00000375377 |
| | | ENST00000381889 |
| | | NM_001006641 |
| | | NM_001008708 |
| | | NM_001010911 |
| | | NM_001010915 |
| | | NM_001011543 |
| | | NM_001012271 |
| | | NM_001017420 |
| | | NM_001017424 |
| | | NM_001017535 |
| | | NM_001040100 |
| | | NM_001040167 |
| | | NM_001040457 |
| | | NM_002263 |
| | | NM_003621 |
| | | NM_014867 |
| | | NM_017577 |
| | | NM_020872 |
| | | NM_020872 |
| | | NM_020872 |
| | | NM_025135 |
| | | NM_032199 |
| | | NM_032532 |
| | | NR_001558 |
| | | THC2274524 |
| | | THC2308675 |
| | | THC2343246 |
| | | THC2347909 |
| | | THC2373845 |
| | | THC2376729 |
| | | THC2398598 |
| | | THC2405710 |
| | | THC2406576 |
| | | THC2407823 |
| | | THC2438492 |
| | | THC2438512 |
| | | THC2442210 |
| | | THC2442586 |
| | | THC2443654 |
| | | THC2455149 |
| | Similar to hypothetical protein LOC231503 | XM_496707 |
| | | XM_932314 |

TABLE 13

| Gene Symbol | Gene Name | Accession No. |
|---|---|---|
| ABCA6 | ATP-binding cassette, sub-family A (ABC1), member 6 | NM_080284 |
| ADAMTS1 | A disintegrin-like and metalloprotease (reprolysin type) | NM_006988 |
| | with thrombospondin type 1 motif, 1 | |
| ADAMTS1 | A disintegrin-like and metalloprotease (reprolysin type) | NM_006988 |
| | with thrombospondin type 1 motif, 1 | |
| ADCY4 | Adenylate cyclase 4 | NM_139247 |
| AFAP | Hypothetical protein LOC254848 | BC014113 |
| AGR2 | Anterior gradient 2 homolog (Xenopus laevis) | NM_006408 |
| ALOX5AP | Arachidonate 5-lipoxygenase-activating protein | NM_001629 |
| AMD1 | Adenosylmethionine decarboxylase 1 | NM_001634 |
| ANGPTL4 | Angiopoietin-like 4 | NM_139314 |
| ANK1 | Ankyrin 1, erythrocytic | NM_020478 |
| ANK3 | Ankyrin 3, node of Ranvier (ankyrin G) | NM_020987 |
| ANLN | Anillin, actin binding protein (scraps homolog, | NM_018685 |
| | Drosophila) | |
| ANXA3 | Annexin A3 | NM_005139 |
| APCDD1 | Adenomatosis polyposis coli down-regulated 1 | NM_153000 |
| APOBEC3B | Apolipoprotein B mRNA editing enzyme, catalytic | NM_004900 |
| | polypeptide-like 3B | |
| APOL6 | Apolipoprotein L, 6 | NM_030641 |
| AREG | Amphiregulin (schwannoma-derived growth factor) | NM_001657 |
| ARHGDIB | Rho GDP dissociation inhibitor (GDI) beta | NM_001175 |
| ARL4A | ADP-ribosylation factor-like 4A | NM_005738 |
| ARRDC4 | Arrestin domain containing 4 | NM_183376 |
| ASB9 | Ankyrin repeat and SOCS box-containing 9 | NM_024087 |
| ASPA | Aspartoacylase (aminoacylase 2, Canavan disease) | NM_000049 |
| ASPM | Asp (abnormal spindle)-like, microcephaly associated | NM_018136 |
| | (Drosophila) | |
| ASPM | Asp (abnormal spindle)-like, microcephaly associated | NM_018136 |
| | (Drosophila) | |
| ASRGL1 | Asparaginase like 1 | BC006267 |
| ASRGL1 | Asparaginase like 1 | NM_025080 |
| ATF3 | Activating transcription factor 3 | NM_004024 |
| BCL11A | B-cell CLL/lymphoma 11A (zinc finger protein) | BU540282 |
| BCL11A | B-cell CLL/lymphoma 11A (zinc finger protein) | NM_022893 |
| BDKRB1 | Bradykinin receptor B1 | NM_000710 |
| BDKRB2 | Bradykinin receptor B2 | NM_000623 |
| BIRC5 | Baculoviral IAP repeat-containing 5 (survivin) | BC007606 |
| BNC1 | Basonuclin 1 | NM_001717 |
| BNC2 | Basonuclin 2 | BC020879 |
| BNC2 | Basonuclin 2 | NM_017637 |
| BUB1 | BUB1 budding uninhibited by benzimidazoles 1 | NM_004336 |
| | homolog (yeast) | |
| C10orf3 | Chromosome 10 open reading frame 3 | NM_018131 |
| C13orf3 | Chromosome 13 open reading frame 3 | BC013418 |
| C18orf11 | Chromosome 18 open reading frame 11 | NM_022751 |
| C20orf103 | Chromosome 20 open reading frame 103 | NM_012261 |
| C5orf13 | Chromosome 5 open reading frame 13 | NM_004772 |
| C6orf176 | Chromosome 6 open reading frame 176 | CR618615 |
| C8orf22 | Chromosome 8 open reading frame 22 | NM_001007176 |
| CAV1 | Caveolin 1, caveolae protein, 22 kDa | NM_001753 |
| CAV1 | Caveolin 1, caveolae protein, 22 kDa | NM_001753 |
| CAV3 | Caveolin 3 | NM_001234 |
| CCL2 | Chemokine (C-C motif) ligand 2 | NM_002982 |
| CCNB1 | Cyclin B1 | NM_031966 |
| CCNB2 | Cyclin B2 | NM_004701 |
| CCR1 | Chemokine (C-C motif) receptor 1 | NM_001295 |
| CD1A | CD1A antigen, a polypeptide | BC031645 |
| CD200 | CD200 antigen | NM_001004196 |
| CD28 | CD28 antigen (Tp44) | NM_006139 |
| CD33 | CD33 antigen (gp67) | NM_001772 |
| CD38 | CD38 antigen (p45) | NM_001775 |
| CD3Z | CD3Z antigen, zeta polypeptide (TiT3 complex) | NM_198053 |
| CDC2 | Cell division cycle 2, G1 to S and G2 to M | NM_001786 |
| CDC20 | CDC20 cell division cycle 20 homolog (S. cerevisiae) | NM_001255 |
| CDC37L1 | Cell division cycle 37 homolog (S. cerevisiae)-like 1 | NM_017913 |
| CDCA1 | Cell division cycle associated 1 | NM_145697 |
| CDCA2 | Cell division cycle associated 2 | NM_152562 |
| CDCA7 | Cell division cycle associated 7 | NM_031942 |
| CDCA8 | Cell division cycle associated 8 | NM_018101 |
| CDH1 | Cadherin 1, type 1, E-cadherin (epithelial) | NM_004360 |
| CDH18 | Cadherin 18, type 2 | NM_004934 |
| CDKN3 | Cyclin-dependent kinase inhibitor 3 (CDK2-associated | NM_005192 |
| | dual specificity phosphatase) | |
| CENPA | Centromere protein A, 17 kDa | NM_001809 |
| CENPF | Centromere protein F, 350/400ka (mitosin) | NM_016343 |
| CGA | Glycoprotein hormones, alpha polypeptide | NM_000735 |
| CGA | Glycoprotein hormones, alpha polypeptide | NM_000735 |
| CH25H | Cholesterol 25-hydroxylase | NM_003956 |
| CHD7 | Chromodomain helicase DNA binding protein 7 | NM_017780 |
| CHSY1 | Carbohydrate (chondroitin) synthase 1 | NM_014918 |
| CISH | Cytokine inducible SH2-containing protein | NM_145071 |
| CITED4 | Cbp/p300-interacting transactivator, with Glu/Asp-rich | NM_133467 |
| | carboxy-terminal domain, 4 | |
| CLDN11 | Claudin 11 (oligodendrocyte transmembrane protein) | AF085871 |
| CLIC3 | Chloride intracellular channel 3 | NM_004669 |
| CMKOR1 | Chemokine orphan receptor 1 | NM_020311 |
| CMRF-35H | Leukocyte membrane antigen | NM_007261 |
| CNIH3 | Cornichon homolog 3 (Drosophila) | NM_152495 |
| COBLL1 | COBL-like 1 | NM_014900 |
| COCH | Coagulation factor C homolog, cochlin (Limulus | NM_004086 |
| | polyphemus) | |
| COL3A1 | Collagen, type III, alpha 1 (Ehlers-Danlos syndrome | NM_000090 |
| | type IV, autosomal dominant) | |
| COL4A6 | Collagen, type IV, alpha 6 | NM_033641 |
| COL8A1 | Collagen, type VIII, alpha 1 | AL359062 |
| CPB2 | Carboxypeptidase B2 (plasma, carboxypeptidase U) | NM_001872 |
| CTGF | Connective tissue growth factor | NM_001901 |
| CTNNAL1 | Catenin (cadherin-associated protein), alpha-like 1 | NM_003798 |
| CTNND2 | Catenin (cadherin-associated protein), delta 2 (neural | NM_001332 |
| | plakophilin-related arm-repeat protein) | |
| CX3CR1 | Chemokine (C—X3—C motif) receptor 1 | NM_001337 |
| CXCL1 | Chemokine (C—X—C motif) ligand 1 (melanoma growth | NM_001511 |
| | stimulating activity, alpha) | |
| CXCR4 | chemokine (C—X—C motif) receptor 4 | NM_001008540 |
| DDC | Dopa decarboxylase (aromatic L-amino acid | NM_000790 |
| | decarboxylase) | |
| DEPDC1 | DEP domain containing 1 | NM_017779 |
| DEPDC1B | DEP domain containing 1B | NM_018369 |
| DKFZP434B061 | DKFZP434B061 protein | AL117481 |
| DKFZP547L112 | Hypothetical protein DKFZp547L112 | AL512723 |
| DKFZP586A0522 | DKFZP586A0522 protein | NM_014033 |
| DKFZP586A0522 | DKFZP586A0522 protein | NM_014033 |
| DKK2 | Dickkopf homolog 2 (Xenopus laevis) | NM_014421 |
| DLG7 | Discs, large homolog 7 (Drosophila) | NM_014750 |
| DMD | Dystrophin (muscular dystrophy, Duchenne and Becker | NM_004010 |
| | types) | |
| DNAJC12 | DnaJ (Hsp40) homolog, subfamily C, member 12 | NM_021800 |
| DNM3 | Dynamin 3 | AK021543 |
| DOK1 | Docking protein 1, 62 kDa (downstream of tyrosine | NM_001381 |
| | kinase 1) | |
| DPPA4 | Developmental pluripotency associated 4 | NM_018189 |
| DUSP6 | Dual specificity phosphatase 6 | NM_001946 |
| ECM2 | Extracellular matrix protein 2, female organ and | NM_001393 |
| | adipocyte specific | |
| EDN1 | Endothelin 1 | NM_001955 |
| EFNB2 | Ephrin-B2 | NM_004093 |
| EGLN3 | Egl nine homolog 3 (C. elegans) | NM_022073 |
| EGR1 | Early growth response 1 | NM_001964 |
| ELF3 | E74-like factor 3 (ets domain transcription factor, | NM_004433 |
| | epithelial-specific) | |
| EN1 | Engrailed homolog 1 | NM_001426 |
| ENC1 | Ectodermal-neural cortex (with BTB-like domain) | NM_003633 |
| ENC1 | Ectodermal-neural cortex (with BTB-like domain) | NM_003633 |
| EPB41L4B | Erythrocyte membrane protein band 4.1 like 4B | NM_018424 |
| EPHA4 | EPH receptor A4 | NM_004438 |
| EPHX2 | Epoxide hydrolase 2, cytoplasmic | NM_001979 |
| EVA1 | Epithelial V-like antigen 1 | NM_144765 |
| EXOSC8 | Exosome component 8 | NM_181503 |
| EXOSC8 | Exosome component 8 | NM_181503 |
| F11 | Coagulation factor XI (plasma thromboplastin | NM_000128 |
| | antecedent) | |
| F3 | Coagulation factor III (thromboplastin, tissue factor) | NM_001993 |
| FA2H | Fatty acid 2-hydroxylase | NM_024306 |
| FADS1 | Fatty acid desaturase 1 | NM_013402 |
| FBXL16 | F-box and leucine-rich repeat protein 16 | NM_153350 |
| FBXO32 | F-box protein 32 | NM_058229 |
| FCGBP | Fc fragment of IgG binding protein | NM_003890 |
| FGA | Fibrinogen, A alpha polypeptide | NM_000508 |
| FGF7 | Galactokinase 2 | NM_002009 |
| FGF7 | Galactokinase 2 | NM_002009 |
| FHL2 | Four and a half LIM domains 2 | NM_201555 |
| FLJ10156 | Hypothetical protein FLJ10156 | NM_019013 |
| FLJ10901 | Hypothetical protein FLJ10901 | NM_018265 |
| FLJ13072 | Hypothetical gene FLJ13072 | AK023134 |
| FLJ13391 | Hypothetical protein FLJ13391 | NM_032181 |
| FLJ13840 | Hypothetical protein FLJ13840 | BC007638 |
| FLJ14712 | Hypothetical protein FLJ14712 | AK027618 |
| FLJ14834 | Hypothetical protein FLJ14834 | NM_032849 |
| FLJ30681 | KIAA1983 protein | NM_133459 |
| FLJ31340 | Hypothetical protein FLJ31340 | NM_152748 |
| FLJ31461 | Hypothetical protein FLJ31461 | NM_152454 |
| FLJ35767 | FLJ35767 protein | NM_207459 |
| FLJ36031 | Hypothetical protein FLJ36031 | AK098422 |
| FLJ37478 | Hypothetical protein FLJ37478 | NM_178557 |
| FLJ37970 | Hypothetical protein FLJ37970 | NM_032251 |
| FLJ39739 | FLJ39739 protein | AK026418 |
| FLJ45273 | FLJ45273 protein | NM_198461 |
| FLRT2 | Fibronectin leucine rich transmembrane protein 2 | NM_013231 |
| FOS | V-fos FBJ murine osteosarcoma viral oncogene homolog | NM_005252 |
| FOXA1 | Forkhead box A1 | NM_004496 |
| FOXA2 | Forkhead box A2 | NM_021784 |
| FOXM1 | Forkhead box M1 | NM_202002 |
| FOXQ1 | Forkhead box Q1 | NM_033260 |
| FRMD3 | FERM domain containing 3 | BG216229 |
| FZD10 | Frizzled homolog 10 (Drosophila) | NM_007197 |
| G2 | G2 protein | U10991 |
| GAJ | GAJ protein | NM_032117 |
| GAS1 | Growth arrest-specific 1 | NM_002048 |
| GATA6 | GATA binding protein 6 | NM_005257 |
| GDF15 | Growth differentiation factor 15 | NM_004864 |
| GDF3 | Growth differentiation factor 3 | NM_020634 |
| GEM | GTP binding protein overexpressed in skeletal muscle | NM_005261 |
| GPR68 | G protein-coupled receptor 68 | NM_003485 |
| GREM1 | Gremlin 1 homolog, cysteine knot superfamily (Xenopus | NM_013372 |
| | laevis) | |
| GSG1 | Germ cell associated 1 | NM_031289 |
| GTSE1 | G-2 and S-phase expressed 1 | NM_016426 |
| HCAP-G | Chromosome condensation protein G | NM_022346 |
| HLF | Hepatic leukemia factor | NM_002126 |
| HMMR | Hyaluronan-mediated motility receptor (RHAMM) | NM_012484 |
| HRH1 | Histamine receptor H1 | NM_000861 |
| HS6ST2 | Heparan sulfate 6-O-sulfotransferase 2 | NM_147175 |
| HSD11B2 | Hydroxysteroid (11-beta) dehydrogenase 2 | NM_000196 |
| HT008 | Uncharacterized hypothalamus protein HT008 | NM_018469 |
| ID1 | Inhibitor of DNA binding 1, dominant negative helix- | NM_002165 |
| | loop-helix protein | |
| IFI44 | Interferon-induced protein 44 | NM_006417 |
| IL10RA | Interleukin 10 receptor, alpha | NM_001558 |
| IL6 | Interleukin 6 (interferon, beta 2) | NM_000600 |
| INSIG2 | Insulin induced gene 2 | NM_016133 |
| INSIG2 | Insulin induced gene 2 | NM_016133 |
| IRF5 | Interferon regulatory factor 5 | NM_002200 |
| IRX4 | Iroquois homeobox protein 4 | NM_016358 |
| JAG1 | Jagged 1 (Alagille syndrome) | NM_000214 |
| KCNH2 | Potassium voltage-gated channel, subfamily H (eag- | NM_000238 |
| | related), member 2 | |
| KCNK6 | Potassium channel, subfamily K, member 6 | NM_004823 |
| KCNMB4 | Potassium large conductance calcium-activated channel, | NM_014505 |
| | subfamily M, beta member 4 | |
| KIAA0101 | KIAA0101 | NM_014736 |
| KIAA1199 | KIAA1199 | NM_018689 |
| KIAA1217 | KIAA1217 | AK022045 |
| KIAA1509 | KIAA1509 | AB040942 |
| KIAA1666 | KIAA1666 protein | BC035246 |
| KIAA1913 | KIAA1913 | BC044246 |
| KIF20A | Kinesin family member 20A | NM_005733 |
| KIF2C | Kinesin family member 2C | NM_006845 |
| KLF2 | Kruppel-like factor 2 (lung) | NM_016270 |
| KLRC3 | Killer cell lectin-like receptor subfamily C, member 2 | NM_002260 |
| KNSL7 | Kinesin-like 7 | NM_020242 |
| KNTC2 | Kinetochore associated 2 | NM_006101 |
| KRTAP1-5 | Keratin associated protein 1-5 | NM_031957 |
| KRTHB6 | Keratin, hair, basic, 6 (monilethrix) | NM_002284 |
| LAD1 | Ladinin 1 | NM_005558 |
| LAMA2 | Laminin, alpha 2 (merosin, congenital muscular | NM_000426 |
| | dystrophy) | |
| LAPTM5 | Lysosomal associated multispanning membrane protein 5 | NM_006762 |
| LASS5 | LAG1 longevity assurance homolog 5 (S. cerevisiae) | NM_147190 |
| LEF1 | Lymphoid enhancer-binding factor 1 | NM_016269 |
| LGALS2 | Lectin, galactoside-binding, soluble, 2 (galectin 2) | NM_006498 |
| LHX2 | LIM homeobox 2 | NM_004789 |
| LOC120224 | Hypothetical protein BC016153 | NM_138788 |
| LOC150166 | Hypothetical protein LOC150166 | AK056836 |
| LOC150271 | Hypothetical LOC388889 | AK098753 |
| LOC150759 | Hypothetical protein LOC150759 | AK057596 |
| LOC222171 | Hypothetical protein LOC222171 | NM_175887 |
| LOC284018 | Hypothetical protein LOC284018 | NM_181655 |
| LOC285733 | Hypothetical protein LOC285733 | AK091900 |
| LOC286254 | Hypothetical protein LOC286254 | AK092751 |
| LOC338773 | Hypothetical protein LOC338773 | NM_181724 |
| LOC92312 | Hypothetical protein LOC92312 | XM_044166 |
| LOXL3 | Lysyl oxidase-like 3 | NM_032603 |
| LPL | Lipoprotein lipase | NM_000237 |
| LRP12 | Low density lipoprotein-related protein 12 | NM_013437 |
| LRP12 | Low density lipoprotein-related protein 12 | NM_013437 |
| LRP8 | Low density lipoprotein receptor-related protein 8, | NM_033300 |
| | apolipoprotein e receptor | |
| LRRC5 | Leucine rich repeat containing 5 | NM_018103 |
| LTBP2 | Latent transforming growth factor beta binding protein 2 | NM_000428 |
| LYPDC1 | LY6/PLAUR domain containing 1 | NM_144586 |
| MAD2L1 | MAD2 mitotic arrest deficient-like 1 (yeast) | NM_002358 |
| MAFB | V-maf musculoaponeurotic fibrosarcoma oncogene | NM_005461 |
| | homolog B (avian) | |
| MAGEA1 | Melanoma antigen, family A, 1 (directs expression of | NM_004988 |
| | antigen MZ2-E) | |
| MAL2 | Mal, T-cell differentiation protein 2 | NM_052886 |
| MAOB | Monoamine oxidase B | NM_000898 |
| MAP7 | Microtubule-associated protein 7 | NM_003980 |
| MASP1 | Mannan-binding lectin serine protease 1 (C4/C2 | NM_139125 |
| | activating component of Ra-reactive factor) | |
| MCM10 | MCM10 minichromosome maintenance deficient 10 (S. cerevisiae) | NM_182751 |
| MEST | Mesoderm specific transcript homolog (mouse) | NM_002402 |
| MGAT3 | Mannosyl (beta-1,4-)-glycoprotein beta-1,4-N- | AK125361 |
| | acetylglucosaminyltransferase | |
| MGC16121 | Hypothetical protein MGC16121 | BC007360 |
| MGC22265 | Hypothetical protein MGC22265 | BC048193 |
| MGC2574 | Hypothetical protein MGC2574 | NM_024098 |
| MGC2610 | Hypothetical protein MGC2610 | NM_144711 |
| MGC27165 | Hypothetical protein MGC27165 | AF343666 |
| MGC33365 | Hypothetical protein MGC33365 | NM_173552 |
| MK2S4 | Protein kinase substrate MK2S4 | NM_052862 |
| MMP12 | Matrix metalloproteinase 12 (macrophage elastase) | NM_002426 |
| MSX1 | Msh homeo box homolog 1 (Drosophila) | NM_002448 |
| MSX1 | Msh homeo box homolog 1 (Drosophila) | NM_002448 |
| MT1B | Metallothionein 1B (functional) | NM_005947 |
| MT1E | Metallothionein 1E (functional) | NM_175617 |
| MT1G | Metallothionein 1G | NM_005950 |
| MT1H | Metallothionein 1H | NM_005951 |
| MT1H | Metallothionein 1H | NM_005951 |
| MT1K | Metallothionein 1K | NM_176870 |
| MT1L | Metallothionein 1L | X97261 |
| MT1X | Metallothionein 1X | NM_005952 |
| MT1X | Metallothionein 1X | NM_005952 |
| MT2A | Metallothionein 2A | NM_005953 |
| MT2A | Metallothionein 2A | NM_005953 |
| MYB | V-myb myeloblastosis viral oncogene homolog (avian) | NM_005375 |
| MYBL1 | V-myb myeloblastosis viral oncogene homolog (avian)- | X66087 |
| | like 1 | |
| MYLIP | Myosin regulatory light chain interacting protein | NM_013262 |
| MYO10 | Myosin X | NM_012334 |
| MYO1G | Myosin IG | NM_033054 |
| MYO5B | Myosin VB | AK025336 |
| MYO5C | Myosin VC | NM_018728 |
| MYRIP | Myosin VIIA and Rab interacting protein | NM_015460 |
| NAP1L1 | Nucleosome assembly protein 1-like 1 | NM_139207 |
| NAV2 | Neuron navigator 2 | NM_182964 |
| NEK2 | NIMA (never in mitosis gene a)-related kinase 2 | NM_002497 |
| NET1 | Neuroepithelial cell transforming gene 1 | NM_005863 |
| NFE2 | Nuclear factor (erythroid-derived 2), 45 kDa | NM_006163 |
| NFE2L3 | Nuclear factor (erythroid-derived 2)-like 3 | NM_004289 |
| NFIL3 | Nuclear factor, interleukin 3 regulated | NM_005384 |
| NGEF | Neuronal guanine nucleotide exchange factor | NM_019850 |
| NID2 | Nidogen 2 (osteonidogen) | NM_007361 |
| NOSTRIN | Nitric oxide synthase trafficker | NM_052946 |
| NOV | Nephroblastoma overexpressed gene | NM_002514 |
| NPTX1 | Neuronal pentraxin I | NM_002522 |
| NR0B1 | Nuclear receptor subfamily 0, group B, member 1 | NM_000475 |
| NR2F1 | Nuclear receptor subfamily 2, group F, member 1 | NM_005654 |
| NSE1 | NSE1 | NM_145175 |
| NSE2 | Breast cancer membrane protein 101 | NM_174911 |
| NTN4 | Netrin 4 | NM_021229 |
| NUP210 | Nucleoporin 210 kDa | NM_024923 |
| NUSAP1 | Nucleolar and spindle associated protein 1 | NM_016359 |
| ODZ3 | Odz, odd Oz/ten-m homolog 3 (Drosophila) | AB040888 |
| ODZ3 | Odz, odd Oz/ten-m homolog 3 (Drosophila) | AB040888 |
| OIP5 | Opa-interacting protein 5 | NM_007280 |
| OLIG1 | Oligodendrocyte transcription factor 1 | NM_138983 |
| OSAP | Ovary-specific acidic protein | NM_032623 |
| OVOS2 | Ovostatin 2 | BC039117 |
| P2RY8 | Purinergic receptor P2Y, G-protein coupled, 8 | NM_178129 |
| PAPPA | Pregnancy-associated plasma protein A, pappalysin 1 | NM_002581 |
| PAQR4 | Progestin and adipoQ receptor family member IV | NM_152341 |
| PASD1 | PAS domain containing 1 | NM_173493 |
| PBEF1 | Pre-B-cell colony enhancing factor 1 | NM_005746 |
| PBEF1 | Pre-B-cell colony enhancing factor 1 | NM_005746 |
| PBEF1 | Pre-B-cell colony enhancing factor 1 | NM_182790 |
| PCSK5 | Proprotein convertase subtilisin/kexin type 5 | NM_006200 |
| PDGFC | Platelet derived growth factor C | NM_016205 |
| PEPP-2 | PEPP subfamily gene 2 | NM_032498 |
| PHLDA1 | Pleckstrin homology-like domain, family A, member 1 | NM_007350 |
| PIK3R1 | Phosphoinositide-3-kinase, regulatory subunit 1 (p85 | NM_181523 |
| | alpha) | |
| PIM1 | Pim-1 oncogene | NM_002648 |
| PITX2 | Paired-like homeodomain transcription factor 2 | NM_153426 |
| PLAC8 | Placenta-specific 8 | NM_016619 |
| PLAC8 | Placenta-specific 8 | NM_016619 |
| PLD1 | Phospholipase D1, phophatidylcholine-specific | NM_002662 |
| PLK2 | Polo-like kinase 2 (Drosophila) | NM_006622 |
| PLOD2 | Procollagen-lysine, 2-oxoglutarate 5-dioxygenase (lysine | NM_182943 |
| | hydroxylase) 2 | |
| PLP1 | Proteolipid protein 1 (Pelizaeus-Merzbacher disease, | M54927 |
| | spastic paraplegia 2, uncomplicated) | |
| PMAIP1 | Phorbol-12-myristate-13-acetate-induced protein 1 | NM_021127 |
| PON3 | Paraoxonase 3 | NM_000940 |
| POSTN | Periostin, osteoblast specific factor | NM_006475 |
| PPP1R1A | Protein phosphatase 1, regulatory (inhibitor) subunit 1A | NM_006741 |
| PPP1R3B | Protein phosphatase 1, regulatory (inhibitor) subunit 3B | AK091994 |
| PRC1 | Protein regulator of cytokinesis 1 | NM_003981 |
| PREX1 | Phosphatidylinositol 3,4,5-trisphosphate-dependent RAC | NM_020820 |
| | exchanger 1 | |
| PSD3 | Pleckstrin and Sec7 domain containing 3 | NM_015310 |
| PSD3 | Pleckstrin and Sec7 domain containing 3 | NM_015310 |
| PSG1 | Pregnancy specific beta-1-glycoprotein 1 | NM_006905 |
| PSG3 | Pregnancy specific beta-1-glycoprotein 3 | NM_021016 |
| PTGFR | Prostaglandin F receptor (FP) | NM_000959 |
| PTGIR | Prostaglandin I2 (prostacyclin) receptor (IP) | NM_000960 |
| PTTG1 | Pituitary tumor-transforming 1 | NM_004219 |
| PTTG2 | Pituitary tumor-transforming 2 | NM_006607 |
| RACGAP1 | Rac GTPase activating protein 1 | NM_013277 |
| RAMP1 | Receptor (calcitonin) activity modifying protein 1 | NM_005855 |
| RANBP9 | RAN binding protein 9 | NM_005493 |
| RANBP9 | RAN binding protein 9 | NM_005493 |
| RASD1 | RAS, dexamethasone-induced 1 | NM_016084 |
| RASGRP1 | RAS guanyl releasing protein 1 (calcium and DAG- | NM_005739 |
| | regulated) | |
| RGS2 | Regulator of G-protein signalling 2, 24 kDa | NM_002923 |
| RIPK3 | Receptor-interacting serine-threonine kinase 3 | NM_006871 |
| RTN4R | Reticulon 4 receptor | NM_023004 |
| S100B | S100 calcium binding protein, beta (neural) | NM_006272 |
| SAMSN1 | SAM domain, SH3 domain and nuclear localisation | NM_022136 |
| | signals, 1 | |
| SECTM1 | Secreted and transmembrane 1 | NM_003004 |
| SEMA3C | Sema domain, immunoglobulin domain (Ig), short basic | NM_006379 |
| | domain, secreted, (semaphorin) 3C | |
| SEMA3D | Sema domain, immunoglobulin domain (Ig), short basic | NM_152754 |
| | domain, secreted, (semaphorin) 3D | |
| SERPINA5 | Serine (or cysteine) proteinase inhibitor, clade A (alpha- | NM_000624 |
| | 1 antiproteinase, antitrypsin), member 5 | |
| SGOL2 | Shugoshin-like 2 (S. pombe) | NM_152524 |
| SIAT7C | Sialyltransferase 7 ((alpha-N-acetylneuraminyl-2,3-beta- | NM_152996 |
| | galactosyl-1,3)-N-acetyl galactosaminide alpha-2,6- | |
| | sialyltransferase) C | |
| SLC24A3 | Solute carrier family 24 (sodium/potassium/calcium | NM_020689 |
| | exchanger), member 3 | |
| SLC27A2 | Solute carrier family 27 (fatty acid transporter), member 2 | NM_003645 |
| SLC2A1 | Solute carrier family 2 (facilitated glucose transporter), | NM_006516 |
| | member 1 | |
| SLC39A8 | Solute carrier family 39 (zinc transporter), member 8 | NM_022154 |
| SLC40A1 | Solute carrier family 40 (iron-regulated transporter), | NM_014585 |
| | member 1 | |
| SLC7A5 | Solute carrier family 7 (cationic amino acid transporter, | NM_003486 |
| | y+ system), member 5 | |
| SMARCA3 | SWI/SNF related, matrix associated, actin dependent | NM_139048 |
| | regulator of chromatin, subfamily a, member 3 | |
| SMOC1 | SPARC related modular calcium binding 1 | NM_022137 |
| SMOC2 | SPARC related modular calcium binding 2 | NM_022138 |
| SNAI2 | Snail homolog 2 (Drosophila) | NM_003068 |
| SNFT | Jun dimerization protein p21SNFT | NM_018664 |
| SNX10 | Sorting nexin 10 | NM_013322 |
| SOCS1 | Suppressor of cytokine signaling 1 | NM_003745 |
| SOCS3 | Suppressor of cytokine signaling 3 | NM_003955 |
| SOX2 | SRY (sex determining region Y)-box 2 | NM_003106 |
| SOX4 | SRY (sex determining region Y)-box 4 | AW946823 |
| SOX4 | SRY (sex determining region Y)-box 4 | NM_003107 |
| SOX4 | SRY (sex determining region Y)-box 4 | NM_003107 |
| SP5 | Sp5 transcription factor | NM_001003845 |
| SPAG5 | Sperm associated antigen 5 | NM_006461 |
| SPHK1 | Sphingosine kinase 1 | NM_021972 |
| SPINT2 | Serine protease inhibitor, Kunitz type, 2 | NM_021102 |
| SPTA1 | Spectrin, alpha, erythrocytic 1 (elliptocytosis 2) | NM_003126 |
| STAC | SH3 and cysteine rich domain | NM_003149 |
| STC2 | Stanniocalcin 2 | NM_003714 |
| STMN1 | Stathmin 1/oncoprotein 18 | NM_203401 |
| SYTL5 | Synaptotagmin-like 5 | BX647688 |
| T3JAM | TRAF3-interacting Jun N-terminal kinase (JNK)- | NM_025228 |
| | activating modulator | |
| TCEAL7 | Transcription elongation factor A (SII)-like 7 | NM_152278 |
| TFPI2 | Tissue factor pathway inhibitor 2 | NM_006528 |
| THSD2 | Thrombospondin, type I, domain containing 2 | NM_032784 |
| TIMP3 | Tissue inhibitor of metalloproteinase 3 (Sorsby fundus | AA837799 |
| | dystrophy, pseudoinflammatory) | |
| TK1 | Thymidine kinase 1, soluble | NM_003258 |
| TM4SF1 | Transmembrane 4 superfamily member 1 | NM_014220 |
| TMSNB | Thymosin, beta, identified in neuroblastoma cells | NM_021992 |
| TNC | Tenascin C (hexabrachion) | NM_002160 |
| TncRNA | Trophoblast-derived noncoding RNA | U60873 |
| TNFRSF17 | Tumor necrosis factor receptor superfamily, member 17 | NM_001192 |
| TOP2A | Topoisomerase (DNA) II alpha 170 kDa | NM_001067 |
| TOPK | T-LAK cell-originated protein kinase | NM_018492 |
| TPD52 | Tumor protein D52 | NM_005079 |
| TPX2 | TPX2, microtubule-associated protein homolog | NM_012112 |
| | (Xenopus laevis) | |
| TRIB1 | Tribbles homolog 1 (Drosophila) | NM_025195 |
| TRIM45 | Tripartite motif-containing 45 | NM_025188 |
| TROAP | Trophinin associated protein (tastin) | NM_005480 |
| TRPS1 | Trichorhinophalangeal syndrome I | NM_014112 |
| TWIST1 | Twist homolog 1 (acrocephalosyndactyly 3; Saethre- | NM_000474 |
| | Chotzen syndrome) (Drosophila) | |
| TYR | Tyrosinase (oculocutaneous albinism IA) | NM_000372 |
| TYRP1 | Tyrosinase-related protein 1 | NM_000550 |
| UAP1 | UDP-N-acteylglucosamine pyrophosphorylase 1 | NM_003115 |
| UBD | Ubiquitin D | NM_006398 |
| UBE2C | Ubiquitin-conjugating enzyme E2C | NM_181803 |
| UTS2 | Urotensin 2 | NM_021995 |
| UTS2 | Urotensin 2 | NM_021995 |
| VCX3 | Variable charge, X-linked | NM_016379 |
| XK | Kell blood group precursor (McLeod phenotype) | NM_021083 |
| YPEL4 | Yippee-like 4 (Drosophila) | NM_145008 |
| ZBTB20 | Zinc finger and BTB domain containing 20 | BC010934 |
| | | A_23_P170719 |
| | | A_23_P28927 |
| | | A_24_P112542 |
| | | A_24_P195454 |
| | | A_24_P290087 |
| | | A_24_P358131 |
| | | A_24_P927205 |
| | | A_32_P225328 |
| | | A_32_P75141 |
| | | AF256215 |
| | MRNA (fetal brain cDNA g6_1g) | AI791206 |
| | Hypothetical gene supported by AK026189 | AK022865 |
| | Hypothetical gene supported by AK026328 | AK026328 |
| | CDNA: FLJ23131 fis, clone LNG08502 | AK026784 |
| | CDNA FLJ31059 fis, clone HSYRA2000832 | AK055621 |
| | Homo sapiens, clone IMAGE: 4214962, mRNA | AK091547 |
| | | AK098506 |
| | Homo sapiens, clone IMAGE: 4512785, mRNA | AK124558 |
| | CDNA FLJ43172 fis, clone FCBBF3007242 | AK125162 |
| | CDNA FLJ26031 fis, clone PNC08078 | AK129542 |
| | Transcribed locus | AW972815 |
| | | BC005081 |
| | Similar to ankyrin repeat domain 20A | BC016022 |
| | Homo sapiens, clone IMAGE: 3869276, mRNA | BC018597 |
| | Homo sapiens, Similar to hect domain and RLD 2, clone | BC018626 |
| | IMAGE: 4581928, mRNA | |
| | Homo sapiens, clone IMAGE: 3357292, mRNA, partial | BC033117 |
| | cds | |
| | CDNA clone MGC: 65154 IMAGE: 5122136, complete | BC056907 |
| | cds | |
| | MRNA; cDNA DKFZp586O0724 (from clone | BF508144 |
| | DKFZp586O0724) | |
| | Transcribed locus | BQ717518 |
| | Transcribed locus, strongly similar to XP_355557.2 | CD048206 |
| | similar to multi sex combs CG12058-PA [Mus | |
| | musculus] | |
| | Full-length cDNA clone CS0DM001YA20 of Fetal liver | CR601260 |
| | of Homo sapiens (human) | |
| | Full-length cDNA clone CS0DJ001YJ05 of T cells | CR601458 |
| | (Jurkat cell line) Cot 10-normalized of Homo sapiens | |
| | (human) | |
| | Full-length cDNA clone CS0DC002YA18 of | CR624517 |
| | Neuroblastoma Cot 25-normalized of Homo sapiens | |
| | (human) | |
| | | CR936791 |
| | | CX788817 |
| | | ENST00000245185 |
| | | ENST00000261569 |
| | | ENST00000369158 |
| | | ENST00000371256 |
| | | ENST00000371327 |
| | | ENST00000371655 |
| | | ENST00000374541 |
| | | ENST00000375077 |
| | | ENST00000375855 |
| | | ENST00000376155 |
| | | ENST00000381889 |
| | | NM_001006641 |
| | | NM_001009954 |
| | | NM_001010911 |
| | | NM_001010915 |
| | | NM_001012271 |
| | | NM_001017424 |
| | | NM_001017535 |
| | | NM_001017915 |
| | | NM_001017978 |
| | | NM_001018115 |
| | | NM_001031716 |
| | | NM_001040100 |
| | | NM_001040167 |
| | | NM_002263 |
| | | NM_003621 |
| | | NM_012454 |
| | | NM_014867 |
| | | NM_020872 |
| | | NM_020872 |
| | | NM_025135 |
| | | NM_032199 |
| | | NM_032521 |
| | | NR_001564 |
| | | THC2270231 |
| | | THC2281706 |
| | | THC2281732 |
| | | THC2282958 |
| | | THC2309960 |
| | | THC2314600 |
| | | THC2317680 |
| | | THC2343936 |
| | | THC2347909 |
| | | THC2364621 |
| | | THC2373845 |
| | | THC2376729 |
| | | THC2381061 |
| | | THC2407823 |
| | | THC2411757 |
| | | THC2434166 |
| | | THC2438492 |
| | | THC2442210 |
| | | THC2446045 |
| | | W95609 |
| | Similar to hypothetical protein LOC231503 | XM_496707 |
| | | XM_934971 |

1-16. (canceled)

17. A method, comprising:
(a) measuring expression of one or more of the intrinsic genes in Table 5 in a test genetic sample obtained from a subject having or suspected of having scleroderma; and
(b) comparing the expression of the one or more intrinsic genes in the test genetic sample to expression of the one or more intrinsic genes in a control sample, and
(c) classifying the scleroderma in the subject based on the result obtained from (b).

18. The method of claim 17, wherein altered expression of the one or more intrinsic genes in the test genetic sample compared to the expression in the control sample classifies the scleroderma in the subject as Diffuse-Proliferation, Inflammatory, Limited, or Normal-Like subtype.

19. The method of claim 18, wherein increased expression of one or more genes selected from ANP32A, APOH, ATAD2, B3GALT6, B3GAT3, C12orf14, C14orf131, CACNG6, CBLL1, CBX8, CDC7, CDT1, CENPE, CGI-90, CLDN6, CREB3L3, CROC4, DDX3Y, DERP6, DJ971N18.2, EHD2, ESPL1, FGF5, FLJ10902, FLJ12438, FLJ12443, FLJ12484, FLJ12572, FLJ20245, FLJ32009, FLJ35757, FXYD2, GABRA2, GATA2, GK, GSG2, HPS3, IKBKG, IL23A, INSIG1, KIAA1509, KIAA1609, KIAA1666, LDLR, LGALS8, LILRB5, LOC123876, LOC128977, LOC153561, LOC283464, LRRIQ2, LY6K, MAC30, ME2, MGC13186, MGC16044, MGC16075, MGC29784, MGC33839, MGC35212, MGC4293, MICB, MLL5, MTRF1L, MUC20, NICN1, NPTX1, OAS3, OGDHL, OPRK1, PCNT2, PDZK1, PITPNC1, PPFIA4, PREB, PRKY, PSMD11, PSPH, PSPHL, PTP4A3, PXMP2, RAB15, RAD51AP1, RIP, RNF121, RPL41, RPS18, RPS4Y1, RPS4Y2, S100P, SORD, SP1, SYMPK, SYT6, TM9SF4, TMOD3, TNFRSF12A, TPRA40, TRIP, TRPM7, TTR, TUBB4, VARS2L, ZNF572, and ZSCAN2 in the test genetic sample compared to the expression in the control sample classifies the scleroderma as the Diffuse-Proliferation subtype.

20. The method of claim 18, wherein decreased expression of one or more genes selected from AADAC, ADAM17, ADH1A, ADH1C, AHNAK, ALG1, ALG5, AMOT, AOX1, AP2A2, ARK5, ARL6IP5, ARMCX1, BECN1, BECN1, BMP8A, BNIP3L, C10orf119, C1orf24, C1orf37, C20orf10, C20orf22, C5orf14, C6orf64, C9orf61, CAPS, CASP4, CASP5, CAST, CAV2, CCDC6, CCNG2, CDC26, CDK2AP1, CDR1, CFHL1, CNTN3, CPNE5, CRTAP, CTNNA1, CTSC, CUTL1, CXCL5, CYBRD1, CYP2R1, DBN1, DCAMKL1, DCL-1, DIAPH2, DKK2, ECHDC3, ECM2, EIF3S7, EMB, EMCN, EMILIN2, ENPP2, EPB41L2, FBLN1, FBLN2, FEM1A, FGL2, FHL5, FKBP7, FLI1, FLJ10986, FLJ20032, FLJ20701, FLJ23861, FLJ34969, FLJ36748, FLJ36888, FLJ43339, FZR1, GABPB2, GARNL4, GHITM, GHR, GIT2, GLYAT, GPM6B, GTPBP5, HELB, HOXB4, IFNA6, IGFBP5, IL13RA1, IL15, KAZALD1, KCNK4, KCNS3, KCTD10, KIAA0232, KIAA0494, KIAA0562, KIAA0870, KIAA1190, KIF25, KLHL18, KLK2, LAMP2, LEPROTL1, LHFP, LMO2, LOC114990, LOC255458, LOC387680, LOC400027, LOC493869, LOC87769, LRBA, MAFB, MAGEH1, MAN2B2, MCCC2, MEGF10, MFAP5, MGC11308, MGC15523, MGC3200, MGC35048, MGC45780, MOGAT3, MPPE1, MPZ, MYO1B, MYOC, NFYC, NIPSNAP3B, OPTN, OSR2, PAM, PBXIP1, PCOLCE2, PDGFC, PDGFRA, PDGFRL, PEX19, PHAX, PIP, PKM2, PKP2, PMP22, POU2F1, PPAP2B, PRAC, PSMA5, PSORS1C1, PTGIS, RECK, RGS11, RGS5, RIMS3, RIPK2, RNASE4, RNF125, RNF13, RNF146, RNF19, ROBO1, ROBO3, RPL7A, SARA1, SAV1, SCGB1D1, SDK1, SECP43, SECTM1, SERPINB2, SGCA, SH3BGRL, SH3GLB1, SH3RF2, SLC10A3, SLC12A2, SLC14A1, SLC39A14, SLC7A7, SLC9A9, SLPI, SMAD1, SMAP1, SMARCE1, SMP1, SNTG2, SNX7, SOCS5, SSPN, STX7, SUMF1, TAS2R10, TDE2, TFAP2B, TGFBR2, THSD2, TM4SF3, TMEM25, TMEM34, TNA, TNKS2, TRAD, TRAF3IP1, TREM4, TRIM35, TRIM9, TTYH2, TUBB1, UBL3, ULK2, URB, USP54, UST, UTRN, UTX, WIF1, WWOX, XG, YPEL5, and ZFHX1B in the test genetic sample compared to the expression in the control sample classifies the scleroderma as the Diffuse-Proliferation subtype.

21. The method of claim 18, wherein increased expression of one or more genes selected from ANP32A, APOH, ATAD2, B3GALT6, B3GAT3, C12orf14, C14orf131, CACNG6, CBLL1, CBX8, CDC7, CDT1, CENPE, CGI-90, CLDN6, CREB3L3, CROC4, DDX3Y, DERP6, DJ971N18.2, EHD2, ESPL1, FGF5, FLJ10902, FLJ12438, FLJ12443, FLJ12484, FLJ12572, FLJ20245, FLJ32009, FLJ35757, FXYD2, GABRA2, GATA2, GK, GSG2, HPS3, IKBKG, IL23A, INSIG1, KIAA1509, KIAA1609, KIAA1666, LDLR, LGALS8, LILRB5, LOC123876, LOC128977, LOC153561, LOC283464, LRRIQ2, LY6K, MAC30, ME2, MGC13186, MGC16044, MGC16075, MGC29784, MGC33839, MGC35212, MGC4293, MICB, MLL5, MTRF1L, MUC20, NICN1, NPTX1, OAS3, OGDHL, OPRK1, PCNT2, PDZK1, PITPNC1, PPFIA4, PREB, PRKY, PSMD11, PSPH, PSPHL, PTP4A3, PXMP2, RAB15, RAD51AP1, RIP, RNF121, RPL41, RPS18, RPS4Y1, RPS4Y2, S100P, SORD, SP1, SYMPK, SYT6, TM9SF4, TMOD3, TNFRSF12A, TPRA40, TRIP, TRPM7, TTR, TUBB4, VARS2L, ZNF572, and ZSCAN2 in the test genetic sample compared to the expression in the control sample, together with decreased expression of one or more genes selected from AADAC, ADAM17, ADH1A, ADH1C, AHNAK, ALG1, ALG5, AMOT, AOX1, AP2A2, ARK5, ARL6IP5, ARMCX1, BECN1, BECN1, BMP8A, BNIP3L, C10orf119, C1orf24, C1orf37, C20orf10, C20orf22, C5orf14, C6orf64, C9orf61, CAPS, CASP4, CASP5, CAST, CAV2, CCDC6, CCNG2, CDC26, CDK2AP1, CDR1, CFHL1, CNTN3, CPNE5, CRTAP, CTNNA1, CTSC, CUTL1, CXCL5, CYBRD1, CYP2R1, DBN1, DCAMKL1, DCL-1, DIAPH2, DKK2, ECHDC3, ECM2, EIF3S7, EMB, EMCN, EMILIN1, ENPP2, EPB41L2, FBLN1, FBLN2, FEM1A, FGL2, FHL5, FKBP7, FLI1, FLJ10986, FLJ20032, FLJ20701, FLJ23861, FLJ34969, FLJ36748, FLJ36888, FLJ43339, FZR1, GABPB2, GARNL4, GHITM, GHR, GIT2, GLYAT, GPM6B, GTPBP5, HELB, HOXB4, IFNA6, IGFBP5, IL13RA1, IL15, KAZALD1, KCNK4, KCNS3, KCTD10, KIAA0232, KIAA0494, KIAA0562, KIAA0870, KIAA1190, KIF25, KLHL18, KLK2, LAMP2, LEPROTL1, LHFP, LMO2, LOC114990, LOC255458, LOC387680, LOC400027, LOC493869, LOC87769, LRBA, MAFB, MAGEH1, MAN2B2, MCCC2, MEGF10, MFAP5, MGC11308, MGC15523, MGC3200, MGC35048, MGC45780, MOGAT3, MPPE1, MPZ, MYO1B, MYOC, NFYC, NIPSNAP3B, OPTN, OSR2, PAM, PBXIP1, PCOLCE2, PDGFC, PDGFRA, PDGFRL, PEX19, PHAX, PIP, PKM2, PKP2, PMP22, POU2F1, PPAP2B, PRAC, PSMA5, PSORS1C1, PTGIS, RECK, RGS11, RGS5, RIMS3, RIPK2, RNASE4, RNF125, RNF13, RNF146, RNF19, ROBO1, ROBO3, RPL7A, SARA1, SAV1, SCGB1D1, SDK1, SECP43, SECTM1, SERPINB2, SGCA, SH3BGRL, SH3GLB1, SH3RF2, SLC10A3, SLC12A2, SLC14A1, SLC39A14, SLC7A7, SLC9A9, SLPI, SMAD1, SMAP1, SMARCE1, SMP1, SNTG2, SNX7, SOCS5, SSPN, STX7, SUMF1, TAS2R10, TDE2, TFAP2B, TGFBR2, THSD2, TM4SF3, TMEM25, TMEM34, TNA, TNKS2, TRAD, TRAF3IP1, TREM4, TRIM35, TRIM9, TTYH2, TUBB1, UBL3, ULK2, URB, USP54, UST, UTRN, UTX, WIF1, WWOX, XG, YPEL5, and ZFHX1B in the test genetic sample compared to the expression in the control sample, classifies the scleroderma as the Diffuse-Proliferation subtype.

22. The method of claim 18, wherein increased expression of one or more genes selected from A2M, AIF1, ALOX5AP, APOL2, APOL3, BATF, BCL3, BIRC1, BTN3A2, C10orf10, C1orf38, C6orf80, CCL2, CCL4, CCR5, CD8A, CDW52, COL6A3, COTL1, CPA3, CPVL, CTAG1B, DDX58, EBI2, EVI2B, F13A1, FAM20A, FAP, FCGR3A, FLJ11259, FLJ22573, FLJ23221, FLJ25200, FYB, GBP1, GBP3, GEM, GIMAP6, GMFG, GZMH, GZMK, HAVCR2, HCLS1, HLA-DMA, HLA-DOA, HLA-DPA1, HLA-DPB1, HLA-DQA1, HLA-DQA2, HLA-DQB1, HLA-DRB1, HLA-DRB5, ICAM2, IFI16, IFIT1, IFIT2, IFITM1, IFITM2, IFITM3, IL10RA, INDO, ITGB2, KIAA0063, LAMB1, LCP1, LGALS2, LGALS9, LILRB2, LOC387763, LOC400759, LUM, LYZ, MARCKS, MFNG, MGC24133, MPEG1, MRC1, MRCL3, MS4A6A, MX1, NNMT, NUP62, PAG, PLAU, PPIC, PTPRC, RAC2, RGS10, RGS16, RSAFD1, SAT, SCGB2A1, SLC20A1, SLCO2B1, SPARC, SULF1, TAP1, TCTEL1, TIMP1, TNFSF4, UBD, VSIG4, and ZFYVE26 in the test genetic sample compared to the expression in the control sample classifies the scleroderma as the Inflammatory subtype.

23. The method of claim 18, wherein increased expression of one or more genes selected from ATP6V1B2, C1orf42, C7orf19, CKLFSF1, CTAGE4, DICER1, DIRC1, DPCD, DPP3, EMR2, EXOSC6, FLJ90661, FN3KRP, GFAP, GPT, IL27, KCTD15, KIAA0664, LMOD1, LOC147645, LOC400581, LOC441245, MAB21L2, MARCH-II, MGC42157, MRPL43, MT, MT1A, NCKAP1, PGM1, POLD4, RAI16, SAMD10, and UHSKerB in the test genetic sample compared to the expression in the control sample classifies the scleroderma as the Limited subtype.

24. The method of claim 17, wherein the measuring comprises hybridizing the test genetic sample to a nucleic acid microarray that is capable of hybridizing at least one of the genes, and detecting hybridization of at least one of the genes when present in the test genetic sample to the nucleic acid microarray with a scanner suitable for reading the microarray.

25. The method of claim 18, wherein the control sample comprises a composite of data derived from a plurality of nucleic acid microarray hybridizations representative of at least one subtype of scleroderma selected from the group consisting of Diffuse-Proliferation, Inflammatory, Limited, and Normal-Like.

26. The method of claim 25, wherein the control sample comprises a composite of data derived from a plurality of nucleic acid microarray hybridizations representative of each subtype of scleroderma selected from the group consisting of Diffuse-Proliferation, Inflammatory, Limited, and Normal-Like.

27. The method of claim 17, wherein the subject having or suspected of having scleroderma is a subject having scleroderma.

28. The method of claim 17, wherein the subject suspected of having scleroderma is a subject having Raynaud's phenomenon.

29. The method of claim 17, further comprising:
(d) determining the prognosis of the scleroderma in the subject based on the result obtained from (c).

30. The method of claim 18, further comprising:
(d) determining the prognosis of the scleroderma in the subject based on the result obtained from (c).

31. The method of claim 17, further comprising:
determining a treatment plan for the subject based on the result obtained from (c).

32. The method of claim 18, further comprising:
determining a treatment plan for the subject based on the result obtained from (c).
